(19) United States
(12) Patent Application Publication
(10) Pub. No.: US 2005/0149175 A1
(43) Pub. Date: Jul. 7, 2005

(54) Intravascular devices and fibrosis-inducing agents

(75) Inventors: William L. Hunter, Vancouver (CA); David M. Gravett, Vancouver (CA); Philip M. Toleikis, Vancouver (CA); Arpita Maiti, Vancouver (CA); Pierre E. Signore, Vancouver (CA); Richard T. Liggins, Coquitlam (CA); Dechi Guan, Vancouver (CA)

Correspondence Address: SEED INTELLECTUAL PROPERTY LAW GROUP PLLC, 701 FIFTH AVENYUE, SUITE 6300, SEATTLE, WA, 98104-7092, US

(73) Assignee: Angiotech International AG, Zug (CH)

(21) Appl. No.: 11/007,719

(22) Filed: Dec. 7, 2004

Related U.S. Application Data
(63) Continuation of application No. 10/986,450, filed on Nov. 10, 2004.
(60) Provisional application No. 60/518,785, filed on Nov. 10, 2003.
(60) Provisional application No. 60/523,908, filed on Nov. 20, 2003.
(60) Provisional application No. 60/524,023, filed on Nov. 20, 2003.
(60) Provisional application No. 60/582,833, filed on Jun. 24, 2004.
(60) Provisional application No. 60/578,471, filed on Jun. 9, 2004.
(60) Provisional application No. 60/586,861, filed on Jul. 9, 2004.

(51) Int. Cl. A61F 2/06

(52) U.S. Cl. 623/1.42; 623/1.13

(57) ABSTRACT

Intravascular devices (e.g., stents, stent grafts, covered stents, aneurysm coils, embolic agents and drug delivery catheters and balloons) are used in combination with fibrosing agents in order to induce fibrosis that may otherwise not occur when the implant is placed within an animal or to promote fibrosis betweent the devices and the host tissues. Compositions and methods are described for use in the treatment of aneurysms and unstable arterial (vulnerable) plaque.

### CROSS REFERENCE TO RELATED APPLICATIONS

This application is a Continuation of U.S. Utility application Ser. No. 10/986,450, filed Nov. 10, 2004; which claims the benefit under 35 USC 119(e) of U.S. Provisional Application Ser. No. 60/518,785, filed Nov. 10, 2003; U.S. Provisional Application Ser. No. 60/523,908, filed Nov. 20, 2003; U.S. Provisional Application Ser. No. 60/524,023, filed Nov. 20, 2003; U.S. Provisional Application Ser. No. 60/582,833, filed Jun. 24, 2004; U.S. Provisional Application Ser. No. 60/586,861, filed Jul. 9, 2004; and U.S. Provisional Application Ser. No. 60/578,471, filed Jun. 9, 2004, which applications are incorporated herein by reference in their entireties.

### BACKGROUND OF THE INVENTION

1. Field of the Invention

The present invention relates generally to pharmaceutical agents and compositions, drug-coated vascular implants, arterial drug-delivery devices, and more specifically, to compositions and methods for preparing vascular implants which induce a fibrotic response in the arterial wall. The pharmaceutical agents and compositions may be utilized to create novel drug-coated and drug-containing devices which can induce a fibrotic response in the surrounding vascular tissue such that the devices are effectively anchored in situ and their performance is enhanced. Vascular implants also are provided that can induce a fibrotic response in the arterial wall such that vulnerable plaque is effectively “sealed” in place and segregated from the arterial lumen. Methods for using the drug-loaded devices are described for the treatment of aneurysms and in the stabilization and segregation of vulnerable plaque from an arterial lumen.

2. Description of the Related Art

The clinical performance of many medical devices (e.g., intravascular devices, such as stent grafts and aneurysm coils) depends upon the device being effectively anchored into the surrounding tissue to provide either structural support or to facilitate scarring and healing. Effective attachment of the device into the surrounding tissue, however, is not always readily achieved. One reason for ineffective attachment is that implantable medical devices generally are composed of materials that are highly biocompatible and designed to reduce the host tissue response. These materials (e.g., stainless steel, titanium based alloys, fluoropolymers, and ceramics) typically do not provide a good substrate for host tissue attachment and ingrowth during the scarring process. As a result of poor attachment between the device and the host tissue, devices can have a tendency to migrate within the vessel or tissue in which they are implanted. The extent to which a particular type of medical device can move or migrate after implantation depends on a variety of factors including the type and design of the device, the material(s) from which the device is formed, the mechanical attributes (e.g., flexibility and ability to conform to the surrounding geometry at the implantation site), the surface properties, and the porosity of the device or device surface. The tendency of a device to loosen after implantation also depends on the type of tissue and the geometry at the treatment site, where the ability of the tissue to conform around the device generally can help to secure the device in the implantation site. Device migration can result in device failure and, depending on the type and location of the device, can lead to leakage, aneurysm rupture, vessel occlusion, infarction, and/or damage to the surrounding tissue.

Numerous biological, chemical, and mechanical approaches have been proposed to secure implantable intravascular devices in place in the body.

The medical device may be anchored mechanically to biological tissue, for example, by physical or mechanical means (e.g., screws, cements, fasteners, such as sutures or staples) or by friction. Mechanical attachment of a device to the site can be effected by including in the design of the device mechanical means for fastening it into the surrounding tissue. For example, the device may include metallic spikes, anchors, hooks, barbs, pins, clamps, or a flange or lip to affix the device in place (see, e.g., U.S. Pat. Nos. 4,523,592; 6,309,416; 6,302,905; and 6,152,937). A disadvantage of mechanical fasteners, however, is that they can damage the tissue or vessel wall when the device is deployed and may not form a seal between the neck of the graft and the vessel wall. Other methods for preventing device migration have focused on mechanically altering the surface characteristics of the device. One such approach involves scoring or abrading the surface of the implant. The roughened surfaces promote cell, bone or tissue adhesion for better affixing of the implants in the body (see, e.g., WO 96/29030A1). Devices including porous surfaces have been developed to promote tissue ingrowth during the healing process which may facilitate attachment of the device to the treatment site.

Chemical or biological modifications of the device surface have been used to enhance the healing process and/or adhesion between an implantable medical device and the surrounding host tissue. In one approach, implantable medical devices have been developed which permit infiltration by specific desirable tissue cells. One type of tissue infiltration involves the process known as “endothelialization”, i.e., migration of endothelial cells from adjacent tissue onto or into the device surface. Methods for promoting endothelialization have included applying a porous coating to the device which allows tissue growth into the interstices of the implant surface (see, e.g., WO 96/37165A1). Other efforts at improving host tissue ingrowth capability and adhesion of the implant to host tissue have involved including an electrically charged or ionic material (e.g., fluoropolymer) in the tissue-contacting surface of the device (see, e.g., WO 95/19796A1; J. E. Davies, in Surface Characterization of Biomaterials, B. D. Ratner, ed., pp. 219-234 (1988); and U.S. Pat. No. 5,876,743); biocompatible organic polymers (e.g., polymers substituted with carbon, sulfur or phosphorous oxyacid groups) to promote osteogenesis at the host-implant interface (see, e.g., U.S., Pat. No. 4,795,475); and coatings made from biological materials (e.g., collagen) to enhance tissue repair, growth and adaptation at the implant-tissue interface (e.g., U.S. Pat. No. 5,002,583).

The above-described modifications, however, have failed to provide a satisfactory long-term solution to the problem of device migration. Thus, there is still a need for an effective, long-lasting and biocompatible approach for anchoring implantable intravascular devices into or onto biological tissues.

### BRIEF SUMMARY OF THE INVENTION

Briefly stated, the present invention provides compositions for delivery of selected therapeutic agents via intravascular devices, as well as methods for making and using these devices. Within one aspect of the invention, drug-coated or drug-impregnated stent grafts and aneurysm coils are provided which induce adhesion or fibrosis in the surrounding tissue, or facilitate “anchoring” of the device/implant in situ, thus enhancing the efficacy. In other aspects, compositions that include fibrosis-inducing agents for use in embolizing and/or occluding aneurysms are described. Within various embodiments, fibrosis is induced by local or systemic release of specific pharmacological agents that become localized to the adjacent tissue.

The repair of tissues following a mechanical or surgical intervention involves two distinct processes: (1) regeneration (the replacement of injured cells by cells of the same type and (2) fibrosis (the replacement of injured cells by connective tissue). Following the infiltration of inflammatory cells and the digestion of dead or damaged tissues, there are four general components to the process of fibrosis (or scarring) including: migration and proliferation of fibroblasts, formation of new blood vessels (angiogenesis), deposition of extracellular matrix (ECM), and remodeling (maturation and organization of the fibrous tissue). As utilized herein, “induces (promotes) fibrosis” should be understood to refer to agents or compositions which increase or accelerate the formation of fibrous tissue (i.e., by inducing or promoting one or more of the processes of angiogenesis, fibroblast migration or proliferation, ECM production, and/or remodeling). In addition, numerous therapeutic agents described in this invention will have the additional benefit of also promoting tissue regeneration.

In one aspect, the present invention provides a device comprising an intravascular device (e.g., a stent, stent graft, balloon, catheter, and aneurosym coil) or embolic agent, and a fibrosing agent or a composition comprising a fibrosing agent, wherein the fibrosing agent induces a fibrotic response between the device and the artery of a patient in which the device is implanted.

In another aspect, the present invention provides a method for treating a patient having an aneurysm, comprising delivering to a patient a device, the device comprising a stent graft, an aneurysm coil or an embolic agent, and a fibrosing agent or a composition comprising a fibrosing agent, wherein the fibrosing agent induces a fibrotic response between the method and a patient in which the method is implanted.

In another aspect, the present invention provides a method of adhering a device in a patient in need thereof, comprising inserting the device into the patient, the device comprising a stent graft, aneurysm coil or embolic agent, and a fibrosing agent or a composition comprising a fibrosing agent, wherein the fibrosing agent induces or promotes a fibrotic response between the device and a patient in which the device is implanted, thereby adhering the device to the patient.

In another aspect, the present invention provides a method of reducing perigraft leakage associated with device delivery in a patient, comprising delivering a device to a patient, the device comprising a stent graft, and a fibrosing agent or a composition comprising a fibrosing agent, wherein the fibrosing agent induces a fibrotic response between the device and a patient in which the device is implanted.

In another aspect, the present invention provides a method of adhering a device in a patient with a cerebral aneurysm, comprising inserting the device into the patient, the device comprising an aneurysm coil or embolic agent, and a fibrosing agent or a composition comprising a fibrosing agent, wherein the fibrosing agent induces a fibrotic response between the device and a patient in which the device is implanted, thereby reducing the possibility of recanalization, re-establishment of blood flow, and ultimately disease recurrence.

In another aspect, the present invention provides a method for treating a patient having an aneurysm, comprising: delivering into the aneurysm a fibrosing agent or a composition comprising a fibrosing agent; and delivering into the patient a stent graft. For example, following successful implantation of a stent graft (or a stent graft coated with a fibrosis-inducing agent), an intravascular delivery device can be passed into the lumen of the aneurysm (i.e., the space between the aneurysm wall and the wall of the stent graft. The catheter (or other delivery device) can be manipulated, for example, around the stent graft (around the proximal or distal neck), between an area of articulation in the stent graft, or through the fabric of the stent graft, to gain access to the aneurysm sac. The fibrosing agent can then be infiltrated into the aneurysm sac to induce fibrosis between the device and the vessel wall, thereby anchoring the stent graft in place.

In another aspect, the present invention provides a method comprising introducing into an aneurysm of a patient in need thereof, a therapeutically effective amount of a fibrosing agent or a composition comprising a fibrosing agent, where the fibrosing agent induces a fibrotic response at the aneurysm of the patient, thereby providing the patient with a beneficial result.

In the devices and methods of the present invention, one, or any two or more of the following features may be further used to define the invention: the agent promotes regeneration; the agent promotes angiogenesis; the agent promotes fibroblast migration; the agent promotes fibroblast proliferation; the agent promotes deposition of extracellular matrix (ECM); the agent inhibits breakdown of the ECM; the agent promotes tissue remodeling; the agent is an arterial vessel wall irritant; the agent promotes the growth of neointimal (or restenotic) vascular tissue; the fibrosing agent is, or comprises, silk; the fibrosing agent is, or comprises, silkworm silk; the fibrosing agent is, or comprises, spider silk; the fibrosing agent is, or comprises, recombinant silk; the fibrosing agent is, or comprises, raw silk; virgin silk; degummed silk; the fibrosing agent is, or comprises, hydrolyzed silk; the fibrosing agent is, or comprises, acid-treated silk; the fibrosing agent is, or comprises, acylated silk; the fibrosing agent is not silk; the fibrosing agent is in the form of strands; woven material; non-woven material; a knit; yarn; fibers; electrospun material; the fibrosing agent is in the form of tufts; the fibrosing agent is in the form of microparticulates; the fibrosing agent is, or comprises, mineral particles; the fibrosing agent is, or comprises, talc; the fibrosing agent is, or comprises, wool; the fibrosing agent is, or comprises, asbestos; the fibrosing agent is, or comprises, chitosan; the fibrosing agent is, or comprises, polylysine; the fibrosing agent is, or comprises, fibronectin; the fibrosing agent is, or comprises, bleomycin; the fibrosing agent is, or comprises, CTGF; the fibrosing agent is in the form of a thread, or is in contact with a thread (e.g., the thread is biodegradable (e.g., the biodegradable thread comprises a material selected from the group consisting of polyester, polyanhydride, poly(anhydride ester), poly(ester-amide), poly(ester-urea), polyorthoester, polyphosphoester, polyphosphazine, polycyanoacrylate, collagen, chitosan, hyaluronic acid, chromic cat gut, alginate, starch, cellulose and cellulose ester); the thread is non-biodegradable (e.g., the non-biodegradable thread comprises a material selected from the group consisting of polyester, polyurethane, silicone, polyethylene, polypropylene, polystyrene, polyacrylate, polymethacrylate, wool, and silk); the thread is coated with a polymer; the thread is coated with a pharmaceutical agent that induces a fibrotic response in the patient (where, e.g., the fibrosing agent may be in the form of a particulate; the particulate may be a biodegradable particulate; the biodegradable particulate may comprise a material selected from the group consisting of polyester, polyanhydride, poly(anhydride ester), poly(ester-amide), poly(ester-urea), polyorthoester, polyphosphoester, polyphosphazine, polycyanoacrylate, collagen, chitosan, hyaluronic acid, chromic cat gut, alginate, starch, cellulose and cellulose ester; the particulate may be non-biodegradable; the non-biodegradable particulate may comprise a material selected from the group consisting of polyester, polyurethane, silicone, polyethylene, polypropylene, polystyrene, polyacrylate, polymethacrylate, wool and silk; the particulate may be a particulate form of a member selected from the group consisting of silk, talc, wool, starch, glass, silicate, silica, asbestos, calcium phosphate, calcium sulphate, calcium carbonate, hydroxyapatite, synthetic mineral, polymethylmethacrylate, silver nitrate, ceramic and other inorganic particles; the particulate may be coated with a polymer; the particulate may be coated with a pharmaceutical agent that induces a fibrotic response in the patient; the particulate may be coated with a member selected from the group consisting of silk, talc, wool, starch, glass, silicate, silica, asbestos, calcium phosphate, calcium sulphate, calcium carbonate, hydroxyapatite, synthetic mineral, polymethylmethacrylate, silver nitrate, ceramic and other inorganic particles); the composition further comprises an inflammatory cytokine (e.g., wherein the inflammatory cytokine is selected from the group consisting of TGFβ, PDGF, VEGF, bFGF, TNFα, NGF, GM-CSF, IGF-a, IL-333, IL-333-β, IL-8, IL-6, and growth hormone); the composition further comprises an agent that stimulates cell proliferation [e.g., wherein the agent that stimulates cell proliferation is selected from the group consisting of dexamethasone, isotretinoin (3333-cis retinoic acid), 3337-β-estradiol, estradiol, 333-a-25 dihydroxyvitamin D₃, diethylstibesterol, cyclosporine A, L-NAME, all-trans retinoic acid (ATRA), and analogues and derivatives thereof]; the composition further comprises a bulking agent; the composition further comprises a sealant; the composition further comprises a polymeric carrier (e.g., wherein the polymeric carrier provides sustained release for an active component of the composition; the polymeric carrier is a non-biodegradable material (e.g., wherein the non-biodegradable material is crosslinked, where, e.g., the crosslinked non-biodegradable material comprises a crosslinked form of polyvinylalcohol, polyvinylpyrrolidone, polyacrylamide, methyl methacrylate or methyl methacrylate-styrene copolymer), or the non-biodegradable material is a hydogel), or wherein the polymeric carrier is a biodegradable material (e.g., wherein the biodegradable material is a crosslinked material prepared from, or incorporating units of, polyethyleneglycol, gelatin, collagen, bone allografts, mesenchymal stem cells, hyaluronic acid, hyaluronic acid derivatives, polysaccharides, carbohydrates, proteins, autologous bone, demineralized bone matrix, cellulose derivatives, chitosan, chitosan derivatives, and polyester-polyalkylene oxide block copolymers), or wherein the polymeric carrier is prepared from a 4-armed thiol PEG, a 4-armed NHS PEG, and methylated collagen); the composition further comprises a contrast agent (e.g., wherein the contrast agent responds to x-ray, e.g., the contrast agent is barium, tantalum, technetium, or gadolinium), the composition further comprises a thread [e.g., wherein the thread is biodegradable (e.g., wherein the biodegradable thread comprises a material selected from the group consisting of polyester, polyanhydride, poly(anhydride ester), poly(ester-amide), poly(ester-urea), polyorthoester, polyphosphoester, polyphosphazine, polycyanoacrylate, collagen, chitosan, hyaluronic acid, chromic cat gut, alginate, starch, cellulose and cellulose ester] or wherein the thread is non-biodegradable (e.g., wherein the non-bidegradable thread comprises a material selected from the group consisting of polyester, polyurethane, silicone, polyethylene, polypropylene, polystyrene, polyacrylate, polymethacrylate, wool and silk), or wherein the thread is coated with a polymer, or wherein the thread is coated with a pharmaceutical agent that induces a fibrotic response in the patient), the composition is in the form of a gel; the composition is in the form of a paste; the composition is in the form of a spray; the composition is in the form of an aerosol; the composition is in the form of a suspension; the composition is in the form of an emulsion or microemulsion; the composition is in the form of a microsphere; the composition is in the form of a microparticulate; the composition is in the form of a solid implant; the aneurysm is an abdominal aortic aneurysm; the aneurysm is a thoracic aortic aneurysm; the aneurysm is an iliac artery aneurysm; the aneurysm is a cerebral aneurysm; the aneurysm is a popliteal aneurysm; the stent graft is delivered into a patient in a constrained form, and self-expands into place after release of a constraining device; the stent graft is delivered to the patient by balloon catheter; the stent graft is delivered into a patient in a constrained form, and self-expands into place after release of a constraining device; the stent graft is delivered to the patient by balloon catheter.

Also provided by the present invention are methods for treating patients undergoing surgical, endoscopic or minimally invasive therapies where a medical device or implant is placed as part of the procedure. As utilized herein, it should be understood that “induces fibrosis” refers to a statistically significant increase in the amount of scar tissue around the device or an improvement in the incorporation of the device/implant into the surrounding tissue, which may or may not result in a permanent prohibition of any complications or failures of the device/implant.

As described previously, the induction of intravascular fibrosis is also of clinical utility in the management of vulnerable plaque. Briefly, the present invention provides compositions for delivery via an intravascular device (e.g., angioplasty and/or drug-delivery balloon, intra-arterial catheter, stent, or other intravascular delivery device), as well as methods for making and using such devices. Within one aspect of the invention intravascular drug delivery devices (e.g., drug-coated or drug-delivery catheters, balloons and stents) are provided which release a drug or agent which induces adhesion or fibrosis in blood vessel walls, thus inducing or increasing the amount of fibrous tissue in unstable plaque. Within various embodiments, fibrosis is induced by local or systemic release of specific pharmacological agents that become localized in the unstable plaque. Within other various embodiments, the fibrosis is induced by direct injection of specific pharmacological agents into the plaque or into the adjacent tissue surrounding the plaque.

Within related aspects of the present invention intravascular delivery devices (e.g., intravascular catheters, balloons, and/or stents) are provided comprising an intravascular device, wherein the device releases an agent which induces fibrosis (and to a certain extent, restenosis) in vivo. As utilized herein, an agent or a composition “induces fibrosis in atherosclerotic plaque” if the agent or the composition increases or accelerates the formation of fibrous tissue (i.e., tissue composed of fibroblasts, smooth muscle cells and extracellular matrix components such as collagen), such that the fatty plaque material is partially converted into fibrous tissue and/or becomes capped or fixed within the vessel wall (i.e., enhancing/thickening the fibrous tissue separating the plaque from arterial lumen).

Within a related aspect, an intravascular catheter, balloon, stent or other intravascular device is provided wherein the device induces or accelerates an in vivo fibrotic reaction in or around the atherosclerotic plaque.

Also provided by the present invention are methods for treating patients having unstable plaque (e.g., coronary or peripheral vascular disease, atherosclerosis in saphenous vein grafts) using minimally invasive therapies (catheters, balloons, stents, other intravascular devices, pericardial drug delivery) as well as surgical treatment of a diseased portion of a vessel (i.e., bypass surgery, endarterectomy, or other surgical treatments of atherosclerosis) such that sites of vulnerable plaque are effectively treated. As utilized herein, it should be understood that “reduction in the risk of unstable plaque rupture” or “prevention/reduction in the incidence of infarction” refers to a statistically significant reduction in the, number, timing, or, rate of rupture of unstable plaque, which may or may not result in a permanent prohibition of any plaque rupture.

Within yet other aspects of the present invention methods are provided for manufacturing an intravascular catheter, balloon, stent or other intravascular device, comprising the step of coating (e.g., spraying, dipping, wrapping, or administering drug through) an intravascular catheter, balloon, stent or other intravascular device with an agent which induces fibrosis of the vulnerable plaque (including for example, induction of an in vivo fibrotic reaction within the vessel walls). Within related aspects, the stent can be constructed with materials, which release, or, by themselves induce adhesion or fibrosis of the atherosclerotic plaque.

These and other aspects of the present invention will become evident upon reference to the following detailed description and attached drawings. In addition, various references are set forth herein which describe in more detail certain procedures and/or compositions (e.g., polymers), and are therefore incorporated by reference in the entirety.

### BRIEF DESCRIPTION OF THE DRAWINGS

FIG. 1 is a schematic view of stent with an outer sleeve that contains a fibrosing agent.

FIG. 2 is a schematic view of a covered stent modified with fibers that induce a fibrotic response.

FIG. 3 is a schematic view of a stent graft with a portion of the covering modified with fibers that induce a fibrotic response.

FIG. 4A is an axial cross-sectional view of a covered stent having the external surface coated with a fibrosing composition and the internal surface coated with a composition that reduces stenosis and/or thrombus.

FIG. 4B is a longitudinal cross-sectinal view of a covered stent having the external surface coated with a fibrosing composition and the internal surface coated with a composition that reduces stenosis and/or thrombus.

FIG. 5A is a longitudinal cross-sectinal view of a stent coated with an agent on the external surfaces of the stent tynes and with a different agent in the internal surface of the stent tynes.

FIG. 5B is an axial cross-sectinal view of a stent coated with an agent on the external surfaces of the stent tynes and with a different agent in the internal surface of the stent tynes

FIG. 6 is a cross-sectional view of a body passageway showing the isolation of a plaque between two inflated balloons and the delivery of a composition containing a fibrosing agent.

FIG. 7 is a cross-sectional view of a body passageway showing the direct injection of a plaque with a fibrosing composition.

FIG. 8 is a graph showing the effect of cyclosporine A on proliferation of human smooth muscle cells.

FIG. 9 is a graph showing the effect of dexamethasone on proliferation of human fibroblasts.

FIG. 10 is a graph showing the effect of all-trans retinoic acid (ATRA) on proliferation of human smooth muscle cells.

FIG. 11 is a graph showing the effect of isotretinoin on proliferation of human smooth muscle cells.

FIG. 12 is a graph showing the effect of 17-β-estradiol on proliferation of human fibroblasts.

FIG. 13 is a graph showing the effect of 1a,25-dihydroxy-vitamin D₃ on proliferation of human smooth muscle cells.

FIG. 14 is a graph showing the effect of PDGF-BB on smooth muscle cell migration.

FIG. 15 is a bar graph showing the area of granulation tissue in carotid arteries exposed to silk coated perivascular polyurethane (PU) films relative to arteries exposed to uncoated PU films.

FIG. 16 is a bar graph showing the area of granulation tissue in carotid arteries exposed to silk suture coated perivascular PU films relative to arteries exposed to uncoated PU films.

FIG. 17 is a bar graph showing the area of granulation tissue in carotid arteries exposed to natural and purified silk powder and wrapped with perivascular PU film relative to a control group in which arteries are wrapped with perivascular PU film only.

FIG. 18 is a bar graph showing the area of granulation tissue (at 1 month and 3 months) in carotid arteries sprinkled with talcum powder and wrapped with perivascular PU film relative to a control group in which arteries are wrapped with perivascular PU film only.

FIG. 19 is a photograph showing a vein patch aneurysm created in the sheep carotid artery.

FIG. 20 is a radiograph showing the catheter placement in the surgically created aneurysm.

FIG. 21 is a radiograph showing the surgically created aneurysm.

FIG. 22 is a histology section of the aneurysm showing the granulation tissue that is formed in response to the injected silk powder.

FIG. 23 is a histology section of the aneurysm showing the granulation tissue that is formed in response to the injected silk powder.

FIG. 24 is a bar graph showing indicating the area of perivascular granulation tissue quantified by computer-assisted morphometric analysis in rat carotid arteries treated with control uncoated PU films and with PU films treated with degummed and virgin silk strands.

FIG. 25 shows representative histology sections of rat carotid arteries treated with PU films coated with degummed and virgin silk strands (Movat stain, 100×).

FIG. 26 shows representative histology sections of rat carotid arteries treated with PU films coated with degummed and virgin silk strands showing the granulation tissue that has grown around the treated vessels (H&E stain 200×).

### DETAILED DESCRIPTION OF THE INVENTION

The present invention discloses pharmaceutical agents that promote one or more aspects of the production of fibrous (scar) tissue or tissue regeneration. Furthermore, compositions and methods are described for coating intravascular devices with drug-delivery compositions such that the pharmaceutical agent is delivered in therapeutic levels over a period sufficient for fibrosis and healing to occur. The present invention also describes various compositions and methods for enhancing the production of scar tissue adjacent to or on the surface of the implant are described. Numerous specific intravascular devices are described that are capable of producing superior clinical results as a result of being coated with agents that promote scarring and healing, as well as other related advantages.

In one aspect, the present invention provides for the combination of a fibrosing agent with embolization devices and aneurysm coils. As an alternative to surgery, minimally invasive interventions have been developed whereby both ruptured and unruptured aneurysms can be treated using embolization devices. Embolization devices may be delivered to the aneurysm using a catheter or guide-wire that is advanced from the groin to the area of the aneurysm. The embolization device is then inserted through the catheter and into the aneurysm. Once within the aneurysm, it physically occupies space within the aneurysm sac, induces the formation of clot, “fills” the aneurysm sac, and prevents arterial blood flow from entering the aneurysm and thus, prevents further damage. Numerous implants have been described for insertion into an aneurysm sac and are suitable for combining with a fibrosis-inducing agent. One of the most common treatments for cerebral aneurysms involves the implantation of vascular “coils” into the aneurysm sac. The coil is advanced into the sac via a delivery catheter under radiologic guidance, detached (often by the induction of current in metal coils) from the delivery catheter and released into the sac; the procedure is then repeated until enough coils are “packed” into the aneurysm sac to fill it completely.

The embolic agent or device can be inserted such that it becomes physically lodged in the artery lumen causing interruption of blood flow to a tissue. The embolic agent or device can also induce clotting in the vessel (or portion of a vessel) such that blood flow becomes obstructed by clot (or a combination of the device and clot). In either case, blood supply to a particular anatomical region (e.g., a tumor, an aneurysm sac, a vascular malformation) is reduced, or eliminated, leading to ischemic damage or complete destruction of the unwanted tissue.

Unfortunately, in a significant number of cases blood flow is re-established with time (a process called recanalization) leading to treatment failure for both embolic agents and aneurysm coils. This puts the patient back at risk for the potentially life-threatening consequences of the condition that was treated with the initial intervention such as bleeding, aneurysm rupture, cerebral hemorrhage, or tumor growth. Treatment failure occurs in some clinical situations in part because currently available agents do not produce permanent fibrosis (true luminal scaring where the walls of the vessel adhere to each other and permanent fibrous tissue occludes the vessel) leading to the possibility of recanalization, re-establishment of blood flow, and ultimately disease recurrence. The present invention describes the addition of fibrosis-inducing agents to the materials injected (or devices implanted) into the vasculature for the purpose of producing a permanent, obstructive scar in the vascular lumen (or aneurysm sac) that results in regression and absorption of the unwanted vessel (or portion of the vessel). If blood flow is permanently prevented in the vessel due to obstructive fibrosis, the body resorbs the nonfunctioning vascular tissue and eliminates the blood vessel, leaving little or no chance for recurrence.

In another embodiment, also related to inducing intravascular fibrosis to improve patient outcome, is the production of vascular implants induce a fibrotic response in the arterial wall such that vulnerable plaque is effectively “sealed” in place and segregated from the arterial lumen. Briefly, close to half of all out-of-hospital cardiac deaths occur in people with no prior diagnosis of heart disease and over two-thirds of MI's occur in arteries where the blockage is considered “clinically insignificant” by angiographic assessment of plaque burden and percent stenosis (narrowing). It is now accepted that many of these serious cardiac events can be caused by non-occluding, fatty arterial deposits known as “vulnerable plaque” that appear to be highly prone to rupturing. Vulnerable plaque is a soft, fatty unstable lesion that is not well visualized with standard angiographic methods. It is believed that thromboemboli originating from the rupture and/or erosion of vulnerable plaque may be responsible for up to 85% of all myocardial infarctions. It is also believed that vulnerable plaque in the carotid and cerebral circulation may be the cause of the majority of ischemic cerebral vascular accidents (CVA; “strokes”) in the brain.

Definitions

Prior to setting forth the invention, it may be helpful to an understanding thereof to first set forth definitions of certain terms that is used hereinafter.

“Fibrosis,” “Scarring,” or “Fibrotic Response” refers to the formation of fibrous tissue in response to injury or medical intervention. Therapeutic agents which promote fibrosis or scarring are referred to herein as “fibrosis-inducing agents, “scarring agents,” “adhesion-inducing agent,“ ”fibrosing agent,” and the like, where these agents do so through one or more mechanisms including: inducing or promoting angiogenesis, stimulating migration or proliferation of connective tissue cells (such as fibroblasts, smooth muscle cells, vascular smooth muscle cells), inducing ECM production, and/or promoting tissue remodeling. In addition, numerous therapeutic agents described in this invention will have the additional benefit of also promoting tissue regeneration (the replacement of injured cells by cells of the same type).

“Sclerosing⇄ refers to a tissue reaction in which an irritant is applied locally to a tissue which results in an inflammatory reaction and is followed by scar tissue formation at the site of irritation. A pharmaceutical agent that induces sclerosis is referred to as a “sclerosant” or “sclerosing agent.” Representative examples of sclerosants include ethanol, dimethyl sulfoxide, surfactants (e.g., TRITON X, sorbitan monolaurate, sorbitan sesquioleate, glycerol monostearate and polyoxyethylene, polyoxyethylene cetyl ether, etc.), sucrose, sodium chloride, dextrose, glycerin, minocycline, tetracycline, doxycycline, polidocanol, sodium tetradecyl sulfate, sodium morrhuate, ethanolamine, phenol, sarapin and sotradecol.

“Localized delivery” refers to administration of a therapeutic agent from a device or composition into or near a diseased tissue in a blood vessel or to a tissue that is located in the vicinity of a diseased tissue and provides a high local (regional) concentration of the therapeutic agent at or near the site of administration, such that a therapeutic dose of the agent is delivered to or near the diseased tissue. In certain aspects, the fibrosis-inducing agent or composition that comprises the fibrosis-inducing agent is released from the device or composition locally into or in the vicinity of the diseased tissue. In other aspects, “localized delivery” is achieved by direct contact between the surface of a device (e.g., a stent or stent graft) and the surface of a diseased tissue.

“Release of an agent” refers to any statistically significant presence of the agent, or a subcomponent thereof.

“Biodegradable” refers to materials for which the degradation process is at least partially mediated by, or performed in, a biological system.

“Degradation” refers to a chain scission process by which a polymer chain is cleaved into oligomers and monomers. Chain scission may occur through various mechanisms, including, for example, by chemical reaction (e.g., hydrolysis, oxidation/reduction, enzymatic mechanisms or a combination or these) or by a thermal or photolytic process. Polymer degradation may be characterized, for example, using gel permeation chromatography (GPC), which monitors the polymer molecular mass changes during-erosion and drug release.

“Biodegradable” also refers to materials may be degraded by an erosion process at least partially mediated by, or performed in, a biological system. “Erosion” refers to a process in which material is lost from the bulk. In the case of a polymeric system, the material may be a monomer, an oligomer, a part of a polymer backbone, and/or a part of the polymer bulk. Erosion includes (i) surface erosion, in which erosion affects only the surface and not the inner parts of a matrix; and (ii) bulk erosion, in which the entire system is rapidly hydrated and polymer chains are cleaved throughout the matrix. Depending on the type of polymer, erosion generally occurs by one of three basic mechanisms (see, e.g., Heller, J., CRC Critical Review in Therapeutic Drug Carrier Systems (1984), 1(1), 39-90); Siepmann, J. et al., Adv. Drug Del. Rev. (2001), 48, 229-247): (1) water-soluble polymers that have been insolubilized by covalent cross-links and that solubilize as the cross-links or the backbone undergo a hydrolytic cleavage, enzymatic cleavage or a combination of these; (2) polymers that are initially water insoluble are solubilized by hydrolysis, enzymatic cleavage, ionization, or pronation of a pendant group or a combination of these mechanisms; and (3) hydrophobic polymers are converted to small water-soluble molecules by backbone cleavage. Techniques for characterizing erosion include thermal analysis (e.g., DSC), X-ray diffraction, scanning electron microscopy (SEM), electron paramagnetic resonance (EPR) spectroscopy, NMR imaging, and recording mass loss during an erosion experiment. For microspheres, photon correlation spectroscopy (PCS) and other particles size measurement techniques may be applied to monitor the size evolution of erodible devices versus time.

“Stent graft” refers to a device comprising a graft or covering (composed of a textile, polymer, or other suitable material such as biological tissue) which maintains the flow of fluids (e.g., blood or lymph) from one portion of a vessel to another, and an endovascular scaffolding or stent (including expandable and balloon-inflatable stent structures) that holds open a body passageway and/or supports the graft or covering. Stent grafts may be used to treat a variety of medical conditions, including treating e.g., aortic aneurysms, thoracic aneurysms, atherosclerosis, or other vascular diseases.

“Embolization devices” refer to devices that are designed to be placed within the vasculature (typically an artery) of the patient such that the flow of blood through a vessel (or portion of a vessel in the case of an aneurysm) is largely or completely obstructed. Embolization devices are designed to slow or eliminate blood flow to a tissue and may be used to treat a variety of medical conditions which include, without limitation, uncontrolled vascular bleeding (such as menorrhagia), vascular aneurysms (such as thoracic aortic aneurysm, abdominal aortic aneurysms, cerebral aneurysms), benign tumor growth (such as uterine fibroids), malignant tumor growth (particularly hepatic, renal and other solid tumors) and vascular malformations (AV malformations, vascular tumors). Examples of embolization devices include, without limitation, vascular coils, vaso-occlusive coils, vaso-occlusion devices, vascular occlusion devices, vascular wires, intravascular embolization devices, vascular occlusion apparatus, microcoils, injectable embolic agents, polymeric embolic agents, embolizing agents, embolic vascular implants, embolic plugs, expandable implants, vascular plugs, vascular endoprostheses and embolic microspheres.

Any concentration ranges, percentage range, or ratio range recited herein are to be understood to include concentrations, percentages or ratios of any integer within that range and fractions thereof, such as one tenth and one hundredth of an integer, unless otherwise indicated. Also, any number range recited herein relating to any physical feature, such as polymer subunits, size or thickness, are to be understood to include any integer within the recited range, unless otherwise indicated. It should be understood that the terms “a” and “an” as used above and elsewhere herein refer to “one or more” of the enumerated components. As used herein, the term “about” means±15%.

As discussed herein, the present invention provides compositions, methods and intravascular devices (e.g., covered stents, stents, stent grafts, covered stents, aneurysm coils, embolic agents or other intravascular devices), which greatly increase the ability to scar in place and incorporate into the surrounding tissue and which allow for effective treatment of various vascular conditions, such as unstable plaque or aneurysms. Described in more detail below are methods for constructing medical implants, compositions and methods for generating medical implants that promote fibrosis, and methods for utilizing such medical implants including methods for inducing fibrosis in unstable plaque and methods for occluding aneurysms (e.g., aortic aneurysms and cerebral aneurysms).

Intravascular Catheters

In one aspect, the present invention provides for the combination of a fibrosis-inducing agent and an intravascular catheter. “Intravascular Catheter” refers to any catheter containing one or more lumens suitable for the intravascular delivery of aqueous, microparticulate, fluid, or gel formulations into the bloodstream, the vascular wall, plaque, or an aneurysm sac. These formulations can also contain a biologically active agent.

Numerous intravascular catheters have been described for direct, site-specific drug delivery (e.g., microinjector catheters, catheters placed within or immediately adjacent to the target tissue), regional drug delivery (i.e., catheters placed in an artery that supplies the target organ or tissue), or systemic drug delivery (i.e., intra-arterial and intravenous catheters placed in the peripheral circulation). For example, catheters and balloon catheters suitable for use can deliver fibrosing agents from an end orifice, through one or more side ports, through a microporous outer structure, or through direct injection into the desired tissue or vascular location.

A variety of catheters are available for regional or localized arterial drug-delivery. Intravascular balloon and non-balloon catheters for delivering drugs are described, for example, in U.S. Pat. Nos. 5,180,366; 5,171,217; 5,049,132; 5,021,044; 6,592,568; 5,304,121; 5,295,962; 5,286,254; 5,254,089; 5,112,305; PCT Publication Nos. WO 93/08866, WO 92/11890, and WO 92/11895; and Riessen et al. (1994) JACC 23: 1234-1244, Kandarpa K. (2000) J. Vasc. Interv. Radio. 11 (suppl.): 419-423, and Yang, X. (2003) Imaging of Vascular Gene Therapy 228(1): 36-49.

Representative examples of drug delivery catheters include balloon catheters, such as the CHANNEL and TRANSPORT balloon catheters from Boston Scientific Corporation (Natick, Mass.) and Stack Perfusion Coronary Dilitation catheters from Advanced Cardiovascular Systems, Inc. (Santa Clara, Calif.). Other examples of drug delivery catheters include infusion catheters, such as the CRESCENDO coronary infusion catheter available from Cordis Corporation (Miami Lakes, Fla.), the Cragg-McNamara Valved Infusion Catheter available from Microtherapeutics, Inc. (San Clemente, Calif.), the DISPATCH catheter from Boston Scientific Corporation, the GALILEO Centering Catheter from Guidant Corporation (Houston, Tex.), and infusion sleeve catheters, such as the INFUSASLEEVE catheter from LocalMed, Inc. (Sunnyvale, Calif.). Infusion sleeve catheters are described in, e.g., U.S. Pat. Nos. 5,318,531; 5,336,178; 5,279,565; 5,364,356; 5,772,629; 5,810,767; and 5,941,868. Catheters that mechanically or electrically enhance drug delivery include, for example, pressure driven catheters (e.g., needle injection catheters having injector ports, such as the INFILTRATOR catheter available from InterVentional Technologies, Inc. (San Diego, Calif.)) (see, e.g., U.S. Pat. No. 5,354,279) and ultrasonically assisted (phonophoresis) and iontophoresis catheters (see, e.g., Singh, J., et al. (1989) Drug Des. Deliv.: 4: 1-12 and U.S. Pat. Nos. 5,362,309; 5,318,014; 5,315,998; 5,304,120; 5,282,785; and 5,267,985).

Drug Delivery Balloons

In another aspect, the present invention provides for the combination of a fibrosis-inducing agent and an intravascular drug delivery balloon. “Drug-Delivery Balloon” refers to an intra-arterial balloon (typically based upon percutaneous angioplasty balloons) suitable for insertion into a peripheral artery (typically the femoral artery) and manipulated via a catheter to the treatment (either in the coronary or peripheral circulation). Numerous drug delivery balloons have been developed for local delivery of therapeutic agents to the arterial wall such as “sweaty balloons,” “channel balloons,” “microinjector balloons,” “double balloons,” “spiral balloons” and other specialized drug-delivery balloons.

In addition, numerous drug delivery balloons have been developed for local delivery of therapeutic agents to the arterial wall. Representative examples of drug delivery balloons include porous (WOLINSKY) balloons, available from Advanced Polymers (Salem, N.H.), described in, e.g., U.S. Pat. No. 5,087,244. Microporous and macroporous balloons (i.e., “sweaty balloons”) for use in infusion catheters are described in, e.g., Lambert, C. R. et al. (1992) Circ. Res. 71: 27-33. Other types of specialized drug delivery balloons include hydrogel coated balloons (e.g., ULTRATHIN GLIDES from Boston Scientific Corporation) (see, e.g., Fram, D. B. et al. (1992) Circulation: 86 Suppl. 1: 1-380), “channel balloons” (see, e.g., U.S. Pat. Nos. 5,860,954; 5,843,033; and 5,254,089, and Hong, M. K., et al. (1992) Circulation: 86 Suppl. 1: 1-380), “microinjector balloons” (see, e.g., U.S. Pat. Nos. 5,681,281 and 5,746,716), “double balloons,” described in, e.g., U.S. Pat. No. 6,544,221, and double-layer channeled perfusion balloons (such as the REMEDY balloon from Boston Scientific Corporation), and “spiral balloons” (see, e.g., U.S. Pat. Nos. 6,527,739 and 6,605,056). Drug delivery catheters that include helical (i.e., spiral) balloons are described in, e.g., U.S. Pat. Nos. 6,190,356; 5,279,546; 5236424, 5,226,888; 5,181,911; 4,824,436; and 4,636,195.

The balloon catheter systems that can be used include systems in which the balloon can be inflated at the desired location where the desired fibrosis-inducing agents can be delivered through holes that are located in the balloon wall. Other balloon catheters that can be used include systems that have a plurality of holes that are located between two balloons. The system can be guided into the desired location such that the inflatable balloon components are located on either side of the specific site that is to be treated. The balloons can then be inflated to isolate the treatment area. The compositions containing the fibrosing agent are then injected into the isolated area through the plurality of holes between the two balloons. Representative examples of these types of drug delivery balloons are described in U.S. Pat. Nos. 5,087,244, 6,623,452, 5,397,307, 4,636,195 and 4,994,033.

The compositions can be delivered using a catheter that has the ability to enhance uptake or efficacy of the compositions of the invention. The stimulus for enhanced uptake can include the use of heat, the use of cooling, the use of electrical fields or the use of radiation (e.g., ultraviolet light, visible light, infrared, microwaves, ultrasound or X-rays). Further representative examples of catheter systems that can be used are described in U.S. Pat. Nos. 5,362,309 and 6,623,444; U.S. patent application Publication Nos. 2002/0138036 and 2002/0068869; and PCT Publication Nos. WO 01/15771; WO 94/05361; WO 96/04955 and WO 96/22111.

Stents

In another aspect, the present invention provides for the combination of a fibrosis-inducing agent and an intravascular stent. “Stent” refers to devices comprising an endovascular scaffolding which maintains the lumen of a body passageway (e.g., an artery) and allows bloodflow. Stents frequently are in the form of a cylindrical tube (composed of a metal, textile, non-degradable or degradable polymer, and/or other suitable material—such as biological tissue) which maintains the flow of blood from one portion of a blood vessel to another.

Stents that can be used in the present invention include metallic stents, polymeric stents, biodegradable stents and covered stents. Stents may be self-expandable or balloon-expandable, composed of a variety of metal compounds and/or polymeric materials, fabricated in innumerable designs, used in coronary or peripheral vessels, composed of degradable and/or nondegradable components, fully or partially covered with vascular graft materials (so called “covered stents”) or “sleeves”, and can be bare metal or drug-eluting.

Stents may be comprise a metal or metal alloy such as stainless steel, spring tempered stainless steel, stainless steel alloys, gold, platinum, super elastic alloys, cobalt-chromium alloys and other cobalt-containing alloys (including ELGILOY (Combined Metals of Chicago, Grove Village, Ill.), PHYNOX (Alloy Wire International, United Kingdom) and CON ICHROME (Carpenter Technology Corporation, Wyomissing, Pa.)), titanium-containing alloys, platinum-tungsten alloys, nickel-containing alloys, nickel-titanium alloys (including nitinol), malleable metals (including tantalum); a composite material or a clad composite material and/or other functionally equivalent materials; and/or a polymeric (non-biodegradable or biodegradable) material. Representative examples of polymers that may be included in the stent construction include polyethylene, polypropylene, polyurethanes, polyesters, such as polyethylene terephthalate (e.g., DACRON or MYLAR (E. I. DuPont De Nemours and Company, Wilmington, Del.)), polyamides, polyaramids (e.g., KEVLAR from E.I. DuPont De Nemours and Company), polyfluorocarbons such as poly(tetrafluoroethylene with and without copolymerized hexafluoropropylene) (available, e.g., under the trade name TEFLON (E. I. DuPont De Nemours and Company), silk, as well as the mixtures, blends and copolymers of these polymers. Stents also may be made with engineering plastics, such as thermotropic liquid crystal polymers (LCP), such as those formed from p,p′-dihydroxy-polynuclear-aromatics or dicarboxy-polynuclear-aromatics.

Further types of stents that can be used with the described therapeutic agents are described, e.g., in PCT Publication No. WO 01/01957 and U.S. Pat. Nos. 6,165, 210; 6,099,561; 6,071,305; 6,063,101; 5,997,468; 5,980,551; 5,980,566; 5,972,027; 5,968,092; 5,951,586; 5,893,840; 5,891,108; 5,851,231; 5,843,172; 5,837,008; 5,766,237; 5,769,883; 5,735,811; 5,700,286; 5,683,448; 5,679,400; 5,665,115; 5,649,977; 5,637,113; 5,591,227; 5,551,954; 5,545,208; 5,500,013; 5,464,450; 5,419,760; 5,411,550; 5,342,348; 5,286,254; and 5,163,952. Removable drug-eluting stents are described, e.g., in Lambert, T. (1993) J. Am. Coll. Cardiol.: 21: 483A. Moreover, the stent may be adapted to release the desired agent at only the distal ends, or along the entire body of the stent.

Self-expanding stents that can be used include the coronary WALLSTENT and the SCIMED RADIUS stent from Boston Scientific Corporation (Natick, Mass.). Examples of balloon expandable stents that can be used include the CROSSFLEX stent, BX-VELOCITY stent and the PALMAZ-SCHATZ Crown and Spiral stents from Cordis Corporation (Miami Lakes, Fla.), the V-FLEX PLUS stent by Cook Group, Inc. (Bloomington, Ind.), the NIR, EXPRESS and LIBRERTE stents from Boston Scientific Corporation, the ACS MULTILINK, MULTILINK PENTA, SPIRIT, and CHAMPION stents from Guidant Corporation, and the Coronary Stent S670 and S7 by Medtronic, Inc.

Balloon over stent devices, such as are described in Wilensky, R. L. (1993) J. Am. Coll. Cardiol.: 21: 185A, also are suitable for local delivery of a fibrosing agent to a treatment site.

In addition to using the more traditional stents, stents that are specifically designed for drug delivery can be used. Examples of these specialized drug delivery stents as well as traditional stents include those from Conor Medsystems (Palo Alto, Calif.) (e.g., U.S. Pat. Nos. 6,527,799; 6,293,967; 6,290,673; 6,241,762; U.S. patent application Publication Nos. 2003/0199970 and 2003/0167085; and PCT Publication No. WO 03/015664).

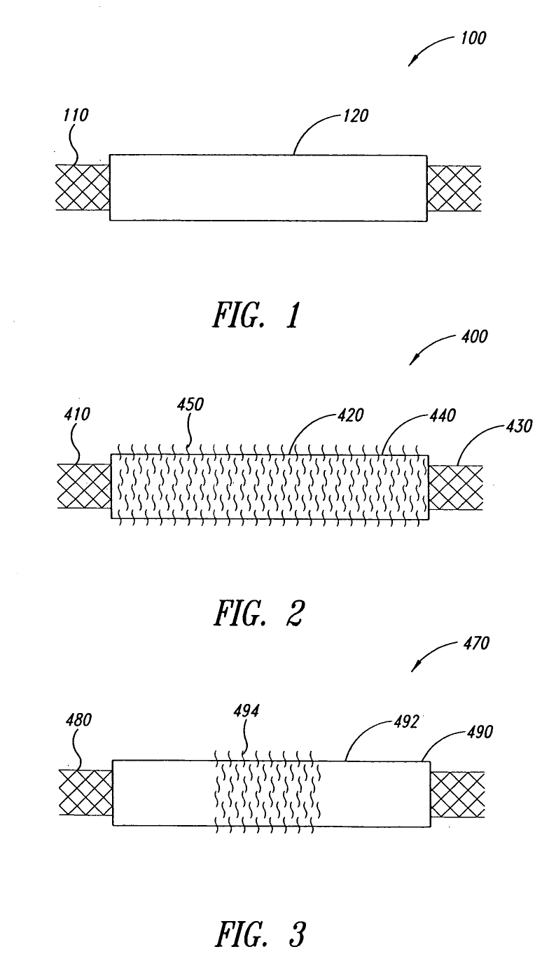

In one aspect of the invention, coated and covered stents can be used as a platform for the delivery of the fibrosing agents. However, in another aspect, the devices of the present invention are devices as disclosed herein excluding stents. The covering for these stents can be in the form of a tube, a sleeve, a mesh, a spiral or a film. These coverings may cover the entire stent or only portions of the stent. For example, referring to FIG. 1, a covered stent 100 is shown having a stent structure 110 with an outer sleeve 120 covering a portion of the stent 110 that contains the fibrosing agent (not shown). The covering can be made from a protein (crosslinked or non-crosslinked), for example collagen or albumin, polyurethanes, PTFE (expanded and woven), polystyrene copolymers (e.g., poly(styrene)-block-poly(isobutylene)-block-poly(styrene), poly(styrene)-poly(isoprene) block copolymers, silicone rubber, poly(ethylene terephthalate), polyamides, polyacrylates, polyvinylidene, degradable polyesters (e.g., poly(lactide), polydioxanone, PLGA, PLA-PCL), crosslinked polyalkylene oxide (e.g., a tetrafunctional “4-armed” PEG, such as described below) as well blends and copolymers thereof. Representative examples of these stents are described in U.S. patent application Publication Nos. 2003/0009213, 2003/0074049, 2003/0191519, 2003/0036792, 2002/0165601, 2002/0072790, 2002/0055768, 2002/0052648, 2001/0056299, and 2001/0053931, and U.S. Pat. Nos. 6,290,722; 6,530,950; 6,248,129; 6,168,619; 6,019,789; 5,954,744; 5,674,242 5,603,722; 6,592,617; 6,579,314; 6,475,234; 6,447,521; 6,395,212; 5,922,393; 5,895,407; 5,824,046; 5,718,159; and 5,713,949.

Stent Grafts

In another aspect, intravascular stents (typically cylindrical metallic scaffolds similar in design to those described above) are provided that also comprise a graft portion (typically a solid, synthetic vascular graft that covers or incorporates the stent scaffold), referred to herein as “stent grafts.”

A stent graft is typically used to bridge a diseased artery (usually an aneurysm), extending from a portion of artery of acceptable caliber above the diseased region to an artery of acceptable caliber below the diseased region. Stent grafts may be used, for example, to bypass an abdominal aortic aneurysm (AAA) or a thoracic aortic aneurysm (TAA). For example, treatment of an AAA with a stent graft typically involves inserting the stent graft over a guide wire, from the femoral or iliac artery, and deploying it within the aneurysm, resulting in maintenance of blood flow from an aorta of acceptable (usually normal) caliber above the aneurysm to a portion of aorta or iliac artery(s) of acceptable (usually normal) caliber below the aneurysm. Blood flow is thereby excluded from entering the aneurysm sac. Blood within this excluded sac thromboses and the aneurysm thus has no flow within it, presumably reducing the pressure and thus its tendency to burst.

Endovascular stent grafts are a significant advance in the treatment of AAA as they offer an alternative to standard surgical therapy, which is a major operation with a significant morbidity, mortality, long hospital stays, and prolonged recovery time. While generally useful, however, presently available stent grafts have a number of shortcomings. For example, current stent grafts are prone to persistent leakage around the area of the stent graft. Hence, pressure within the aneurysm sac stays at or near arterial pressure, and there remains a risk that the sac will rupture. There are three common types of perigraft leakage. The first type is direct leakage around the proximal end (the end closest to the heart) of the stent graft. This can be persistent from the time of insertion because of poor sealing between the stent graft and vessel wall, or can develop later because the seal is subsequently lost. Typically when a leak develops after an initially successful implantation, it is because the stent graft has migrated “downstream” into the aneurysm (which is wider and allows blood to flow around the top of the stent graft) or because the aneurysm continues to grow or elongate with time after treatment (such that the aneurysm now extends beyond the top of the stent graft). A second type of perigraft leak can occur due to retrograde blood flow through arterial branches that come off of the aorta in the segment treated by the stent graft. Once the device excludes the aneurysm, flow can reverse within these blood vessels and continue to fill the aneurysm sac around the stent graft. The third type of perigraft leak can occur due to device failure, either because of disarticulation of the device (in the case of modular devices) or because of the development of holes within the graft material. The continuous pulsation of the vessel can cause wear in the graft material from constant rubbing against the metallic stent scaffold that supports the graft fabric, leading to hole formation, leakage and eventual graft failure. In addition, disarticulation of the device can develop due to dynamic changes in shape of the aneurysm as it grows, expands in diameter, elongates or changes shape with time after treatment—a phenomenon that current iterations of stent grafts do nothing to address.

To achieve a long lasting seal between a stent graft and the arterial wall, the artery of above the diseased region (“proximal neck”) should be of acceptable caliber and at least 1.5 cm long without a major branch vessel arising from it. The artery below the diseased region (“distal neck”) should be of acceptable caliber and at least 1.0 cm long without a major branch vessel arising within that 1 cm length of vessel. Shorter “necks” at either end of the diseased segment, necks which are sloping rather than cylindrical, or necks which are smaller than the aneurysm but still dilated in comparison to the normal diameter for a vessel in this location predispose to failure of sealing around the stent graft or delayed perigraft leaks.

Current stent graft technology is only applicable to certain patients with AAA or TAA, because (a) they lack a suitable route of access via the blood vessels to the intended site of deployment and prevents insertion of the device and (b) the patient's aneurysm or vessel anatomy is not suitable to treatment with a stent graft. Implantation of a stent graft into a patient requires surgical exposure of the insertion site (usually a cutdown of the common femoral artery). Due to the thickness of the stent graft material, their delivery devices are typically about 24 to 27 French (8 to 9 millimeter diameter) and occasionally up to 32 French in size. These larger delivery devices are difficult to manipulate through the iliac artery to the intended site of delivery. Even “low profile” devices, which use thinner graft material, are of a sufficient size that a femoral cutdown is required for insertion. If the iliac arteries or aorta are very tortuous, (as is frequently the case in AAA or TAA), or heavily calcified and diseased (another frequent association with AAA), this may be a contraindication to treatment, or cause of failure of attempted treatment, because of inability to advance a device to the site of deployment or potential for iliac artery rupture.

The scaffold (stent) portion of the stent graft may include a metal or metal alloy, or a polymeric (non-biodegradable or biodegradable) material as described above for stents in general. The scaffold may comprise a biodegradable polymer, such as, for example, collagen, poly(esters) [e.g., polyester that comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, ?-decanolactone, d-decanolactone, trimethylene carbonate, 1,4-dioxane-2-one or 1, 5-dioxepan-2one], poly(ester-carbonate)s. Biodegradable scaffolds are capable of dissolving over time, such that wear to the graft materials which cover them may be reduced. By diminishing wear and destruction of the graft material, leakage through the graft material into the aneurysm sac may be minimized.

In one aspect, stent grafts are provided having an external stent portion which may be formed in many configurations. For example, configurations of stent portions may include, but are not limited to, braids (open lattice or closely woven), helical structural strands, sinusoidal structural strands, mesh-like materials, diamond-shaped mesh, rectangular shaped mesh, functional equivalents thereof and/or combinations thereof. External stent portions may be composed of a variety of materials that are sufficiently strong, biocompatible and fatigue-resistant. The stent portion may, in certain embodiments, include fibrous or tufted extensions which may further increase the thrombogenicity of the device.

The graft portion of a stent graft may be made from a textile, polymer, or other suitable material such as biological tissue. In order to effectively exclude an aneurysm, the graft material needs to be of certain strength and durability, or else it will tear. Typically, in order to achieve these properties, a polyester (e.g., polyester sold, e.g., under the trade name DACRON (E. I. DuPont De Nemours and Company) or poly(tetrafluoroethylene) (PTFE)) graft material of conventional “surgical” thickness may be used. This level of thickness is used so as to convey adequate strength to the material; however, in the practice of the invention, thinner materials also may be utilized. Representative examples of graft materials include textiles (including, e.g., woven and non-woven materials) made from polymeric fibers. Polymeric fibers for use in textiles may be formed from a variety of polymers, including, for example, nylon, acrylonitrile polymers and copolymers (available, e.g., under the trade name ORLON (E. I. DuPont De Nemours and Company)), polyethers or polyesters, such as polyethylene terephthalate (e.g., DACRON or MYLAR), poly(tetrafluoroethylene) (e.g., TEFLON), and polyaramids (e.g., KEVLAR). Other representative examples of graft materials include non-textiles, such as polyolefins such as polyproplylene, or elastomeric materials such as polyurethane or silicone rubber, and expanded polytetrafluroethylene (ePTFE). Biological tissues that may be used include, but are not limited to, umbilical cord tissue, and collagenous tissue. Mammalian intestinal submucosa derived from sheep, bovine, porcine or other sources can also be utilized as graft material.

The graft or covering may be woven within a stent, contained within the lumen of a stent and/or be located exterior to a stent. The graft portion may be a graft sleeve in the form of a continuous sheet, interwoven textile strands, multiple filament yarns (twisted or nontwisted), monofilament yarns and/or combinations thereof.

Representative examples of stent grafts suitable for use in one or more aspects of the invention, and methods for making and utilizing such grafts are described in more detail in U.S. Pat. No. 5,810,870 entitled “Intraluminal Stent Graft”; U.S. Pat. No. 5,776,180 entitled “Bifurcated Endoluminal Prosthesis”; U.S. Pat. No. 5,755,774 entitled “Bistable Luminal Graft Endoprosthesis”; U.S. Pat. Nos. 5,735,892 and 5,700,285 entitled “Intraluminal Stent Graft”; U.S. Pat. No. 5,723,004 entitled “Expandable Supportive Endoluminal Grafts”; U.S. Pat. No. 5,718,973 entitled “Tubular Intraluminal Graft”; U.S. Pat. No. 5,716,365 entitled “Bifurcated Endoluminal Prosthesis”; U.S. Pat. No. 5,713,917 entitled “Apparatus and Method for Engrafting a Blood Vessel”; U.S. Pat. No. 5,693,087 entitled “Method for Repairing an Abdominal Aortic Aneurysm”; U.S. Pat. No. 5,683,452 entitled “Method for Repairing an Abdominal Aortic Aneurysm”; U.S. Pat. No. 5,683,448 entitled “Intraluminal Stent and Graft”; U.S. Pat. No. 5,653,747 entitled “Luminal Graft Endoprosthesis and Manufacture Thereof”; U.S. Pat. No. 5,643,208 entitled “Balloon Device of Use in Repairing an Abdominal Aortic Aneurysm”; U.S. Pat. No. 5,639,278 entitled “Expandable Supportive Bifurcated Endoluminal Grafts”; U.S. Pat. No. 5,632,772 entitled “Expandable Supportive Branched Endoluminal Grafts”; U.S. Pat. No. 5,628,788 entitled “Self-Expanding Endoluminal Stent-Graft”; U.S. Pat. No. 5,591,229 entitled “Aortic Graft for Repairing an Abdominal Aortic Aneurysm”; U.S. Pat. No. 5,591,195 entitled “Apparatus and Methods for Engrafting a Blood Vessel”; U.S. Pat. No. 5,578,072 entitled “Aortic Graft and Apparatus for Repairing an Abdominal Aortic Aneurysm”; U.S. Pat. No. 5,578,071 entitled “Aortic Graft”; U.S. Pat. No. 5,571,173 entitled “Graft to Repair a Body Passageway”; U.S. Pat. No. 5,571,171 entitled “Method for Repairing an Artery in a Body”; U.S. Pat. No. 5,522,880 entitled “Method for Repairing an Abdominal Aortic Aneurysm”; U.S. Pat. No. 5,405,377 entitled “Intraluminal Stent”; U.S. Pat. No. 5,360,443 entitled “Aortic Graft for Repairing an Abdominal Aortic Aneurysm”; U.S. Pat. No. 6,488,701 entitled “Stent-graft assembly with thin-walled graft component and method of manufacture”; U.S. Pat. No. 6,482,227 entitled “Stent graft having improved attachment within a body vessel”; U.S. Pat. No. 6,458,152 entitled “Coiled sheet graft for single and bifurcated lumens and methods of making and use”; U.S. Pat. No. 6,451,050 entitled “Stent graft and method”; U.S. Pat. No. 6,395,018 entitled “Endovascular graft and process for bridging a defect in a main vessel near one of more branch vessels”; U.S. Pat. No. 6,390,098 entitled “Percutaneous bypass with branching vessel”; U.S. Pat. No. 6,361,637 entitled “Method of making a kink resistant stent-graft”; U.S. Pat. No. 6,348,066 entitled “Modular endoluminal stent-grafts and methods for the use”; U.S. Pat. No. 6,344,054 entitled “Endoluminal prosthesis comprising stent and overlying graft cover, and system and method for deployment thereof”; U.S. Pat. No. 6,325,820 entitled “Coiled-sheet stent-graft with exo-skeleton”; U.S. Pat. No. 6,322,585 entitled “Coiled-sheet stent-graft with slidable exo-skeleton”; U.S. Pat. No. 6,319,278 entitled “Low profile device for the treatment of vascular abnormalities”; U.S. Pat. No. 6,296,661 entitled “Self-expanding stent-graft”; U.S. Pat. No. 6,245,100 entitled “Method for making a self-expanding stent-graft”; U.S. Pat. No. 6,238,432 entitled “Stent graft device for treating abdominal aortic aneurysms”; U.S. Pat. No. 6,214,039 entitled “Covered endoluminal stent and method of assembly”; U.S. Pat. No. 6,168,610 entitled “Method for endoluminally excluding an aortic aneurysm”; U.S. Pat. No. 6,165,213 entitled “System and method for assembling an endoluminal prosthesis”; U.S. Pat. No. 6,165,210 entitled “Self-expandable helical intravascular stent and stent-graft”; U.S. Pat. No. 6,143,022 entitled “Stent-graft assembly with dual configuration graft component and method of manufacture”; U.S. Pat. No. 6,123,722 entitled “Stitched stent grafts and methods for the fabrication”; U.S. Pat. No. 6,117,167 entitled “Endoluminal prosthesis and system for joining”; U.S. Pat. No. 6,099,559 entitled “Endoluminal support assembly with capped ends”; U.S. Pat. No. 6,042,605 entitled “Kink resistant stent-graft”; U.S. Pat. No. 6,015,431 entitled “Endolumenal stent-graft with leak-resistant seal”; U.S. Pat. No. 5,957,974 entitled “Stent graft with braided polymeric sleeve”; U.S. Pat. No. 5,916,264 entitled “Stent graft”; U.S. Pat. No. 5,906,641 entitled “Bifurcated stent graft”; U.S. Pat. No. 5,891,191 entitled “Cobalt-chromium-molybdenum alloy stent and stent-graft”; U.S. Pat. No. 5,824,037 entitled “Modular intraluminal prostheses construction and methods”; U.S. Pat. No. 5,824,036 entitled “Stent for intraluminal grafts and device and methods for delivering and assembling same”; U.S. patent application Publication Nos. 2003/0120331; 2003/120338; and 2003/0125797; U.S. Pat. Nos. 6,165,210 and 6,334,867, and PCT Publication No. WO 99/37242.

Stent grafts, which may be combined with one or more drugs according to the present invention, include commercially available products. For example, the TALENT AAA Stent Graft System and the ANEURX AAA Stent Graft System (both from Medtronic, Inc., Minneapolis, Minn.), which has a unique modular design allowing for customization in vivo to accommodate different anatomies; the EXCLUDER Bifurcated Endoprosthesis device made of durable ePTFE bifurcated graft with an outer self-expanding nitinol support structure (W. L. Gore & Associates, Inc., Flagstaff, Aria.); the LIFEPATH AAA System from Edwards Lifesciences Corp. (Irvine, Calif.); the ZENITH AAA Stent Graft from Cook Group, Inc. (Bloomington, Ind.); the JOSTENT Coronary Stent Graft from Abbott Laboratories, Inc. (Abbott Park, Ill.); the POWERLINK Aortic Aneurysm Therapy System from Endologix, Inc. (Irvine, Calif.), and stent grafts that may be delivered through the skin such as are being developed by Trivascular, Inc. (Santa Rosa, Calif.).

Other Intravascular Devices

Other intravascular devices can be used to deliver the fibrosing agents to an aneurysm or a vulnerable plaque. “Other Intravascular Device” refers to any intravascularly (e.g., intra-arterially) delivered medical device that is not considered a catheter, balloon, stent graft, or stent that can be used to deliver the fibrosis-inducing therapeutic agents to a blood vessel. Examples include, but are not restricted to, shunts, vascular grafts (synthetic and autologous), anastomotic connector devices, IVUS (intravascular ultrasound devices), lasers, cryotherapy devices, radiofrequency devices, thermography devices, angioscopes, embolic protection devices, coronary drug infusion guidewires, such as those available from TherOx, Inc., and other specialized intravascular devices.

Another example of an intraluminal device is an intraluminal graft absent an endovascular scaffold or stent. For example, the graft material may possess enough radial strength to prevent collapse of the intraluminal device such that an additional scaffold or stent is not required. Devices having such constructions may be used, for example, in the treatment of aneurysms. In another embodiment, a scaffold may be formed in situ. A polymeric material can be injected into the graft material once the graft is deployed intraluminally. Once the polymer sets, the polymer loaded graft material can provide a scaffold for the device.

A. Therapeutic Agents

Briefly, a wide variety of agents (also referred to herein as ‘therapeutic agents’ or ‘drugs’) can be utilized within the context of the present invention. Within one aspect, the therapeutic agent is a fibrosis-inducing (i.e., scarring) agent. Within another aspect, the therapeutic agent can induce adhesion between a device and tissue proximate to the device. The agent may be formulated with one or more other materials, e.g., a polymeric carrier, where formulations are discussed later herein. Many suitable therapeutic agents are specifically identified herein, and others may be readily determined based upon in vitro and in vivo (animal) models such as those provided in Examples 13-25; 38-39; and 46-47. Theraeutic agents which promote fibrosis can be identified through in vivo models such as the rat carotid artery model (Examples 22-25), the sheep aneurysm model (Example 47), and the animal AAA model (Example 46).

In one aspect, the fibrosis or adhesion-inducing agent is silk. Silk refers to a fibrous protein and may be obtained from a number of sources; typically spiders and silkworms. Typical silks contain about 75% of actual fiber, referred to as fibroin, and about 35% sericin, which is a gummy protein that holds the filaments together. Silk filaments are generally very fine and long—as much as 300-900 meters long. There are several species of domesticated silkworm that are used in commercial silk production, however, Bombyx mori is the most common, and most silk comes from this source. Other suitable silkworms include Philosamia ricini, Antheraea yamamai, Antheraea pernyi, and Antheraea mylitta. Spider silk is relatively more difficult to obtain, however, recombinant techniques hold promise as a means to obtain spider silk at economical prices (see, e.g., U.S. Pat. Nos. 6,268,169; 5,994,099; 5,989,894; and 5,728,810, which are exemplary only). Biotechnology has allowed researchers to develop other sources for silk production, including animals (e.g., goats) and vegetables (e.g., potatoes). Silk from any of these sources may be used in the present invention.

A commercially available silk protein is available from Croda, Inc., of Parsippany, N.J., and is sold under the trade names CROSILK LIQUID (silk amino acids), CROSILK 10,000 (hydrolyzed silk), CROSILK POWDER (powdered silk), and CROSILKQUAT (cocodiammonium hydroxypropyl silk amino acid). Another example of a commercially available silk protein is SERICIN, available from Pentapharm, LTD, a division of Kordia, BV, of the Netherlands. Further details of such silk protein mixtures can be found in U.S. Pat. No. 4,906,460, to Kim, et al., assigned to Sorenco. Silk useful in the present invention includes natural (raw) silk, degummed silk, hydrolyzed silk, and modified silk, i.e., silk that has undergone a chemical, mechanical, or vapor treatment, e.g., acid treatment or acylation (see, e.g., U.S. Pat. No. 5,747,015).

Raw silk is typically twisted into a strand sufficiently strong for weaving or knitting. Four different types of silk thread may be produced by this procedure: organzine, crepe, tram and thrown singles. Organzine is a thread made by giving the raw silk a preliminary twist in one direction and then twisting two of these threads together in the opposite direction. Crepe is similar to organzine but is twisted to a much greater extent. Twisting in only one direction two or more raw silk threads makes tram. Thrown singles are individual raw silk threads that are twisted in only one direction. Any of these types of silk threads may be used in the present invention.

The silk used in the present invention may be in any suitable form that allows the silk to be joined with the medical implant, e.g., the silk may be in thread or powder-based forms. Furthermore, the silk may have any molecular weight, where various molecular weights are typically obtained by the hydrolysis of natural silk, where the extent and harshness of the hydrolysis conditions determines the product molecular weight. For example, the silk may have an average (number or weight) molecular weight of 200 to 5,000. See, e.g., JP-B-59-29199 (examined Japanese patent publication) for a description of conditions that may be used to hydrolyze silk.

A discussion of silk may be found in the following documents, which are exemplary only: Hinman, M. B., et al. “Synthetic spider silk: a modular fibre” Trends in Biotechnology, 2000, 18(9) 374-379; Vollrath, F. and Knight, D. P. “Liquid crystalline spinning of spider silk” Nature, 2001, 410(6828) 541-548; and Hayashi, C. Y., et al. “Hypotheses that correlate the sequence, structure, and mechanical properties of spider silk proteins” Int J. Biol. Macromolecules, 1999, 24(2-3), 265-270; and U.S. Pat. No. 6,427,933.

In certain other aspects or embodiments, the fibrosing agent is not silk, or the composition comprising the fibrosing agent does not contain silk.

Other representative examples of fibrosis and adhesion-inducing agents include irritants (e.g., talc, wool (including animal wool, wood wool, and synthetic wool), talcumpowder, copper, metallic beryllium (or its oxides), asbestos, wool quartz dust, silica, crystalline silicates), polymers (e.g., polylysine, polyurethanes, poly(ethylene terephthalate), polymers comprising multiple amino groups, PTFE, poly(alkylcyanoactylates), and poly(ethylene-co-vinylacetate)); crosslinked hydrogels made from multifunctional terminal amino derivatized poly(ethylene glycol) and multifunctional terminal hydroxysuccinimidyl derivatized poly(ethylene glycol), crosslinked hydrogels made from multifunctional terminal amino derivatized poly(ethylene glycol), multifunctional termirial thio derivatized poly(ethylene glycol) and multifunctional terminal hydroxysuccinimidyl derivatized poly(ethylene glycol), hydroxyl ine A, vinyl chloride and polymers of vinyl chloride; peptides with high lysine content; growth factors and inflammatory cytokines involved in angiogenesis, fibroblast migration, fibroblast proliferation, ECM synthesis and tissue remodeling, such as epidermal growth factor (EGF) family, transforming growth factor-α (TGF-α), transforming growth factor-β (TGF-9-1, TGF-9-2, TGF-9-3, platelet-derived growth factor (PDGF), fibroblast growth factor (acidic—aFGF; and basic—bFGF), fibroblast stimulating factor-1, activins, vascular endothelial growth factor (including VEGF-2, VEGF-3, VEGF-A, VEGF-B, VEGF-C, placental growth factor—PIGF), angiopoietins, insulin-like growth factors (IGF), hepatocyte growth factor (HGF), connective tissue growth factor (CTGF), myeloid colony-stimulating factors (CSFs), monocyte chemotactic protein, granulocyte-macrophage colony-stimulating factors (GM-CSF), granulocyte colony-stimulating factor (G-CSF), macrophage colony-stimulating factor (M-CSF), erythropoietin, interleukins (particularly IL-1, IL-8, IL-6), tumor necrosis factor-α (TNF9), nerve growth factor (NGF), interferon-α, interferon-β, histamine, endothelin-1, angiotensin II, growth hormone (GH), and synthetic peptides, analogues or derivatives of these factors are also suitable for release from specific intravascular devices. Other examples include inflammatory microcrystals (e.g., crystalline minerals such as crystalline silicates); monocyte chemotactic protein, fibroblast stimulating factor 1, histamine, endothelin-1, angiotensin II, bovine collagen, bromocriptine, methylsergide, methotrexate, chitosan, N-carboxybutyl chitosan, carbon tetrachloride, thioacetamide, fibrosin, ethanol, naturally occurring or synthetic peptides containing the Arg-Gly-Asp (RGD) sequence, generally at one or both termini, described, e.g., in U.S. Pat. No. 5,997,895, bleomycin, and tissue adhesives, such as cyanoacrylate and crosslinked poly(ethylene glycol)—methylated collagen compositions, such as described below. Other examples of fibrosis-inducing agents include bone morphogenic proteins (e.g., BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16 (of these, BMP-2, BMP-3, BMP-4, BMP-5, BMP-6, and BMP-7 are of particular utility. Bone morphogenic proteins are described, for example, in U.S. Pat. Nos. 4,877,864; 5,013,649; 5,661,007; 5,688,678; 6,177,406; 6,432,919; and 6,534,268 and Wozney, J. M., et al. (1988) Science: 242(4885); 1528-1534.

Other representative examples of fibrosis-inducing agents include components of extracellular matrix (e.g., fibronectin, fibrin, fibrinogen, collagen, including fibrillar and non-fibrillar collagen, adhesive glycoproteins, proteoglycans (e.g., heparin sulphate, chondroitin sulphate, dermatan sulphate), hyaluronan, secreted protein acidic and rich in cysteine (SPARC), thrombospondins, tenacin, and cell adhesion molecules (including integrins, vitronectin, fibronectin, laminin, hyaluronic acid, elastin, bitronectin), proteins found in basement membranes, and fibrosin) and inhibitors of matrix metalloproteinases, such as tissue inhibitors of matrix metalloproteinases (TIMPs) and synthetic TIMPs, such as, e.g., marimistat, batimistat, doxycycline, tetracycline, minocycline, cipemastat (Ro-32-3555), sold under the tradename TROCADE (F. Hoffman-La Roche Ltd., Switzerland), Ro-1130830, CGS 27023A, AND BMS-275291.

Within various embodiments of the invention, a device is coated with a first composition that promotes fibrosis and a second composition or compound which acts to have an inhibitory effect on pathological processes in or around the treatment site. Representative examples of agents which can inhibit pathological processes in the treatment site include, but not limited to, the following classes of compounds: anti-inflammatory agents (e.g., dexamethasone, cortisone, fludrocortisone, prednisone, prednisolone, 6α-methylprednisolone, triamcinolone, betamethasone); MMP inhibitors (e.g., batimistat, marimistat, nimesulide, PKF-241-466, PKF-242-484, CGS-27023A, SAR-943, primomastat, SC-77964, PNU-171829, AG-3433, PNU-142769, SU-5402, nemesulide, dexlipotam, TIMP's (tissue inhibitors of matrix metalloproteinases; representative examples are included in U.S. Pat. Nos. 5,665,777; 5,985,911; 6,288,261; 5,952,320; 6,441,189; 6,235,786; 6,294,573; 6,294,539; 6,563,002; 6,071,903; 6,358,980; 5,852,213; 6,124,502; 6,160,132; 6,197,791; 6,172,057; 6,288,086; 6,342,508; 6,228,869; 5,977,408; 5,929,097; 6,498,167; 6,534,491; 6,548,524; 5,962,481; 6,197,795; 6,162,814; 6,441,023; 6,444,704; 6,462,073; 6,162,821; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 6,444,639; 6,262,080; 6,486,193; 6,329,550; 6,544,980; 6,352,976; 5,968,795; 5,789,434; 5,932,763; 6,500,847; 5,925,637; 6,225,314; 5,804,581; 5,863,915; 5,859,047; 5,861,428; 5,886,043; 6,288,063; 5,939,583; 6,166,082; 5,874,473; 5,886,022; 5,932,577; 5,854,277; 5,886,024; 6,495,565; 6,642,255; 6,495,548; 6,479,502; 5,696,082; 5,700,838; 5,861,436; 5,691,382; 5,763,621; 5,866,717; 5,902,791; 5,962,529; 6,017,889; 6,022,873; 6,022,898; 6,103,739; 6,127,427; 6,258,851; 6,310,084; 6,358,987; 5,872,152; 5,917,090; 6,124,329; 6,329,373; 6,344,457; 5,698,706; 5,872,146; 5,853,623; 6,624,144; 6,462,042; 5,981,491; 5,955,435; 6,090,840; 6,114,372; 6,566,384; 5,994,293; 6,063,786; 6,469,020; 6,118,001; 6,187,924; 6,310,088; 5,994,312; 6,180,611; 6,110,896; 6,380,253; 5,455,262; 5,470,834; 6,147,114; 6,333,324; 6,489,324; 6,362,183; 6,372,758; 6,448,250; 6,492,367; 6,380,258; 6,583,299; 5,239,078; 5,892,112; 5,773,438; 5,696,147; 6,066,662; 6,600,057; 5,990,158; 5,731,293; 6,277,876; 6,521,606; 6,168,807; 6,506,414; 6,620,813; 5,684,152; 6,451,791; 6,476,027; 6,013,649; 6,503,892; 6,420,427; 6,300,514; 6,403,644; 6,177,466; 6,569,899; 5,594,006; 6,417,229; 5,861,510; 6,156,798; 6,387,931; 6,350,907; 6,090,852; 6,458,822; 6,509,337; 6,147,061; 6,114,568; 6,118,016; 5,804,593; 5,847,153; 5,859,061; 6,194,451; 6,482,827; 6,638,952; 5,677,282; 6,365,630; 6,130,254; 6,455,569; 6,057,369; 6,576,628; 6,110,924; 6,472,396; 6,548,667; 5,618,844; 6,495,578; 6,627,411; 5,514,716; 5,256,657; 5,773,428; 6,037,472; 6,579,890; 5,932,595; 6,013,792; 6,420,415; 5,532,265; 5,639,746; 5,672,598; 5,830,915; 6,630,516; 5,324,634; 6,277,061; 6,140,099; 6,455,570; 5,595,885; 6,093,398; 6,379,667; 5,641,636; 5,698,404; 6,448,058; 6,008,220; 6,265,432; 6,169,103; 6,133,304; 6,541,521; 6,624,196; 6,307,089; 6,239,288; 5,756,545; 6,020,366; 6,117,869; 6,294,674; 6,037,361; 6,399,612; 6,495,568; 6,624,177; 5,948,780; 6,620,835; 6,284,513; 5,977,141; 6,153,612; 6,297,247; 6,559,142; 6,555,535; 6,350,885; 5,627,206; 5,665,764; 5,958,972; 6,420,408; 6,492,422; 6,340,709; 6,022,948; 6,274,703; 6,294,694; 6,531,499; 6,465,508; 6,437,177; 6,376,665; 5,268,384; 5,183,900; 5,189,178; 6,511,993; 6,617,354; 6,331,563; 5,962,466; 5,861,427; 5,830,869; and 6,087,359), cytokine inhibitors (chlorpromazine, mycophenolic acid, rapamycin, TNF-484A, PD-172084, CP-293121, CP-353164, PD-1 68787, and 1α-hydroxy vitamin D₃), IMPDH inhibitors (e.g., mycophenolic acid, ribaviran, aminothiadiazole, thiophenfurin, tiazofurin, viramidine) (Representative examples are included in U.S. Pat. Nos. 5,536,747; 5,807,876; 5,932,600; 6,054,472; 6,128,582; 6,344,465; 6,395,763; 6,399,773; 6,420,403; 6,479,628; 6,498,178; 6,514,979; 6,518,291; 6,541,496; 6,596,747; 6,617,323; and 6,624,184, U.S. patent application Publication Nos. 2002/0040022A1, 2002/0052513A1, 2002/0055483A1, 2002/0068346A1, 2002/0111378A1, 2002/0111495A1, 2002/0123520A1, 2002/0143176A1, 2002/0147160A1, 2002/0161038A1, 2002/0173491A1, 2002/0183315A1, 2002/0193612A1, 2003/0027845A1, 2003/0068302A1, 2003/0105073A1, 2003/0130254A1, 2003/0143197A1, 2003/0144300A1, 2003/0166201A1, 2003/0181497A1, 2003/0186974A1, 2003/0186989A1, and 2003/0195202A1, and PCT Publication Nos. WO 00/24725A1, WO 00/25780A1, WO 00/26197A1, WO 00/51615A1, WO 00/56331A1, WO 00/73288A1, WO 01/00622A1, WO 01/66706A1, WO 01/79246A2, WO 01/81340A2, WO 01/85952A2, WO 02/16382A1, WO 02/18369A2, WO 02/051814A1, WO 02/057287A2, WO 02/057425A2, WO 02/060875A1, WO 02/060896A1, WO 02/060898A1, WO 02/068058A2, WO 03/020298A1, WO 03/037349A1, WO 03/039548A1, WO 03/045901A2, WO 03/047512A2, WO 03/053958A1, WO 03/055447A2, WO 03/059269A2, WO 03/063573A2, WO 03/087071A1, WO 99/001545A1, WO 97/40028A1, WO 97/41211A1, WO 98/40381A1, and WO 99/55663A1), p38 MAP kinase inhibitors (e.g., GW-2286, CGP-5241 1, BIRB-798, SB220025, RO-320-1195, RWJ-67657, RWJ-68354, and SCIO-469), representative of which are included in U.S. Pat. Nos. 6,300,347; 6,316,464; 6,316,466; 6,376,527; 6,444,696; 6,479,507; 6,509,361; 6,579,874, and 6,630,485, and U.S. patent application Publication Nos. 2001/0044538A1, 2002/0013354A1, 2002/0049220A1, 2002/0103245A1, 2002/0151491A1, 2002/0156114A1, 2003/0018051A1, 2003/0073832A1, 2003/0130257A1, 2003/0130273A1, 2003/0130319A1, 2003/0139388A1, 2003/0139462A1, 2003/0149031A1, 2003/0166647A1, and 2003/0181411A1, and PCT Publication Nos. WO 00/63204A2, WO 01/21591A1, WO 01/35959A1, WO 01/74811A2, WO 02/18379A2, WO 02/064594A2, WO 02/083622A2, WO 02/094842A2,WO 02/096426A1, WO 02/101015A2, WO 02/103000A2, WO 03/008413A1, WO 03/016248A2, WO 03/020715A1, WO 03/024899A2, WO 03/031431A1, WO 03/040103A1, WO 03/053940A1, WO 03/053941A2, WO 03/063799A2, WO 03/079986A2, WO 03/080024A2, WO 03/082287A1, WO 97/44467A1, WO 99/01449A1, and WO 99/58523A1, and immunomodulatory agents (rapamycin, everolimus, ABT-578, azathioprine, tacrolimus, and azithromycin, and analogues and derivatives of these agents). Analogues of rapamycin include tacrolimus and derivatives thereof (e.g., EP 0184162B1 and those described in U.S. Pat. No. 6,258,823) and everolimus and derivatives thereof (e.g., U.S. Pat. No. 5,665,772). Further representative examples of sirolimus analogues and derivatives include ABT-578 and those found in PCT Publication Nos. WO 97/10502, WO 96/41807, WO 96/35423, WO 96/03430, WO 96/00282, WO 95/16691, WO 95/15328, WO 95/07468, WO 95/04738, WO 95/04060, WO 94/25022, WO 94/21644, WO 94/18207, WO 94/10843, WO 94/09010, WO 94/04540, WO 94/02485, WO 94/02137, WO 94/02136, WO 93/25533, WO 93/18043, WO 93/13663, WO 93/11130, WO 93/10122, WO 93/04680, WO 92/14737, and WO 92/05179 and in U.S. Pat. Nos. 6,342,507; 5,985,890; 5,604,234; 5,597,715; 5,583,139; 5,563,172; 5,561,228; 5,561,137; 5,541,193; 5,541,189; 5,534,632; 5,527,907; 5,484,799; 5,457,194; 5,457,182; 5,362,735; 5,324,644; 5,318,895; 5,310,903; 5,310,901; 5,258,389; 5,252,732; 5,247,076; 5,225,403; 5,221,625; 5,210,030; 5,208,241; 5,200,411; 5,198,421; 5,147,877; 5,140,018; 5,116,756; 5,109,112; 5,093,338; and 5,091,389). Other examples of immunosuppressants include argyrin B, macrocyclic lactone, ADZ-62-826, CCl-779, tilomisole, amcinonide, FK-778, AVE-1726, and MDL-28842. Other examples of drugs that may be included in the compositions and in or on devices of the invention include tyrosine kinase inhibitors, such as imantinib, ZK-222584, CGP-5241 1, CGP-53716, NVP-AAK980-NX, CP-127374, CP-564959, PD-171026, PD-173956, PD-180970, SU-0879, and SKI-606; NFKB Inhibitors, such as, AVE-0547, AVE-0545, and IPL-576092; HMGCOA reductase inhibitors such as pravestatin, atorvastatin, fluvastatin, dalvastatin, glenvastatin, pitavastatin, CP-83101, U-20685, apoptosis antagonist (e.g., troloxamine, TCH-346 (N-methyl-N-propargyl-10-aminomethyl-dibenzo(b,f)oxepin), caspase inhibitor (e.g., PF-5901 (benzenemethanol, alpha-pentyl-3-(2-quinolinylmethoxy)-), and JNK Inhibitor (e.g., AS-602801).

Within various embodiments of the invention, a device is incorporates or is coated with a composition which promotes fibrosis (and/or restenosis), as well as a composition or compound which acts to stimulate cellular proliferation. Representative examples of agents that stimulate cellular proliferation include dexamethasone, isotretinoin (13-cis retinoic acid), 17-β-estradiol, estradiol, 1-a-25 dihydroxyvitamin D₃, diethylstibesterol, cyclosporine A, L-NAME, all-trans retinoic acid (ATRA), and analogues and derivatives thereof. Other examples of agents that stimulate cellular proliferation include: sphingosine 1-phosphate receptor agonist (e.g., FTY-720 (1,3-propanediol, 2-amino-2-(2-(4-octylphenyl)ethyl)-, hydrochloride; immunostimulants, such as imupedone (methanone, [5-amino-2-(4-methyl-1-piperidinyl)phenyl](4-chlorophenyl)-), synthetic peptides such as DIAPEP 227 (Peptor Ltd., Israel); and nerve growth factor agonist, such as, e.g., NG-012 (5H,9H,13H,21H,25H,-dibenzo[k,u][1,5,9,15,19]pentaoxacyclotetracosin-5,9,13,21,25-pentone, 7,8,11,12,15,16,23,24,27,28-decahydro-2,4,18,20-tetrahydroxy-11-(hydroxymethyl)-7,15,23,27-tetramethyl-), NG-121, SS-701 (2,2′:6′,2″-terpyridine, 4′-(4-methylphenyl)-, trihydrochloride), piperidine, 1-(6-quinoxalinylcarbonyl)-sold under the tradename AMPALEX (Cortex Pharmaceuticals, Inc.; Irvine, Calif.), RGH-2716 (8-[4,4-bis(4-fluorophenyl)butyl]-3-(1,1-dimethylethyl)-4-methylene-1-oxa-3,8-diaza-spiro[4.5]decan-2-one), and TDN-345 (1-Oxa-3,8-diazaspiro[4.5]decan-2-one, 8-[4,4-bis(4-fluorophenyl)butyl]-3-(1,1-dimethylethyl)-4-methylene-).

Other examples of compounds which are capable of stimulating cellular processes which result in tissue growth include pyruvic acid, hyaluronic acid, naltrexone, estrogen, leptin, statins, D-glucose, insulin, sphingosine 1-phosphate, amlodipine, alginate oligosaccharides, and minoxidil, including analogues and derivatives of these.

Within various embodiments of the invention, a device is coated on one aspect with a composition which promotes fibrosis, neointimal hyperplasia and/or restenosis (typically on the adluminal surface of the device), as well as being coated with a composition or compound which prevents scarring, neointimal hyperplasia or restenosis on another aspect of the device (typically on the luminal surface of the device). Representative examples of agents that inhibit restenosis include paclitaxel, sirolimus, everolimus, tacrolimus, vincristine, biolimus mycophenolic acid, ABT-578, cervistatin, simvastatin, methylprednisolone, dexamethasone, actinomycin-D, angiopeptin, L-arginine, estradiol, 17-β-estradiol, tranilast, methotrexate, batimistat, halofuginone, BCP-671, QP-2, lantrunculin D, cytochalasin A, nitric oxide and analogues and derivatives thereof.

The medical implant may include a fibrosing agent as well as an anti-thrombotic agent and/or antiplatelet agent, which reduces the likelihood of thrombotic events upon implantation of a medical implant within the lumen of the blood vessel. Within various embodiments of the invention, a device (e.g., a stent graft or stent) is coated on one aspect with a composition which promotes fibrosis and/or restenosis (typically on the adluminal aspect of the device), as well as being coated with a composition or compound which prevents thrombosis on another aspect of the device (typically the luminal aspect of the device). Representative examples of anti-thrombotic and/or antiplatelet agents include heparin, heparin fragments, organic salts of heparin, heparin complexes (e.g., benzalkonium heparinate, tridodecylammonium heparinate), dextran, sulfonated carbohydrates such as dextran sulphate, coumadin, coumarin, heparinoid, danaparoid, argatroban chitosan sulfate, chondroitin sulfate, danaparoid, lepirudin, hirudin, AMP, adenosine, 2-chloroadenosine, aspirin, phenylbutazone, indomethacin, meclofenamate, hydrochloroquine, dipyridamole, iloprost, factor Xa inhibitors, such as DX9065a, magnesium, and tissue plasminogen activator. In one aspect, the anti-thrombotic agent is a modified heparin compound, such as a hydrophobically modified heparin or modified hirudin compound (e.g., stearylkonium heparin, benzalkonium heparin, cetylkonium heparin, or trdodecylmethyl ammonium heparin). Further examples of anti-thrombotic agents include plasminogen, lys-plasminogen, ticlopidine, clopidogrel, glycoprotein lib/IIIa inhibitors such as abcixamab, eptifibatide, and tirogiban. Other agents capable of affecting the rate of clotting include glycosaminoglycans, danaparoid, 4-hydroxycourmarin, warfarin sodium, dicumarol, phenprocoumon, indan-1,3-dione, acenocoumarol, anisindione, and rodenticides including bromadiolone, brodifacoum, diphenadione, chlorophacinone, and pidnone. The thrombogenicity of a medical implant may be reduced by coating the implant with a polymeric formulation that has anti-thrombogenic properties. For example, a medical device may be coated with a hydrophilic polymer gel. The polymer gel can comprise a hydrophilic, biodegradable polymer that is physically removed from the surface of the device over time, thus reducing adhesion of platelets to the device surface. The gel composition can include a polymer or a blend of polymers. Representative examples include alginates, chitosan and chitosan sulfate, hyaluronic acid, dextran sulfate, PLURONIC polymers (e.g., F-127 or F87) and chain extended PLURONIC polymers (BASF Corporation, Mt. Olive, N.J.), various polyester-polyether block copolymers of various configurations (e.g., AB, ABA, or BAB, where A is a polyester such as PLA, PGA, PLGA, PCL or the like), examples of which include MePEG-PLA, PLA-PEG-PLA, and the like). In one embodiment, the anti-thrombotic composition can include a crosslinked gel formed from a combination of molecules (e.g., PEG) having two or more terminal electrophilic groups and two or more nucleophilic groups.

Within various embodiments of the invention, a device is coated on one aspect with a composition which promotes fibrosis (and/or restenosis), as well as being coated with a composition or compound which promotes fibrinolysis and/or thrombolysis on another aspect of the device. Representative examples of agents which promote fibrinolysis and/or thrombolysis include plasminogen, alpha-2-antiplasmin, streptokinase, tissue plasminogen activator (t-PA), urokinase, aminocaproic acid, and analogues and derivatives.

The medical implant may include a fibrosing agent and an agent that reduces the likelihood of infection upon implantation of a medical implant. Within various embodiments of the invention, a device is coated on one aspect with a composition which promotes fibrosis (and/or restenosis), as well as being coated with a composition or compound which prevents infection on another aspect of the device.

In one aspect, the present invention also provides for the combination of a medical implant (as well as compositions and methods for making medical implants) that includes a fibrosing agent and an anti-infective agent, which reduces the likelihood of infections in medical implants. Infection is a common complication of the implantation of foreign bodies such as medical devices. Foreign materials provide an ideal site for micro-organisms to attach and colonize. It is also hypothesized that there is an impairment of host defenses to infection in the microenvironment surrounding a foreign material. These factors make medical implants particularly susceptible to infection and make eradication of such an infection difficult, if not impossible, in most cases.

The present invention provides agents (e.g., chemotherapeutic agents) that can be released from an implantable device, and which have potent antimicrobial activity at extremely low doses. A wide variety of anti-infective agents can be utilized in combination with a fibrosing agent according to the invention. Discussed in more detail below are several representative examples of agents that can be used: (A) anthracyclines (e.g., doxorubicin and mitoxantrone), (B) fluoropyrimidines (e.g., 5-FU), (C) folic acid antagonists (e.g., methotrexate), (D) podophylotoxins (e.g., etoposide), (E) camptothecins, (F) hydroxyureas, and (G) platinum complexes (e.g., cisplatin).

B. Anthracyclines

Anthracyclines have the following general structure, where the R groups may be a variety of organic groups:

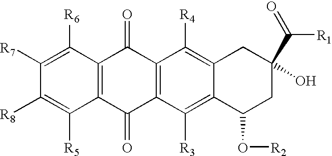

According to U.S. Pat. No. 5,594,158, suitable R groups are as follows: R₁ is CH₃ or CH₂OH; R₂ is daunosamine or H; R₃ and R₄ are independently one of OH, NO₂, NH₂, F, Cl, Br, I, CN, H or groups derived from these; R₅ is hydrogen, hydroxyl, or methoxy; and R₆₋₈ are all hydrogen. Alternatively, R₅ and R₆ are hydrogen and R₇ and R₈ are alkyl or halogen, or vice versa.

According to U.S. Pat. No. 5,843,903, R₁ may be a conjugated peptide. According to U.S. Pat. No. 4,296,105, R₅ may be an ether linked alkyl group. According to U.S. Pat. No. 4,215,062, R₅ may be OH or an ether linked alkyl group. R₁ may also be linked to the anthracycline ring by a group other than C(O), such as an alkyl or branched alkyl group having the C(O) linking moiety at its end, such as —CH₂CH(CH₂—X)C(O)—R₁, wherein X is H or an alkyl group (see, e.g., U.S. Pat. No. 4,215,062). R₂ may alternately be a group linked by the functional group ═N—NHC(O)—Y, where Y is a group such as a phenyl or substituted phenyl ring. Alternately R₃ may have the following structure: 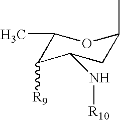
in which R₉ is OH either in or out of the plane of the ring, or is a second sugar moiety such as R₃. R₁₀ may be H or form a secondary amine with a group such as an aromatic group, saturated or partially saturated 5 or 6 membered heterocyclic having at least one ring nitrogen (see U.S. Pat. No. 5,843,903). Altemately, R₁₀ may be derived from an amino acid, having the structure —C(O)CH(NHR₁₁)(R₁₂), in which R₁₁ is H, or forms a C₃₋₄ membered alkylene with R₁₂. R₁₂ may be H, alkyl, aminoalkyl, amino, hydroxyl, mercapto, phenyl, benzyl or methylthio (see U.S. Pat. No. 4,296,105).

Exemplary anthracyclines are doxorubicin, daunorubicin, idarubicin, epirubicin, pirarubicin, zorubicin, and carubicin. Suitable compounds have the structures:

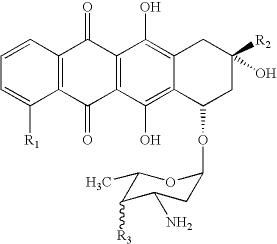

| | R₁ | R₂ | R₃ |
|---|---|---|---|
| Doxorubicin: | OCH₃ | C(O)CH₂OH | OH out of ring plane |
| Epirubicin: | OCH₃ | C(O)CH₂OH | OH in ring plane |
| (4′ epimer of | | | |
| doxorubicin) | | | |
| Daunorubicin: | OCH₃ | C(O)CH₃ | OH out of ring plane |
| Idarubicin: | H | C(O)CH₃ | OH out of ring plane |
| Pirarubicin: | OCH₃ | C(O)CH₂OH | 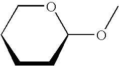 |
| Zorubicin: | OCH₃ | C(CH₃)(═N)NHC(O)C₆H₅ | OH |
| Carubicin: | OH | C(O)CH₃ | OH out of ring plane |

Other suitable anthracyclines are anthramycin, mitoxantrone, menogaril, nogalamycin, aclacinomycin A, olivomycin A, chromomycin A₃, and plicamycin having the structures:

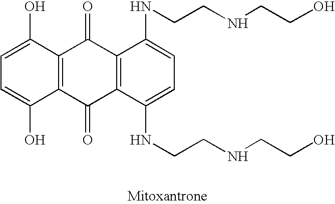

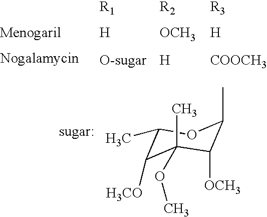

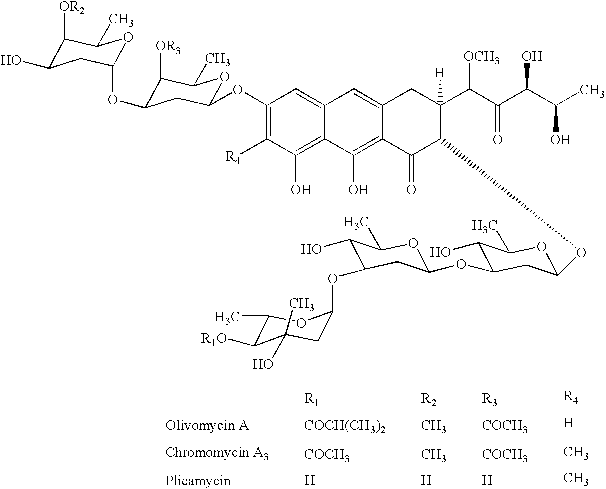

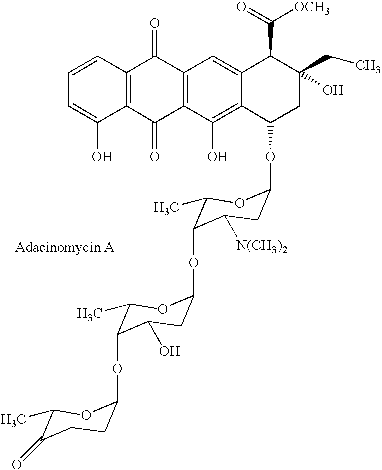

Other representative anthracyclines include, FCE 23762, a doxorubicin derivative (Quaglia et al., J. Liq. Chromatogr. 17(18):3911-3923, 1994), annamycin (Zou et al., J. Pharm. Sci. 82(11):1151-1154, 1993), ruboxyl (Rapoport et al., J. Controlled Release 58(2):153-162, 1999), anthracycline disaccharide doxorubicin analogue (Pratesi et al., Clin. Cancer Res. 4(11):2833-2839,1998), N-(trifluoroacetyl)doxorubicin and 4′-O-acetyl-N-(trifluoroacetyl)doxorubicin (Berube & Lepage, Synth. Commun. 28(6):1109-1116, 1998), 2-pyrrolinodoxorubicin (Nagy et al., Proc. Natl Acad. Sci. U.S.A. 95(4):1794-1799,1998), disaccharide doxorubicin analogues (Arcamone et al., J. Nat'l Cancer Inst. 89(16):1217-1223,1997), 4-demethoxy-7-O-[2,6-dideoxy-4-O-(2,3,6-trideoxy-3-amino-α-L-lyxo-hexopyranosyl)-α-L-lyxo-hexopyranosyl]-adriamicinone doxorubicin disaccharide analogue (Monteagudo et al., Carbohydr. Res. 300(1):11-16,1997), 2-pyrrolinodoxorubicin (Nagy et al., Proc. Nat'l Acad. Sci. U.S.A. 94(2):652-656, 1997), morpholinyl doxorubicin analogues (Duran et al., Cancer Chemother. Pharmacol. 38(3):210-216, 1996), enaminomalonyl-β-alanine doxorubicin derivatives (Seitz et al., Tetrahedron Lett. 36(9):1413-16, 1995), cephalosporin doxorubicin derivatives (Vrudhula et al., J. Med. Chem. 38(8):1380-5, 1995), hydroxyrubicin (Solary et al., Int J. Cancer 58(1):85-94, 1994), methoxymorpholino doxorubicin derivative (Kuhl et al., Cancer Chemother. Pharmacol. 33(1):10-16, 1993), (6-maleimidocaproyl)hydrazone doxorubicin derivative (Willner et al., Bioconjugate Chem. 4(6):521-7, 1993), N-(5,5-diacetoxypent-1-yl) doxorubicin (Cherif & Farquhar, J. Med. Chem. 35(17):3208-14, 1992), FCE 23762 methoxymorpholinyl doxorubicin derivative (Ripamonti et al., Br. J. Cancer 65(5):703-7, 1992), N-hydroxysuccinimide ester doxorubicin derivatives (Demant et al., Biochim. Biophys. Acta 1118(1):83-90,1991), polydeoxynucleotide doxorubicin derivatives (Ruggiero et al., Biochim. Biophys. Acta 1129(3):294-302, 1991), morpholinyl doxorubicin derivatives (EPA 434960), mitoxantrone doxorubicin analogue (Krapcho et al., J. Med. Chem. 34(8):2373-80. 1991), AD198 doxorubicin analogue (Traganos et al., Cancer Res. 51(14):3682-9, 1991), 4-demethoxy-3′-N-trifluoroacetyldoxorubicin (Horton et al., Drug Des. Delivery 6(2):123-9,1990), 4′-epidoxorubicin (Drzewoski et al., Pol. J Pharmacol. Pharm. 40(2):159-65,1988; Weenen et al., Eur. J. Cancer Clin. Oncol. 20(7):919-26, 1984), alkylating cyanomorpholino doxorubicin derivative (Scudder et al., J. Nat'l Cancer Inst 80(16):1294-8, 1988), deoxydihydroiodooxorubicin (EPA 275966), adriblastin (Kalishevskaya et al., Vestn. Mosk. Univ., 16(Biol. 1):21-7, 1988), 4′-deoxydoxorubicin (Schoelzel et al., Leuk. Res. 10(12):1455-9, 1986), 4-demethyoxy-4′-o-methyldoxorubicin (Giuliani et al., Proc. Int. Congr. Chemother. 16:285-70-285-77,1983), 3′-deamino-3′-hydroxydoxorubicin (Horton et al., J. Antibiot 37(8):853-8, 1984), 4-demethyoxy doxorubicin analogues (Barbieri et al., Drugs Exp. Clin. Res. 10(2):85-90, 1984), N-L-leucyl doxorubicin derivatives (Trouet et al., Anthracyclines (Proc. Int. Symp. Tumor Pharmacother.), 179-81, 1983), 3′-deamino-3′-(4-methoxy-1-piperidinyl) doxorubicin derivatives (U.S. Pat. No. 4,314,054), 3′-deamino-3′-(4-mortholinyl) doxorubicin derivatives (U.S. Pat. No. 4,301,277), 4′-deoxydoxorubicin and 4′-o-methyldoxorubicin (Giuliani et al., Int. J. Cancer 27(1):5-13, 1981), aglycone doxorubicin derivatives (Chan & Watson, J. Pharm. Sci. 67(12):1748-52, 1978), SM 5887 (Pharma Japan 1468:20,1995), MX-2 (Pharma Japan 1420:19, 1994), 4′-deoxy-13(S)-dihydro-4′-iododoxorubicin (EP 275966), morpholinyl doxorubicin derivatives (EPA 434960), 3′-deamino-3′-(4-methoxy-1-piperidinyl) doxorubicin derivatives (U.S. Pat. No. 4,314,054), doxorubicin-14-valerate, morpholinodoxorubicin (U.S. Pat. No. 5,004,606), 3′-deamino-3′-(3″-cyano-4″-morpholinyl doxorubicin; 3′-deamino-3′-(3″-cyano-4″-morpholinyl)-13-dihydoxorubicin; (3′-deamino-3′-(3″-cyano-4″-morpholinyl) daunorubicin; 3′-deamino-3′-(3″-cyano-4″-morpholinyl)-3-dihydrodaunorubicin; and 3′-deamino-3′-(4″-morpholinyl-5-iminodoxorubicin and derivatives (U.S. Pat. No. 4,585,859), 3′-deamino-3′-(4-methoxy-1-piperidinyl) doxorubicin derivatives (U.S. Pat. No. 4,314,054) and 3-deamino-3-(4-morpholinyl) doxorubicin derivatives (U.S. Pat. No. 4,301,277).

C. Fluoropyrimidine Analogues

In another aspect, the therapeutic agent is a fluoropyrimidine analog, such as 5-fluorouracil, or an analogue or derivative thereof, including carmofur, doxifluridine, emitefur, tegafur, and floxuridine. Exemplary compounds have the structures:

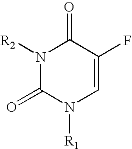

| | R₁ | R₂ |
|---|---|---|
| 5-Fluorouracil | H | H |
| Carmofur | C(O)NH(CH₂)₅CH₃ | H |
| Doxifluridine | A₁ | H |
| Floxuridine | A₂ | H |
| Emitefur | CH₂OCH₂CH₃ | B |
| Tegafur | C | H |

B

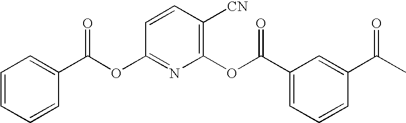

C

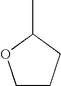

Other suitable fluoropyrimidine analogues include 5-FudR (5-fluoro-deoxyuridine), or an analogue or derivative thereof, including 5-iododeoxyuridine (5-IudR), 5-bromodeoxyuridine (5-BudR), fluorouridine triphosphate (5-FUTP), and fluorodeoxyuridine monophosphate (5-dFUMP). Exemplary compounds have the structures:

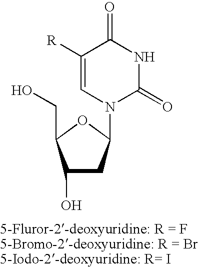

Other representative examples of fluoropyrimidine analogues include N3-alkylated analogues of 5-fluorouracil (Kozai et al., J. Chem. Soc., Perkin Trans. 1(19):3145-3146, 1998), 5-fluorouracil derivatives with 1,4-oxaheteroepane moieties (Gomez et al., Tetrahedron 54(43):13295-13312, 1998), 5-fluorouracil and nucleoside analogues (Li, Anticancer Res. 17(1A):21-27, 1997), cis- and trans-5-fluoro-5,6-dihydro-6-alkoxyuracil (Van der Wilt et al., Br. J. Cancer 68(4):702-7, 1993), cyclopentane 5-fluorouracil analogues (Hronowski & Szarek, Can. J. Chem. 70(4):1162-9,1992), A-OT-fluorouracil (Zhang et al., Zongguo Yiyao Gongye Zazhi 20(11):513-15, 1989), N4-trimethoxybenzoyl-5′-deoxy-5-fluorocytidine and 5′-deoxy-5-fluorouridine (Miwa et al., Chem. Pharm. Bull. 38(4):998-1003, 1990), 1-hexylcarbamoyl-5-fluorouracil (Hoshi et al., J. Pharmacobio-Dun. 3(9):478-81, 1980; Maehara et al., Chemotherapy (Basel) 34(6):484-9, 1988), B-3839 (Prajda et al., In Vivo 2(2):151-4, 1988), uracil-1-(2-tetrahydrofuryl)-5-fluorouracil (Anai et al., Oncology 45(3):144-7, 1988), 1-(2′-deoxy-2′-fluoro-β-D-arabinofuranosyl)-5-fluorouracil (Suzuko et al., Mol. Pharmacol. 31(3):301-6, 1987), doxifluridine (Matuura et al., Oyo Yakuri 29(5):803-31, 1985), 5′-deoxy-5-fluorouridine (Bollag & Hartmann, Eur. J. Cancer 16(4):427-32, 1980), 1-acetyl-3-O-toluyl-5-fluorouracil (Okada, Hiroshima J. Med. Sci. 28(1):49-66,1979), 5-fluorouracil-m-formylbenzene-sulfonate (JP 55059173), N′-(2-furanidyl)-5-fluorouracil (JP 53149985) and 1-(2-tetrahydrofuryl)-5-fluorouracil (JP 52089680).

These compounds are believed to function as therapeutic agents by serving as antimetabolites of pyrimidine.

D. IFolic Acid Antagonists

In another aspect, the therapeutic agent is a folic acid antagonist, such as methotrexate or derivatives or analogues thereof, including edatrexate, trimetrexate, raltitrexed, piritrexim, denopterin, tomudex, and pteropterin. Methotrexate analogues have the following general structure:

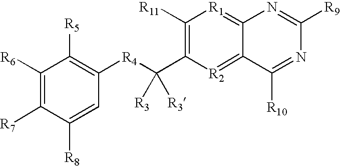

The identity of the R group may be selected from organic groups, particularly those groups set forth in U.S. Pat. Nos. 5,166,149 and 5,382,582. For example, R₁ may be N, R₂ may be N or C(CH₃), R₃ and R₃′ may H or alkyl, e.g., CH₃, R₄ may be a single bond or NR, where R is H or alkyl group. R_{5,6,8} may be H, OCH₃, or alternately they can be halogens or hydro groups. R₇ is a side chain of the general structure: 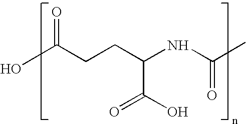
wherein n=1 for methotrexate, n=3 for pteropterin. The carboxyl groups in the side chain may be esterified or form a salt such as a Zn²⁺ salt. R₉ and R₁₀ can be NH₂ or may be alkyl substituted.

Exemplary folic acid antagonist compounds have the structures:

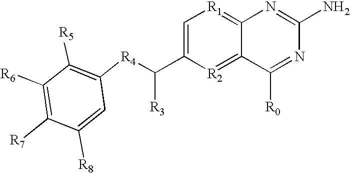

| | R₀ | R₁ | R₂ | R₃ | R₄ | R₅ | R₆ | R₇ | R₈ |
|---|---|---|---|---|---|---|---|---|---|
| Methotrexate | NH₂ | N | N | H | N(CH₃) | H | H | A (n = 1) | H |
| Edatrexate | NH₂ | N | N | H | CH(CH₂CH₃) | H | H | A (n = 1) | H |
| Trimetrexate | NH₂ | CH | C(CH₃) | H | NH | H | OCH₃ | OCH₃ | OCH₃ |
| Pteropterin | OH | N | N | H | NH | H | H | A (n = 3) | H |
| Denopterin | OH | N | N | CH₃ | N(CH₃) | H | H | A(n = 1) | H |
| Peritrexim | NH₂ | N | C(CH₃) | H | single bond | OCH₃ | H | H | OCH₃ |

A:

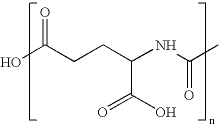

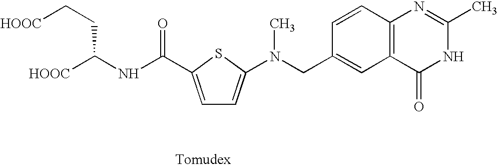

Other representative examples include 6-S-aminoacyloxymethyl mercaptopurine derivatives (Harada et al., Chem. Pharm. Bull. 43(10):793-6, 1995), 6-mercaptopurine (6-MP) (Kashida et al., Biol. Pharm. Bull. 18(11):1492-7, 1995), 7,8-polymethyleneimidazo-1,3,2-diazaphosphorines (Nilov et al., Mendeleev Commun. 2:67,1995), azathioprine (Chifotides et al., J. Inorg. Biochem. 56(4):249-64, 1994), methyl-D-glucopyranoside mercaptopurine derivatives (Da Silva et al., Eur. J. Med. Chem. 29(2):149-52, 1994) and s-alkynyl mercaptopurine derivatives (Ratsino et al., Khim.-Farm. Zh. 15(8):65-7,1981); indoline ring and a modified ornithine or glutamic acid-bearing methotrexate derivatives (Matsuoka et al., Chem. Pharm. Bull. 45(7):1146-1150,1997), alkyl-substituted benzene ring C bearing methotrexate derivatives (Matsuoka et al., Chem. Pharm. Bull. 44(12):2287-2293, 1996), benzoxazine or benzothiazine moiety-bearing methotrexate derivatives (Matsuoka et al., J. Med. Chem. 40(1):105-111, 1997), 10-deazaaminopterin analogues (DeGraw et al., J. Med. Chem. 40(3):370-376, 1997), 5-deazaaminopterin and 5,10-dideazaaminopterin methotrexate analogues (Piper et al., J. Med. Chem. 40(3):377-384, 1997), indoline moiety-bearing methotrexate derivatives (Matsuoka et al., Chem. Pharm. Bull. 44(7):1332-1337, 1996), lipophilic amide methotrexate derivatives (Pignatello et al., World Meet. Pharm. Biopharm. Pharm. Technol., 563-4,1995), L-threo-(2S,4S)-4-fluoroglutamic acid and DL-3,3-difluoroglutamic acid-containing methotrexate analogues (Hart et al., J. Med. Chem. 39(1):56-65, 1996), methotrexate tetrahydroquinazoline analogue (Gangjee, et al., J. Heterocycl. Chem. 32(1):243-8,1995), N-(α-aminoacyl) methotrexate derivatives (Cheung et al., Pteridines 3(1-2):101-2, 1992), biotin methotrexate derivatives (Fan et al., Pteridines 3(1-2):131-2, 1992), D-glutamic acid or D-erythrou, threo-4-fluoroglutamic acid methotrexate analogues (McGuire et al., Biochem. Pharmacol. 42(12):2400-3, 1991), β,γ-methano methotrexate analogues (Rosowsky et al., Pteridines 2(3):133-9, 1991), 10-deazaaminopterin (10-EDAM) analogue (Braakhuis et al., Chem. Biol. Pteridines, Proc. Int. Symp. Pteridines Folic Acid Deriv., 1027-30, 1989), γ-tetrazole methotrexate analogue (Kalman et al., Chem. Biol. Pteridines, Proc. Int. Symp. Pteridines Folic Acid Deriv., 1154-7, 1989), N-(L-α-aminoacyl) methotrexate derivatives (Cheung et al., Heterocycles 28(2):751-8, 1989), meta and ortho isomers of aminopterin (Rosowsky et al., J. Med. Chem. 32(12):2582, 1989), hydroxymethylmethotrexate (DE 267495), γ-fluoromethotrexate (McGuire et al., Cancer Res. 49(16):4517-25, 1989), polyglutamyl methotrexate derivatives (Kumar et al., Cancer Res. 46(10):5020-3, 1986), gem-diphosphonate methotrexate analogues (WO 88/06158), α- and γ-substituted methotrexate analogues (Tsushima et al., Tetrahedron 44(17):5375-87, 1988), 5-methyl-5-deaza methotrexate analogues (U.S. Pat. No. 4,725,687), N8-acyl-Na-(4-amino-4-deoxypteroyl)-L-ornithine derivatives (Rosowsky et al., J. Med. Chem. 31(7):1332-7, 1988), 8-deaza methotrexate analogues (Kuehl et al., Cancer Res. 48(6):1481-8, 1988), acivicin methotrexate analogue (Rosowsky et al., J. Med. Chem. 30(8):1463-9, 1987), polymeric platinol methotrexate derivative (Carraher et al., Polym. Sci. Technol. (Plenum), 35(Adv. Biomed. Polym.):311-24, 1987), methotrexate-γ-dimyristoylphophatidylethanolamine (Kinsky et al., Biochim. Biophys. Acta 917(2):211-18, 1987), methotrexate polyglutamate analogues (Rosowsky et al., Chem. Biol. Pteridines, Pteridines Folid Acid Deriv., Proc. Int. Symp. Pteridines Folid Acid Deriv.: Chem., Biol. Clin. Aspects: 985-8, 1986), poly-γ-glutamyl methotrexate derivatives (Kisliuk et al., Chem. Biol. Pteridines, Pteridines Folid Acid Deriv., Proc. Int. Symp. Pteridines Folid Acid Deriv.: Chem., Biol. Clin. Aspects: 989-92, 1986), deoxyuridylate methotrexate derivatives (Webber et al., Chem. Biol. Pteridines, Pteridines Folid Acid Deriv., Proc. Int. Symp. Pteridines Folid Acid Deriv.: Chem., Biol. Clin. Aspects: 659-62,1986), iodoacetyl lysine methotrexate analogue (Delcamp et al., Chem. Biol. Pteridines, Pteridines Folid Acid Deriv., Proc. Int. Symp. Pteridines Folid Acid Deriv.: Chem., Biol. Clin. Aspects: 807-9, 1986), 2,.omega.-diaminoalkanoid acid-containing methotrexate analogues (McGuire et al., Biochem. Pharmacol. 35(15):2607-13, 1986), polyglutamate methotrexate derivatives (Kamen & Winick, Methods Enzymol. 122(Vitam. Coenzymes, Pt. G):339-46, 1986), 5-methyl-5-deaza analogues (Piper et al., J. Med. Chem. 29(6):1080-7, 1986), quinazoline methotrexate analogue (Mastropaolo et al., J. Med. Chem. 29(1):155-8, 1986), pyrazine methotrexate analogue (Lever & Vestal, J. Heterocycl. Chem. 22(1):5-6, 1985), cysteic acid and homocysteic acid methotrexate analogues (4,490,529), γ-tert-butyl methotrexate esters (Rosowsky et al., J. Med. Chem. 28(5):660-7, 1985), fluorinated methotrexate analogues (Tsushima et al., Heterocycles 23(1):45-9, 1985), folate methotrexate analogue (Trombe, J. Bacteriol. 160(3):849-53, 1984), phosphonoglutamic acid analogues (Sturtz & Guillamot, Eur. J. Med. Chem.-Chim. Ther. 19(3):267-73, 1984), poly (L-lysine) methotrexate conjugates (Rosowsky et al., J. Med. Chem. 27(7):888-93, 1984), dilysine and trilysine methotrexate derivates (Forsch & Rosowsky, J. Org. Chem. 49(7):1305-9, 1984), 7-hydroxymethotrexate (Fabre et al., Cancer Res. 43(10):4648-52, 1983), poly-γ-glutamyl methotrexate analogues (Piper & Montgomery, Adv. Exp. Med. Biol., 163(Folyl Antifolyl Polyglutamates):95-100, 1983), 3′,5′-dichloromethotrexate (Rosowsky & Yu, J. Med. Chem. 26(10):1448-52, 1983), diazoketone and chloromethylketone methotrexate analogues (Gangjee et al., J. Pharm. Sci. 71(6):717-19, 1982), 10-propargylaminopterin and alkyl methotrexate homologs (Piper et al., J. Med. Chem. 25(7):877-80, 1982), lectin derivatives of methotrexate (Lin et al., JNCI 66(3):523-8, 1981), polyglutamate methotrexate derivatives (Galivan, Mol. Pharmacol. 17(1):105-10, 1980), halogentated methotrexate derivatives (Fox, JNCI 58(4):J955-8, 1977), 8-alkyl-7,8-dihydro analogues (Chaykovsky et al., J. Med. Chem. 20(10):J1323-7, 1977), 7-methyl methotrexate derivatives and dichloromethotrexate (Rosowsky & Chen, J. Med. Chem. 17(12):J1308-11, 1974), lipophilic methotrexate derivatives and 3′,5′-dichloromethotrexate (Rosowsky, J. Med. Chem. 16(10):J1 190-3, 1973), deaza amethopterin analogues (Montgomery et al., Ann. N.Y. Acad. Sci. 186:J227-34, 1971), MX068 (Pharma Japan, 1658:18, 1999) and cysteic acid and homocysteic acid methotrexate analogues (EPA 0142220);

These compounds are believed to act as antimetabolites of folic acid.

E. Podophyllotoxins

In another aspect, the therapeutic agent is a Podophyllotoxin, or a derivative or an analogue thereof. Exemplary compounds of this type are etoposide or teniposide, which have the following structures:

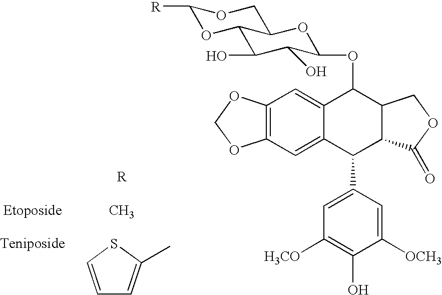

Other representative examples of podophyllotoxins include Cu(lI)-VP-16 (etoposide) complex (Tawa et al., Bioorg. Med. Chem. 6(7):1003-1008, 1998), pyrrolecarboxamidino-bearing etoposide analogues (Ji et al., Bioorg. Med. Chem. Lett. 7(5):607-612, 1997), 4β-amino etoposide analogues (Hu, University of North Carolina Dissertation, 1992), γ-lactone ring-modified arylamino etoposide analogues (Zhou et al., J. Med. Chem. 37(2):287-92, 1994), N-glucosyl etoposide analogue (Allevi et al., Tetrahedron Leff. 34(45):7313-16, 1993), etoposide A-ring analogues (Kadow et al., Bioorg. Med. Chem. Lett. 2(1):17-22, 1992), 4′-deshydroxy-4′-methyl etoposide (Saulnier et al., Bioorg. Med. Chem. Lett. 2(10):1213-18, 1992), pendulum ring etoposide analogues (Sinha et al., Eur. J. Cancer 26(5):590-3, 1990) and E-ring desoxy etoposide analogues (Saulnier et al., J. Med. Chem. 32(7):1418-20, 1989).

These compounds are believed to act as topoisomerase II inhibitors and/or DNA cleaving agents.

F. Camptothecins

In another aspect, the therapeutic agent is camptothecin, or an analogue or derivative thereof. Camptothecins have the following general structure.

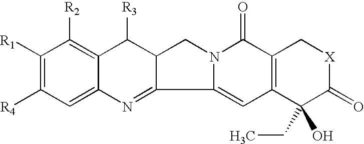

In this structure, X is typically O, but can be other groups, e.g., NH in the case of 21-lactam derivatives. R₁ is typically H or OH, but may be other groups, e.g., a terminally hydroxylated C₁₋₃ alkane. R₂ is typically H or an amino containing group such as (CH₃)₂NHCH₂, but may be other groups e.g., NO₂, NH₂, halogen (as disclosed in, e.g., U.S. Pat. No. 5,552,156) or a short alkane containing these groups. R₃ is typically H or a short alkyl such as C₂H₅. R₄ is typically H but may be other groups, e.g., a methylenedioxy group with R_{1.}

Exemplary camptothecin compounds include topotecan, irinotecan (CPT-11), 9-aminocamptothecin, 21-lactam-20(S)-camptothecin, 10,11-methylenedioxycamptothecin, SN-38, 9-nitrocamptothecin, 10-hydroxycamptothecin. Exemplary compounds have the structures:

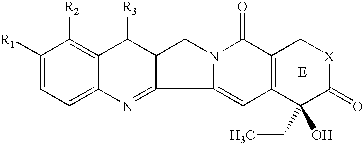

| | R₁ | R₂ | R₃ |
|---|---|---|---|
| Camptothecin: | H | H | H |
| Topotecan: | OH | (CH₃)₂NHCH₂ | H |
| SN-38: | OH | H | C₂H₅ |

X: O for most analogs, NH for 21-lactam analogs

Camptothecins have the five rings shown here. The ring labeled E must be intact (the lactone rather than carboxylate form) for maximum activity and minimum toxicity.

Camptothecins are believed to function as topoisomerase I inhibitors and/or DNA cleavage agents.

G. Hydroxyureas

The therapeutic agent of the present invention may be a hydroxyurea. Hydroxyureas have the following general structure:

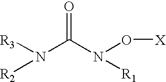

Suitable hydroxyureas are disclosed in, for example, U.S. Pat. No. 6,080,874, wherein R₁ is: 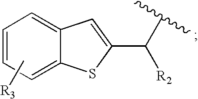
and R₂ is an alkyl group having 1-4 carbons and R₃ is one of H, acyl, methyl, ethyl, and mixtures thereof, such as a methylether.

Other suitable hydroxyureas are disclosed in, e.g., U.S. Pat. No. 5,665,768, wherein R₁ is a cycloalkenyl group, for example N-[3-[5-(4-fluorophenylthio)-furyl]-2-cyclopenten-1-yl]N-hydroxyurea; R₂ is H or an alkyl group having 1 to 4 carbons and R₃ is H; X is H or a cation.

Other suitable hydroxyureas are disclosed in, e.g., U.S. Pat. No. 4,299,778, wherein R₁ is a phenyl group substituted with one or more fluorine atoms; R₂ is a cyclopropyl group; and R₃ and X is H.

Other suitable hydroxyureas are disclosed in, e.g., U.S. Pat. No. 5,066,658, wherein R₂ and R₃ together with the adjacent nitrogen form: 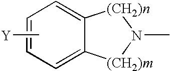
wherein m is 1 or 2, n is 0-2 and Y is an alkyl group.

In one aspect, the hydroxyurea has the structure:

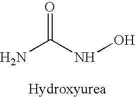

These compounds are thought to function by inhibiting DNA synthesis.

H. Platinum complexes

In another aspect, the therapeutic agent is a platinum compound. In general, suitable platinum complexes may be of Pt(II) or Pt(IV) and have this basic structure: 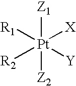
wherein X and Y are anionic leaving groups such as sulfate, phosphate, carboxylate, and halogen; R₁ and R₂ are alkyl, amine, amino alkyl any may be further substituted, and are basically inert or bridging groups. For Pt(II) complexes Z₁ and Z₂ are non-existent. For Pt(IV) Z₁ and Z₂ may be anionic groups such as halogen, hydroxyl, carboxylate, ester, sulfate or phosphate. See, e.g., U.S. Pat. Nos. 4,588,831 and 4,250,189.

Suitable platinum complexes may contain multiple Pt atoms. See, e.g., U.S. Pat. Nos. 5,409,915 and 5,380,897. For example bisplatinum and triplatinum complexes of the type:

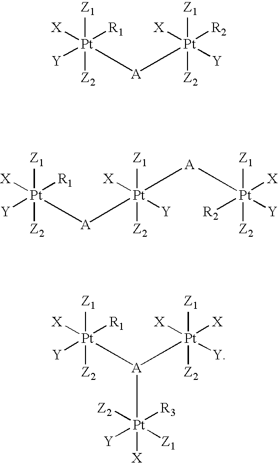

Exemplary platinum compounds are cisplatin,. carboplatin, oxaliplatin, and miboplatin having the structures:

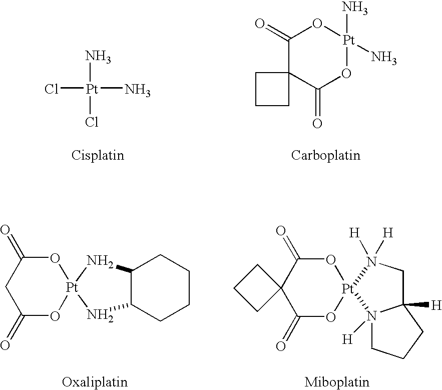

Other representative platinum compounds include (CPA)₂Pt[DOLYM] and (DACH)Pt[DOLYM]cisplatin (Choi et al., Arch. Pharmacal Res. 22(2):151-156, 1999), Cis-[PtCI₂(4,7-H-5-methyl-7-oxo]1,2,4[triazolo[1,5-a]pyrimidine)₂] Navarro et al., J. Med. Chem. 41(3):332-338, 1998), [Pt(cis-1,4-DACH)(trans-Cl₂)(CBDCA)].½MeOH cisplatin (Shamsuddin et al., Inorg. Chem. 36(25):5969-5971, 1997), 4-pyridoxate diammine hydroxyl platinum (Tokunaga et al., Pharm. Sci. 3(7):353-356, 1997), Pt(II) . . . Pt(II) (Pt₂[NHCHN(C(CH₂)(CH₃))]₄) (Navarro et al., Inorg. Chem. 35(26):7829-7835, 1996), 254-S cisplatin analogue (Koga et al., Neurol. Res. 18(3):244-247, 1996), o-phenylenediamine ligand bearing cisplatin analogues (Koeckerbauer & Bednarski, J. Inorg. Biochem. 62(4):281-298, 1996), trans, cis-[Pt(Oac)₂I₂(en)] (Kratochwil et al., J. Med. Chem. 39(13):2499-2507, 1996), estrogenic 1,2-diarylethylenediamine ligand (with sulfur-containing amino acids and glutathione) bearing cisplatin analogues (Bednarski, J. Inorg. Biochem. 62(1):75, 1996), cis-1,4-diaminocyclohexane cisplatin analogues (Shamsuddin et al., J. Inorg. Biochem. 61(4):291-301, 1996), 5′ orientational isomer of cis-[Pt(NH₃)(4-aminoTEMP-O){d(GpG)}] (Dunham & Lippard, J. Am. Chem. Soc. 117(43):10702-12, 1995), chelating diamine-bearing cisplatin analogues (Koeckerbauer & Bednarski, J. Pharm. Sci. 84(7):819-23,1995), 1,2-diarylethyleneamine ligand-bearing cisplatin analogues (Otto et al., J. Cancer Res. Clin. Oncol. 121(1):31-8, 1995), (ethylenediamine)platinum(II) complexes (Pasini et al., J. Chem. Soc., Dalton Trans. 4:579-85, 1995), Cl-973 cisplatin analogue (Yang et al., Int. J. Oncol. 5(3):597-602, 1994), cis-diaminedichloroplatinum(II) and its analogues cis-1,1-cyclobutanedicarbosylato(2R)-2-methyl-1,4-butanediamineplatinum(II) and cis-diammine(glycolato)platinum (Claycamp & Zimbrick, J. Inorg. Biochem. 26(4):257-67, 1986; Fan et al., Cancer Res. 48(11):3135-9, 1988; Heiger-Bernays et al., Biochemistry 29(36):8461-6, 1990; Kikkawa et al., J. Exp. Clin. Cancer Res. 12(4):233-40, 1993; Murray et al., Biochemistry 31(47):11812-17, 1992; Takahashi et al., Cancer Chemother. Pharmacol. 33(1):31-5, 1993), cis-amine-cyclohexylamine-dichloroplatinum(II) (Yoshida et al., Biochem. Pharmacol. 48(4):793-9, 1994), gem-diphosphonate cisplatin analogues (FR 2683529), (meso-1,2-bis(2,6-dichloro-4-hydroxyplenyl)ethylenediamine) dichloroplatinum(II) (Bednarski et al., J. Med. Chem. 35(23):4479-85, 1992), cisplatin analogues containing a tethered dansyl group (Hartwig et al., J. Am. Chem. Soc. 114(21):8292-3, 1992), platinum(II) polyamines (Siegmann et al., Inorg. Met.-Containing Polym. Mater., (Proc. Am. Chem. Soc. Int. Symp.), 335-61,1990), cis-(3H)dichloro(ethylenediamine)platinum(II) (Eastman, Anal. Biochem. 197(2):311-15, 1991), trans-diamminedichloroplatinum(II) and cis-(Pt(NH₃)₂(N₃-cytosine)Cl) (Bellon & Lippard, Biophys. Chem. 35(2-3):179-88, 1990), 3H-cis-1,2-diaminocyclohexanedichloroplatinum(II) and 3H-cis-1,2-diaminocyclohexane-malonatoplatinum (II) (Oswald et al., Res. Commun. Chem. Pathol. Pharmacol. 64(1):41-58, 1989), diaminocarboxylatoplatinum (EPA 296321), trans-(D,1)-1,2-diaminocyclohexane carrier ligand-bearing platinum analogues (Wyrick & Chaney, J. Labelled Compd. Radiopharm. 25(4):349-57,1988), aminoalkylaminoanthraquinone-derived cisplatin analogues (Kitov et al., Eur. J. Med. Chem. 23(4):381-3, 1988), spiroplatin, carboplatin, iproplatin and JM40 platinum analogues (Schroyen et al., Eur. J. Cancer Clin. Oncol. 24(8):1309-12, 1988), bidentate tertiary diamine-containing cisplatinum derivatives (Orbell et al., Inorg. Chim. Acta 152(2):125-34, 1988), platinum(II), platinum(IV) (Liu & Wang, Shandong Yike Daxue Xuebao 24(1):35-41, 1986), cis-diammine(1,1-cyclobutanedicarboxylato-)platinum(II) (carboplatin, JM8) and ethylenediammine-malonatoplatinum(II) (JM40) (Begg et al., Radiother. Oncol. 9(2):157-65, 1987), JM8 and JM9 cisplatin analogues (Harstrick et al., Int. J. Androl. 10(1); 139-45, 1987), (NPr4)2((PtCL4).cis-(PtCl2-(NH2Me)2)) (Brammer et al., J. Chem. Soc., Chem. Commun. 6:443-5, 1987), aliphatic tricarboxylic acid platinum complexes (EPA 185225), and cis-dichloro(amino acid)(tert-butylamine)platinum(II) complexes (Pasini & Bersanetti, Inorg. Chim. Acta 107(4):259-67, 1985). These compounds are thought to function by binding to DNA, i.e., acting as alkylating agents of DNA.

As medical implants are made in a variety of configurations and sizes, the exact dose administered will vary with device size, surface area, design and portions of the implant coated. However, certain principles can be applied in the application of this art. Drug dose can be calculated as a function of dose per unit area (of the portion of the device being coated), total drug dose administered can be measured and appropriate surface concentrations of active drug can be determined. Regardless of the method of application of the drug to the intravascular device or implant, the preferred anticancer agents, used alone or in combination, should be administered under the following dosing guidelines:

(a) Anthracyclines. Utilizing the anthracycline doxorubicin as an example, whether applied as a polymer coating, incorporated into the polymers which make up the implant components, or applied without a carrier polymer, the total dose of doxorubicin applied to the implant should not exceed 25 mg (range of 0.1 μg to 25 mg). In a particularly preferred embodiment, the total amount of drug applied should be in the range of 1 μg to 5 mg. The dose per unit area (i.e., the amount of drug as a function of the surface area of the portion of the implant to which drug is applied and/or incorporated) should fall within the range of 0.01 μg-100 μg per mm² of surface area. In a particularly preferred embodiment, doxorubicin should be applied to the implant surface at a dose of 0.1 μg/mm²-10 μg/mm². As different polymer and non-polymer coatings will release doxorubicin at differing rates, the above dosing parameters should be utilized in combination with the release rate of the drug from the implant surface such that a minimum concentration of 10⁻⁷-10⁻⁴ M of doxorubicin is maintained on the surface. It is necessary to insure that surface drug concentrations exceed concentrations of doxorubicin known to be lethal to multiple species of bacteria and fungi (i.e., are in excess of 10⁻⁴ M; although for some embodiments lower concentrations are sufficient). In a preferred embodiment, doxorubicin is released from the surface of the implant such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of doxorubicin (as described previously) with similar functional activity can be utilized for the purposes of this invention; the above dosing parameters are then adjusted according to the relative potency of the analogue or derivative as compared to the parent compound (e.g., a compound twice as potent as doxorubicin is administered at half the above parameters, a compound half as potent as doxorubicin is administered at twice the above parameters, etc.).

Utilizing mitoxantrone as another example of an anthracycline, whether applied as a polymer coating, incorporated into the polymers which make up the implant, or applied without a carrier polymer, the total dose of mitoxantrone applied should not exceed 5 mg (range of 0.01 μg to 5 mg). In a particularly preferred embodiment, the total amount of drug applied should be in the range of 0.1 μg to 1 mg. The dose per unit area (i.e., the amount of drug as a function of the surface area of the portion of the implant to which drug is applied and/or incorporated) should fall within the range of 0.01 μg-20 μg per mm² of surface area. In a particularly preferred embodiment, mitoxantrone should be applied to the implant surface at a dose of 0.05 μg/mm²-3 μg/mm². As different polymer and non-polymer coatings will release mitoxantrone at differing rates, the above dosing parameters should be utilized in combination with the release rate of the drug from the implant surface such that a minimum concentration of 10⁻⁵-10⁻⁶ M of mitoxantrone is maintained. It is necessary to insure that drug concentrations on the implant surface exceed concentrations of mitoxantrone known to be lethal to multiple species of bacteria and fungi (i.e., are in excess of 10⁻⁵ M; although for some embodiments lower drug levels will be sufficient). In a preferred embodiment, mitoxantrone is released from the surface of the implant such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of mitoxantrone (as described previously) with similar functional activity can be utilized for the purposes of this invention; the above dosing parameters are then adjusted according to the relative potency of the analogue or derivative as compared to the parent compound (e.g., a compound twice as potent as mitoxantrone is administered at half the above parameters, a compound half as potent as mitoxantrone is administered at twice the above parameters, etc.).

(b) Fluoropyrimidines Utilizing the fluoropyrimidine 5-fluorouracil as an example, whether applied as a polymer coating, incorporated into the polymers which make up the implant, or applied without a carrier polymer, the total dose of 5-fluorouracil applied should not exceed 250 mg (range of 1.0 μg to 250 mg). In a particularly preferred embodiment, the total amount of drug applied should be in the range of 10 μg to 25 mg. The dose per unit area (i.e., the amount of drug as a function of the surface area of the portion of the implant to which drug is applied and/or incorporated) should fall within the range of 0.1 μg-1 mg per mm² of surface area. In a particularly preferred embodiment, 5-fluorouracil should be applied to the implant surface at a dose of 1.0 μg/mm²-50 μg/mm². As different polymer and non-polymer coatings will release 5-fluorouracil at differing rates, the above dosing parameters should be utilized in combination with the release rate of the drug from the implant surface such that a minimum concentration of 10⁻⁴-10⁻⁷ M of 5-fluorouracil is maintained. It is necessary to insure that surface drug concentrations exceed concentrations of 5-fluorouracil known to be lethal to numerous species of bacteria and fungi (i.e., are in excess of 10⁻⁴ M; although for some embodiments lower drug levels will be sufficient). In a preferred embodiment, 5-fluorouracil is released from the implant surface such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of 5-fluorouracil (as described previously) with similar functional activity can be utilized for the purposes of this invention; the above dosing parameters are then adjusted according to the relative potency of the analogue or derivative as compared to the parent compound (e.g., a compound twice as potent as 5-fluorouracil is administered at half the above parameters, a compound half as potent as 5-fluorouracil is administered at twice the above parameters, etc.).

(c) Podophylotoxins Utilizing the podophylotoxin etoposide as an example, whether applied as a polymer coating, incorporated into the polymers which make up the cardiac implant, or applied without a carrier polymer, the total dose of etoposide applied should not exceed 25 mg (range of 0.1 μg to 25 mg). In a particularly preferred embodiment, the total amount of drug applied should be in the range of 1 μg to 5 mg. The dose per unit area (i.e., the amount of drug as a function of the surface area of the portion of the implant to which drug is applied and/or incorporated) should fall within the range of 0.01 μg-100 μg per mm² of surface area. In a particularly preferred embodiment, etoposide should be applied to the implant surface at a dose of 0.1 μg/mm²-10 μg/mm². As different polymer and non-polymer coatings will release etoposide at differing rates, the above dosing parameters should be utilized in combination with the release rate of the drug from the implant surface such that a concentration of 10⁻⁵-10⁻⁶ M of etoposide is maintained. It is necessary to insure that surface drug concentrations exceed concentrations of etoposide known to be lethal to a variety of bacteria and fungi (i.e., are in excess of 10⁻⁵M; although for some embodiments lower drug levels will be sufficient). In a preferred embodiment, etoposide is released from the surface of the implant such that anti-infective activity is maintained for a period ranging from several hours to several months. In a particularly preferred embodiment the drug is released in effective concentrations for a period ranging from 1 week-6 months. It should be readily evident based upon the discussions provided herein that analogues and derivatives of etoposide (as described previously) with similar functional activity can be utilized for the purposes of this invention; the above dosing parameters are then adjusted according to the relative potency of the analogue or derivative as compared to the parent compound (e.g., a compound twice as potent as etoposide is administered at half the above parameters, a compound half as potent as etoposide is administered at twice the above parameters, etc.).

(d) Combination therapy. It should be readily evident based upon the discussions provided herein that combinations of anthracyclines (e.g., doxorubicin or mitoxantrone), fluoropyrimidines (e.g., 5-fluorouracil), folic acid antagonists (e.g., methotrexate and/or podophylotoxins (e.g., etoposide) can be utilized to enhance the antibacterial activity of the implant coating. Similarly anthracyclines (e.g., doxorubicin or mitoxantrone), fluoropyrimidines (e.g., 5-fluorouracil), folic acid antagonists (e.g., methotrexate and/or podophylotoxins (e.g., etoposide) can be combined with traditional antibiotic and/or antifungal agents to enhance efficacy. The anti-infective agent may be further combined with antithrombotic and/or antiplatelet agents (for example, heparin, dextran sulphate, danaparoid, lepirudin, hirudin, AMP, adenosine, 2-chloroadenosine, aspirin, phenylbutazqne, indomethacin, meclofenamate, hydrochloroquine, dipyridamole, iloprost, ticlopidine, clopidogrel, abcixamab, eptifibatide, tirofiban, streptokinase, and/or tissue plasminogen activator) to enhance efficacy.

I. Methods for Generating Intravascular Devices which Include and Release a Fibrosis-Inducing Agent

In the practice of this invention, drug-coated or drug-impregnated intravascular devices are provided which induce adhesion or fibrosis in the surrounding tissue, or facilitate “anchoring” of the device/implant in situ, thus enhancing the efficacy. In the treatment of vulnerable plaque lesions, intravascular devices are provided which induce fibrosis in the plaque such the risk of plaque rupture is reduced. Within various embodiments, fibrosis is induced by local or systemic release of specific pharmacological agents that become localized to the tissue adjacent to the device or implant. Within various other embodiments, fibrosis is induced locally by incorporating the specific pharmacological agent into or onto the intravascular device (such as a stent, stent graft aneurysm coil or embolic agent) in a manner such that the majority of the pharmacological agent in not released from the device. There are numerous methods available for optimizing delivery of the fibrosis-inducing agent to the site of the intervention and several of these are described below.

1) Intravascular Devices that Include and/or Release Fibrosis-Inducing Agents

A wide variety of intravascular devices may be utilized within the context of the present invention, depending on the site and nature of treatment desired. Methods for manufacturing Intravascular devices, such as stents, stent grafts, aneurysm coils, embolic agents and other types of devices may comprise the step of coating (e.g., spraying, dipping, wrapping, or administering drug through) a medical device or implant. Additionally, the implant or medical device can be constructed so that the device itself is comprised of materials, which induce fibrosis in or around the implant or the materials which induce fibrosis in or around the implant can be physically attached or otherwise associated with the device.

Intravascular devices (e.g., stents, stent grafts, aneurysm coils, embolic agents) may be coated with, or otherwise adapted to contain and/or release an agent which induces fibrosis or adhesion to the surrounding tissue. In one aspect, the present invention provides compositions and stent grafts that include a fibrosing agent, where the agent may encourage scar formation to strengthen and improve adhesion between the surgically implanted stent graft and the host tissue. In another aspect, the present invention provides compositions and aneurysm coils that include a fibrosing agent, where the agent may encourage scar formation to fill or shrink the cerebral aneurysm. In another aspect, the present invention provides compositions and embolic agents that include a fibrosing agent, where the agent may encourage scar formation to occlude a blood vessel (or part of a blood vessel) such that blood flow is reduced or prevented. In another aspect, the present invention provides compositions and stents, drug delivery balloons and catheters that include a fibrosing agent, where the agent may encourage scar formation between the surgically implanted device and the host tissue to stabilize vulnerable plaque. Intravascular devices may be adapted to have incorporated into or onto their structure a fibrosis-inducing agent, adapted to have a surface coating of a fibrosis-inducing agent and/or adapted to release a fibrosis-inducing agent by (a) directly affixing to the implant or device a desired fibrosis-inducing agent or composition containing the fibrosis-inducing agent (e.g., by either spraying the medical implant with a drug and/or carrier (polymeric or non-polymeric)-drug composition to create a film or coating on all, or parts of the internal or external surface of the device; by dipping the implant or device into a drug and/or carrier (polymeric or non-polymeric)-drug solution to coat all or parts of the device or implant; or by other covalent or non-covalent (e.g., mechanically attached via knotting or the use of an adhesive or thermal treatment, electrostatic, ionic, hydrogen bonded or hydrophobic interactions) attachment of the therapeutic agent to the device or implant surface); (b) by coating the medical device or implant with a substance such as a hydrogel that either contains or which will in turn absorb the desired fibrosis-inducing agent or composition; (c) by interweaving a “thread” composed of, or coated with, the fibrosis-inducing agent into the medical implant or device (e.g., a polymeric strand composed of materials that induce fibrosis (e.g., silk, wool, collagen, EVA, PLA, polyurethanes, polymerized drug compositions) or polymers which comprise and/or release a fibrosis-inducing agent from the thread); (d) by covering all, or portions of the device or implant with a sleeve, cover or mesh containing a fibrosis-inducing agent (i.e., a covering comprised of a fibrosis-inducing agent—polymers such as silk, wool, collagen, EVA, PLA, polyurethanes, DACRON, ePTFE, or polymerized compositions containing fibrosis-inducing agents); (e) constructing all, or parts of the device or implant itself with the desired agent or composition (e.g., constructing it from polymers such as silk, collagen, EVA, PLA, DACRON, ePTFE, polyurethanes, wool or polymerized compositions of fibrosis-inducing agents); (f) otherwise impregnating the device or implant with the desired fibrosis-inducing agent or composition; (g) scoring (i.e., creating ridges or indentations) on all, or parts, of the device or implant surface to produce irritation and ultimately fibrosis; (h) composing all, or parts, of the device or implant from metal alloys that induce fibrosis (e.g., copper); (i) constructing all, or parts of the device or implant itself from a degradable or non-degradable polymer that releases one or more fibrosis-inducing agents; (j) incorporating the scarring agent into a specialized multi-drug releasing medical device system such as is described, e.g., in U.S. Pat. No. 6,562,065; U.S. patent application Nos. 2003/0199970 and 2003/0167085; and in WO 03/015664 and WO 02/32347, to deliver fibrosis-inducing agents alone or in combination. In one aspect, an intravascular medical device (e.g., a stent, stent graft, catheter, aneurysm coil, embolic agent or drug delivery balloon) may include a plurality of reservoirs within its structure, each reservoir configured to house and protect a therapeutic drug. Examples of such devices include the multi-drug releasing systems described above and those described in U.S. Pat. Nos. 6,527,799; 6,293,967; 6,290,673; 6,241,762). The reservoirs may be formed from divets in the device surface or micropores or channels in the device body. In one aspect, the reservoirs are formed from voids in the structure of the device. The reservoirs may house a single type of therapeutic agent (e.g., silk) or more than one type of therapeutic agent. The drug(s) may be formulated with a carrier (e.g., a polymeric or non-polymeric material) that is loaded into the reservoirs. The filled reservoir can function as a drug delivery depot which can release drug over a period of time dependent on the release kinetics of the drug from the carrier. In certain embodiments, the reservoir may be loaded with a plurality of layers. Each layer may include a different drug having a particular amount (dose) of drug, and each layer may have a different composition to further tailor the amount of drug that is released from the substrate. The multi-layered carrier may further include a barrier layer that prevents release of the drug(s). The barrier layer can be used, for example, to control the direction that the drug elutes from the void.

In one aspect, a medical device may be modified by attaching fibers (threads) to the surface of the device. The intravascular device may include polymeric threads, such that the presence of the polymeric threads results in an enhanced cellular and extracellular matrix response to the exterior of the device (e.g., stent graft, aneurysm coil). The polymeric threads can be made from any polymer that results in an enhanced cellular and/or fibrotic response. The fibers may be polymeric and/or may be formed of or coated with a fibrosing material, such as silk or wool. The threads may be a silk suture material or another type of biocompatible polymer which is coated with a polymer that results in an enhanced cellular response. In one aspect, the fibers are formed from or are coated with starch.

The threads can be coated with a material that delays the time it takes for the thread material to come into contact with the surrounding tissue and blood, thus allowing placement of the device without concern of thrombotic events due to the presence of the polymeric threads. Examples of materials that can be used to prepare coatings capable of degrading or dissolving upon implantation include gelatin, polyesters (e.g., PLGA, PLA, MePEG-PLGA, PLGA-PEG-PLGA, and blends thereof), lipids, fatty acids, sugar esters, nucleic acid esters, polyanhydrides, polyorthoesters, and PVA. The coating may further contain a fibrosing agent and/or a biologically active agent that may, for example, reduce the probability of an immediate thrombotic event (e.g., heparin and heparin derivatives, such as hydrophobic quaternary amine heparin complexes (e.g., heparin/benzylalkonium chloride complex, and the like). In addition to the polymeric threads, all or a portion of the device may be coated with a polymeric carrier that contains a fibrosis-inducing agent.

The fibers (threads) may further comprise a coating or composition that is affected by an applied magnetic field. For example, a device such as a stent graft may be coated with polymeric threads that are coated, contain, or are formed from a fibrosing agent (e.g., silk suture, wool fibers). A magnetic field can be applied to the coated device to orient and align the polymeric fibers relative to each other and the surface of the device to increase the surface area of the fibers exposed to biological mediators which would stimulate a fibrotic reaction. The magnetically active component can be associated with the polymeric fiber using a variety of methods. The magnetically active component may be incorporated during manufacture of the fiber, for example, by incorporating a magnetically active material such as magnetite into a polymer feed prior to extrusion of the polymeric fiber. The magnetically active component can be coated onto the entire fiber or a portion of the fiber using, for example, an adhesive or a polymeric coating. The polymeric fiber (or a portion thereof) can be heated or plasticized with a solvent and then rolled in the magnetically active component, such that the magnetic material protrudes above the surface of the fiber or is embedded into the surface of the fiber.

The threads can be attached to the device by using any one or a combination of the following methods, including use of an adhesive, thermal welding, stitching, wrapping, weaving, knotting, and the like. The threads (either with or without a magnetic component) may be attached to the device in various configurations that can result in either partial or complete coverage of the exterior of the device. The polymeric threads may be affixed to the ends of a device or to the central portion of a device, and the attachment may be in a vertical, horizontal, or diagonal manner.

In one aspect, the intravascular device may be adapted to include a fibrosing agent by covering all, or portions of the stent with a sleeve or cover (i.e., a continuous covering that isolates the plaque from the circulation (see, e.g., U.S. Pat. Nos. 5,603,722; 5,674,242; 6,019,789; 6,168,619; 6,248,129; and 6,530,950, assigned to Quanam Medical Corporation (Mountain View, Calif.); U.S. Pat. No. 6,290,722) or a mesh (i.e., a discontinuous covering such that portions of the plaque are not isolated and arterial side branches are not obstructed) which is composed of a fibrosing agent (e.g., polymers such as silk, collagen, wool, EVA, PLA, DACRON, ePTFE, polyurethanes, or polymerized compositions of fibrosing agents), contains or is coated with the desired fibrosing therapeutic agent or composition.

In another aspect, the fibrosing agent may be associated with a stent or other intravascular device by directly affixing to the adluminal (outer) stent or stent graft surface a desired fibrosing therapeutic agent or composition containing the fibrosing agent (e.g., by either spraying the stent or stent graft with a polymer/drug to create a film on all, or parts, of the adluminal stent surface; spraying the adluminal stent or stent graft surface with a polymerized version of the drug to create a film on all, or parts, of the outer stent surface; by dipping the stent or stent graft into a polymer/drug solution to coat all, or parts of the adluminal stent or stent graft surface; by dipping the device into a solution of polymerized drug to coat all, or parts, of the adluminal stent or stent graft surface; or by other covalent or non-covalent attachment of the therapeutic agent to the adluminal stent or stent graft surface) and also directly affixing (in the manners just described) to the luminal (inner) stent or stent graft surface a therapeutic agent or composition that inhibits restenosis (such as paclitaxel, vincristine, sirolimus, everolimus, biolimus, mycophenolic acid, ABT-578, cervistatin, simvastatin, methylprednisolone, dexamethasone, actinomycin-D, angiopeptin, L-arginine, estradiol, 17-β-estradiol, tranilast, methotrexate, batimistat, halofuginone, BCP-671, QP-2, lantrunculin D, cytochalasin A, nitric oxide and analogues and derivatives thereof), and/or thrombosis (such as heparin, aspirin, or dipyridamole); and/or (k) utilizing specialized multi-drug releasing stent systems (described, e.g., in U.S. Pat. No. 6,562,065, U.S. patent application Nos. 2003/0199970 and 2003/0167085, and WO 03/015664 and WO 02/32347) to preferentially deliver fibrosing agents to arterial plaque (i.e., the adluminal surface of the stent) while preventing restenotic tissue from growing on the luminal surface of the stent by releasing anti-restenotic drugs (e.g., paclitaxel, vincristine, sirolimus, everolimus, biolimus, mycophenolic acid, ABT-578, cervistatin, simvastatin, methylprednisolone, dexamethasone, actinomycin-D, angiopeptin, L-arginine, estradiol, 17-β-estradiol, tranilast, methotrexate, batimistat, halofuginone, BCP-671, QP-2, lantrunculin D, cytochalasin A, nitric oxide and analogues and derivatives thereof) and/or thrombosis (such as heparin, aspirin, dipyridamole) on the inner surface.

Referring to FIG. 2, a covered stent 400 is shown that includes a stent 410 and a sleeve 420 surrounding the exterior surface 430 of the stent 410. The outer surface 440 of the sleeve 420 is coated with a composition 450 that induces fibrous tissue formation. The composition may be in the form, for example, of fibers, however, other configurations are also possible. The inner surface (not shown) of the stent 410 is coated with one or more agents that inhibit restenosis and/or thrombus formation.

Referring to FIG. 3, a stent graft 470 is shown that includes a stent 480 and graft material 490. The outer surface 492 of the stent graft 470 is coated with a composition 494 that induces fibrous tissue formation. The composition may be in the form, for example, of fibers, however, other configurations are also possible. The inner surface (not shown) of the stent 480 is coated with one or more agents that inhibit thrombus formation.

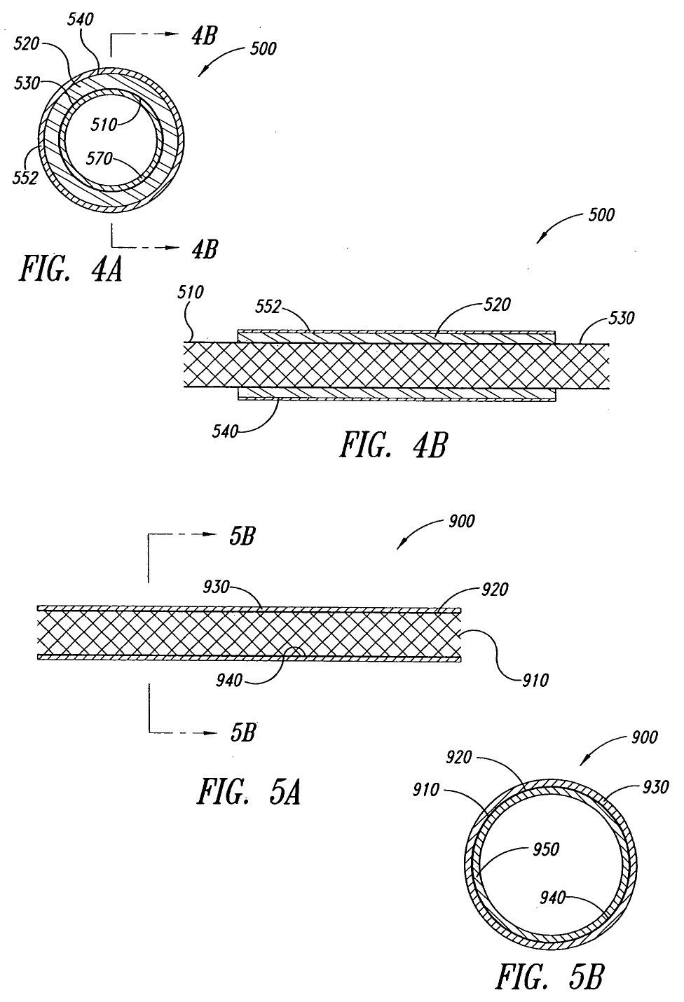

Referring to FIG. 4A and FIG. 4B, a covered stent 500 is shown that includes a stent 510 and a sleeve 520 surrounding the exterior surface 530 of the stent 510. The outer surface 540 of the sleeve 520 is coated with a composition 552 that induces fibrin formation. The inner surface 570 of the stent 510 is coated with one or more agents that inhibit restenosis and/or thrombus formation.

Referring to FIG. 5A and FIG. 5B, a stent 900 is shown that includes a plurality of tynes 910. The outer surface 920 of the stent tynes 910 is coated with a first composition 930 that induces fibrosis in plaque. The inner surface 940 of the stent tynes 910 is coated with a second composition 950 that may include an agent that induces fibrosis in plaque, which may be the same or a different agent than that included in the first composition 930, or another type of therapeutic agent, such as described herein (e.g., an agent that inhibits restenosis and/or thrombus formation). Typically, the coating composition 930 or 950 does not fill the voids between the stent tynes 910, however, in certain embodiments, the coating composition 930 or 950 may fill the voids between the stent tynes 910. Multi-drug releasing stent systems can release one or more of the fibrosing agents at the same time or over different intervals since these devices have the ability for one to include one or more agents at different locations on the device as well as to coat/fill the same location on the device with one or more compositions that are either of the same or different composition. For example, the fibrosing agent can be incorporated into a composition (e.g., PDLLA, PCL, PLLA, and PLGA) that will release the agent over a specific time period (e.g., weeks to months). The same or a different fibrosing agent can be incorporated into a carrier (e.g., PLGA, PLLA, polyurethane, polyanhydrides) and can be coated onto the device, such that it will release the agent over a different time period compared to the first composition. The release can be shorter relative to the first composition or it can be longer relative to the first composition.

For many of the aforementioned embodiments, localized sustained delivery of the fibrosis-inducing agent may be required optimize the treatment of the medical condition. For example, a desired fibrosis-inducing agent may be admixed with, blended with, conjugated to, or, otherwise modified to contain a polymer composition (which may be either biodegradable or non-biodegradable) in order to release the therapeutic agent over a prolonged period of time. Accordingly, other various types of intravascular devices (e.g., catheters, aneurysm coils, stent grafts, drug delivery balloons, embolic agents and stents) may be coated with or otherwise adapted to release an agent, which induces fibrosis or adhesion between the device and the surrounding tissue, as described above.

The therapeutic agent (with or without a carrier composition) can be a) incorporated directly into or onto the device, b) incorporated into a solution, c) incorporated into the composition used for coating the device or d) incorporated into or onto the device following coating of the device with a coating composition.

2) Systemic, Regional and Local Delivery of Fibrosis-Inducing Agents

A variety of drug-delivery technologies are available for systemic, regional and local delivery of therapeutic agents. Several of these techniques are suitable to achieve preferentially elevated levels of fibrosis-inducing agents in the vicinity of the medical device or implant, including: (a) using drug-delivery catheters for local, regional or systemic delivery of fibrosing agents to the tissue surrounding the device or implant (typically, drug delivery catheters are advanced through the circulation or inserted directly into tissues under radiological guidance until they reach the desired anatomical location; the fibrosing agent can then be released from the catheter lumen in high local concentrations in order to deliver therapeutic doses of the drug to the tissue surrounding the device or implant); (b) drug localization techniques such as magnetic, ultrasonic or MRI-guided drug delivery; (c) chemical modification of the fibrosis-inducing drug or formulation designed to increase uptake of the agent into damaged tissues (e.g., antibodies directed against damaged or healing tissue components such as macrophages, neutrophils, smooth muscle cells, fibroblasts, extracellular matrix components, neovascular tissue); (d) chemical modification of the fibrosis-inducing drug or formulation designed to localize the drug to areas of bleeding or disrupted vasculature; and/or (e) direct injection of the fibrosis-inducing agent, for example under endoscopic vision.

3) Infiltration of Fibrosis-Inducing Agents into the Tissue Surrounding a Device or Implant

Alternatively, the tissue cavity into which the device or implant is placed can be treated with a fibrosis-inducing agent prior to, during, or after implantation of the device. This can be accomplished in several ways including: (a) direct application of the fibrosing agent into the anatomical space where the device will be placed (particularly useful for this embodiment is the use of polymeric carriers which release the fibrosing agent over a period ranging from several hours to several weeks—fluids, suspensions, emulsions, microemulsions, microspheres, pastes, gels, microparticulates, sprays, aerosols, solid implants and other formulations which release a fibrosing agent can be delivered into the region where the device or implant will be inserted via specialized delivery catheters or other applicators) such as, for example, injection/infiltration of the agent into the vulnerable plaque or into the aneurysm sac; (b) microparticulate silk and/or silk strands (linear, branched, and/or coiled) are also useful for directed delivery into the vulnerable plaque or aneurysm sac; microparticulate wool and/or wool fibers (linear, branched, and/or coiled) are also useful for directed delivery into the vulnerable plaque or aneurysm sac; (c) sprayable collagen-containing formulations such as COSTASIS (Angiotech Pharmaceuticals, Inc., Canada) or materials made from 4-armed thiol PEG (10K), a 4-armed NHS PEG(10K) and methylated collagen, such as are described below, either alone, or loaded with a fibrosis-inducing agent, injected or infiltrated into the vulnerable plaque, aneurysm sac or implantation site (or the implant/device surface); (d) sprayable PEG-containing formulations such as COSEAL (Angiotech Pharmaceuticals, Inc.), FOCALSEAL (Genzyme Corporation, Cambridge, Mass.), SPRAYGEL or DURASEAL (both from Confluent Surgical, Inc., Waltham, Mass.), either alone, or loaded with a fibrosis-inducing agent, injected or infiltrated into the vulnerable plaque, aneurysm sac or implantation site (or the implant/device surface); (e) fibrinogen-containing formulations such as FLOSEAL or TISSEEL (Baxter Healthcare Corporation, Fremont, Calif.), either alone, or loaded with a fibrosis-inducing agent, injected or infiltrated into the vulnerable plaque, aneurysm sac or implantation site (or the implant/device surface); (f) hyaluronic acid-containing formulations such as PERLANE or RESTYLANE (both from Q-Med AB, Sweden), HYLAFORM (Inamed Corporation; Santa Barbara, Calif.), SYNVISC (Biomatrix, Inc., Ridgefied, N.J.), SEPRAFILM or SEPRACOAT (both from Genzyme Corporation), loaded with a fibrosis-inducing agent injected or infiltrated into the vulnerable plaque, aneurysm sac or implantation site (or the implant/device surface); (g) polymeric gels for surgical implantation such as REPEL (Life Medical Sciences, Inc., Princeton, N.J.) or FLOWGEL (Baxter Healthcare Corporation), or poly(ethylene oxide)/carboxymethylcellulose complexes (e.g., OXIPLEX from Fziomed, Inc.) loaded with a fibrosis-inducing agent injected or infiltrated into the vulnerable plaque, aneurysm sac or implantation site (or the implant/device surface); (h) surgical adhesives containing cyanoacrylates such as DERMABOND (Johnson & Johnson, Inc., New Brunswick, N.J.), INDERMIL (United States Surgical, Norwalk, Conn.), GLUSTITCH (Blacklock Medical Company, Canada), TISSUMEND II (Veterinary Products Laboratories, Phoenix, Ariz.), VETBOND (3M Company, St. Paul, Minn.), HISTOACRYL BLUE (Davis & Geck; St. Louis, Mo.), TISSUEMEND (TEI Biosciences, Inc., Boston, Mass.) and ORABASE SOOTHE-N-SEAL LIQUID PROTECTANT (Colgate-Palmolive Company, New York; N.Y.) or as described above, either alone, or loaded with a fibrosis-inducing agent, injected or infiltrated into the vulnerable plaque, aneurysm sac or implantation site (or the implant/device surface); (i) other biocompatible tissue fillers loaded with a fibrosis-inducing agent, such as those made by BioCure, Inc. (Norcross, Ga.), 3M Company and Neomend, Inc. (Sunnyvale, Calif.), loaded with a fibrosis-inducing agent injected or infiltrated into the vulnerable plaque, aneurysm sac or implantation site (or the implant/device surface); (j) polysaccharide gels such as the ADCON series of gels (Gliatech, Inc.; Cleveland, Ohio) either alone, or loaded with a fibrosis-inducing agent, injected or infiltrated into the vulnerable plaque, aneurysm sac or implantation site (or the implant/device surface); and (k) films, sponges or meshes such as INTERCEED, VICRYL mesh (Johnson & Johnson, Inc.), and GELFOAM (Pharmacia & Upjohn Company, Kalamazoo, Mich.) loaded with a fibrosis-inducing agent injected or infiltrated into the vulnerable plaque, aneurysm sac or implantation site (or the implant/device surface).

In one aspect, the fibrosing agent may be delivered into an anatomical space (such as an aneurysm sac) or a fluid environment (such as the center of a vulnerable plaque) as a solution. The fibrosing agent can be incorporated directly into the solution to provide a homogeneous solution or dispersion. In certain embodiments, the solution is an aqueous solution (e.g., a saline solution). The aqueous solution may further include buffer salts, as well as viscosity modifying agents (e.g., hyaluronic acid, alginates, CMC, and the like). In certain embodiments (for example when the agent is insoluble in water and it will be injected into a lipid plaque), the injectable is a lipid soluble solution (e.g., a fat emulsion, oil emulsion, triglycerides). In another aspect of the invention, the solution can include a biocompatible solvent, such as ethanol, DMSO, glycerolor NMP, or liquid oligomers such as PEG-200 or PEG-300.

4) Coating and Sustained-Release Preparations of Fibrosis-Inducing Agents

For many of the aforementioned embodiments, the fibrosis-inducing agent can be incorporated into, or coated onto, the device. The coating process can be performed in such a manner as to (a) coat the surfaces of the device that is in contact with the blood vessel tissue (e.g., the adluminal surface), (b) coat the surfaces of the device that are not in contact with the blood vessel tissue (e.g., the luminal surface) or (c) coat all or parts of both the blood vessel tissue-contacting (adluminal) and non-contacting (luminal) surfaces of the device. For example, a desired fibrosis-inducing agent may be admixed with, blended with, conjugated to, or, otherwise modified to contain a polymeric composition (which may be either biodegradable or non-biodegradable) or non-polymeric composition that can be used to coat the device or otherwise incorporate the agent into the device, or as a component of the materials used to manufacture the device. In other embodiments, the localized sustained delivery of the fibrosis-inhibiting agent may be desired. The fibrosing agent may or may not be released from the device.

Representative examples of biodegradable polymers and compositions suitable for the use in conjunction with fibrosing agents and/or for the delivery of fibrosis-inducing agents include albumin, collagen, gelatin, hyaluronic acid, starch, cellulose and cellulose derivatives (e.g., methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextrans, dextran sulfates, polysaccharides, sulfonated polysaccharides, fibrinogen, poly(ether ester) multiblock copolymers, based on poly(ethylene glycol) and poly(butylene terephthalate), tyrosine-derived polycarbonates (see, e.g., U.S. Pat. No. 6,120,491), poly(hydroxyl acids), poly(D,L-lactide), poly(D,L-lactide-co-glycolide), poly(glycolide), poly(hydroxybutyrate), poly(hydroxyvalerate), polydioxanone, poly(alkylcarbonate) and poly(orthoesters), aliphatic polyesters, poly(hydroxyvaleric acid), polydioxanone, poly(malic acid), poly(tartronic acid), poly(acrylamides), polyanhydrides, poly(ester-amides), poly(ester-imides), poly(ester-ureas), poly(ester-urethane-ureas), poly(anhydride-esters), poly(anhydride-imides), polyphosphazenes, poly(amino acids), poly(alkylene oxide)-poly(ester) block copolymers (e.g., X—Y, X—Y—X or Y—X—Y, R—(Y—X)ₙ, R—(X—Y)ₙ where X is a polyalkylene oxide and Y is a polyester (e.g., polyester can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, ?-decanolactone, d-decanolactone, trimethylene carbonate, 1,4-dioxane-2-one or 1,5-dioxepan-2one.), R is a multifunctional initiator and copolymers as well as blends thereof. (see generally, Ilium, L., Davids, S. S. (eds.) “Polymers in Controlled Drug Delivery” Wright, Bristol, 1987; Arshady, J. Controlled Release 17:1-22,1991; Pift, Int. J. Phar. 59:173-196,1990; Holland et al., J. Controlled Release 4:155-0180, 1986).

Representative examples of non-degradable polymers suitable for the use with, and delivery of, fibrosis-inducing agents include poly(ethylene-co-vinyl acetate) (“EVA”) copolymers, silicone rubber, acrylic polymers (e.g., polyacrylic acid, polymethylacrylic acid, polymethylmethacrylate, poly(butyl methacrylate)), poly(alkylcyanoacrylate) (e.g., poly(ethylcyanoacrylate), poly(butylcyanoacrylate), poly(hexylcyanoacrylate), and poly(octylcyanoacrylate)), polyethylene, polypropylene, polyamides (nylon 6,6), polyurethanes (including hydrophilic polyurethanes), poly(ester-urethanes), poly(ether-urethanes), poly(ester-urea), poly(carbonate urethane)s, polyethers (poly(ethylene oxide), poly(propylene oxide), polyoxyalkylene ether block copolymers based on ethylene oxide and propylene oxide such as PLURONIC and PLURONIC R polymers, poly(tetramethylene glycol)), styrene-based polymers (polystyrene, poly(styrene sulfonic acid), poly(styrene)-block-poly(isobutylene)-block-poly(styrene), poly(styrene)-poly(isoprene) block copolymers], and vinyl polymers (polyvinylpyrrolidone, poly(vinyl alcohol), poly(vinyl acetate phthalate), as well as copolymers and blends thereof.

Polymers may also be developed which are either anionic (e.g., alginate, carrageenan, carboxymethyl cellulose, poly(acrylamido-2-methyl propane sulfonic acid) and copolymers thereof, poly(methacrylic acid) and copolymers thereof, and poly(acrylic acid) and copolymers thereof, as well as blends thereof) or cationic (e.g., chitosan, poly-L-lysine, polyethylenimine, and poly(allyl amine) and blends thereof (see generally, Dunn et al., J. Applied Polymer Sci. 50:353-365, 1993; Cascone et al., J. Materials Sci.: Materials in Medicine 5:770-774,1994; Shiraishi et al., Biol. Pharm. Bull. 16(11):1164-1168,1993; Thacharodi and Rao, Int'l J. Pharm. 120:115-118, 1995; Miyazaki et al., Int'l J. Pharm. 118:257-263, 1995).

Preferred polymers (i.e., polymeric carriers) (including copolymers and blends of these polymers) include poly(ethylene-co-vinyl acetate), cellulose esters (nitrocellulose), poly(hydroxymethacrylate), poly(methylmethacrylate), poly(ethylene-co-acrylic acid), poly(vinylpyrrolidone) polyurethanes (e.g., CHRONOFLEX AL and CHRONOFLEX AR (both from CardioTech International, Inc., Woburn, Mass.) and BIONATE (Polymer Technology Group, Inc., Emeryville, Calif.)), poly(hydroxyl acids) (e.g., poly (D,L-lactic acid) oligomers and polymers, poly (L-lactic acid) oligomers and polymers, poly (glycolic acid), copolymers of lactic acid and glycolic acid, poly (caprolactone), and poly (valerolactone)), poly(anhydrides), poly(anhydride esters), poly(ester-amides), poly(ester-ureas), copolymers of poly (caprolactone) or poly (lactic acid) with a polyethylene glycol (e.g., MePEG), silicone rubbers, poly(styrene)block-poly(isobutylene)-block-poly(styrene), poly(acrylate) polymers, and blends, admixtures, or co-polymers of any of the above. Other examples (including copolymers and blends of these polymers) include poly(carbonate urethanes), poly(D-lactic acid) oligomers and polymers, copolymers of lactide and glycolide, copolymers of lactide or glycolide and ε-caprolactone, copolymers prepared from caprolactone and/or lactide and/or glycolide and/or polyethylene glycol. Other preferred polymers include collagen, poly(alkylene oxide)-based polymers, polysaccharides such as hyaluronic acid, chitosan and fucans, and copolymers of polysaccharides with degradable polymers, as well as crosslinked compositions of the above.

Further representative polymers for use in conjunction with a fibrosing agent and that are capable of sustained localized delivery of fibrosis-inducing agents include carboxylic polymers, polyacetates, polyacrylamides, polycarbonates, polyethers, substituted polyethylenes, polyvinylbutyrals, polysilanes, polyureas, polyoxides, polystyrenes, polysulfides, polysulfones, polysulfonides, polyvinylhalides, pyrrolidones, isoprene rubbers, thermal-setting polymers, cross-linkable acrylic and methacrylic polymers, ethylene acrylic acid copolymers, styrene acrylic copolymers, vinyl acetate polymers and copolymers, vinyl acetal polymers and copolymers, epoxies, melamines, other amino resins, phenolic polymers, and copolymers thereof, water-insoluble cellulose ester polymers (including cellulose acetate propionate, cellulose acetate, nitrocellulose, cellulose acetate butyrate, cellulose nitrate, cellulose acetate phthalate, and mixtures thereof), polyvinylpyrrolidone (pvp), polyethylene glycols, polyethylene oxides, polyvinyl alcohol, polyethers, poly(ethylene terephthalate), polyhydroxyacrylate, dextran, xanthan, hydroxypropyl cellulose, methyl cellulose, and homopolymers and copolymers of N-vinylpyrrolidone, N-vinyllactam, N-vinyl butyrolactam, N-vinyl caprolactam, other vinyl compounds having polar pendant groups, acrylate and methacrylate having hydrophilic esterifying groups, hydroxyacrylate, and acrylic acid, and combinations thereof; cellulose esters and ethers, ethyl cellulose, nitro-cellulose, hydroxyethyl cellulose, cellulose nitrate, cellulose acetate, cellulose acetate butyrate, cellulose acetate propionate, polyacrylate, natural and synthetic elastomers, acetal, styrene polybutadiene, acrylic resin, polyvinylidene chloride, polycarbonate, homopolymers and copolymers of vinyl compounds, polyvinylchloride, and polyvinylchloride acetate.

Representative examples of patents relating to drug-delivery polymers and their preparation include PCT Publication Nos. WO 98/19713, WO 01/17575, WO 01/41821, WO 01/41822, and WO 01/15526 (as well as the corresponding U.S. applications), and U.S. Pat. Nos. 4,500,676, 4,582,865, 4,629,623, 4,636,524, 4,713,448, 4,795,741, 4,913,743, 5,069,899, 5,099,013, 5,128,326, 5,143,724, 5,153,174, 5,246,698, 5,266,563, 5,399,351, 5,525,348, 5,800,412, 5,837,226, 5,942,555, 5,997,517, 6,007,833, 6,071,447, 6,090,995, 6,106,473, 6,110,483, 6,121,027, 6,156,345, 6,214,901, 6,368,611 6,630,155, 6,528,080, RE37,950, 6,46,1631, U.S. Pat. Nos. 6,143,314, 5,990,194, 5,792,469, 5,780,044, 5,759,563, 5,744,153, 5,739,176, 5,733,950, 5,681,873, 5,599,552, 5,340,849, 5,278,202, 5,278,201, 6,589,549, 6,287,588, 6,201,072, 6,117,949, 6,004,573, 5,702,717, 6,413,539, and 5,714,159, 5,612,052 and U.S. Published patent application Nos. 2003/0068377, 2002/0192286, 2002/0076441, and 2002/0090398.

Polymeric carriers may be fashioned to release a fibrosis-inducing agent upon exposure to a specific triggering event such as pH (see, e.g., Heller et al., “Chemically Self-Regulated Drug Delivery Systems,” in Polymers in Medicine III, Elsevier Science Publishers B.V., Amsterdam, 1988, pp. 175-188; Kang et al., J. Applied Polymer Sci. 48:343-354,1993; Dong et al., J. Controlled Release 19:171-178, 1992; Dong and Hoffman, J. Controlled Release 15:141-152,1991; Kim et al., J. Controlled Release 28:143-152,1994; Cornejo-Bravo et al., J. Controlled Release 33:223-229, 1995; Wu and Lee, Pharm. Res. 10(10):1544-1547, 1993; Serres et al., Pharm. Res. 13(2):196-201,1996; Peppas, “Fundamentals of pH- and Temperature-Sensitive Delivery Systems,” in Gurny et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polymers include poly(acrylic acid) and its derivatives (including for example, homopolymers such as poly(aminocarboxylic acid); poly(acrylic acid); poly(methyl acrylic acid), copolymers of such homopolymers, and copolymers of poly(acrylic acid) and acrylmonomers such as those discussed above. Other pH sensitive polymers include polysaccharides such as cellulose acetate phthalate; hydroxypropylmethylcellulose phthalate; hydroxypropylmethylcellulose acetate succinate; cellulose acetate trimellilate; and chitosan. Yet other pH sensitive polymers include any mixture of a pH sensitive polymer and a water-soluble polymer.

Likewise, fibrosis-inducing agents can be delivered to a treatment site, such as a vulnerable plaque or an aneurysm, via polymeric carriers which are temperature sensitive (see, e.g., Chen et al., “Novel Hydrogels of a Temperature-Sensitive PLURONIC Grafted to a Bioadhesive Polyacrylic Acid Backbone for Vaginal Drug Delivery,” in Proceed. Intern. Symp. Control. Rel. Bioact Mater. 22:167-168, Controlled Release Society, Inc., 1995; Okano, “Molecular Design of Stimuli-Responsive Hydrogels for Temporal Controlled Drug Delivery,” in Proceed. Intern. Symp. Control. Rel. Bioact. Mater. 22:111-112, Controlled Release Society, Inc., 1995; Johnston et al., Pharm. Res. 9(3):425-433, 1992; Tung, Int'l J. Pharm. 107:85-90, 1994; Harsh and Gehrke, J. Controlled Release 17:175-186,1991; Bae et al., Pharm. Res. 8(4):531-537, 1991; Dinarvand and D'Emanuele, J. Controlled Release 36:221-227, 1995; Yu and Grainger, “Novel Thermo-sensitive Amphiphilic Gels: Poly N-isopropylacrylamide-co-sodium acrylate-co-n-N-alkylacrylamide Network Synthesis and Physicochemical Characterization,” Dept. of Chemical & Biological Sci., Oregon Graduate Institute of Science & Technology, Beaverton, Oreg., pp. 820-821; Zhou and Smid, “Physical Hydrogels of Associative Star Polymers,” Polymer Research Institute, Dept. of Chemistry, College of Environmental Science and Forestry, State Univ. of New York, Syracuse, N.Y., pp. 822-823; Hoffman et al., “Characterizing Pore Sizes and Water ‘Structure’ in Stimuli-Responsive Hydrogels,” Center for Bioengineering, Univ. of Washington, Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9(3):283-290,1992; Bae et al., Pharm. Res. 8(5):624-628, 1991; Kono et al., J. Controlled Release 30:69-75,1994; Yoshida et al., J. Controlled Release 32:97-102,1994; Okano et al., J. Controlled Release 36:125-133, 1995; Chun and Kim, J. Controlled Release 38:39-47, 1996; D'Emanuele and Dinarvand, Int'l J. Pharm. 118:237-242,1995; Katono et al., J. Controlled Release 16:215-228, 1991; Hoffman, “Thermally Reversible Hydrogels Containing Biologically Active Species,” in Migliaresi et al. (eds.), Polymers in Medicine III, Elsevier Science Publishers B.V., Amsterdam, 1988, pp.161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27, 1987, pp. 297-305; Gutowska et al., J. Controlled Release 22:95-104,1992; Palasis and Gehrke, J. Controlled Release 18:1-12, 1992; Paavola et al., Pharm. Res. 12(12):1997-2002, 1995).

Representative examples of thermogelling polymers, and the gelatin temperature [LCST (° C.)] include homopolymers such as poly(N-methyl-N-n-propylacrylamide), 19.8; poly(N-n-propylacrylamide), 21.5; poly(N-methyl-N-isopropylacrylamide), 22.3; poly( N-n-propylmethacrylamide), 28.0; poly(N-isopropylacrylamide), 30.9; poly(N, n-diethylacrylamide), 32.0; poly(N-isopropylmethacrylamide), 44.0; poly(N-cyclopropylacrylamide), 45.5; poly(N-ethylmethyacrylamide), 50.0; poly(N-methyl-N-ethylacrylamide), 56.0; poly(N-cyclopropylmethacrylamide), 59.0; poly(N-ethylacrylamide), 72.0. Moreover thermogelling polymers may be made by preparing copolymers between (among) monomers of the above, or by combining such homopolymers with other water-soluble polymers such as acrylmonomers (e.g., acrylic acid and derivatives thereof such as methylacrylic acid, acrylate and derivatives thereof such as butyl methacrylate, acrylamide, and N-n-butyl acrylamide).

Other representative examples of thermogelling polymers include cellulose ether derivatives such as hydroxypropyl cellulose, 41° C.; methyl cellulose, 55° C.; hydroxypropylmethyl cellulose, 66° C.; and ethylhydroxyethyl cellulose, polyalkylene oxide-polyester block copolymers of the structure X—Y, Y—X—Y and X—Y—X wherein X in a polyalkylene oxide and Y is a biodegradable polyester (e.g., PLG-PEG-PLG) and PLURONICs such as F-127, 10-15° C.; L-122, 19° C.; L-92, 26° C.; L-81, 20° C.; and L-61, 24° C.

Representative examples of patents relating to thermally gelling polymers and the preparation include U.S. Pat. Nos. 6,451,346; 6,201,072; 6,117,949; 6,004,573; 5,702,717; and 5,484,610; and PCT Publication Nos. WO 99/07343; WO 99/18142; WO 03/17972; WO 01/82970; WO 00/18821; WO 97/15287; WO 01/41735; WO 00/00222 and WO 00/38651.

Within further aspects of the present invention, polymeric carriers are provided which are adapted to contain and release a hydrophobic fibrosing compound, and/or the carrier containing the hydrophobic compound in combination with a carbohydrate, protein or polypeptide. Within certain embodiments, the polymeric carrier contains or comprises regions, pockets, or granules of one or more hydrophobic compounds. For example, within one embodiment of the invention, hydrophobic compounds may be incorporated within a matrix which contains the hydrophobic fibrosing compound, followed by incorporation of the matrix within the polymeric carrier. A variety of matrices can be utilized in this regard, including for example, carbohydrates and polysaccharides such as starch, cellulose, dextran, methylcellulose, sodium alginate, heparin, chitosan and hyaluronic acid, proteins or polypeptides such as albumin, collagen and gelatin. Within alternative embodiments, hydrophobic compounds may be contained within a hydrophobic core, and this core contained within a hydrophilic shell.

Within further aspects, polymeric carriers can be materials that are formed in situ. In one embodiment, the precursors can be monomers or macromers that contain unsaturated groups that can be polymerized or crosslinked. The monomers or macromers can then, for example, be injected into the treatment area or onto the surface of the treatment area and polymerized or crosslinked in situ using a radiation source (e.g., visible light, UV light) or a free radical system (e.g., potassium persulfate and ascorbic acid or iron and hydrogen peroxide). The polymerization or crosslinking step can be performed immediately prior to, simultaneously to or post injection of the reagents into the treatment site. Representative examples of compositions that undergo free radical polymerization or crosslinking reactions are described in PCT Publication Nos. WO 01/44307, WO 01/68720, WO 02/072166, WO 03/043552, WO 93/17669, and WO 00/64977, U.S. Pat. Nos. 5,900,245; 6,051,248; 6,083,524; 6,177,095; 6,201,065; 6,217,894; 6,639,014; 6,352,710; 6,410,645; 6,531,147; 5,567,435; 5,986,043; and 6,602,975, and U.S. patent application Publication Nos. 2002/012796, 2002/0127266, 2002/0151650, 2003/0104032, 2002/0091229, and 2003/0059906.

In another embodiment, the reagents can undergo an electrophilic-nucleophilic reaction to produce a crosslinked matrix. Polymers terminated with nucleophilic groups such as amine, sulfhydryl, hydroxyl, —PH₂ or CO—NH—NH₂ can be used as the nucleophilic reagents and polymers terminated with electrophilic groups such as succinimidyl, carboxylic acid, aldehyde, epoxide, isocyanate, vinyl, vinyl sulfone, maleimid, —S—S—(C₅H₄N) or activated esters used in peptide synthesis can be used as the electrophilic reagents. For example, a 4-armed thiol derivatized poly(ethylene glycol) (e.g., pentaerythritol poly(ethylene glycol)ether tetra-succinimidyl glutarate) can be reacted with a 4 armed NHS-derivatized polyethylene glycol (e.g., pentaerythritol poly(ethylene glycol)ether tetra-sulfhydryl) under basic conditions (pH>about 8). Representative examples of compositions that undergo electrophilic-nucleophilic crosslinking reactions are described in U.S. Pat. Nos. 5,752,974; 5,807,581; 5,874,500; 5,936,035; 6,051,648; 6,165,489; 6,312,725; 6,458,889; 6,495,127; 6,534,591; 6,624,245; 6,566,406; 6,610,033; 6,632,457; U.S. patent application Publication No. 2003/0077272A1, and PCT Publication Nos. WO 2004/060405A2 and WO 2004/060346A2

In another embodiment, the electrophilic- or nucleophilic-terminated polymers can further comprise a polymer that can enhance the mechanical and/or adhesive properties of the in situ forming compositions. This polymer can be a degradable or non-degradable polymer. For example, the polymer may be collagen or a collagen derivative, for example methylated collagen. An example of an in situ forming composition uses pentaerythritol poly(ethylene glycol)ether tetra-sulfhydryl (4-armed thiol PEG), pentaerythritol poly(ethylene glycol)ether tetra-succinimidyl glutarate (4-armed NHS PEG ) and methylated collagen as the reactive reagents. This composition, when mixed with the appropriate buffers will produce a crosslinked hydrogel.

In another embodiment, the polymer can be a polyester. Polyesters that can be used include the poly(hydroxyesters). In another embodiment, the polyester can comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, ?-decanolactone, d-decanolactone, trimethylene carbonate, 1,4-dioxane-2-one or 1,5-dioxepan-2one. Representative examples of these types of compositions are described in U.S. Pat. Nos. 5,874,500; 5,936,035; 6,312,725; 6,495,127 and PCT Publication Nos. WO 2004/028547.

In another embodiment, the electrophilic-terminated polymer can be partially or completely replaced by a small molecule or oligomer that comprises an electrophilic group (e.g., disuccinimidyl glutarate).

In another embodiment, the nucleophilic-terminated polymer can be partially or completely replaced by a small molecule or oligomer that comprises a nucleophilic group (e.g., dicysteine, dilysine, trilysine etc).

Other examples of in situ forming materials that can be used include those based on the crosslinking of proteins (described in U.S. Pat. No. RE38158; U.S. Pat. Nos. 4,839,345; 5,514,379, 5,583,114; 6,310,036; 6,458,147; 6,371,975; U.S. patent application Publication Nos. 2004/0063613A1; 2002/0161399A1; 2001/0018598A1 and PCT Publication Nos. WO 03/090683; WO 01/45761; WO 99/66964 and WO 96/03159) and those based on isocyanate or isothiocyanate capped polymers (described in PCT Publication No. WO 04/021983).

Other examples of in situ forming materials can include reagents that comprise one or more cyanoacrylate groups. These reagents can be used to prepare a poly(alkylcyanoacrylate) or poly(carboxyalkylcyanoacrylate) (e.g., poly(ethylcyanoacrylate), poly(butylcyanoacrylate), poly(isobutylcyanoacrylate), poly(hexylcyanoacrylate), poly(methoxypropylcyanoacrylate) and poly(octylcyanoacrylate).

Examples of commercially available cyanoacrylates that can be used in conjunction with a fibrosing agent include DERMABOND, INDERMIL, GLUSTITCH, TISSUEMEND, VETBOND, TISSUMEND II, HISTOACRYL BLUE and ORABASE SOOTHE-N-SEAL LIQUID PROTECTANT or others as described above.

In another embodiment, the cyanoacrylate compositions can further comprise one or more additives to stabilize the reagents, or alter the rate of reaction of the cyanoacrylate, or alter the mechanical properties of the polymer or a combination thereof. For example, a trimethylene carbonate based polymer or an oxalate polymer of poly(ethylene glycol), or a ε-caprolactone based copolymer can be mixed with a 2-alkoxyalkylcyanoacrylate (e.g., 2-methoxypropylcyanoacrylate). Representative examples of these compositions are described in U.S. Pat. Nos. 5,350,798 and 6,299,631.

In another embodiment, the cyanoacrylate composition can be prepared by capping heterochain polymers with a cyanoacrylate group. The cyanoacrylate-capped heterochain polymer preferably has at least two cyanoacrylate ester groups per chain. The heterochain polymer can comprise an absorbable poly(ester), poly(ester-carbonate), poly(ether-carbonate) and poly(ether-ester). The poly(ether-ester)s described in U.S. Pat. Nos. 5,653,992 and 5,714,159 can also be used as the heterochain polymers. A triaxial poly(ε-caprolactone-co-trimethylene carbonate) is an example of a poly(ester-carbonate) that can be used. The heterochain polymer may be a polyether. Examples of polyethers that can be used include poly(ethylene glycol), poly(propylene glycol) and block copolymers of poly(ethylene glycol) and poly(propylene glycol) (e.g., PLURONICs polymers including, but not limited to, F127 or F68). Representative examples of these compositions are described in U.S. Pat. No. 6,699,940.

In addition to the coating compositions and methods described above, there are various other coating compositions and methods that are known in the art. Representative examples of these coating compositions and methods are described in U.S. Pat. Nos. 6,610,016, 6,358,557, 6,306,176, 6,110,483, 6,106,473, 5,997,517, 5,800,412, 5,525,348, 5,331,027, 5,001,009; 6,562,136; 6,406,754; 6,344,035; 6,254,921; 6,214,901; 6,077,698; 6,603,040; 6,278,018; 6,238,799; 6,096,726, 5,766,158, 5,599,576, 4,119,094; 4,100,309; 6,599,558; 6,369,168; 6,521,283; 6,497,916; 6,251,964; 6,225,431; 6,087,462; 6,083,257; 5,739,237; 5,739,236; 5,705,583; 5,648,442; 5,645,883; 5,556,710; 5,496,581; 4,689,386; 6,214,115; 6,090,901; 6,599,448; 6,054,504; 4,987,182; 4,847,324; and 4,642,267, U.S. patent application Publication Nos. 2003/0129130, 2001/0026834; 2003/0190420; 2001/0000785; 2003/0059631; 2003/0190405; 2002/0146581; 2003/020399; 2003/0129130, 2001/0026834; 2003/0190420; 2001/0000785; 2003/0059631; 2003/0190405; 2002/0146581; and 2003/020399, and PCT Publication Nos. WO 02/055121; WO 01/57048; WO 01/52915; and WO 01/01957.

It should be obvious to one of skill in the art that the polymers as described herein can also be blended or copolymerized in various compositions as required to deliver therapeutic doses of fibrosis-inducing agents to blood vessels in the treatment site.

Other carriers that may likewise be utilized to contain and deliver fibrosing agents described herein include: hydroxypropyl cyclodextrin (Cserhati and Hollo, Int. J. Pharm. 108:69-75, 1994), liposomes (see, e.g., Sharma et al., Cancer Res. 53:5877-5881, 1993; Sharma and Straubinger, Pharm. Res. 11(60):889-896, 1994; WO 93/18751; U.S. Pat. No. 5,242,073), liposome/gel (WO 94/26254), nanocapsules (Bartoli et al., J. Microencapsulation 7(2):191-197, 1990), micelles (Alkan-Onyuksel et al., Pharm. Res. 11(2):206-212, 1994), implants (Jampel et al., Invest. Ophthalm. Vis. Science 34(11):3076-3083, 1993; Walter et al., Cancer Res. 54:22017-2212, 1994), nanoparticles (Violante and Lanzafame PAACR), nanoparticles—modified (U.S. Pat. No. 5,145,684), nanoparticles (surface modified) (U.S. Pat. No. 5,399,363), micelle (surfactant) (U.S. Pat. No. 5,403,858), synthetic phospholipid compounds (U.S. Pat. No. 4,534,899), gas borne dispersion (U.S. Pat. No. 5,301,664), liquid emulsions, foam, spray, gel, lotion, cream, ointment, dispersed vesicles, particles or droplets solid- or liquid-aerosols, microemulsions (U.S. Pat. No. 5,330,756), polymeric shell (nano- and micro-capsule) (U.S. Pat. No. 5,439,686), emulsion (Tarr et al., Pharm Res. 4: 62-165, 1987), nanospheres (Hagan et al., Proc. Intern. Symp. Control Rel. Bioact. Mater. 22, 1995; Kwon et al., Pharm Res. 12(2):192-195; Kwon et al., Pharm Res. 10(7):970-974; Yokoyama et al., J. Contr. Rel. 32:269-277, 1994; Gref et al., Science 263:1600-1603, 1994; Bazile et al., J. Pharm. Sci. 84:493-498, 1994) and implants (U.S. Pat. No. 4,882,168).

Within another aspect of the invention, the biologically active agent can be delivered with non-polymeric agents. These non-polymeric agents can include sucrose derivatives (e.g., sucrose acetate isobutyrate, sucrose oleate), sterols such as cholesterol, stigmasterol, β-sitosterol, and estradiol; cholesteryl esters such as cholesteryl stearate; C₁₂-C₂₄ fatty acids such as lauric acid, myristic acid, palmitic acid, stearic acid, arachidic acid, behenic acid, and lignoceric acid; C₁₈-C₃₆ mono-, di- and triacylglycerides such as glyceryl monooleate, glyceryl monolinoleate, glyceryl monolaurate, glyceryl monodocosanoate, glyceryl monomyristate, glyceryl monodicenoate, glyceryl dipalmitate, glyceryl didocosanoate, glyceryl dimyristate, glyceryl didecenoate, glyceryl tridocosanoate, glyceryl trimyristate, glyceryl tridecenoate, glycerol tristearate and mixtures thereof; sucrose fatty acid esters such as sucrose distearate and sucrose palmitate; sorbitan fatty acid esters such as sorbitan monostearate, sorbitan monopalmitate and sorbitan tristearate; C₁₆-C₁₈ fatty alcohols such as cetyl alcohol, myristyl alcohol, stearyl alcohol, and cetostearyl alcohol; esters of fatty alcohols and fatty acids such as cetyl palmitate and cetearyl palmitate; anhydrides of fatty acids such as stearic anhydride; phospholipids including phosphatidylcholine (lecithin), phosphatidylserine, phosphatidylethanolamine, phosphatidylinositol, and lysoderivatives thereof; sphingosine and derivatives thereof; spingomyelins such as stearyl, palmitoyl, and tricosanyl spingomyelins; ceramides such as stearyl and palmitoyl ceramides; glycosphingolipids; lanolin and lanolin alcohols, calcium phosphate, sintered and unscintered hydoxyapatite, zeolites; and combinations and mixtures thereof.

Representative examples of patents relating to non-polymeric delivery systems and the preparation include U.S. Pat. Nos. 5,736,152; 5,888,533; 6,120,789; 5,968,542; and 5,747,058.

Polymeric carriers for fibrosis-inducing agents can be fashioned in a variety of forms, with desired release characteristics and/or with specific properties depending upon the device, composition or implant being utilized.

Fibrosis-inducing agents may be linked by occlusion in the matrices of a polymer, bound by covalent linkages, bound by ionic interactions, or encapsulated in microcapsules. Within certain embodiments of the invention, therapeutic compositions are provided in non-capsular formulations such as microspheres (ranging from nanometers to micrometers in size), pastes, gels, threads of various size, films, meshes, and sprays.

Within certain aspects of the present invention, therapeutic compositions may be fashioned in any size ranging from 20 nm to 1500 μm, depending upon the particular use. These compositions can be in the form of microspheres (porous or non-porous), microparticles and/or nanoparticles. These compositions can be formed by spray-drying methods, milling methods, coacervation methods, W/O (water-oil) emulsion methods, W/O/W emulsion methods, and solvent evaporation methods. In another embodiment, these compositions can include microemulsions, emulsions, liposomes and micelles. Alternatively, such compositions may also be readily applied as a “spray”, which solidifies into a film or coating for use as a device/implant surface coating or to line the tissues of the implantation site. Such sprays may be prepared from microspheres of a wide array of sizes, including for example, from 0.1 μm to 3 μm, from 10 μm to 30 μm, and from 30 μm to 100 μm.

Therapeutic compositions of the present invention may also be prepared in a variety of “paste” or gel forms. For example, within one embodiment of the invention, therapeutic compositions are provided which are liquid at one temperature (e.g., temperature greater than 37° C., such as 40° C., 45° C., 50° C., 55° C. or 60° C.), and solid or semi-solid at another temperature (e.g., ambient body temperature, or any temperature lower than 37° C.). Such “thermopastes” may be readily made utilizing a variety of techniques (see, e.g., PCT Publication WO 98/24427). Other pastes may be applied as a liquid, which solidify in vivo due to dissolution of a water-soluble component of the paste and precipitation of encapsulated drug into the aqueous body environment. These “pastes” and “gels” containing fibrosing agents are particularly useful for application to the surface of tissues that will be in contact with the implant or device; for example, for direct injection into the aneurysm sac.

In one aspect, the fibrosing agent is incorporated into a film, which may, depending on the application, be formed into the shape of a tube. These films or tubes can be porous or non-porous. Generally, films are less than 5, 4, 3, 2, or 1 mm thick, more preferably less than 0.75 mm, 0.5 mm, 0.25 mm, or, 0.10 mm thick. Films can also be generated of thicknesses less than 50 μm, 25 μm or 10 μm. Films generally are flexible with a good tensile strength (e.g., greater than 50, preferably greater than 100, and more preferably greater than 150 or 200 N/cm²), good adhesive properties (i.e., adheres to moist or wet surfaces), and have controlled permeability. Fibrosing agents contained in polymeric films are particularly useful for application to the surface of a device (e.g., a stent, stent graft, aneurysm coil or embolic agent), as well as to the surface of the tissue, artery, aneurysm sac, plaque, cavity or organ.

In another aspect, the fibrosing agent is incorporated into, or coated onto, a mesh. A mesh, as used herein, is a material composed of a plurality of fibers or filaments (i.e., a fibrous material), where the fibers or filaments are arranged in such a manner (e.g., interwoven, knotted, braided, overlapping, looped, knitted, interlaced, intertwined, webbed, felted, and the like) so as to form a porous structure. The mesh may be capable of providing support to the structure (e.g., the vessel or cavity wall) and may be adapted to release an amount of the therapeutic agent. Fibrosing agents contained in or on meshes are useful for application to the surface of a stent or stent graft, as well as to the surface of a tissue, cavity or an organ.

Mesh materials may take a variety of forms. For example, the mesh may be in a woven, knit, or non-woven form and may include fibers or filaments that are randomly oriented relative to each other or that are arranged in an ordered array or pattern. In one embodiment, for example, a mesh may be in the form of a fabric, such as, for example, a knitted, braided, crocheted, woven, non-woven (e.g., a melt-blown, electrospun, electrosprayed, or wet-laid) or webbed fabric. In one embodiment, a mesh may include a natural or synthetic biodegradable polymer that may be formed into a knit mesh, a weave mesh, a sprayed mesh, a web mesh, a braided mesh, a looped mesh, and the like. Preferably, a mesh or wrap has intertwined threads that form a porous structure, which may be, for example, knitted, woven, or webbed.

The structure and properties of the mesh used in a device depend on the application and the desired mechanical (i.e., flexibility, tensile strength, and elasticity), degradation properties, and the desired loading and release characteristics for the selected therapeutic agent(s). Factors that affect the flexibility and mechanical strength of the mesh include, for example, the porosity, fabric thickness, fiber diameter, polymer composition (e.g., type of monomers and initiators), process conditions, and the additives that are used to prepare the material.

Typically, the mesh possesses sufficient porosity to permit the flow of fluids through the pores of the fiber network and/or to facilitate tissue ingrowth. Generally, the interstices of the mesh should be sufficiently wide apart to allow light visible by eye, or fluids, to pass through the pores. However, materials having a more compact structure also may be utilized. The flow of fluid through the interstices of the mesh depends on a variety of factors, including, for example, the stitch count or thread density. The porosity of the mesh may be further tailored by, for example, filling the interstices of the mesh with another material (e.g., particles or polymer) or by processing the mesh (e.g., by heating) in order to reduce the pore size and to create non-fibrous areas. Fluid flow through the mesh will vary depending on the properties of the fluid, such as viscosity, hydrophilicity/hydrophobicity, ionic concentration, temperature, elasticity, pseudoplasticity, particulate content, and the like. Preferably, the interstices do not prevent the release of impregnated or coated therapeutic agent(s) from the mesh, and the interstices preferably do not prevent the exchange of tissue fluid at the application site.

Typically, the mesh materials are sufficiently flexible so as to be capable of being wrapped around all or a portion of the external surface of a device (e.g., a stent graft) or a surface of a body passageway or cavity or a portion thereof. Flexible mesh materials are typically in the form of flexible woven or knitted sheets having a thickness ranging from about 25 microns to about 3000 microns; preferably from about 50 to about 1000 microns.

The diameter and length of the fibers or filaments may range in size depending on the form of the material (e.g., knit, woven, or non-woven), and the desired elasticity, porosity, surface area, flexibility, and tensile strength. The fibers may be of any length, ranging from short filaments to long threads (i.e., several microns to hundreds of meters in length). Depending on the application, the fibers may have a monofilament or a multifilament construction.

The mesh may include fibers that are of same dimension or of different dimensions, and the fibers may be formed from the same or different types of materials (e.g., biodegradable polymers). Woven materials, for example, may include a regular or irregular array of warp and weft strands and may include one type of polymer in the weft direction and another type (having the same or a different degradation profile from the first polymer) in the warp direction. The degradation profile of the weft polymer may be different than or the same as the degradation profile of the warp polymer. Similarly, knit materials may include one or more types (e.g., monofilament, multi-filament) and sizes of fibers and may include fibers made from the same or from different types of biodegradable polymers.

The structure of the mesh (e.g., fiber density and porosity) may impact the amount of therapeutic agent that may be loaded into or onto the device. For example, a fabric having a loose weave characterized by a low fiber density and high porosity will have a lower thread count, resulting in a reduced total fiber volume and surface area. As a result, the amount of agent that may be loaded into or onto a loosely woven fabric will be lower than for a fabric having a high fiber density and lower porosity.

It is also preferable that the mesh should not invoke biologically detrimental inflammatory or toxic response, should be capable of being fully metabolized in the body, have an acceptable shelf life, and be easily sterilized. Accordingly, the mesh or film may include a biodegradable polymer or a non-biodegradable polymer or a combination of biodegradable and non-degradable polymers.

Biodegradable compositions that may be used to prepare the mesh or film include polymers that comprise albumin, collagen, hyaluronic acid and derivatives, sodium alginate and derivatives, chitosan and derivatives, gelatin, starch, cellulose polymers (for example methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextran and derivatives, polysaccharides, poly(caprolactone), fibrinogen, poly(hydroxyl acids), such as poly(L-lactide) poly(D,L lactide), poly(D,L-lactide-co-glycolide), poly(L-lactide-co-glycolide), copolymers of lactic acid and glycolic acid, copolymers of ε-caprolactone and lactide, copolymers of glycolide and ε-caprolactone, copolymers of lactide and 1,4-dioxane-2-one, polymers and copolymers that include one or more of the residue units of the monomers D-lactide, L-lactide, D,L-lactide, glycolide, ε-caprolactone, trimethylene carbonate, 1,4-dioxane-2-one or 1,5-dioxepan-2-one, poly(glycolide), poly(hydroxybutyrate), poly(alkylcarbonate) and poly(orthoesters), polyesters, poly(hydroxyvaleric acid), polydioxanone, poly(malic acid), poly(tartronic acid), poly(anhydrides), polyphosphazenes, poly(amino acids). These compositions include copolymers of the above polymers as well as blends and combinations of the above polymers. (see, generally, Ilium, L., Davids, S. S. (eds.) “Polymers in Controlled Drug Delivery” Wright, Bristol, 1987; Arshady, J. Controlled Release 17:1-22, 1991; Pitt, Int J. Phar. 59:173-196, 1990; Holland et al., J. Controlled Release 4:155-0180, 1986).

In one aspect, the mesh or film includes a biodegradable polymer that is formed from one or more monomers selected from the group consisting of lactide, glycolide, e-caprolactone, trimethylene carbonate, 1,4-dioxan-2-one, 1,5-dioxepan-2-one, 1,4-dioxepan-2-one, hydroxyvalerate, and hydroxybutyrate. In one aspect, the polymer may include, for example, a copolymer of a lactide and a glycolide. In another aspect, the polymer includes a poly(caprolactone). In yet another aspect, the polymer includes a poly(lactic acid). In yet another aspect, the polymer includes a copolymer of lactide and e-caprolactone. In yet another aspect, the polymer includes a polyester (e.g., a poly(lactide-co-glycolide). The poly(lactide-co-glycolide) may have a lactide:glycolide ratio ranges from about 20:80 to about 2:98, a lactide:glycolide ratio of about 10:90, or a lactide:glycolide ratio of about 5:95. In one aspect, the poly(lactide-co-glycolide) is poly(L-lactide-co-glycolide).

Representative examples of non-biodegradable compositions for use in meshes and films include silk, wool, ethylene-co-vinyl acetate copolymers, acrylic-based and methacrylic-based polymers (e.g., poly(acrylic acid), poly(methylacrylic acid), poly(methylmethacrylate), poly(hydroxyethylmethacrylate), poly(alkylcynoacrylate), poly(alkyl acrylates), poly(alkyl methacrylates)), poly(ethylene), poly(propylene), poly(ethylene terephthalate), polyamides (e.g., nylon 6,6), poly(urethanes) (e.g., poly(ester urethanes), poly(ether urethanes), poly(carbonate urethanes), poly(ester-urea)), polyethers (poly(ethylene oxide), poly(propylene oxide), poly(ethylene oxide)-poly(propylene

Within another aspect of the invention, the fibrosing agent can further comprise a secondary carrier. The secondary carrier can be in the form of microspheres or embolic particles (e.g., PLGA, PLLA, PDLLA, PCL, gelatin, polydioxanone, or poly(alkylcyanoacrylate)), nanospheres (e.g., PLGA, PLLA, PDLLA, PCL, gelatin, polydioxanone, or poly(alkylcyanoacrylate)), liposomes, emulsions, microemulsions, micelles (e.g., SDS, block copolymers of the form X—Y, X—Y—X or Y—X—Y where X is a poly(alkylene oxide) or alkyl ether thereof and Y is a polyester (e.g., PLGA, PLLA, PDLLA, PCL polydioxanone)), zeolites or cyclodextrins. The fibrosing agent/secondary carrier compositions can be (a) incorporated directly into or onto the device, (b) incorporated into a solution, (c) incorporated into a gel or viscous solution, (d) incorporated into the composition used for coating the device (e.g., fibrosing agent loaded PLGA microspheres may be incorporated into a polyurethane coating solution which is then coated onto the device), or (e) incorporated into or onto the device following coating of the device with a coating composition.

In yet another aspect, a particulate form of the active agent (e.g., silk, wool, cyanoacrylate particles or chitosan) may be coated onto the device. In one embodiment, the particulate form may be incorporated into a polymeric carrier (e.g., PLG, PLA, polyurethane). Alternatively, or in addition, particles of the active agent can be applied onto a polymer-coated device. For example, a device can be coated with a polymer (e.g., a polyurethane) and then allowed to partially dry such that the surface is still tacky. A particulate form of the fibrosing agent or a fibrosing agent and secondary carrier, such as described above, can then be applied to all or a portion of the tacky coating after which the device is dried.

In yet another aspect, a device having a polymeric coating with or without a fibrosing agent can be subjected to a thermal treatment process to soften the coating. A fibrosing agent or a fibrosing agent and secondary carrier then is applied to all or a portion of the softened coating.

The coated device may be further coated with an additional composition and/or be treated to alter the release characteristics of the coating composition and/or fibrosing agent.

In one aspect, the device having a fibrosing agent or fibrosing composition incorporated into or coated onto the device may be further coated with a composition or compound which delays the onset of activity of the fibrosing agent for a period of time after implantation. Protection of a biologically active surface can be achieved by coating the device surface with an inert molecule that prevents access to the active site through steric hindrance. Representative examples of such compositions or compounds include biologically inert materials such as gelatin, PLGA/MePEG film, PLA, polyurethanes, silicone rubbers, surfactants, lipids, or polyethylene glycol, as well as biologically active materials such as heparin (e.g., to induce coagulation). In one embodiment, the active agent (e.g., poly-L-lysine, fibronectin, chitosan, silk, wool, bleomycin, cyclosporine A, or CTGF) on the device is top-coated with a physical barrier that does not contain a fibrosing agent. The barrier layer can include non-degradable materials or biodegradable materials such as, e.g., gelatin, PLGA/MePEG film, PLA, PLG, or polyethylene glycol. The barrier layer (e.g., dissolves slowly or degrades once implanted into the host. As the top layer dissolves or degrades, the active agent becomes exposed to the surrounding tissue and/or can be released from the coating.

In one embodiment, the rate of diffusion of the therapeutic agent in the barrier coat is slower that the rate of diffusion of the therapeutic agent in the coating layer. In the case of PLGA/MePEG, once the PLGA/MePEG becomes exposed to the bloodstream, the MePEG will dissolve out of the PLGA, leaving channels through the PLGA to an underlying layer containing the fibrosing agent (e.g., silk), which then can then diffuse into the vessel wall and initiate fibrosis.

Within yet another embodiment, the outer layer of the coated device (e.g., a stent or stent graft), which is capable of inducing an in vivo fibrotic response, is further treated to crosslink or functionalize the outer layer of the coating. Crosslinking of the coating (and/or additional surface modification) can be accomplished using a variety of methods, including, for example, subjecting the coated device to a plasma treatment process. The degree of crosslinking and nature of the surface modification can be altered by changing the RF power setting, the location with respect to the plasma, the duration of treatment, as well as the gas composition introduced into the plasma chamber.

Protection of a biologically active surface can also be achieved by coating the surface with an inactive form of the fibrosing agent, which is later activated. The fibrosing implant or device may be activated before, during, or after deployment (e.g., an inactive agent on the device may be first activated to one that induces or accelerates an in vivo fibrotic reaction).

In one embodiment, the intravascular device can be coated with an inactive form of the fibrosis-inducing agent, such as poly-L-lysine, fibronectin, chitosan, silk, wool, bleomycin, cyclosporine A, or CTGF, applied as described herein, which is then activated once the device is deployed. Activation can be achieved by injecting an activating agent (e.g., an enzyme) or a composition that includes an activating agent into the tissue or area surrounding the device after deployment of the device or after the fibrosis-inducing agent has been administered to the tissue (via drug delivery catheters or balloons).

In one embodiment, an intravascular device includes a first coating layer that includes a biologically active fibrosis-inducing agent, such as poly-L-lysine, fibronectin, or chitosan, bleomycin, silk, wool, cyclosporine A, or CTGF, and a first reactive component. In one embodiment, the first reactive component is capable of reaction with a polyethylene glycol. The coated device can be further coated with a second composition that includes a second reactive component (e.g., polyethylene glycol) that is capable of reaction with the first reactive component in the first coating layer. The reactive components of the first and second coating layers can be bonded via a condensation reaction through formation of ester bonds. Prior to the deployment of the intra-arterial segment of the device, an esterase is injected into the treatment site around the outside of the intravascular device, which can cleave the ester linkages, thus allowing the agent to become available to initiate fibrosis.

In other embodiments, the intravascular device may further include an agent that delay coagulation, such as heparin. The anti-coagulant can be coated on top of the fibrosis-inducing agent (e.g., poly-l-lysine, fibronectin, chitosan, silk, wool, bleomycin, cyclosporine A, or CTGF) or composition comprising the fibrosis-inducing agent. As the anti-coagulant dissolves away, its anti-coagulant activity ceases, such that the fibrosing agent can initiate a fibrotic response.

Within certain embodiments of the invention, the therapeutic compositions may also comprise additional ingredients such as surfactants (e.g., PLURONICS, such as F-127, L-122, L-101, L-92, L-81, and L-61), anti-inflammatory agents, anti-thrombotic agents, anti-infective agents, preservatives, anti-oxidants and/or anti-platelet agents.

Within certain embodiments of the invention, the therapeutic agent or carrier can also comprise radio-opaque, echogenic materials and magnetic resonance imaging (MRI) responsive materials (i.e., MRI contrast agents) to aid in visualization of the device under ultrasound, fluoroscopy and/or MRI. For example, a device may be made with or coated with a composition which is echogenic or radiopaque (e.g., made with echogenic or radiopaque with materials such as powdered tantalum, tungsten, barium carbonate, bismuth oxide, barium sulfate, metrazimide, iopamidol, iohexol, iopromide, iobitridol, iomeprol, iopentol, ioversol, ioxilan, iodixanol, iotrolan, acetrizoic acid derivatives, diatrizoic acid derivatives, iothalamic acid derivatives, ioxithalamic acid derivatives, metrizoic acid derivatives, iodamide, lypophylic agents, iodipamide and ioglycamic acid or, by the addition of microspheres or bubbles which present an acoustic interface). Visualization of a device by ultrasonic imaging may be achieved using an echogenic coating. Echogenic coatings are described in, e.g., U.S. Pat. Nos. 6,106,473 and 6,610,016. For visualization under MRI, contrast agents (e.g., gadolinium (III) chelates or iron oxide compounds) may be incorporated into or onto the device, such as, for example, as a component in a coating or within the void volume of the device (e.g., within a lumen, reservoir, or within the structural material used to form the device). In some embodiments, a medical device may include radio-opaque or MRI visible markers (e.g., bands) that may be used to orient and guide the device during the implantation procedure.

Medical implants may, alternatively, or in addition, be visualized under visible light, using fluorescence, or by other spectroscopic means. Visualization agents that can be included for this purpose include dyes, pigments, and other colored agents. In one aspect, the medical implant may further include a colorant to improve visualization of the implant in vivo and/or ex vivo. Frequently, implants can be difficult to visualize upon insertion, especially at the margins of implant. A coloring agent can be incorporated into a medical implant to reduce or eliminate the incidence or severity of this problem. The coloring agent provides a unique color, increased contrast, or unique fluorescence characteristics to the device. In one aspect, a solid implant is provided that includes a colorant such that it is readily visible (under visible light or using a fluorescence technique) and easily differentiated from its implant site. In another aspect, a colorant can be included in a liquid or semi-solid composition. For example, a single component of a two component mixture may be colored, such that when combined ex-vivo or in-vivo, the mixture is sufficiently colored.

The coloring agent may be, for example, an endogenous compound (e.g., an amino acid or vitamin) or a nutrient or food material and may be a hydrophobic or a hydrophilic compound. Preferably, the colorant has a very low or no toxicity at the concentration used. Also preferred are colorants that are safe and normally enter the body through absorption such as β-carotene. Representative examples of colored nutrients (under visible light) include fat soluble vitamins such as Vitamin A (yellow); water soluble vitamins such as Vitamin B12 (pink-red) and folic acid (yellow-orange); carotenoids such as β-carotene (yellow-purple) and lycopene (red). Other examples of coloring agents include natural product (berry and fruit) extracts such as anthrocyanin (purple) and saffron extract (dark red). The coloring agent may be a fluorescent or phosphorescent compound such as α-tocopherolquinol (a Vitamin E derivative) or L-tryptophan. Derivatives, analogues, and isomers of any of the above colored compound also may be used. The method for incorporating a colorant into an implant or therapeutic composition may be varied depending on the properties of and the desired location for the colorant. For example, a hydrophobic colorant may be selected for hydrophobic matrices. The colorant may be incorporated into a carrier matrix, such as micelles. Further, the pH of the environment may be controlled to further control the color and intensity.

In one aspect, the composition and devices of the present invention include one or more coloring agents, also referred to as dyestuffs, which will be present in an effective amount to impart observable coloration to the composition, e.g., the gel. Examples of coloring agents include dyes suitable for food such as those known as F. D. & C. dyes and natural coloring agents such as grape skin extract, beet red powder, beta carotene, annato, carmine, turmeric, paprika, and so forth. Derivatives, analogues, and isomers of any of the above colored compound also may be used. The method for incorporating a colorant into an implant or therapeutic composition may be varied depending on the properties of and the desired location for the colorant. For example, a hydrophobic colorant may be selected for hydrophobic matrices. The colorant may be incorporated into a carrier matrix, such as micelles. Further, the pH of the environment may be controlled to further control the color and intensity.

In one aspect, the compositions and devices of the present invention include one or more preservatives or bacteriostatic agents present in an effective amount to preserve the composition and/or inhibit bacterial growth in the composition, for example, bismuth tribromophenate, methyl hydroxybenzoate, bacitracin, ethyl hydroxybenzoate, propyl hydroxybenzoate, erythromycin, chlorocresol, benzalkonium chlorides, and the like. Examples of additional preservative include paraoxybenzoic acid esters, chlorobutanol, benzylalcohol, phenethyl alcohol, dehydroacetic acid, and sorbic acid. In one aspect, the compositions of the present invention include one or more bactericidal (also known as bacteriacidal) agents.

In one aspect, the compositions and devices of the present invention include one or more antioxidants, present in an effective amount. Examples of the antioxidant include sulfites, alpha-tocopherol and ascorbic acid.

Within related aspects of the present invention, intravascular devices (e.g., stents, stent grafts, aneurysm coils, embolic agents, drug delivery catheters or balloons) and compositions are provided that may or may not be associated with a device, which release an agent which induces fibrosis in vivo upon deployment of the device or administration of the composition. In certain aspects, the fibrosis-inducing agent or composition that comprises the fibrosis-inducing agent is delivered locally or regionally to the treatment site from the device or composition.

Within certain aspects of the present invention, the therapeutic composition should be biocompatible, and release one or more fibrosing agents over a period ranging from several hours, to several days, or over a period of many months. The scarring agent that is on, in or near the device may be released from the composition and/or device in a time period that may be measured from the time of implantation, which ranges from about less than 1 day to about 180 days. Generally, the release time may also be from about less than 1 day to about 7 days; from 7 days to about 14 days; from 14 days to about 28 days; from 28 days to about 56 days; from 56 days to about 90 days; from 90 days to about 180 days.

The devices of the present invention may be configured to release the scarring agent at one or more phases, the one or more phases having similar or different performance (e.g., release) profiles. The therapeutic agent may be made available to the tissue at amounts which may be sustainable, intermittent, or continuous; in one or more phases; and/or rates of delivery; effective to increase or promote any one or more components of fibrosis (or scarring), including: formation of new blood vessels (angiogenesis), migration and proliferation of connective tissue cells (such as fibroblasts or smooth muscle cells), deposition of extracellular matrix (ECM), and remodeling (maturation and organization of the fibrous tissue); or the agent can act as a vascular wall irritant.

Thus, the release rate may be programmed to impact fibrosis (or scarring) by releasing the scarring agent at a time such that at least one of the components of fibrosis is promoted or increased. Moreover, the predetermined release rate may reduce agent loading and/or concentration as well as potentially providing minimal drug washout and thus, increases efficiency of drug effect. In one embodiment, the rate of release may provide a sustainable level of the scarring agent to the susceptible vascular wall site. In another embodiment, the rate of release is substantially constant. The rate may decrease and/or increase over time, and it may optionally include a substantially non-release period. The release rate may comprise a plurality of rates. In an embodiment, the plurality of release rates may include rates selected from the group consisting of substantially constant, decreasing, increasing, and substantially non-releasing.

The total amount of scarring agent made available on, in or near the device may be in an amount ranging from about 0.01 μg (micrograms) to about 2500 mg (milligrams). Generally, the scarring agent may be in the amount ranging from 0.01 μg to about 10 μg; or from 10 μg to about 1 mg; or from 1 mg to about 10 mg; or from 10 mg to about 100 mg; or from 100 mg to about 500 mg; or from 500 mg to about 2500 mg.

The surface amount of scarring agent on, in or near the device may be in an amount ranging from less than 0.01 μg to about 250 μg per mm² of device surface area. Generally, the scarring agent may be in the amount ranging from less than 0.01 μg/mm²; or from 0.01 μg to about 10 pg/mm²; or from 10 μg to about 25 μg/mm²; or from 25 μg to about 250 μg/mm².

In one aspect, “quick release” or “burst” therapeutic compositions are provided that release greater than 10%, 20%, or 25% (w/v) of a fibrosis-inducing agent over a period of 7 to 10 days. Such “quick release” compositions should, within certain embodiments, be capable of releasing therapeutic levels (where applicable) of a desired fibrosing agent. Within other embodiments, “slow release” therapeutic compositions are provided that release less than 1% (w/v) of a fibrosis-inducing agent over a period of 7 to 10 days. Within other embodiments therapeutic compositions are provided that release either less than 1% (w/v) of a fibrosing-inducing agent over a period longer than 10 days or do not release the therapeutic composition at all, but maintain the composition for a very long period of time such as for the entire duration of the device placement in the body.

The amount of scarring agent released from the composition and/or device as a function of time may be determined based on the in vitro release characteristics of the agent from the composition. The in vitro release rate may be determined by placing the scarring agent within the composition or device in an appropriate buffer such as 0.1M phosphate buffer (pH 7.4)) at 37° C. Samples of the buffer solution are then periodically removed for analysis by either HPLC or by gravimetric means, and the buffer is replaced to avoid any saturation effects.

Based on the in vitro release rates, the release of scarring agent per day may range from an amount ranging from about 0.0 μg (micrograms) to about 2500 mg (milligrams). Generally, the scarring agent that may be released in a day may be in the amount ranging from 0.0 to 0.01 μg; 0.01 μg to about 10 μg; or from 10 pg to about 1 mg; or from 1 mg to about 10 mg; or from 10 mg to about 100 mg; or from 100 mg to about 500 mg; or from 500 mg to about 2500 mg. In one embodiment, the scarring agent is made available to the susceptible tissue site in a constant but substantially unchanging manner so that the agent remains at the tissue essentially permanently. In another embodiment, the scarring agent is made available to the susceptible tissue in a sustained and/or controlled manner which results in increased efficiency and/or efficacy. Further, the release rates may vary during either or both of the initial and subsequent release phases. There may also be additional phase(s) for release of the same substance(s) and/or different substance(s).

Further, therapeutic compositions of the present invention should preferably be have a stable shelf-life for at least several months and capable of being produced and maintained under sterile conditions. The composition may be sterile either by preparing them under aseptic environment and/or they may be terminally sterilized using methods available in the art. Many pharmaceuticals are manufactured to be sterile and this criterion is defined by the USP XXII <1211>. The term “USP” refers to U.S. Pharmacopeia (see www.usp.org, Rockville, Md.). Sterilization may be accomplished by a number of means accepted in the industry and listed in the USP XXII <1211>, including gas sterilization, ionizing radiation or, when appropriate, filtration. Sterilization may be maintained by what is termed aseptic processing, defined also in USP XXII <1211>. Acceptable gases used for gas sterilization include ethylene oxide. Acceptable radiation types used for ionizing radiation methods include gamma, for instance from a cobalt 60 source and electron beam. A typical dose of gamma radiation is 2.5 MRad. Sterilization may also occur by terminally using gamma radiation or electron beam sterilization methods. Filtration may be accomplished using a filter with suitable pore size, for example 0.22 μm and of a suitable material, for instance polytetrafluoroethylene (e.g., TEFLON). A combination of these methods may also be used to prepare the composition in the sterile form.

In another aspect, the compositions and devices of the present invention are contained in a container that allows them to be used for their intended purpose. Properties of the container that are important are a volume of empty space to allow for the addition of a constitution medium, such as water or other aqueous medium, e.g., saline, acceptable light transmission characteristics in order to prevent light energy from damaging the composition in the container (refer to USP XXII <661>), an acceptable limit of extractables within the container material (refer to USP XXII), an acceptable barrier capacity for moisture (refer to USP XXII <671>) or oxygen. In the case of oxygen penetration, this may be controlled by including in the container, a positive pressure of an inert gas, such as high purity nitrogen, or a noble gas, such as argon.

Typical materials used to make containers for pharmaceuticals include USP Type I through III and Type NP glass (refer to USP XXII <661>), polyethylene, TEFLON, silicone, and gray-butyl rubber.

It should be readily evident to one of skill in the art that any of the previously described fibrosis inducing agents, or derivatives and analogues thereof, can be utilized to create variations of the above compositions without deviating from the spirit and scope of the invention. It should also be apparent that the agent can be utilized in a composition with or without polymer carrier and that altering the carrier does not deviate from the scope of this invention.

For all the previously described embodiments, examples of suitable fibrosing agents include tissue irritants such tissue as silk, wool, asbestos, silica, bleomycin, neomycin, talcum powder, metallic beryllium, and copper are particularly suitable for the practice of this invention. Other agents which may be incorporated into or onto the implant or device or released from the implant or device include extracellular matrix components such as fibrous structural proteins (e.g., fibrillar collagens, nonfibrillar collagen and elastins), adhesive glycoproteins (e.g., laminin and fibronectin), proteoglycans (e.g., heparin sulphate, chondroitin sulphate, dermatan sulphate), hyaluronan (e.g., hyaluronic acid), secreted protein acidic and rich in cysteine (SPARC), thrombospondins, tenacin, inhibitors of matrix metalloproteinases (e.g., TIMPs and synthetic TIMPs such as marimistat, batimistat, doxycycline, tetracycline, minocycline, TROCADE, Ro-1130830, CGS 27023A, BMS-275291) and polylysine. Growth factors and inflammatory cytokines involved in angiogenesis, fibroblast migration, fibroblast proliferation, ECM synthesis and tissue remodeling such as epidermal growth factor (EGF) family, transforming growth factor-α (TGF-α), transforming growth factor-β (TGF-9-1, TGF-9-2, TGF-9-3), platelet-derived growth factor (PDGF), fibroblast growth factor (acidic—aFGF; and basic—bFGF), bone morphogenic proteins, activins, vascular endothelial growth factor (VEGF, VEGF-B, VEGF-C, placental growth factor—PIGF), angiopoietins, insulin-like growth factors (IGF), hepatocyte growth factor (HGF), connective tissue growth factor (CTGF), myeloid colony-stimulating factors (CSFs), granulocyte-macrophage colony-stimulating factors (GM-CSF), granulocyte colony-stimulating factor (G-CSF), macrophage colony-stimulating factor (M-CSF), erythropoietin, interleukins (particularly IL-1, IL-8, IL-6), tumor necrosis factor-α (TNF9), nerve growth factor (NGF), interferon-α, interferon-β, and growth hormone (GH) are also suitable for incorporation and release from specific intravascular devices. Other agents which may be coated onto or released by the implant or device include adhesives such as cyanoacrylate or materials made from 4-armed thiol PEG (10K), a 4-armed NHS PEG(10K) and methylated collagen.

5) Coating of Devices with Fibrosing Agents

As described above, a range of polymeric and non-polymeric materials can be used to incorporate the fibrosing agent onto, or into, a device such as a stent, stent graft, aneurysm coil or embolic agent. In one aspect, the fibrosing agent can be coated onto a surface of a medical device. Coating of the device with these fibrosing agent containing compositions or with the fibrosing agent, however, only is one process that can be used to incorporate the fibrosing agent into or onto the device. The fibrosing agent or a composition comprising a fibrosing agent may be coated onto the entire device or a portion of the device. This can be accomplished, for example, using a variety of methods known in the art such as by dipping, spraying, electrospinning, painting or by vacuum deposition.

a) Dip Coating

Dip coating is one coating process that can be used to coat the device. In one embodiment, the fibrosing agent is dissolved in a solvent for the fibrosing agent and is then coated onto the device.

Fibrosing Agent with an Inert-Solvent

In one embodiment, the solvent is an inert solvent for the device such that the solvent does not dissolve the medical device to any great extent and is not absorbed by the device to any great extent. The device can be immersed, either partially or completely, in the fibrosing agent/solvent solution for a specific period of time. The rate of immersion into the fibrosing agent/solvent solution can be altered (e.g., 0.001 cm per sec to 50 cm per sec). The device can then be removed from the solution. The rate at which the device can be withdrawn from the solution can be altered (e.g., 0.001 cm per sec to 50 cm per sec). The coated device can be air-dried. The dipping process can be repeated one or more times depending on the specific application. The device can be dried under vacuum to reduce residual solvent levels. This process will result in the fibrosing agent being coated on the surface of the device.

Fibrosing Agent with a Swelling Solvent

In one embodiment, the solvent is one that will not dissolve the device but will be absorbed by the device. These solvents can thus swell the device to some extent. The device can be immersed, either partially or completely, in the fibrosing agent/solvent solution for a specific period of time (seconds to days). The rate of immersion into the fibrosing agent/solvent solution can be altered (e.g., 0.001 cm per sec to 50 cm per sec). The device can then be removed from the solution. The rate at which the device can be withdrawn from the solution can be altered (e.g., 0.001 cm per sec to 50 cm per sec). The coated device can be air-dried. The dipping process can be repeated one or more times depending on the specific application. The device can be dried under vacuum to reduce residual solvent levels. This process will result in the fibrosing agent being adsorbed into the medical device. The fibrosing agent may also be present on the surface of the device. The amount of surface associated fibrosing agent may be reduced by dipping the coated device into a solvent for the fibrosing agent or by spraying the coated device with a solvent for the fibrosing agent.

Fibrosing Agent with a Solvent

In one embodiment, the solvent is one that will be absorbed by the device and that will dissolve the device. The device can be immersed, either partially or completely, in the fibrosing agent/solvent solution for a specific period of time (seconds to hours). The rate of immersion into the fibrosing agent/solvent solution can be altered (e.g., 0.001 cm per sec to 50 cm per sec). The device can then be removed from the solution. The rate at which the device can be withdrawn from the solution can be altered (e.g., 0.001 cm per sec to 50 cm per sec). The coated device can be air-dried. The dipping process can be repeated one or more times depending on the specific application. The device can be dried under vacuum to reduce residual solvent levels. This process will result in the fibrosing agent being adsorbed into the medical device as well as being surface associated. In the preferred embodiment, the exposure time of the device to the solvent would be such that the device does not undergo significant permanent dimensional changes. The fibrosing agent may also be present on the surface of the device. The amount of surface associated fibrosing agent may be reduced by dipping the coated device into a solvent for the fibrosing agent or by spraying the coated device with a solvent for the fibrosing agent.

In the above description the device can be a device that has not been modified as well as a device that has been further modified by coating with a polymer (e.g., parylene), surface treated by plasma treatment, flame treatment, corona treatment, surface oxidation or reduction, surface etching, mechanical smoothing or roughening, or grafting prior to the coating process.

In one embodiment, the fibrosing agent and a polymer are dissolved in a solvent, for both the polymer and the fibrosing agent, and are then coated onto the device.

Fibrosing Agent/Polymer with an Inert-Solvent

In one embodiment, the solvent is an inert solvent for the device such that the solvent does not dissolve the medical device to any great extent and is not absorbed by the device to any great extent. The device can be immersed, either partially or completely, in the fibrosing agent/polymer/solvent solution for a specific period of time. The rate of immersion into the fibrosing agent/polymer/solvent solution can be altered (e.g., 0.001 cm per sec to 50 cm per sec). The device can then be removed from the solution. The rate at which the device can be withdrawn from the solution can be altered (e.g., 0.001 cm per sec to 50 cm per sec). The coated device can be air-dried. The dipping process can be repeated one or more times depending on the specific application. The device can be dried under vacuum to reduce residual solvent levels. This process will result in the fibrosing agent/polymer being coated on the surface of the device.

Fibrosing Agent/Polymer with a Swelling Solvent

In one embodiment, the solvent is one that will not dissolve the device but will be absorbed by the device. These solvents can thus swell the device to some extent. The device can be immersed, either partially or completely, in the fibrosing agent/polymer/solvent solution for a specific period of time (seconds to days). The rate of immersion into the fibrosing agent/polymer/solvent solution can be altered (e.g., 0.001 cm per sec to 50 cm per sec). The device can then be removed from the solution. The rate at which the device can be withdrawn from the solution can be altered (e.g., 0.001 cm per sec to 50 cm per sec). The coated device can be air-dried. The dipping process can be repeated one or more times depending on the specific application. The device can be dried under vacuum to reduce residual solvent levels. This process will result in the fibrosing agent/polymer being coated onto the surface of the device as well as the potential for the fibrosing agent being adsorbed into the medical device. The fibrosing agent may also be present on the surface of the device. The amount of surface associated fibrosing agent may be reduced by dipping the coated device into a solvent for the fibrosing agent or by spraying the coated device with a solvent for the fibrosing agent.

Fibrosing Agent/Polymer with a Solvent

In one embodiment, the solvent is one that will be absorbed by the device and that will dissolve the device. The device can be immersed, either partially or completely, in the fibrosing agent/solvent solution for a specific period of time (seconds to hours). The rate of immersion into the fibrosing agent/solvent solution can be altered (e.g., 0.001 cm per sec to 50 cm per sec). The device can then be removed from the solution. The rate at which the device can be withdrawn from the solution can be altered (e.g., 0.001 cm per sec to 50 cm per sec). The coated device can be air-dried. The dipping process can be repeated one or more times depending on the specific application. The device can be dried under vacuum to reduce residual solvent levels. In the preferred embodiment, the exposure time of the device to the solvent would be such that there is not significant permanent dimensional change to the device (other than those associated with the coating itself). The fibrosing agent may also be present on the surface of the device. The amount of surface associated fibrosing agent may be reduced by dipping the coated device into a solvent for the fibrosing agent or by spraying the coated device with a solvent for the fibrosing agent.

In the above description the device can be a device that has not been modified as well as a device, such as a stent, stent graft, aneurysm coil or embolic agent, that has been further modified by coating with a polymer (e.g., parylene), surface treated by plasma treatment, flame treatment, corona treatment, surface oxidation or reduction, surface etching, mechanical smoothing or roughening, or grafting prior to the coating process.

In any one the above dip coating methods, the surface of the device can be treated with a plasma polymerization method prior to coating of the scarring agent or scarring agent containing composition, such that a thin polymeric layer is deposited onto the device surface. Examples of such methods include parylene coating of devices and the use of various monomers such hydrocyclosiloxane monomers. Parylene coating may be especially advantageous if the device, or portions of the device, is composed of materials (e.g., stainless steel, nitinol) that do not allow incorporation of the therapeutic agent(s) into the surface layer using one of the above methods. A parylene primer layer may be deposited onto the electrical device using a parylene coater (e.g., PDS 2010 LABCOTER2 from Cookson Electronics, Inc., Foxborough, Mass.) and a suitable reagent (e.g., di-p-xylylene or dichloro-di-p-xylylene) as the coating feed material. Parylene compounds are commercially available, for example, from Specialty Coating Systems, Indianapolis, Ind.), including PARYLENE N (di-p-xylylene), PARYLENE C (a monchlorinated derivative of PARYLENE N, and PARYLENE D, a dichlorinated derivative of PARYLENE N).

In another embodiment, a suspension of the fibrosing agent in a polymer solution can be prepared. The suspension can be prepared by choosing a solvent that can dissolve the polymer but not the fibrosing agent or a solvent that can dissolve the polymer and in which the fibrosing agent is above its solubility limit. In similar processes described above, a device can be dipped into the suspension of the fibrosing agent and polymer solution such that the device is coated with the suspension.

b) Spray Coating

Spray coating is another coating process that can be used. In the spray coating process, a solution or suspension of the fibrosing agent, with or without a polymeric or non-polymeric carrier, is nebulized and directed to the device to be coated by a stream of gas. One can use spray devices such as an air-brush (for example models 2020, 360, 175, 100, 200, 150, 350, 250, 400, 3000, 4000, 5000, 6000 from Badger Air-brush Company, Franklin Park, Ill.), spray painting equipment, TLC reagent sprayers (for example Part #14545 and 14654, Alltech Associates, Inc. Deerfield, Ill., and ultrasonic spray devices (for example those available from Sono-Tek, Milton, N.Y.). One can also use powder sprayers and electrostatic sprayers.

In one embodiment, the fibrosing agent is dissolved in a solvent for the fibrosis agent and is then sprayed onto the device.

Fibrosing Agent with an Inert-Solvent

In one embodiment, the solvent is an inert solvent for the device such that the solvent does not dissolve the medical device to any great extent and is not absorbed by the device to any great extent. The device can be held in place or the device can be mounted onto a mandrel or rod that has the ability to move in an X, Y or Z plane or a combination of these planes. Using one of the above described spray devices, the device can be spray coated such that the device is either partially or completely coated with the fibrosing agent/solvent solution. The rate of spraying of the fibrosing agent/solvent solution can be altered (e.g., 0.001 ml per sec to 10 ml per sec) to ensure that a good coating of the fibrosing agent is obtained. The coated device can be air-dried. The spray coating process can be repeated one or more times depending on the specific application. The device can be dried under vacuum to reduce residual solvent levels. This process will result in the fibrosing agent being coated on the surface of the device.

Fibrosing Agent with a Swelling Solvent

In one embodiment, the solvent is one that will not dissolve the device but will be absorbed by the device. These solvents can thus swell the device to some extent. The device can be spray coated, either partially or completely, in the fibrosing agent/solvent solution. The rate of spraying of the fibrosing agent/solvent solution can be altered (e.g., 0.001 ml per sec to 10 ml per sec) to ensure that a good coating of the fibrosing agent is obtained. The coated device can be air-dried. The spray coating process can be repeated one or more times depending on the specific application. The device can be dried under vacuum to reduce residual solvent levels. This process will result in the fibrosing agent being adsorbed into the medical device. The fibrosing agent may also be present on the surface of the device. The amount of surface associated fibrosing agent may be reduced by dipping the coated device into a solvent for the fibrosing agent or by spraying the coated device with a solvent for the fibrosing agent.

Fibrosing Agent with a Solvent

In one embodiment, the solvent is one that will be absorbed by the device and that will dissolve the device. The device can be spray coated, either partially or completely, in the fibrosing agent/solvent solution. The rate of spraying of the fibrosing agent/solvent solution can be altered (e.g., 0.001 ml per sec to 10 ml per sec) to ensure that a good coating of the fibrosing agent is obtained. The coated device can be air-dried. The spray coating process can be repeated one or more times depending on the specific application. The device can be dried under vacuum to reduce residual solvent levels. This process will result in the fibrosing agent being adsorbed into the medical device as well as being surface associated. In one embodiment, the exposure time of the device to the solvent would be such that the device would incur no significant permanent dimensional changes. The fibrosing agent may also be present on the surface of the device. The amount of surface associated fibrosing agent may be reduced by dipping the coated device into a solvent for the fibrosing agent or by spraying the coated device with a solvent for the fibrosing agent.

In the above description the device can be a device that has not been modified as well as a device that has been further modified by coating with a polymer (e.g., parylene), surface treated by plasma treatment, flame treatment, corona treatment, surface oxidation or reduction, surface etching, mechanical smoothing or roughening, or grafting prior to the coating process.

In one embodiment, the fibrosing agent and a polymer are dissolved in a solvent, for both the polymer and the fibrosing agent, and are then spray coated onto the device.

Fibrosing Agent/Polymer with an Inert-Solvent

In one embodiment, the solvent is an inert solvent for the device such that the solvent does not dissolve the medical device to any great extent and is not absorbed by the device to any great extent. The device can be spray coated, either partially or completely, in the fibrosing agent/polymer/solvent solution for a specific period of time. The rate of spraying of the fibrosing agent/solvent solution can be altered (e.g., 0.001 ml per sec to 10 ml per sec) to ensure that a good coating of the fibrosing agent is obtained. The coated device can be air-dried. The spray coating process can be repeated one or more times depending on the specific application. The device can be dried under vacuum to reduce residual solvent levels. This process will result in the fibrosing agent/polymer being coated on the surface of the device.

Fibrosing Agent/Polymer with a Swelling Solvent

In one embodiment, the solvent is one that will not dissolve the device but will be absorbed by the device. These solvents can thus swell the device to some extent. The device can be spray coated, either partially or completely, in the fibrosing agent/polymer/solvent solution. The rate of spraying of the fibrosing agent/solvent solution can be altered (e.g., 0.001 ml per sec to 10 ml per sec) to ensure that a good coating of the fibrosing agent is obtained. The coated device can be air-dried. The spray coating process can be repeated one or more times depending on the specific application. The device can be dried under vacuum to reduce residual solvent levels. This process will result in the fibrosing agent/polymer being coated onto the surface of the device as well as the potential for the fibrosing agent being adsorbed into the medical device. The fibrosing agent may also be present on the surface of the device. The amount of surface associated fibrosing agent may be reduced by dipping the coated device into a solvent for the fibrosing agent or by spraying the coated device with a solvent for the fibrosing agent.

Fibrosing Agent/Polymer with a Solvent

In one embodiment, the solvent is one that will be absorbed by the device and that will dissolve the device. The device can be spray coated, either partially or completely, in the fibrosing agent/solvent solution. The rate of spraying of the fibrosing agent/solvent solution can be altered (e.g., 0.001 ml per sec to 10 ml per sec) to ensure that a good coating of the fibrosing agent is obtained. The coated device can be air-dried. The spray coating process can be repeated one or more times depending on the specific application. The device can be dried under vacuum to reduce residual solvent levels. In the preferred embodiment, the exposure time of the device to the solvent would be such that there are not significant permanent dimensional changes to the device (other than those associated with the coating itself). The fibrosing agent may also be present on the surface of the device. The amount of surface associated fibrosing agent may be reduced by dipping the coated device into a solvent for the fibrosing agent or by spraying the coated device with a solvent for the fibrosing agent.

In the above description the device can be a device that has not been modified as well as a device that has been further modified by coating with a polymer (e.g., parylene), surface treated by plasma treatment, flame treatment, corona treatment, surface oxidation or reduction, surface etching, mechanical smoothing or roughening, or grafting prior to the coating process.

J. Methods for Using Intravascular Devices

The intravascular devices of the invention may be used to treat a variety of medical conditions, including, but not limited to, the occlusion of aneurysms and the stabilization of vulnerable plaque.

Treatment of Aortic Aneurysms

In one aspect, the intravascular device is an endovascular prosthesis such as a stent graft for use in treating patients having aneurysms (e.g., abdominal aortic aneurysms, thoracic aortic aneurysms, or iliac artery aneurysms). A stent graft is used clinically for bypassing a diseased portion of a vessel on its inner (luminal) aspect. The graft is inserted into a diseased vessel (typically an artery which has formed an aneurismal dilatation as a result of atherosclerosis), such that it connects a section of normal (nondiseased) artery above the aneurysm to a section of normal artery below it. The stent and the graft material exclude the aneurysm from the circulation, eliminate arterial blood pressure from being exerted against the weakened aneurysm wall and reduce the risk the aneurysm will rupture. In one embodiment, the stent graft is delivered into a patient (e.g., percutaneously inserted via the femoral artery, maneuvered into place via the arterial system under radiologic guidance) in a constrained form and self-expands into place after release of a constraining device. The methods utilize the stent grafts of the present invention. As utilized herein, it should be understood that “reduction in the risk of rupture” or “prevention of the risk of rupture” refers to a statistically significant reduction in the number, timing, or, rate of rupture, and not to a permanent prohibition of any rupture. Likewise, a “reduction in the risk of perigraft leakage” refers to statistically significant enhancement in the effectiveness and/or effective lifetime of a stent graft, which may or may not result in a permanent or complete cessation of perigraft leakage.

The stent grafts of the present invention may be utilized to induce a perigraft reaction, induce neointimal formation in the wall of the aneurysm, or to otherwise create a tight adhesive bond between an endovascular prosthesis and the vascular wall in a host. Such stent grafts are capable of providing a solution to the following common problems associated with endovascular stent graft technology.

1. Persistent Perigraft Leaks—The practice of this invention results in the formation of a fibrotic response, adhesion or tight adhesive bond between the proximal and distal ends of the stent graft and the vessel wall. Incorporation of the graft into the vessel wall (by encouraging fibrous tissue growth from the arterial wall into, and around, the graft) results in a more efficacious, biological and permanent sealing around the device that prevents late perigraft leaks from arising at either end of the device even if there is a change in aneurysm morphology. Moreover, formation of a fibrous response or tight adhesion between the body of the graft and the aneurysm itself may result in occlusion of, or prevention of a perigraft leak due to retrograde flow (i.e., persistence of, or late reopening of the inferior mesenteric artery or lumbar arteries extending into the aneurysm). If the aneurysm sac becomes filled fibrous tissue, there is no anatomical space for the lumbar arteries to “backflow” into, thereby reducing the possibility that this complication will occur.

2. Size of the Delivery Device—One difficulty with present stent grafts and their delivery devices is that they are quite large due to the required thickness of the stent graft. By inducing a reaction in the wall, which in itself conveys strength to the graft portion of the stent graft prosthesis, a thinner graft material may be utilized in stent grafts of the present invention compared to standard stent grafts (also, adherence of the graft to the vessel wall will maintain the lumen of the graft and lessen the need for mechanical support from the stent scaffold—which could also potentially be reduced in size). Thus, in the various aspects of the invention, the silk stent graft has a thickness of less than 24 French, or less than 23 French, or less than 22 French, or less than 21 French, or less than 20 French.

3. Anatomic Factors which limit Patients with Aneurismal Disease who are Candidates for Treatment with Endovascular Stent Grafts—By inducing a fibrotic reaction, or creating a tight durable adhesive bond between the prosthesis and the vascular wall at the proximal and distal margins of the grafted portion of the prosthesis, the length of the neck of the stent graft (particularly the proximal neck) can be shorter than the presently suggested 1.5 centimeters. This benefit is realized because the fibrotic reaction or tight adhesion between graft and vessel wall will enhance sealing of the graft even when there is a short length of contact between the graft and vessel wall. In an aneurysm, the walls are dilated and thus extend away from the graft. When there is a long neck, apposition between graft material and vessel wall is only between the portion of vessel wall of “normal” diameter. In some cases, the portion of the vessel to which the device is to be anchored is dilated, e.g., a dilated iliac artery distal to an abdominal aortic aneurysm. If this segment of the vessel is too dilated, it tends to continue expansion after graft insertion, resulting in late perigraft leaks. Patients with dilated iliac arteries or aortic neck might be denied therapy with uncoated devices but can advantageously receive a fibrosis-promoting stent graft of the present invention. Creation of a firm bond between the graft and the vessel wall will prevent the neck from expanding further.

4. Stent Graft Migration—Since the fibrosis-inducing stent graft of the present invention becomes firmly fixed against the vessel wall by more than just mechanical means (such as hooks or force of expansion between the stent graft and the vessel wall), migration of the stent graft or portions of the stent graft is prevented or reduced.

5. Aneurysm Rupture—Aneurysm rupture can occur after placement of a stent graft for several reasons: continued leakage into the sac due to device migration, leakage around the graft, leakage through the graft, retrograde vascular flow, or continued aneurysmal dilatation. The induction of a fibrous reaction between the graft and the vascular wall has the potential to reduce all of these problems. Anchoring the graft in place prevents stent graft migration and leakage around the graft (endoleaks). The formation of neointima into, and over, the graft has the effect of “biologically resurfacing” the graft lumen and making the problem of fabric wear (including the formation of holes) less problematic (since the fabric becomes covered by vascular wall tissue). Filling the aneurysmal sac with fibrous tissue closes the anatomical space between the stent graft and the vessel wall and eliminates the potential for blood to accumulate (whether due to leaks or retrograde flow), exert pressure on the wall, and increase the risk of rupture. Lastly, the natural history of scar tissue is to gradually contract with time. This will have the effect of pulling the aneurysm wall towards the graft and contracting the sac (analogous to removing the air from a balloon). The net effect is to shrink the diameter of the aneurysm, make it less likely to rupture (the risk of rupture increases as a function of increased diameter), and act counter to the natural tendency for aneurysms to progressively increase in size with time.

A. Abdominal Aortic Aneurysms

In one representative example, fibrosing stent grafts may be inserted into an abdominal aorta aneurysm (AAA), in order to treat or prevent rupture of the abdominal aorta. Briefly, using sterile conditions, under appropriate anesthesia and analgesia, the common femoral artery is surgically exposed and an arteriotomy is performed after clamping of the artery. A guide wire is manipulated through the iliac arterial system and over this a catheter is inserted into the proximal abdominal aorta and an angiogram or intravascular ultrasound is performed. Subsequently, the diagnostic catheter is exchanged over a guide wire for a delivery system, usually a sheath, containing the aortic portion of the stent graft system. In an articulated bifurcated system (the most common iteration), the ipsilateral iliac portion of the prosthesis is connected to the aortic portion of the prosthesis. In the case of a stent graft composed of self-expanding stents, the device is deployed by releasing it from its constrained configuration. If the stent graft skeleton is composed of balloon expandable stents, it is released by withdrawal of the sheath and inflating a balloon to expand the stent graft in place. After release of the aortic and ipsilateral iliac portion of the prosthesis, surgical exposure and cut down of the opposite iliac artery is performed and a guide wire is manipulated so that it passes through the deployed portion of the prosthesis. A similar delivery device containing the contralateral iliac limb of the prosthesis is then manipulated into the deployed aortic portion of the prosthesis and under fluoroscopic guidance is released in an appropriate position. The position is chosen so that the entire grafted portion of the stent graft sits below the renal arteries and preferably is deployed above the internal iliac arteries although one or both may be occluded. Depending on the patient's anatomy, further limb extensions may be inserted on either side. If the device is a tube graft, or a one piece bifurcated device, insertion via only one femoral artery may be required. A final angiogram is normally obtained by an angiographic catheter position with its distal portion in the upper abdominal aorta.

In another aspect, the fibrosing agent may be incorporated into a surgical sealant or adhesive (e.g., fibrin glue) that can be used to hold the stent graft in place. For example, a stent graft may be coated adluminally with an inactive fibrin-based sealant. After deployment of the stent graft, the fibrin sealant is then activated to glue the device to the vessel wall. Various therapeutic agents may be loaded into the sealant for controlled release in the vicinity of the stent graft (e.g., fibrosis inducing agents, thrombolytic agents, and thrombogenic agents).

B. Thoracic Aortic Aneurysm or Dissection

In another representative example, a fibrosing stent graft may be utilized to treat or prevent a thoracic aortic aneurysm. Briefly, under appropriate anesthesia and analgesia, using sterile technique, a catheter is inserted via the right brachial artery into the ascending thoracic aorta and an angiogram performed. Once the proximal and distal boundaries of the diseased segment of the aorta to be treated are defined, an operative exposure and arteriotomy of one of the common femoral arteries (usually the right) is performed. A guide wire is manipulated through the diseased segment of the aorta and over this, the delivery device, usually a sheath, is advanced so that the device is positioned across the diseased segment with the grafted portion of the stent immediately below the origin of the left subclavian artery. After contrast is injected to define the precise position of the stent graft, the device is deployed by withdrawing an outer sheath (in the case of self-expanding stents) so that the device is positioned immediately distal to the left subclavian artery with its distal portion extending beyond the diseased portion of the thoracic aorta but above the celiac axis. A final angiogram is performed via the catheter inserted by the right brachial artery. The vascular access wounds are then closed.

C. Vascular Embolization

In certain procedures, a stent graft may be used in conjunction with an embolization device or an embolic agent to occlude an aortic aneurysm. Embolization devices are designed to be placed within the vasculature (typically an artery) of the patient such that the flow of blood through a vessel (or portion of a vessel in the case of an aneurysm) is largely or completely obstructed. Embolization devices are designed to slow or eliminate blood flow to a tissue and may be used to treat a variety of medical conditions including vascular aneurysms (such as thoracic aortic aneurysm and abdominal aortic aneurysms) and vascular malformations (AV malformations, vascular tumors). For example, even after the initial successful placement of a stent graft (as described above), a catheter can be advanced into the aneurysm sac (between the vessel wall and the stent graft) and an embolic (or vascular filling) agent can be infiltrated into the aneurysm sac. The embolic agent will induce thrombosis, while the fibrosing agent will induce fibrosis in the aneurysmal sac as described previously.

An embolic agent or device can be inserted such that it becomes physically lodged in the artery lumen causing interruption of blood flow to a tissue. The embolic agent or device can also induce clotting in the vessel (or portion of a vessel) such that blood flow becomes obstructed by clot (or a combination of the device and clot). In either case, blood supply to a particular anatomical region (e.g., an aneurysm sac or a vascular malformation) is reduced, or eliminated, leading to ischemic damage or complete destruction of the unwanted tissue.

The embolic materials that are injected (or devices implanted) into the vasculature are capable of producing a permanent, obstructive scar in the aneurysm sac that results in regression and absorption of the unwanted vessel (or portion of the vessel). Permanent prevention of blood flow in the vessel can be achieved due to obstructive fibrosis, and the body resorbs the nonfunctioning vascular tissue and eliminates the blood vessel, leaving little or no chance for recurrence.

Numerous particles, microspheres and injectable polymer systems may be used as embolic agents, including injectable embolic agents, polymeric embolic agents, and embolic microspheres may be used. Embolization agents, which may be combined with one or more fibrosing agents according to the present invention, include several commercially available products. For example, the TRUFILL n-butyl Cyanoacrylate (n-BCA) Liquid Embolic System (Cordis, a division of Johnson and Johnson, Miami, Fla.); EMBOSPHERE Microspheres and EMBOGOLD Microspheres (Biosphere Medical, Inc., Rockland, Mass.); and the ONYX Liquid Embolic System (Micro Therapeutics, Irvine, Calif.) are all polymeric embolization systems suitable for combining with a fibrosing agent. Other examples of embolization devices include polymer/solvent systems containing a fibrosing agent in which the solvent diffuses from the polymer matrix once it has been injected at the treatment site (e.g., the degradable polymeric systems from Atrix, non-degradable polymeric compositions such as ONYX and EMBOLYX, and in situ forming materials such as those available from Biocure, Inc., Angiotech Pharmaceuticals, Inc., 3M Company and Neomend, Inc.). Other types of commercially available embolic agents that can be loaded or made with a fibrosing agent include PVA particles (Cook Group, Inc; Angiodynamics, Inc., Queensbury, N.Y.) and microsphere formulations (e.g., EMBOSPHERE from Biosphere, Inc., CONTOUR SE from Boston Scientific Corporation and BEAD BLOCK from Biocompatibles, Ltd., United Kingdom).

In one aspect, the present invention provides embolization agents combined with a fibrosing agent directly, or a composition (e.g., a polymeric or non-polymeric carrier) that includes a fibrosing agent, for the purpose of permanently occluding an aneurysm. The fibrosing agent can be delivered with the embolization agent in several ways, including: (a) fluids, suspensions, emulsions, microemulsions, microspheres, pastes, gels, microparticulates, sprays, aerosols, solid implants and other formulations (see those described above) which release a fibrosing agent(s); (b) microparticulate silk and/or silk strands (linear, branched, and/or coiled) either alone, or loaded with an additional fibrosing agent (or embolic material) and injected as an embolic agent; microparticulate wool and/or wool fibers (linear, branched, and/or coiled) either alone, or loaded with an additional fibrosing agent (or embolic material) and injected as an embolic agent (c) gels, microspheres, or microparticles formed from polymeric formulations of fibrosing agents (e.g., polymeric drugs such as those described by Polymerix Corporation); (d) fibrosing agents coated on the surface of microspheres or microparticles, with or without a polymeric carrier; (e) fibrosing agents loaded into one or more phases of a liquid embolic system (see descriptions above); (f) fibrosing agents delivered in the aqueous phase (ie., as an infusion into the treated tissue) in conjunction with (before, during or after) an embolization procedure; (g) for in situ forming embolic compositions, the fibrosing agents can be incorporated directly into the formulation as a suspension or a solution (e.g., silk powder, bleomycin), or loaded into a secondary carrier (e.g., micelles, liposomes, microspheres, microparticles, nanospheres, microparticulates, emulsions and/or microemulsions) that is then incorporated into the in situ forming compositions; (h) the fibrosing agent can be electrostatically or covalently bound to one or more of the polymeric components of the in situ forming embolization composition; and/or (i) the fibrosing agent can be mixed with the materials that are used to make the device such that the fibrosing agent is incorporated into the embolic agent during manufacturing (for example, silk powder can be added as a reagent during the manufacture of microspheres).

In one embodiment, an injectable polymer system is combined with a biologically active agent (e.g., fibrosing agents such as talc, silk, chitosan, polylysine, fibronectin, bleomycin, CTGF; sclerosing agents such as ethanol, DMSO, surfactants, sucrose, sodium morrhuate, ethanolamine oleate NaCl, dextrose, glycerin, minocycline, tetracycline, doxycycline, polidocanol, sodium tetradecyl sulfate, sodium morrhuate, sotradecol; growth factors such as transforming growth factor, platelet-derived growth factor, fibroblast growth factor, and bone morphogenic proteins; and/or analogues and derivatives of these compounds) and injected into an aneurysm sac. The injectable polymer system may further comprise agents such as glycerol, glycerin, PEG 200, triethyl citrate, and triacetin as plasticizers. It should be apparent to one of skill in the art that potentially any fibrosing agent described above may be utilized alone, or in combination, in the practice of this embodiment. Exemplary fibrosing agents for use in embolization devices and compositions include talc, silk, chitosan, polylysine, fibronectin, bleomycin, and CTGF, as well as analogues and derivatives of the aforementioned.

In certain embodiments, the fibrosing agent may be delivered directly to the site of an aneurysm via a specialized catheter delivery system. The agent (such as silk in a particulate form, i.e., silk partiles) or a composition that includes the agent may be delivered directly into an aneurysm sac. Within one embodiment, the fibrosing agent (e.g., particulate silk, particulate wool) is in an aqueous solution (e.g., saline) that may, optionally, include a contrast agent. The agent or composition comprising the agent may be injected into the aneurysm sac using, for example, a catheter, or using other means known to those skilled in the art to promote scarring of the aneurysm. In certain embodiments, the fibrosing agent or composition including the agent may be used in conjunction with a stent graft to repair an aneurysm.

A variety of other embodiments are suitable for the practice of this invention, including: (1) a “thermopaste” containing a fibrosing agent that is applied to a desired site as a fluid, and hardens to a solid of the desired shape at a specified temperature (e.g., body temperature); (2) as a spray (i.e., “nanospray”) containing a fibrosing agent that can be delivered to the aneurysm via a catheter and then subsequently hardens to a solid that adheres to the vascular wall; (3) as an adherent, pliable, resilient, polymer film containing a fibrosing agent applied to the aneurysm wall, and which preferably adheres to the site; and/or (4) as a fluid composed of a suspension of microspheres containing a fibrosing agent in an appropriate carrier medium, which is injected into the aneurysm sac, and which leaves a layer of microspheres at the application site.

In one aspect, the walls of the aneurysm sac can be treated with a fibrosing agent combined with a composition that forms a gel in situ. These can be crosslinked gels, thermogels, or traditional gel compositions. For the in situ forming gels, thermogel and gel compositions, the fibrosing agent(s) can be incorporated directly into the formulation to produce a suspension or a solution (e.g., silk powder, wool particles, bleomycin) or it can be incorporated into a secondary carrier (e.g., micelles, liposomes, microspheres, microparticles, nanospheres, micropaticulates, emulsions and/or microemulsions) that is then incorporated into the in situ forming gel compositions. In another embodiment, the fibrosing agent can be electrostatically or covalently bound to one or more of the polymeric components of the in situ forming gel composition.

In another embodiment, the fibrosing agent can be in an injectable or sprayable form that can be delivered directly into the aneurysm. The fibrosing agent(s) can be incorporated directly into the formulation to produce a suspension or a solution (e.g., silk powder, bleomycin) or incorporated into a secondary carrier (e.g., micelles, liposomes, microspheres, microparticles, nanospheres, micropaticulates, emulsions and/or microemulsions) that is then incorporated into the injectable or sprayable composition. In another embodiment, the fibrosing agent can be electrostatically or covalently bound to one or more of the polymeric components of the injectable or sprayable composition. These injectable and sprayable compositions can further comprise a polymer to enhance the viscosity of the solution. Polymers that can be used for this purpose include hyaluronic acid, CMC, PLURONICS, such as PLURONIC F127, as well as gels (normal and thermo gels) of the form X—Y, X—Y—X, or Y—X—Y (where X is a degradable polyester and Y is a polyalkylene oxide—preferably polyethylene glycol or the mono-methyl ether thereof). In another embodiment, the injectable or sprayable formulation can further comprise a biocompatible solvent. These can include ethanol, DMSO, NMP, poly(ethylene glycol)-200, and/or poly(ethylene glycol)-300.

One material that is of particular interest for direct injection into an aneurysm sac, either alone or in combination with a fibrosing agent, is a composition prepared from a 4-armed thiol PEG (10K), a 4-armed NHS PEG(10K) and methylated collagen. In a preferred embodiment, a material made from 4-armed thiol PEG (10K), a 4-armed NHS PEG(10K) and methylated collagen is loaded with a fibrosing agent injected directly into a cerebral or aortic aneurysm, to induce fibrosis.

In another example, a composition that comprises the reaction product of a 4-armed amino derivatized poly(ethylene glycol) and a 4-armed succinimidyl derivatized poly(ethylene glycol) is suitable for use as an injectable composition containing a fibrosing agent. In another example, a portion of the 4-armed amino derivatized poly(ethylene glycol) is substituted by a 4-armed thio derivatized poly(ethylene glycol). In each of the above examples, collagen or a collagen derivative (e.g., methylated collagen) can be added during the crosslinking process.

D. Aneurysm Coils for Cerebral Aneurysms

Numerous other types of vascular occlusion devices can be utilized with fibrosing agents in the practice of the invention, including, for example, vascular coils, vaso-occlusive coils, vaso-occlusion devices, vascular occlusion devices, vascular wires, intravascular embolization devices, vascular occlusion apparatus, microcoils, embolic vascular implants, embolic plugs, expandable implants, vascular plugs, and vascular endoprostheses.

Aneurysm coils, implants and injectable “fillers” are often used in the management of cerebral aneurysms. Aneurysm rupture in the brain can have catastrophic consequences including subarachnoid hemorrhage, stroke, permanent neurological deficits, and death. Surgical procedures to treat this condition, especially if located in the brain (known as anurysm “clipping”), can be extremely risky or even impossible, depending upon the anatomical location of the aneurysm. As an alternative to surgery, minimally invasive interventions have been developed whereby both ruptured and unruptured aneurysms can be treated using embolization devices. Embolization devices may be delivered to the aneurysm using a catheter or guide-wire that is advanced from the groin to the area of the aneurysm. The embolization device is then inserted through the catheter and into the aneurysm. Once within the aneurysm, it physically occupies space within the aneurysm sac, induces the formation of clot, “fills” the aneurysm sac, and prevents arterial blood flow from entering the aneurysm and thus, prevents further damage. Numerous implants have been described for insertion into an aneurysm sac and are suitable for combining with a fibrosis-inducing agent. One of the most common treatments for cerebral aneurysms involves the implantation of vascular “coils” into the aneurysm sac. The coil is advanced into the sac via a delivery catheter under radiologic guidance, detached (often by the induction of current in metal coils) from the delivery catheter and released into the sac; the procedure is then repeated until enough coils are “packed” into the aneurysm sac to fill it completely. Although a significant advancement in the treatment of aneurysms, detachable coils are not without their limitations. Complications associated with these procedures include inadvertent occlusion of the parent artery (occurs approximately 21% of the time), persistent filling of the aneurysm lumen (incomplete occlusion), and a recanalization (i.e., return of blood flow into the aneurysm following initially successful occlusion) rate of 2-5% per year. The consequences of incomplete occlusion (occurs in 38% of cases for small necked aneurysms, 60-85% of cases for broad necked aneurysms) and recanalization are that there is an increased risk that the aneurysm will rebleed. Specifically, the coil-thrombus complex formed after initial successful deployment is thought to be unstable. Recanalization can be due to compression of the coil bundle and rearrangement of individual coil loops which have a tendency to revert back to their original helical form (especially when not densely packed). The clinical result of recanalization is that the patient is at risk for aneurysm rupture and bleeding (subarachnoid hemorrhage) which is associated with a high mortality rate (25-50%) and high morbidity rate (50% of survivors have a significant neurologic deficit). In contrast, completely occluded aneurysms are thought to have a low (or no) risk of rebleeding. The addition of a fibrosis-inducing agent to an aneurysm coil can help reduce the risk of failure by stabilizing the coil-thrombus complex with fibrous tissue (preventing incomplete occlusion) and filling the sac with permanent scar tissue (preventing recanalization).

A variety of aneurysm coils can be combined with a fibrosis-inducing agent for the purposes of this invention. It should be obvious to one of skill in the art that the exact physical shape of the coil is not critical to the practice of this invention, however, numerous coil designs are presented by way of illustration. In one aspect, the aneurysm coil may be composed of a biocompatible metal alloy (e.g., platinum or tungsten) and/or a biocompatible polymer, which may or may not be biodegradable. The vascular aneurysm coil may be coated or uncoated, and/or may include other elements (e.g., strands, filaments, meshes and/or other particles) along the coil. The vascular coil may be composed of a bioactive component or may be biologically inert. Since vascular coils may be delivered through a microcatheter to the vascular site, they may be designed to have both a primary phase and a secondary phase. The secondary phase of the vascular coil may be a different shape, composition, physical state and/or level of bioactivity. For example, the vascular coil may be designed as an outer helically wound device having a stretch-resistant polymeric filament in which a secondary shape is formed and heat-treated to preserve that form. See e.g., U.S. Pat. No. 6,193,728. The vascular coil may be designed to be a linear helical configuration when stretched, and a folded, convoluted configuration when relaxed. See e.g., U.S. Pat. No. 4,994,069. The vascular coil may be composed of a flexible, helically wound coil having two primary coil ends and a primary diameter which in a relaxed secondary configuration comprises at least two longitudinal focal axes. See e.g., U.S. Pat. No. 5,639,277. The vascular coil may have attached fibrous elements which extend in a sinusoidal fashion down the length of the coil and thus, produce a variety of secondary shapes. See e.g., U.S. Pat. No. 5,304,194. The vascular coil may be a metal coil that has one or more fiber bundles having a serpentine configuration in which the loops extend about the individual windings of the coil. See e.g., U.S. Pat. No. 5,226,911. The embolization device (e.g., vascular coil) may be composed of a helical coil having a multiplicity of windings that define a lumen and a plug of thermoplastic biocompatible polymer that is located at the ends of the coil into the lumen space. See e.g., U.S. Pat. No. 5,690,667. The vascular coil may be composed of an elongated helical coil of a biocompatible metal having a plurality of axial spaced windings and a plurality of strands of a polymeric, bioactive, occlusion-causing material extending axially through the coil. See e.g., U.S. Pat. No. 5,658,308. The embolization device may be an expandable support element having a relaxed expanded state and a stretched collapsed state, and an embolization element which is mounted on the support element which serves to substantially prevent the blood flow (e.g., polymer mesh). See e.g., U.S. Pat. No. 6,554,849. The embolization device may be composed of an elongated, flexible filamentous carrier and an embolizing element in the form of an expansile polymer (e.g., porous hydrogel) which is fixed to the carrier. See e.g., U.S. Pat. No. 6,602,261. The vascular coil may contain a positive charge, electric current, or magnetic field on the coil which promotes embolization. See e.g., U.S. Pat. No. 5,122,136, 6,066,133 and 6,603,994. Other vascular coils are described in U.S. Pat. No. 5,133,731, 5,312,415, 5,354,294, 5,382,259, 5,382,260, 5,417,708, 5,423,849, 5,476,472, 5,578,074, 5,582,619, 5,624,461, 5,645,558 and 5,718,711.

Aneurysm coils, which may be combined with one or more fibrosis-inducing agents according to the present invention, include several commercially available products. For example, the GDC (GUGLIELMI DETACHABLE COIL) and the MATRIX detachable coils (from Boston Scientific, Natick, Mass.) are particularly useful for the practice of this embodiment. The MICROPLEX and HYDROCOIL (from MicroVention, Inc., Aliso Viejo, Calif.) are also suitable.

In another aspect, aneurysm coils and wires are provided that are made from a biodegradable material, such as a polymer, which is flexible (malleable) and strong. The polymer may be capable of expanding in size after deployment. Representative examples of expansible polymers for use in aneurysm coils and wires are poly(hydroxyethyl methacrylate), poly(acrylamide) and copolymers thereof. Degradation of the polymeric coil in the days to weeks following deployment has several advantages. For example, polymeric aneurysm coils, in contrast to metallic coils, may reduce the risk of aneurysm performation during deployment. Since the coils do not persist, they also may be less likely to migrate into the parent vessel circulation. Further, degradable coils can become incorporated into the thrombus-coil complex, thus reducing the incidence of recanalization.

The vascular aneurysm coil may be coated or uncoated, and/or may include other elements (e.g., strands, filaments, meshes and/or other particles) along the coil. In one aspect, aneurysm coils can be coated with or contain a non-thrombogenic substance (e.g., heparin, antithrombin, antithrombin-heparin complex), which prevents thrombus from occurring prior to final placement of the device. This temporary coating can be designed to persist for minutes to hours depending upon the time required to deploy the device.

E. Delaying Onset of Activity

The time it takes to insert a stent, stent graft, aneurysm coil or embolic material can be very long. For instance with stent grafts, it theoretically could be hours between the time that the first part of a device (usually the aortic segment) is deployed and the second part of the device is deployed. It is not until all the parts of the device are inserted that an adequate exclusion of the aneurysm is achieved. Similarly, it can take hours to pack an aneurysm with multiple coils (occasionally more than 20 can be required for larger aneurysms). In other words, the coating on the device may cause blood clots to form on or around the device before it is fully deployed. Because blood is rushing around as well as through the device until it is fully deployed, thereby excluding the aneurysm, such blood clots could be dislodged and washed downstream, or, might propagate distally. This could result in the inadvertent and undesirable occlusion or partial occlusion of blood vessels downstream from the intended site of insertion of the device, which the operator had intended to keep open. Several strategies may be employed to address such difficulties.

For example, as discussed in more detail above, stent grafts (and using the same approach, stents, aneurysm coils and embolic agents) may be constructed which are designed to delay the onset of activity of the fibrosis inducing, and/or fibrosis forming response to the silk (e.g., by coating the implant with a material such as heparin or PLGA which delays adhesion or fibrosis).

F. Dosages

It should be apparent to one of skill in the art that potentially any fibrosing agent described above may be utilized alone, or in combination, in the practice of this embodiment. Exemplary fibrosing agents for use with stent grafts, stents, balloons, catheters, aneurysm coils and embolic agents devices include talc, silk, wool, chitosan, polylysine, fibronectin, silver nitrate, bleomycin, and CTGF, as well as analogues and derivatives of the aforementioned. Other materials for promoting adhesion of the vascular wall to an intravascular device or the stabilization of vulnerable plaque include microemulsions formed from caprylocaproyl macrogol-8 glycerides, such as those sold under the trade name LABRASOL (Gattefosse, France), PEG-PLGA polymers, PLURONICs, sucrose, starch (e.g., corn starch or maize starch) and other materials that are known to induce the formation of surgical adhesions when administered in vivo.

As the above described intravascular devices and implants are made in a variety of configurations and sizes depending upon the location and anatomy of the lesion, the exact dose administered will vary with implant size, surface area and design. However, certain principles can be applied in the application of this art. Drug dose can be calculated as a function of dose per unit area (or volume) of the device or implant being coated, total drug dose administered can be measured and appropriate surface concentrations of active drug can be determined. Regardless of the method of application of the drug to the blood vessel or the intravascular implant (or device), the exemplary fibrosing agents, used alone or in combination, should be administered under the following dosing guidelines:

Utilizing talc as an exemplary fibrosing agent, whether it is applied using a polymer coating, incorporated into the polymers which make up the device or implant, or applied without a polymeric carrier, the total dose of talc delivered from an intravascular device (e.g., stent graft, stent, balloon, catheter, aneurysm coil) and/or embolic agent, should not exceed 2500 mg (range of 1 μg to 2500 mg)). In one embodiment, the total amount of talc released from the implant should be in the range of 10 μg to 50 mg. In another embodiment, the total amount of talc released from the implant should be in the range of 50 mg to 100 mg. In another embodiment, the total amount of talc released from the implant should be in the range of 100 mg to 500 mg. In another embodiment, the total amount of talc released from the implant should be in the range of 500 mg to 1000 mg. In another embodiment the total amount of talc released from the implant should be in the range of 1000 mg to 2500 mg. For embolic agents and injectables, the dose per unit volume of the implant (i.e., the dosage of talc as a function of the volume of the portion of the implant to which drug is applied and/or incorporated) should fall within the range of 0.05 μg-10 μg per mm³ of material implanted. In another embodiment, talc should be applied to a device or implant (e.g., stent graft, stent, balloon, catheter, aneurysm coil) surface at a dose of 0.05 μg/mm²-10 μg/mm² of surface area coated. In another embodiment, talc should be applied to a device surface at a dose of 10.0 μg/mm²-100 μg/mm² of surface area coated. In another embodiment, talc should be applied to a device surface at a dose of 100 μg/mm²-500 μg/mm² of surface area coated. As specific (polymeric and non-polymeric) drug delivery vehicles and specific medical implants will release talc at differing rates, the above dosing parameters should be utilized in combination with the release rate of the drug from the device (e.g., stent graft, stent, balloon, catheter, aneurysm coil) and/or embolic agent such that a minimum concentration of 0.01 ng to a maximum of 2500 mg of talc is delivered to the tissue or in the area of the tissue. In one embodiment, talc is released from the surface of the device or implant such that fibrosis in the tissue is promoted for a period ranging from several hours to several months to approximately one year or longer. For example, talc may be released in effective concentrations for a period ranging from 1 to 12 months. It should be readily evident given the discussions provided herein that analogues and derivatives of talc (as described previously) with similar functional activity can be utilized for the purposes of this invention; the above dosing parameters are then adjusted according to the relative potency of the analogue or derivative as compared to the parent compound (e.g., a compound twice as potent as talc is administered at half the above parameters, a compound half as potent as talc is administered at twice the above parameters, etc.).

Utilizing silk as an exemplary fibrosing agent, whether it is applied using a polymer coating, incorporated into the polymers which make up the device or implant, or applied without a polymeric carrier, the total dose of silk delivered from an intravascular device (e.g., stent graft, stent, balloon, catheter, aneurysm coil) and/or embolic agent, should not exceed 100 mg (range of 1 μg to 100 mg). In one embodiment, the total amount of silk released from the implant should be in the range of 1 82 g to 500 μg. In another embodiment, the total amount of silk released from the implant should be in the range of 500 μg to 1 mg. In another embodiment, the total amount of silk released from the implant should be in the range of 1 mg to 100 mg. For embolic agents and injectables, the dose per unit volume of the implant (i.e., the dosage of silk as a function of the volume of the portion of the implant to which drug is applied and/or incorporated) should fall within the range of 0.05 μg-10 μg per mm³ of material implanted. In another embodiment, silk should be applied to a device (e.g., stent graft, stent, or aneurysm coil) surface at a dose of 0.05 μg/mm²-10 μg/mm² of surface area coated. In another embodiment, silk should be applied to a device surface at an amount of 10.0 μg/mm²-100 μg/mm² of surface area coated. In another embodiment, silk should be applied to a device surface at a dose of 100 μg/mm²-500 μg/mm² of surface area coated. In one embodiment the concentration of silk may be evenly distributed on the surface of the device while in other embodiments the concentration of silk may vary in different areas of the device. As specific (polymeric and non-polymeric) drug delivery vehicles and specific medical implants will release silk at differing rates, the above dosing parameters should be utilized in combination with the release rate of the drug from the device (e.g., stent graft, stent, balloon, catheter, aneurysm coil) and/or embolic agent such that a minimum concentration of 0.01 nM to 1000 μM of silk is delivered to the tissue or in the area of the tissue. In one embodiment, silk remains on the device and is not released, while in other embodiments, silk is released from the device. In one embodiment, silk is released from the surface of a device or implant such that fibrosis in the tissue is promoted for a period ranging from several hours to a number of months. For example, silk may be released in effective concentrations for a period ranging from 1 to 12 months. It should be readily evident given the discussions provided herein that analogues and derivatives of silk (as described previously) with similar functional activity can be utilized for the purposes of this invention; the above dosing parameters are then adjusted according to the relative potency of the analogue or derivative as compared to the parent compound (e.g., a compound twice as potent as silk is administered at half the above parameters, a compound half as potent as silk is administered at twice the above parameters, etc.).

Utilizing chitosan as an exemplary fibrosing agent, whether it is applied using a polymer coating, incorporated into the polymers which make up the device or implant, or applied without a polymeric carrier, the total dose of chitosan delivered from a device (e.g., stent graft, stent, balloon, catheter, aneurysm coil) and/or embolic agent, should not exceed 100 mg (range of 1 μg to 100 mg). In one embodiment, the total amount of chitosan released from the device or implant should be in the range of 10 μg to 50 mg. For embolic agents and injectables, the dose per unit volume of the implant (i.e., the dosage of chitosan as a function of the volume of the portion of the implant to which drug is applied and/or incorporated) should fall within the range of 0.05 μg-10 μg per mm³ of material implanted. In another embodiment, chitosan should be applied to a device (e.g., stent, stent graft, or aneurysm coil) surface at a dose of 0.05 μg/mm²-10 μg/mm² of surface area coated. In another embodiment, chitosan should be applied to a device surface at an amount of 10.0 μg/mm²-100 μg/mm² of surface area coated. In another embodiment, chitosan should be applied to a device surface at a dose of 100 μg/mm²-500 μg/mm² of surface area coated. In one embodiment, the concentration of chitosan may be evenly distributed on the surface of the device while in other embodiments the concentration of chitosan may vary in different areas of the device. As specific (polymeric and non-polymeric) drug delivery vehicles and specific medical devices and implants will release chitosan at differing rates, the above dosing parameters should be utilized in combination with the release rate of the drug from the device (e.g., stent graft, stent, balloon, catheter, aneurysm coil) and/or embolic agent, such that a minimum concentration of 0.01 nM to 1000 μM of chitosan is delivered to the tissue or in the area of the tissue. In one embodiment, chitosan remains on the device and is not released, while in other embodiments, chitosan is released from the device. In one embodiment, chitosan is released from the surface of the device or implant such that fibrosis in the tissue is promoted for a period ranging from several hours to several months. For example, chitosan may be released in effective concentrations for a period ranging from 1 to 12 months. It should be readily evident given the discussions provided herein that analogues and derivatives of chitosan (as described previously) with similar functional activity can be utilized for the purposes of this invention; the above dosing parameters are then adjusted according to the relative potency of the analogue or derivative as compared to the parent compound (e.g., a compound twice as potent as chitosan is administered at half the above parameters, a compound half as potent as chitosan is administered at twice the above parameters, etc.).

Utilizing polylysine as an exemplary fibrosing agent, whether it is applied using a polymer coating, incorporated into the polymers which make up the device or implant, or applied without a polymeric carrier, the total dose of polylysine delivered from an intravascular device (e.g., stent graft, stent, balloon, catheter, aneurysm coil) and/or embolic agent, should not exceed 100 mg (range of 1 μg to 100 mg). In one embodiment, the total amount of polylysine released from the device or implant should be in the range of 10 μg to 50 mg. For embolic agents and injectables, the dose per unit volume of the implant (i.e., the dosage of polylysine as a function of the volume of the portion of the implant to which drug is applied and/or incorporated) should fall within the range of 0.05 μg-10 μg per mm³ of material implanted. In another embodiment, polylysine should be applied to a device (e.g., stent graft, stent or aneurysm coil) surface at a dose of 0.05 μg/mm²-10 μg/mm² of surface area coated. In another embodiment, polylysine should be applied to a device surface at an amount of 10.0 μg/mm²-100 μg/mm² of surface area coated. In another embodiment, polylysine should be applied to a device surface at a dose of 100 μg/mm²-500 μg/mm² of surface area coated. In one embodiment, the concentration of polylysine may be evenly distributed on the surface of the device while in other embodiments the concentration of polylysine may vary in different areas of the device. As specific (polymeric and non-polymeric) drug delivery vehicles and specific medical devices and implants will release polylysine at differing rates, the above dosing parameters should be utilized in combination with the release rate of the drug from the device (e.g., stent graft, stent, balloon, catheter, aneurysm coil) and/or embolic agent such that a minimum concentration of 0.01 nM to 1000 μM polylysine is delivered to the tissue. In one embodiment, polylysine is released from the surface of the device or implant such that fibrosis in the tissue is promoted for a period ranging from several hours to several months. For example, polylysine may be released in effective concentrations for a period ranging from 1 to 12 months. It should be readily evident given the discussions provided herein that analogues and derivatives of polylysine (as described previously) with similar functional activity can be utilized for the purposes of this invention; the above dosing parameters are then adjusted according to the relative potency of the analogue or derivative as compared to the parent compound (e.g., a compound twice as potent as polylysine is administered at half the above parameters, a compound half as potent as polylysine is administered at twice the above parameters, etc.).

Utilizing fibronectin as an exemplary fibrosing agent, whether it is applied using a polymer coating, incorporated into the polymers which make up the device or implant, or applied without a polymeric carrier, the total dose of fibronectin delivered from an intravascular device (e.g., stent graft, stent, balloon, catheter, aneurysm coil) and/or embolic agent, should not exceed 100 mg (range of 1 μg to 100 mg). In one embodiment, the total amount of fibronectin released from the device or implant should be in the range of 10 μg to 50 mg. For embolic agents and injectables, the dose per unit volume of the implant (i.e., the dosage of fibronectin as a function of the volume of the portion of the implant to which drug is applied and/or incorporated) should fall within the range of 0.05 μg-10 μg per mm³ of material implanted. In another embodiment, fibronectin should be applied to a device (e.g., stent graft, stent or aneurysm coil) surface at a dose of 0.05 μg/mm²-10 μg/mm² of surface area coated. In another embodiment, fibronectin should be applied to a device surface at an amount of 10.0 μg/mm²-100 μg/mm² of surface area coated. In another embodiment, fibronectin should be applied to a device surface at a dose of 100 μg/mm²-500 μg/mm² of surface area coated. In one embodiment, the concentration of fibronectin may be evenly distributed on the surface of the device, while in other embodiments the concentration of fibronectin may vary in different areas of the device. As specific (polymeric and non-polymeric) drug delivery vehicles and specific medical implants will release fibronectin at differing rates, the above dosing parameters should be utilized in combination with the release rate of the drug from the stent graft, stent, balloon, catheter, aneurysm coil and/or embolic agent such that a minimum concentration of 0.01 nM to 1000 μM of fibronectin is delivered to the tissue or in the area of the tissue. In one embodiment, fibronectin remains on the device and is not released, while in other embodiments, fibronectin is released from the device. In one embodiment, fibronectin is released from the surface of the device or implant such that fibrosis in the tissue is promoted for a period ranging from several hours to several months. For example, fibronectin may be released in effective concentrations for a period ranging from 1 to 12 months. It should be readily evident given the discussions provided herein that analogues and derivatives of fibronectin (as described previously) with similar functional activity can be utilized for the purposes of this invention; the above dosing parameters are then adjusted according to the relative potency of the analogue or derivative as compared to the parent compound (e.g., a compound twice as potent as fibronectin is administered at half the above parameters, a compound half as potent as fibronectin is administered at twice the above parameters, etc.).

Utilizing bleomycin as an exemplary fibrosing agent, whether it is applied using a polymer coating, incorporated into the polymers which make up the device or implant, or applied without a polymeric carrier, the total dose of bleomycin delivered from an intravascular device (e.g., stent graft, stent, balloon, catheter, aneurysm coil) and/or embolic agent, should not exceed 100 mg (range of 0.001 μg to 100 mg). In one embodiment, the total amount of bleomycin released from the device and implant should be in the range of 0.010 μg to 50 mg. For embolic agents and injectables, the dose per unit volume of the implant (i.e., the dosage of bleomycin as a function of the volume of the portion of the implant to which drug is applied and/or incorporated) should fall within the range of 0.005 μg-10 μg per mm³ of material implanted. In another embodiment, bleomycin should be applied to a device (e.g., stent graft, stent or aneurysm coil) surface at a dose of 0.005 μg/mm²-10 μg/mm² of surface area coated. As specific (polymeric and non-polymeric) drug delivery vehicles and specific medical implants will release bleomycin at differing rates, the above dosing parameters should be utilized in combination with the release rate of the drug from the device (e.g., stent graft, stent, balloon, catheter, aneurysm coil) and/or embolic agent such that a minimum concentration of 0.001 nM to 1000 μM of bleomycin is delivered to the tissue. In one embodiment, bleomycin is released from the surface of the device or implant such that fibrosis in the tissue is promoted for a period ranging from several hours to several months. For example, bleomycin may be released in effective concentrations for a period ranging from 1 to 12 months. It should be readily evident given the discussions provided herein that analogues and derivatives of bleomycin (as described previously) with similar functional activity can be utilized for the purposes of this invention; the above dosing parameters are then adjusted according to the relative potency of the analogue or derivative as compared to the parent compound (e.g., a compound twice as potent as bleomycin is administered at half the above parameters, a compound half as potent as bleomycin is administered at twice the above parameters, etc.).

Utilizing CTGF as an exemplary fibrosing agent, whether it is applied using a polymer coating, incorporated into the polymers which make up the device or implant, or applied without a polymeric carrier, the total dose of CTGF delivered from an intravascular device (e.g., stent graft, stent, balloon, catheter, aneurysm coil) and/or embolic agent, should not exceed 100 mg (range of 0.01 μg to 100 mg). In one embodiment, the total amount of CTGF released from the device or implant should be in the range of 0.10 μg to 50 mg. For embolic agents and injectables, the dose per unit volume of the implant (i.e., the dosage of CTGF as a function of the volume of the portion of the implant to which drug is applied and/or incorporated) should fall within the range of 0.005 μg-10 μg per mm³ of material implanted. In another embodiment, CTGF should be applied to a device (e.g., stent graft, stent or aneurysm coil) surface at a dose of 0.005 μg/mm²-10 μg/mm² of surface area coated. As specific (polymeric and non-polymeric) drug delivery vehicles and specific medical devices and implants will release CTGF at differing rates, the above dosing parameters should be utilized in combination with the release rate of the drug from the device (e.g., stent graft, stent, balloon, catheter, aneurysm coil) and/or embolic agent such that a minimum concentration of 0.001 nM to 1000 μM of CTGF is delivered to the tissue. In one embodiment, CTGF is released from the surface of a device or implant such that fibrosis in the tissue is promoted for a period ranging from several hours to several months. For example, CTGF may be released in effective concentrations for a period ranging from 1 to 12 months. It should be readily evident given the discussions provided herein that analogues and derivatives of CTGF (as described previously) with similar functional activity can be utilized for the purposes of this invention; the above dosing parameters are then adjusted according to the relative potency of the analogue or derivative as compared to the parent compound (e.g., a compound twice as potent as CTGF is administered at half the above parameters, a compound half as potent as CTGF is administered at twice the above parameters, etc.).

Optionally, the implant or device may alone, or additionally, comprise an inflammatory cytokine (e.g., TGFβ, PDGF, VEGF, bFGF, TNFα, NGF, GM-CSF, IGF-a, IL-1, IL-1-β, IL-8, IL-6, and growth hormone).

Inflammatory cytokines may be used in formulations at concentrations that range from 0.0001 μg/ml to approximately 20 mg/ml depending on the specific clinical application, formulation type (e.g., gel, liquid, solid, semi-solid), formulation chemistry, duration of required application, type of medical device interface and formulation volume and or surface area coverage required. Preferably, the inflammatory cytokine is released in effective concentrations for a period ranging from 1-180 days. The total dose for a single application is typically not to exceed 500 mg (range of 0.0001 μg to 100 mg); preferred 0.001 μg to 50 mg. When used as a device coating, the dose is per unit area of 0.0001 μg-500 μg per mm²; with a preferred dose of 0.001 μg/mm²-200 μg/mm². Minimum concentration of 10⁻¹⁰-10⁻⁴ g/ml of inflammatory cytokine is to be maintained on the device surface.

Furthermore, the device may alone or additionally comprise an agent that stimulates cellular proliferation. Examples include: dexamethasone, isotretinoin (13-cis retinoic acid), 17-β-estradiol, estradiol, 1-a-25 dihydroxyvitamin D₃, diethylstibesterol, cyclosporine A, L-NAME, all-trans retinoic acid (ATRA), and analogues and derivatives thereof. Doses used are those concentrations which are demonstrated to stimulate cell proliferation (see, e.g., Examples 17-22). The proliferative agents are to be used in formulations at concentrations that range from 0.0000001 to 25 mg/ml depending on the specific clinical application, formulation type (e.g., gel, liquid, solid, semi-solid), formulation chemistry, duration of required application, type of medical device interface and formulation volume and or surface area coverage required. Preferably, the proliferative agent is released in effective concentrations for a period ranging from 1-180 days. The total dose for a single application is typically not to exceed 500 mg (range of 0.0001 μg to 200 mg); preferred 0.001 μg to 100 mg. When used as a device coating, the dose is per unit area of 0.00001 μg-500 μg per mm²; with a preferred dose of 0.0001 μg/mm²-200 μg/mm². Minimum concentration of 10⁻¹¹-10⁻⁶ M of proliferative agent is to be maintained on the device surface.

K. Methods for Inducing Fibrosis in Arterial Plaque

The present invention discloses novel compositions, methods for preparing them, and devices such as catheters, balloons, stents, and other devices suitable for the localized delivery of therapeutic agents designed to induce a fibrotic response in the arterial wall such that vulnerable plaque is more effectively separated from the arterial lumen. Administration of fibrosis-inducing agents to the vulnerable plaque can serve several functions including conversion of some (or all) of the lipid core to fibrous tissue (fibroblasts, smooth muscle) and increasing the stability the fibrous cap. Either of these results can have the effect of stabilizing the vulnerable plaque and reducing the likelihood of rupture and infarction. In one aspect, methods are described for delivering a therapeutic agent that induces fibrosis in arterial plaque.

Coronary Artery Disease (“CAD”) affects over 12.5 million Americans and results in over 1 million heart attacks (myocardial infarctions—“MI”) and 500,000 deaths annually. Traditionally, CAD was thought to be due to the gradual accumulation of atherosclerotic plaque in the arterial wall that eventually impedes arterial blood flow to the muscle of the heart leading to chest pain (angina). With further progression or rupture of the plaque, blood flow becomes completely obstructed and myocardial infarction results. However, close to half of all out-of-hospital cardiac deaths occur in people with no prior diagnosis of heart disease, and over two-thirds of MI's occur in arteries where the blockage is considered “clinically insignificant” by angiographic assessment of plaque burden and percent stenosis (narrowing). It is now accepted that many of these serious cardiac events can be caused by vulnerable plaque which appear to be highly prone to rupturing.

“Vulnerable plaque” refers to non-occluding, fatty arterial deposits that form a soft, unstable lesion which is prone to rupturing. Vulnerable plaques are comprised of soft, biologically active, thrombogenic fatty material covered by a thin fibrous layer which produces an eccentric, poorly calcified lesion that is frequently hemodynamically insignificant (i.e., a stenosis of less than 75%). The central core of the plaque is composed primarily of lipid and contains a large infiltration of activated macrophages, inflammatory cells and inflammatory cell byproducts (cytokines, matrix metalloproteinases, low pH, oxidative reactants). The fibrous cap is thin, contains very little collagen, is often fissured, and is frequently incompletely covered by endothelium. The thin fibrous cap provides a very weak barrier between the lipid core and the arterial circulation and contributes to the tendency for unstable plaque to rupture. It is thought that the risk of plaque rupture is greatest when the fibrous cap is very thin and/or the plaque lipid pool is very large.

As vulnerable plaque is a soft, fatty, unstable lesion, it is not well visualized with standard angiographic methods. However, it is most often located using imaging and radiological methods including, for example, magnetic resonance imaging, elastography, thermal sensors, optical coherence tomography, and near-infrared and infrared light techniques. Visualization of vulnerable plaque may be further enhanced by the use of a contrast agent or a radiopaque material. It is believed that thromboemboli originating from the rupture and/or erosion of vulnerable plaque may be responsible for up to 85% of all myocardial infarctions. It is also believed that vulnerable plaque in the carotid and cerebral circulation may be the cause of the majority of ischemic cerebral vascular accidents (CVA; “strokes”) in the brain.

Microscopically, vulnerable plaque differs from stable atherosclerotic plaque. The core of a stable plaque is composed of small amounts of lipid, few macrophages, numerous “foam cells,” necrotic cellular debris, cholesterol crystals and abundant calcification. The stable plaque is covered by a highly organized, thick fibrous capsule composed of fibroblasts, macrophages, smooth muscle cells, elastin, collagen (and other extracellular matrix components) and an intact endothelial surface. The mature atheromatous plaque tends to cause concentric remodeling and progressive luminal narrowing that results in hemostatic complications (i.e., causes a stenosis greater than 75%) and produces symptoms such as angina.

In one aspect, the described catheters, balloons, stents and other intravascular devices can be used to deliver a therapeutic agent which induces fibrosis in arterial plaque.

Numerous drug-delivery catheters are available for local, regional or systemic delivery of fibrosing agents to vulnerable plaque. Typically, intravascular catheters are inserted into the femoral artery in the groin and advanced through the circulation under radiological guidance until they reach the anatomical location of the plaque in the coronary or peripheral circulation. The fibrosing agent, with or without a carrier, can then be released from the catheter lumen in high local concentrations in order to deliver therapeutic doses of the drug to the vulneable plaque. Several additional steps can be taken to further localize and concentrate the drug in the vulnerable plaque, including, but not restricted to: (a) the use of microinjection catheters, which are capable of direct injection of the fibrosing agent (or sustained release preparations of agent plus carrier (e.g., polymer) or polymerized versions of the therapeutic agent) into the plaque and/or the arterial wall; (b) drug localization techniques such as ultrasonic or MRI-guided drug delivery, electroporation, magnetic field assisted or radio-frequency assisted delivery; (c) chemical modification of the fibrosing drug or formulation designed to increase uptake of the agent into the plaque such as linking the drug to antibodies (directed against components of the plaque such as macrophages, lipids, smooth muscle cells, extracellular matrix components); (d) chemical modification of the fibrosing drug or formulation designed to localize the drug to areas of endothelial denudation; (e) direct injection of the fibrosing agent into the plaque, or applying a surface covering to the plaque with an surface-adherent formulation of drug and polymer under direct (angioscopic) vision; and/or (f) “endoluminal paving” (see, e.g., U.S. Pat. No. 5,213,580; 5,749,915; 6,372,229; 6,443,941; 6,290,729; 5,947,977; 5,800,538; and 5,749,922) of the surface of the plaque with the fibrosing agent and the endoluminal paving composition.

In another aspect of the invention, the compositions of the invention can be delivered to the treatment site (e.g., into unstable arterial plaque and/or into the tissue surrounding the plaque) by using catheter systems that have one or more injectors that can penetrate the plaque and/or the surrounding tissue. Following insertion into the appropriate vessel, the catheter can be maneuvered into the desired position such that the injectors are aligned with or adjacent to the plaque. The injector(s) enter into the desired location, for example, by direct insertion into the tissue, by inflating the balloon or by mechanical rotation of the injector, and the composition of the invention is injected into the desired location. Representative examples of catheters that can be used for this application are described in and U.S. patent application No. 2002/0082594 and U.S. Pat. Nos. 6,443,949; 6,488,659; 6,569,144; 5,609,151; 5,385,148; 5,551,427; 5,746,716; 5,681,281; and 5,713,863.

Compositions for delivery by catheter systems and other devices may be, for example, thermoreversible polymers. For the site-specific delivery of these materials, a catheter delivery system that has the ability to either heat the composition to above body temperature or to cool the composition to below body temperature such that the composition remains in a fluent state within the catheter delivery system. The catheter delivery system can be guided to the desired location and the composition of the invention can be delivered to the surface of the plaque or can be injected directly into the plaque or surrounding tissue. A representative example of a catheter delivery system for direct injection of a thermoreversible material is described in U.S. Pat. No. 6,488,659. Representative examples of catheter delivery systems that can deliver the thermoreversible compositions to the surface of the plaque are described in U.S. Pat. Nos. 6,443,941; 6,290,729; 5,947,977; 5,800,538; and 5,749,922.

Numerous drug-delivery balloons are available for local or regional delivery of fibrosing agents to vulnerable plaque. Drug delivery balloons developed for the local delivery of therapeutic agents to the arterial wall have been described herein and include, but are not limited to “sweaty balloons,” “channel balloons,” “microinjector balloons,” “double balloons,” “spiral balloons” and other specialized drug-delivery balloons. Typically, intravascular drug-delivery balloons are inserted into the femoral artery in the groin and advanced through the circulation under radiological guidance until they reach the anatomical location of the plaque in the coronary or peripheral circulation. If required, the balloons can be inflated and the fibrosing agent can then be released from the drug-delivery balloon in high local concentrations in order to deliver therapeutic doses of the fibrosing agent to the vulnerable plaque. This can be accomplished through several methods including, but restricted to, administration to the luminal surface of the plaque, direct injection into the plaque wall, direct injection into the arterial wall adjacent to the plaque, adherence of the fibrosing agent to the surface of the plaque, chemical targeting of the fibrosing agent to the vulnerable plaque (e.g., a fibrosing agent linked to an antibody or other drug-targeting technology which localizes the drug to a component of the plaque such as smooth muscle cells, inflammatory cells, endothelial cells, or extracellular matrix components), and/or movement of the fibrosing agent down a magnetic, hydrostatic, osmotic or concentrational gradient from the lumen into the vessel wall. These agents can also be delivered using catheter delivery systems that use magnetic, ultrasound (see, e.g., U.S. patent application Publication No. 2002/0068869; PCT Publication Nos. WO 94/05361, WO 96/04955, WO 02/076547, and WO 96/22111; U.S. Pat. Nos. 5,362,309; 5,318,014; 5,315,98; 5,269,291; 5,197,946; 6,001,069; 6,024718; 5,735,811; 5,197,946; and 6,623,444) or radio-frequency and electrical fields (see, e.g., U.S. Pat. Nos. 5,286,254 and 5,628,730, and PCT Publication Nos. WO 94/05361, WO 96/22111, and WO 96/04955) to assist the passage of the agents into the tissue.

One purpose of localized delivery of the fibrosing agent to the vascular wall via a specialized drug-delivery balloon is to increase the amount of fibrous tissue present in the plaque, ideally through the conversion of “fatty” tissue into fibrotic tissue. Topical or luminal application of the fibrosing agent can be used to increase the thickness and stability of the thin fibrous layer which covers the vulnerable plaque. Direct injection into, or diffusion of the fibrosing agent into, the parenchyma of the plaque can be utilized to “fill” the vulnerable plaque with drug. Particularly useful for this embodiment is the use of polymeric carriers and/or non-polymeric carriers which release the fibrosing agent over a period ranging from several hours to several weeks. Microspheres (solid and porous), pastes, gels, liquids, nanoparticulates, in situ forming materials and microparticulate (solid and porous) formulations which release a fibrosing agent can be delivered into the vulnerable plaque via specialized drug-delivery balloons to gradually convert the plaque into contracted, hemodynamically stable fibrous tissue. Soluble silk proteins, microparticulate silk and/or silk strands (linear, branched, and/or coiled) are also useful for directed delivery into the plaque via specialized drug-delivery balloons. In addition to the agents that enhance the formation of fibrous tissue, the compositions that are injected directly into the plaque can further include a contrast agent. This contrast agent will allow visualization of the injected material via ultrasound, MRI, fluoroscopy or standard x-ray.

In another aspect, the present invention provides stents for local or regional delivery of fibrosing agents to vulnerable plaque. Stents developed for the local delivery of therapeutic agents to the arterial wall have been described herein and include, but are not limited to, metallic stents, polymeric stents, biodegradable stents, covered stents, and drug-eluting stents.

The stent may be self-expanding or balloon expandable (e.g., the PALMAZ stent from Cordis Corporation and STRECKER stent by Medi-Tech/Boston Scientific Corporation), or implanted by a change in temperature (e.g., nitinol stent). Self-expanding stents that can be used include the coronary WALLSTENT and the SCIMED RADIUS stent from Boston Scientific Corporation (Natick, Mass.) and the GIANTURCO stents from Cook Group, Inc. (Bloomington, Ind.). Examples of balloon expandable stents that can be used include the CROSSFLEX stent, BX-VELOCITY stent and the PALMAZ-SCHATZ crown and spiral stents from Cordis Corporation (Miami Lakes, Fla.), the V-FLEX PLUS stent by Cook Group, Inc., the NIR, EXPRESS and LIBRERTE stents from Boston Scientific Corporation, the ACS MULTILINK, MULTILINK PENTA, SPIRIT, and CHAMPION stents from Guidant Corporation, and the Coronary Stent S670 and S7 by Medtronic, Inc. (Minneapolis, Minn.).

Other examples of stents that can be combined with a fibrosing agent in accordance with the invention include those from Boston Scientific Corporation, (e.g., the drug-eluting TAXUS EXPRESS² Paclitaxel-Eluting Coronary Stent System; over the wire stent stents such as the Express² Coronary Stent System and NIR Elite OTW Stent System; rapid exchange stents such as the EXPRESS² Coronary Stent System and the NIR ELITE MONORAIL Stent System; and self-expanding stents such as the MAGIC WALLSTENT Stent System and RADIUS Self Expanding Stent); Medtronic, Inc. (Minneapolis, Minn.) (e.g., DRIVER ABT578-eluting stent, DRIVER ZIPPER MX Multi-Exchange Coronary Stent System and the DRIVER Over-the-Wire Coronary Stent System; the S7 ZIPPER MX Multi-Exchange Coronary Stent System; S7, S670, S660, and BESTENT2 with Discrete Technology Over-the-Wire Coronary Stent System); Guidant Corporation (e.g., cobalt chromium stents such as the MULTI-LINK VISION Coronary Stent System; MULTI-LINK ZETA Coronary Stent System; MULTI-LINK PIXEL Coronary Stent System; MULTI-LINK ULTRA Coronary Stent System; and the MULTI-LINK FRONTIER); Johnson & Johnson/Cordis Corporation (e.g., CYPHER sirolimus-eluting Stent; PALMAZ-SCHATZ Balloon Expandable Stent; and S.M.A.R.T. Stents); Abbott Vascular (Redwood City, Calif.) (e.g., MATRIX LO Stent; TRIMAXX Stent; and DEXAMET stent); Connor Medsystems (Menlo Park, Calif.) (e.g., MEDSTENT and COSTAR stent); AMG GmbH (Germany) (e.g., PICO Elite stent); Biosensors International (Singapore) (e.g., MATRIX stent, CHAMPION Stent (formerly the S-STENT), and CHALLENGE Stent); Biotronik (Switzerland) (e.g., MAGIC AMS stent); Clearstream Technologies (Ireland) (e.g., CLEARFLEX stent); Cook Inc. (Bloomington, Ind.) (e.g., V-FLEX PLUS stent, ZILVER PTX self-expanding vascular stent coating, LOGIX PTX stent (in development); Devax (e.g., AXXESS stent) (Irvine, Calif.); DISA Vascular (Pty) Ltd (South Africa) (e.g., CHROMOFLEX Stent, S-FLEX Stent, S-FLEX Micro Stent, and TAXOCHROME DES); Intek Technology (Baar, Switzerland) (e.g., APOLLO stent); Orbus Medical Technologies (Hoevelaken, The Netherlands) (e.g., GENOUS); Sorin Biomedica (Saluggia, Italy) (e.g., JANUS and CARBOSTENT); and stents from Bard/Angiomed GmbH Medizintechnik KG (Murray Hill, N.J.), and Blue Medical Supply & Equipment (Mariettta, Ga.), Aachen Resonance GmbH (Germany); Eucatech AG (Germany), Eurocor GmbH (Bonn, Gemany), Prot, Goodman, Terumo (Japan), Translumina GmbH (Germany), MIV Therapeutics (Canada), Occam International B.V. (Eindhoven, The Netherlands), Sahajanand Medical Technologies PVT LTD. (India); AVI Biopharma/Medtronic/Interventional Technologies (Portland, Oreg.) (e.g., RESTEN NG-coated stent); and Jomed (e.g., FLEXMASTER drug-eluting stent) (Sweden).

Generally, stents are inserted in a similar fashion regardless of the site or the disease being treated. Briefly, a preinsertion examination, usually a diagnostic imaging procedure, endoscopy, or direct visualization at the time of surgery, is generally first performed in order to determine the appropriate positioning for stent insertion. A guidewire is then advanced through the lesion or proposed site of insertion, and over this is passed a delivery catheter which allows a stent in its collapsed form to be inserted. Intravascular stents may be inserted into an artery such as the femoral artery in the groin and advanced through the circulation under radiological guidance until they reach the anatomical location of the plaque in the coronary or peripheral circulation. Typically, stents are capable of being compressed, so that they can be inserted through tiny cavities via small catheters, and then expanded to a larger diameter once they are at the desired location. The delivery catheter then is removed, leaving the stent standing on its own as a scaffold. Once expanded, the stent physically forces the walls of the passageway apart and holds them open. A post insertion examination, usually an x-ray, is often utilized to confirm appropriate positioning.

Stents are typically maneuvered into place under, radiologic or direct visual control, taking particular care to place the stent precisely within the vessel being treated. In certain aspects, the stent can further include a radio-opaque, echogenic material, or MRI responsive material (e.g., MRI contrast agent) to aid in visualization of the device under ultrasound, fluoroscopy and/or magnetic resonance imaging. The radio-opaque or MRI visible material may be in the form of one or more markers (e.g., bands of material that are disposed on either end of the stent) that may be used to orient and guide the device during the implantation procedure.

The fibrosing agent can be delivered into the vulnerable plaque via specialized drug-delivery stents to gradually convert the unstable plaque into contracted, hemodynamically stable fibrous tissue. Luminal application of the fibrosing agent also can be used to increase the thickness and stability of the thin fibrous layer which covers the vulnerable plaque.

In certain aspects, the fibrosing agent is released from the drug-delivery stent in concentrations in order to deliver therapeutic doses of the drug to the atherosclerotic plaque. In one aspect, the stent may be coated with a polymeric composition which releases the fibrosing agent over a period ranging from several hours to several weeks to several months after deployment of the device within the diseased vessel.

It is important to note that unstable or vulnerable plaque tends to form asymmetrically in the vessel wall. Therefore, all of the above described embodiments need not be applied to all aspects of the device (e.g., stent or balloon). It is possible to preferentially deliver fibrosing therapies only to those portions of the device which will be in contact with the vulnerable plaque, while leaving the rest of the device in its native state.

In another aspect, catheters, stents, balloons, and other intravascular devices may be delivered to an anatomical site containing vulnerable plaque in order to treat or prevent plaque rupture. Briefly, using sterile conditions, under appropriate anesthesia and analgesia, the common femoral artery is located and cannulated. A guide wire is manipulated through the arterial system to the site of the vulnerable plaque (e.g., the coronary and carotid arteries are commonly affected) and an angiogram, intravascular ultrasound (IVUS) or other diagnostic test is performed to identify the exact location of the lesion. The vulnerable plaque may also be dilated by inflating an angioplasty balloon at some point during the procedure. Subsequently, the diagnostic catheter (or angioplasty balloon) is exchanged over a guide wire for a drug delivery catheter, drug delivery balloon, drug-coated stent or other drug-coated intravascular device. For drug delivery catheters, the fibrosing agent is delivered via the lumen of the catheter at sufficient doses in the vicinity of the vulnerable plaque. For drug-delivery balloons, the balloon is typically advanced across the lesion and inflated not only to dilate the plaque, but also to facilitate localized delivery of the fibrosing agent into the plaque wall.

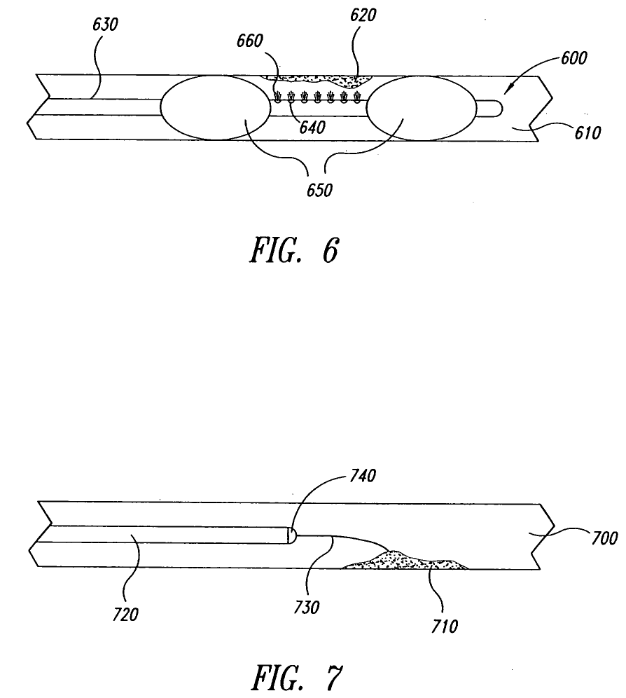

Referring to FIG. 6, a dual balloon catheter 600 is shown that has been inserted into a body passageway (e.g., an artery) 610 which contains a deposit of vulnerable plaque 620. The dual balloon catheter 600 includes a catheter 630 having a plurality of drug delivery ports 640. The dual balloon catheter 600 further includes two balloons 650, which once inflated (as shown in FIG. 6) flank the plaque 620 on either side. A fibrosing agent 660 or a composition containing the fibrosing agent 660 can be delivered to the plaque 620 through the catheter delivery holes 640 of the catheter 630. The composition may incubate the plaque for a period of time, or the composition may change from a fluent state to a non-fluent state, such that the plaque is coated with the fibrosing composition. If a drug-coated stent is deployed, it is positioned across the lesion and then expanded in place by inflating a balloon (this not required for “self-expanding” stents). At the completion of the procedure, an angiogram or IVUS is performed to confirm location and the introduction catheter is removed.

In some clinical situations it may be appropriate to deliver the fibrosing agent during open or endoscopic vascular surgery procedures. For example, during coronary or peripheral arterial bypass surgery, the fibrosing agent could be placed directly on the adventitia (outer vascular wall) of segments of the artery that contain unstable plaque. Alternatively, the fibrosing agent could be directly injected into the unstable plaque through the arterial wall.

In certain other embodiments, the fibrosing composition is directly injected into a vulnerable plaque through a guidable multi-lumen needle of a catheter. Referring to FIG. 7, a cross section of a body passageway (e.g., an artery) 700 that includes a deposit of vulnerable plaque 710 is shown into which catheter 720 has been inserted. The fibrosing agent (not shown) can be delivered to the plaque 710 directly from the catheter through a guidable multi-lumen needle 730 that is located at the tip 740 of the catheter 720. The fibrosing agent may be in a fluent state before and after delivery or it may be in a fluent state before delivery and in a non-fluent state after delivery. If the affected artery is accessed by less invasive procedures, such as endoscopic bypass or pericardial access devices, the fibrosing agent can be applied regionally (e.g., into the pericardial space) or locally (e.g., direct application or injection into the affected artery).

It should be apparent to one of skill in the art that potentially any fibrosing agent described above may be utilized alone, or in combination, in the practice of this embodiment. Suitable fibrosing agents may be readily determined based upon the exemplary animal models provided herein. Animal models for detection of vulnerable plaque and a model for testing agents also are described in U.S. patent application No. 2001/001 8042A1. Exemplary fibrosing agents for use with stent, drug delivery balloon, and catheter devices include talc, silk, chitosan, polylysine, fibronectin, silver nitrate, bleomycin, and CTGF, as well as analogues and derivatives of the aforementioned. Other materials for promoting adhesion of stents to biological tissue include microemulsions formed from caprylocaproyl macrogol-8 glycerides, such as those sold under the trade name LABRASOL, PEG-PLGA polymers, PLURONICs, sucrose, starch (e.g., corn starch or maize starch) and other materials that are known to induce the formation of surgical adhesions when administered in vivo.

As stent, drug delivery balloon, and catheter devices are made in a variety of configurations and sizes depending upon the location and the degree of the injury, the exact dose administered will vary with implant size, surface area and design. However, certain principles can be applied in the application of this art. Drug dose can be calculated as a function of dose per unit area (or volume) of the device or implant being coated, total drug dose administered can be measured and appropriate surface concentrations of active drug can be determined. Regardless of the method of application of the drug to the blood vessel (typically the aorta), or devices, the exemplary fibrosing agents, used alone or in combination, should be administered under the following dosing guidelines:

Utilizing talc as a preferred fibrosing agent, whether it is applied using a polymer coating, incorporated into the polymers which make up the device, or applied without a polymeric carrier, the total dose of talc delivered from a catheter or drug delivery balloon, or coated onto the surface of a stent or other intravascular device, should not exceed 100 mg (range of 1 μg to 100 mg). In a particularly preferred embodiment, the total amount of talc delivered to the vulnerable plaque via catheter, balloon, stent or other intravascular device should be in the range of 10 μg to 50 mg. The dose per unit area of the device (i.e., the dosage of talc as a function of the surface area of the portion of the device to which drug is applied and/or incorporated) should fall within the range of 0.05 μg-10 μg per mm² of surface area coated. In a particularly preferred embodiment, talc should be applied to a stent or other intravascular device surface at a dose of 0.05 μg/mm²-10 μg/mm² of surface area coated. As specific (polymeric and non-polymeric) drug delivery vehicles and specific medical devices will release talc at differing rates, the above dosing parameters should be utilized in combination with the release rate of the drug from the catheter, balloon, stent or other intravascular device such that a minimum concentration of 0.01 nM to 1000 μM of talc is delivered to the vulnerable plaque. Excessive dosing is also to be avoided as this can lead to narrowing of the arterial lumen (restenosis). In a preferred embodiment, talc is released from the surface of a stent or injected into the body of the plaque such that fibrosis of the vulnerable plaque is promoted for a period ranging from several hours to several months. In a particularly preferred embodiment, talc is released in effective concentrations for a period ranging from 1 hour-30 days. It should be readily evident given the discussions provided herein that analogues and derivatives of talc (as described previously) with similar functional activity can be utilized for the purposes of this invention; the above dosing parameters are then adjusted according to the relative potency of the analogue or derivative as compared to the parent compound (e.g., a compound twice as potent as talc is administered at half the above parameters, a compound half as potent as talc is administered at twice the above parameters, etc.).

Utilizing silk as a preferred fibrosing agent, whether it is applied using a polymer coating, incorporated into the polymers which make up the device, or applied without a polymeric carrier, the total dose of silk delivered from a catheter or drug delivery balloon, or coated onto the surface of a stent or other intravascular device, should not exceed 100 mg (range of 1 μg to 100 mg). In a particularly preferred embodiment, the total amount of talc delivered to the vulnerable plaque via catheter, balloon, stent or other intravascular device should be in the range of 10 μg to 50 mg. The dose per unit area of the device (i.e., the dosage of silk as a function of the surface area of the portion of the device to which drug is applied and/or incorporated) should fall within the range of 0.05 μg-10 μg per mm² of surface area coated. In a particularly preferred embodiment, silk should be applied to a stent or other intravascular device surface at a dose of 0.05 μmm²-10 μg/mm² of surface area coated. As specific (polymeric and non-polymeric) drug delivery vehicles and specific medical devices will release talc at differing rates, the above dosing parameters should be utilized in combination with the release rate of the drug from the catheter, balloon, stent or other intravascular device such that a minimum concentration of 0.01 nM-1000 μM of silk is delivered to the vulnerable plaque. Excessive dosing is also to be avoided as this can lead to narrowing of the arterial lumen (restenosis). In a preferred embodiment, silk is released from the surface of a stent or injected into the body of the plaque such that fibrosis of the vulnerable plaque is promoted for a period ranging from several hours to several months. In a particularly preferred embodiment, silk is released in effective concentrations for a period ranging from 1 hour-30 days. It should be readily evident given the discussions provided herein that analogues and derivatives of talc (as described previously) with similar functional activity can be utilized for the purposes of this invention; the above dosing parameters are then adjusted according to the relative potency of the analogue or derivative as compared to the parent compound (e.g., a compound twice as potent as silk is administered at half the above parameters, a compound half as potent as silk is administered at twice the above parameters, etc.).

Utilizing chitosan as a preferred fibrosing agent, whether it is applied using a polymer coating, incorporated into the polymers which make up the device, or applied without a polymeric carrier, the total dose of chitosan delivered from a catheter or drug delivery balloon, or coated onto the surface of a stent or other intravascular device, should not exceed 100 mg (range of 1 μg to 100 mg). In a particularly preferred embodiment, the total amount of chitosan delivered to the vulnerable plaque via catheter, balloon, stent or other intravascular device should be in the range of 10 μg to 50 mg. The dose per unit area of the device (i.e., the dosage of chitosan as a function of the surface area of the portion of the device to which drug is applied and/or incorporated) should fall within the range of 0.05 μg-10 μg per mm² of surface area coated. In a particularly preferred embodiment, chitosan should be applied to a stent or other intravascular device surface at a dose of 0.05 μg/mm²-10 μg/mm² of surface area coated. As specific (polymeric and non-polymeric) drug delivery vehicles and specific medical devices will release chitosan at differing rates, the above dosing parameters should be utilized in combination with the release rate of the drug from the catheter, balloon, stent or other intravascular device such that a minimum concentration of 0.01 nM-1000 μM of chitosan is delivered to the vulnerable plaque. Excessive dosing is also to be avoided as this can lead to narrowing of the arterial lumen (restenosis). In a preferred embodiment, chitosan is released from the surface of a stent or injected into the body of the plaque such that fibrosis of the vulnerable plaque is promoted for a period ranging from several hours to several months. In a particularly preferred embodiment, chitosan is released in effective concentrations for a period ranging from 1 hour-30 days. It should be readily evident given the discussions provided herein that analogues and derivatives of talc (as described previously) with similar functional activity can be utilized for the purposes of this invention; the above dosing parameters are then adjusted according to the relative potency of the analogue or derivative as compared to the parent compound (e.g., a compound twice as potent as chitosan is administered at half the above parameters, a compound half as potent as chitosan is administered at twice the above parameters, etc.).

Utilizing polylysine as a preferred fibrosing agent, whether it is applied using a polymer coating, incorporated into the polymers which make up the device, or applied without a polymeric carrier, the total dose of polylysine delivered from a catheter or drug delivery balloon, or coated onto the surface of a stent or other intravascular device, should not exceed 100 mg (range of 1 μg to 100 mg). In a particularly preferred embodiment, the total amount of polylysine delivered to the vulnerable plaque via catheter, balloon, stent or other intravascular device should be in the range of 10 μg to 50 mg. The dose per unit area of the device (i.e., the dosage of polylysine as a function of the surface area of the portion of the device to which drug is applied and/or incorporated) should fall within the range of 0.05 μg-10 μg per mm² of surface area coated. In a particularly preferred embodiment, polylysine should be applied to a stent or other intravascular device surface at a dose of 0.05 μg/mm²-10 μg/mm² of surface area coated. As specific (polymeric and non-polymeric) drug delivery vehicles and specific medical devices will release polylysine at differing rates, the above dosing parameters should be utilized in combination with the release rate of the drug from the catheter, balloon, stent or other intravascular device such that a minimum concentration of 0.01 nM-1000 μM of polylysine is delivered to the vulnerable plaque. Excessive dosing is also to be avoided as this can lead to narrowing of the arterial lumen (restenosis). In a preferred embodiment, polylysine is released from the surface of a stent or injected into the body of the plaque such that fibrosis of the vulnerable plaque is promoted for a period ranging from several hours to several months. In a particularly preferred embodiment, polylysine is released in effective concentrations for a period ranging from 1 hour-30 days. It should be readily evident given the discussions provided herein that analogues and derivatives of polylysine (as described previously) with similar functional activity can be utilized for the purposes of this invention; the above dosing parameters are then adjusted according to the relative potency of the analogue or derivative as compared to the parent compound (e.g., a compound twice as potent as polylysine is administered at half the above parameters, a compound half as potent as polylysine is administered at twice the above parameters, etc.).

Utilizing fibronectin as a preferred fibrosing agent, whether it is applied using a polymer coating, incorporated into the polymers which make up the device, or applied without a polymeric carrier, the total dose of fibronectin delivered from a catheter or drug delivery balloon, or coated onto the surface of a stent or other intravascular device, should not exceed 100 mg (range of 1 μg to 100 mg). In a particularly preferred embodiment, the total amount of fibronectin delivered to the vulnerable plaque via catheter, balloon, stent or other intravascular device should be in the range of 10 μg to 50 mg. The dose per unit area of the device (i.e., the dosage of fibronectin as a function of the surface area of the portion of the device to which drug is applied and/or incorporated) should fall within the range of 0.05 μg-10 μg per mm² of surface area coated. In a particularly preferred embodiment, fibronectin should be applied to a stent or other intravascular device surface at a dose of 0.05 μg/mm²-10 μg/mm² of surface area coated. As specific (polymeric and non-polymeric) drug delivery vehicles and specific medical devices will release fibronectin at differing rates, the above dosing parameters should be utilized in combination with the release rate of the drug from the catheter, balloon, stent or other intravascular device such that a minimum concentration of 0.01 nM-1000 μM of fibronectin is delivered to the vulnerable plaque. Excessive dosing is also to be avoided as this can lead to narrowing of the arterial lumen (restenosis). In a preferred embodiment, fibronectin is released from the surface of a stent or injected into the body of the plaque such that fibrosis of the vulnerable plaque is promoted for a period ranging from several hours to several months. In a particularly preferred embodiment, fibronectin is released in effective concentrations for a period ranging from 1 hour-30 days. It should be readily evident given the discussions provided herein that analogues and derivatives of fibronectin (as described previously) with similar functional activity can be utilized for the purposes of this invention; the above dosing parameters are then adjusted according to the relative potency of the analogue or derivative as compared to the parent compound (e.g., a compound twice as potent as fibronectin is administered at half the above parameters, a compound half as potent as fibronectin is administered at twice the above parameters, etc.).

Utilizing bleomycin as a preferred fibrosing agent, whether it is applied using a polymer coating, incorporated into the polymers which make up the device, or applied without a polymeric carrier, the total dose of bleomycin delivered from a catheter or drug delivery balloon, or coated onto the surface of a stent or other intravascular device, should not exceed 100 mg (range of 0.01 μg to 100 mg). In a particularly preferred embodiment, the total amount of bleomycin delivered to the vulnerable plaque via catheter, balloon, stent or other intravascular device should be in the range of 0.10 μg to 50 mg. The dose per unit area of the device (i.e., the dosage of bleomycin as a function of the surface area of the portion of the device to which drug is applied and/or incorporated) should fall within the range of 0.005 μg-10 μg per mm² of surface area coated. In a particularly preferred embodiment, bleomycin should be applied to a stent or other intravascular device surface at a dose of 0.005 μg/mm²-10 μg/mm² of surface area coated. As specific (polymeric and non-polymeric) drug delivery vehicles and specific medical devices will release bleomycin at differing rates, the above dosing parameters should be utilized in combination with the release rate of the drug from the catheter, balloon, stent or other intravascular device such that a minimum concentration of 0.001 nM-1000 μM of bleomycin is delivered to the vulnerable plaque. Excessive dosing is also to be avoided as this can lead to narrowing of the arterial lumen (restenosis). In a preferred embodiment, bleomycin is released from the surface of a stent or injected into the body of the plaque such that fibrosis of the vulnerable plaque is promoted for a period ranging from several hours to several months. In a particularly preferred embodiment, bleomycin is released in effective concentrations for a period ranging from 1 hour-30 days. It should be readily evident given the discussions provided herein that analogues and derivatives of bleomycin (as described previously) with similar functional activity can be utilized for the purposes of this invention; the above dosing parameters are then adjusted according to the relative potency of the analogue or derivative as compared to the parent compound (e.g., a compound twice as potent as bleomycin is administered at half the above parameters, a compound half as potent as bleomycin is administered at twice the above parameters, etc.).

Utilizing CTGF (connective tissue growth factor) as a preferred fibrosing agent, whether it is applied using a polymer coating, incorporated into the polymers which make up the device, or applied without a polymeric carrier, the total dose of CTGF (connective tissue growth factor) delivered from a catheter or drug delivery balloon, or coated onto the surface of a stent or other intravascular device, should not exceed 100 mg (range of 0.01 μg to 100 mg). In a particularly preferred embodiment, the total amount of CTGF (connective tissue growth factor) delivered to the vulnerable plaque via catheter, balloon, stent or other intravascular device should be in the range of 0.10 μg to 50 mg. The dose per unit area of the device (i.e., the dosage of CTGF (connective tissue growth factor) as a function of the surface area of the portion of the device to which drug is applied and/or incorporated) should fall within the range of 0.005 μg-10 μg per mm² of surface area coated. In a particularly preferred embodiment, CTGF (connective tissue growth factor) should be applied to a stent or other intravascular device surface at a dose of 0.005 μg/mm²-10 μg/mm² of surface area coated. As specific (polymeric and non-polymeric) drug delivery vehicles and specific medical devices will release CTGF (connective tissue growth factor) at differing rates, the above dosing parameters should be utilized in combination with the release rate of the drug from the catheter, balloon, stent or other intravascular device such that a minimum concentration of 0.001 nM-1000 μM of CTGF (connective tissue growth factor) is delivered to the vulnerable plaque. Excessive dosing is also to be avoided as this can lead to narrowing of the arterial lumen (restenosis). In a preferred embodiment, CTGF (connective tissue growth factor) is released from the surface of a stent or injected into the body of the plaque such that fibrosis of the vulnerable plaque is promoted for a period ranging from several hours to several months. In a particularly preferred embodiment, CTGF (connective tissue growth factor) is released in effective concentrations for a period ranging from 1 hour-30 days. It should be readily evident given the discussions provided herein that analogues and derivatives of CTGF (connective tissue growth factor) (as described previously) with similar functional activity can be utilized for the purposes of this invention; the above dosing parameters are then adjusted according to the relative potency of the analogue or derivative as compared to the parent compound (e.g., a compound twice as potent as CTGF (connective tissue growth factor) is administered at half the above parameters, a compound half as potent as CTGF (connective tissue growth factor) is administered at twice the above parameters, etc.).

Other Applications of Intravascular Devices that Include a Fibrosing Agent

In addition to the methods described above, intravascular devices, which are adapted to include and/or release a fibrosing agent or fibrosing composition, can be utilized in a wide variety of other therapeutic applications.

In one aspect, a stent graft may be used as an extravascular or even extra-anatomic conduit such as, but not limited to, between arteries, between an artery and a vein, or between veins, or between a vein and the peritoneal cavity. The expansion of stent grafts for these purposes heretofore has been limited at least partially by the risk of leak of bodily fluid such as blood because of poor sealing at the site where the stent graft enters of leaves a body tube such as a blood vessel) or cavity. The stent grafts of the present invention, in contrast, can be utilized to connect one artery to another, either intra-anatomically, e.g., to bypass aneurysms (e.g., carotid artery, thoracic aorta, abdominal aorta, subclavian artery, iliac artery, coronary artery, venous); to treat dissections (e.g., carotid artery, coronary artery, iliac artery, subclavian artery); to bypass long segment disease (e.g., carotid artery, coronary artery, aorta, iliac artery, femoral artery, popliteal artery), or to treat local rupture (e.g., carotid artery, aorta, iliac artery, renal artery, femoral artery). Stent grafts containing a fibrosing agent may also be utilized extra-anatomically, for example, for arterial-to-arterial dialysis fistula; or for percutaneous bypass grafts and to connect an artery to a vein (e.g., a dialysis fistula), or one vein to another (e.g., a portacaval shunt or venous bypass).

Specific Intravascular Device Embodiments

As described above, the present invention provides intravascular devices such as stents, stent grafts, drug delivery catheters and drug delivery balloons that comprise a fibrosis-inducing agent or a composition that comprises a fibrosis-inducing agent. The intravascular device may comprise i) an intravascular device and ii) an agent or a composition comprising an agent, wherein the agent induces fibrosis. The intravascular device may be, e.g., an intraluminal stent, an intravascular catheter, a drug delivery balloon, aneurysm coil, embolic agent or a stent graft. Also provided are compositions for delivery via an intravascular device (e.g., angioplasty and/or drug-delivery balloon, intra-arterial catheter, stent, or other intravascular delivery device), as well as methods for making and using such devices. Various specific embodiments of the invention are described below.

Stents, Catheters, Balloons, and Other Intravascular Devices

Within one aspect of the invention, intravascular drug delivery devices (e.g., drug-coated or drug-delivery catheters, balloons and stents) are provided which release a drug or agent which induces adhesion or fibrosis in blood vessel walls, thus inducing or increasing the amount of fibrous tissue in unstable plaque. For example, fibrosis may be induced by local or systemic release of specific pharmacological agents that become localized in the unstable plaque. Within other embodiments, the fibrosis is induced by direct injection of specific pharmacological agents into the plaque or into the adjacent tissue surrounding the plaque.

Within related aspects of the present invention, intravascular delivery devices (e.g., intravascular catheters, balloons, stent grafts, covered stents and/or stents) are provided comprising an intravascular device, wherein the device releases an agent which induces or promotes fibrosis in atherosclerotic plaque (and to a certain extent, restenosis) in vivo. Within a related aspect, an intravascular catheter, balloon, stent or other intravascular device is provided wherein the device induces or accelerates an in vivo fibrotic reaction in or around the atherosclerotic plaque. As utilized herein, “induces fibrosis in atherosclerotic plaque” should be understood to refer to agents or compositions which increase or accelerates the formation of fibrous tissue (i.e., tissue composed of fibroblasts, smooth muscle cells and extracellular matrix components such as collagen), such that the fatty plaque material is partially converted into fibrous tissue and/or becomes capped or fixed within the vessel wall (i.e., enhancing/thickening the fibrous tissue separating the plaque from arterial lumen).

Within certain embodiments, an intravascular catheter, balloon, stent or other intravascular device is coated with a compound or material that induces fibrosis in or around the atherosclerotic plaque. Within related aspects, an intravascular catheter, balloon, stent or other intravascular device is constructed so that the device itself is comprised of materials, which induce fibrosis in or around the atherosclerotic plaque. Within related aspects, an intravascular catheter or balloon comprising a fibrosing agent or fibrosing composition is adapted to delivery the fibrosing agent or fibrosing composition in or around the atherosclerotic plaque.

Within one embodiment of the invention, the intravascular catheter, balloon, stent or other intravascular device is adapted to comprise or release an arterial vessel wall irritant. Representative examples of such irritants include talcum powder, metallic beryllium, copper, silk, wool, quartz dust, crystalline silicates and silica. Other agents which may be released by the intravascular catheter, balloon, stent or other intravascular device include components of extracellular matrix, vitronectin, fibronectin, chondroitin sulphate, laminin, hyaluronic acid, elastin, fibrin, fibrinogen, bitronectin, proteins found in basement membrane, fibrosin, collagen, polylysine, cyclosporine A, polyvinyl chloride, poly(ethylene-co-vinylacetate), polyurethane, silk, dacron, and inflammatory cytokines such as TGFβ, PDGF, VEGF (including VEGF-2, VEGF-3, VEGF-A, VEGF-B and VEGFC), aFGF, bFGF, TNFα, NGF, GM-CSF, IGF-a, IL-1, IL-8, IL-6, growth hormone, EDGF (epidermal growth factor), and CTGF (connective tissue growth factor), and analogues and derivatives thereof and adhesives, such as cyanoacrylate or a crosslinked poly(ethylene glycol)—methylated collagen composition. Additional agents suitable for release by the intravascular catheter, balloon, stent or other intravascular device include naturally occurring or synthetic peptides containing the RGD (arginine-glycine-aspartic acid) residue sequence, and foctors produced by immune cells such as interleukin-2 (IL-2), interleukin-4 (IL-4), interleukin-1 (IL-1), interleukin-8 (IL-8), interleukin-6 (IL-6), granulocyte-monocyte colony-stimulating-factor (GM-CSM), monocyte chemotactic protein, bleomycin, histamine and cell adhesion molecules including integrins, and bone morphogenic molecules including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15 and BMP-16. Of these, BMP-2, BMP-3, BMP-4, BMP-5, BMP-6 and BMP-7 are of particular utility.

Within one embodiment of the invention, the intravascular catheter, balloon, stent or other intravascular device is adapted to comprise or release a fibrosing agent from a polymeric and/or non-polymeric carrier, which is in the form of a microsphere (solid or porous) or particulate (e.g., solid or porous microparticulate or nanoparticulate), a paste, gel, liquid, or an in situ forming material. In certain embodiments, the fibrosing agent may be soluble silk protein, microparticulate silk, and/or silk strands (linear, branched, and/or coiled).

Within various embodiments of the invention, an intravascular catheter, balloon, stent or other intravascular device is coated on one aspect with a composition which promotes fibrosis (and/or restenosis), as well as being coated with a composition or compound which acts to have an inhibitory effect on pathological processes in or around the vulnerable plaque. Representative examples of agents which can inhibit pathological processes in the vulnerable plaque include but not limited to the following classes of compounds: anti-inflammatory agents (e.g., dexamethasone, cortisone, fludrocortisone, prednisone, prednisolone, 6α-methylprednisolone, triamcinolone, betamethasone), MMP inhibitors (e.g., batimistat, marimistat, TIMP's (tissue inhibitors of matrix metalloproteinases)), cytokine inhibitors (chlorpromazine, mycophenolic acid, rapamycin, 1α-hydroxy vitamin D₃), IMPDH inhibitors (e.g., mycophenolic acid, ribaviran, aminothiadiazole, thiophenfurin, tiazofurin, viramidine), p38MAP kinase inhibitors (e.g., GW-2286, CGP-52411, BIRB-798, SB220025, RO-320-1195, RWJ-67657, RWJ-68354, CGH-2466, PD-98-59, SCIO-469) and immunosuppressive agents (rapamycin, everolimus, ABT-578) and analogues and derivatives thereof.

Within various embodiments of the invention, an intravascular catheter, balloon, stent or other intravascular device is coated on one aspect with a composition which promotes fibrosis (and/or restenosis), as well as being coated with a composition or compound which acts to stimulate cellular proliferation within the unstable plaque to aid healing of the unstable plaque. Representative examples of agents that stimulate cellular proliferation and include, without limitation, dexamethasone, isotretinoin, 17-β-estradiol, diethylstibesterol, cyclosporine A and all-trans retinoic acid (ATRA) and analogues and derivatives thereof.

Within various embodiments of the invention, an intravascular catheter, balloon, stent or other intravascular device is coated on one aspect, portion or surface with a composition which promotes fibrosis (and/or restenosis), as well as being coated with a composition or compound which prevents restenosis on another aspect, portion or surface of the device. Representative examples of agents that inhibit restenosis (subsequent narrowing of the vascular lumen following initial treatment to open up the obstructed artery by balloon angioplasty, stenting, surgery, cutting balloon, and other plaque ablation therapies) include paclitaxel, sirolimus, everolimus, vincristine, biolimus, mycophenolic acid, ABT-578, cervistatin, simvastatin, methylprednisolone, dexamethasone, actinomycin-D, angiopeptin, L-arginine, estradiol, 17-β-estradiol, tranilast, methotrexate, batimistat, halofuginone, BCP-671, QP-2, lantrunculin D, cytochalasin A, nitric oxide and analogues and derivatives thereof.

Within various embodiments of the invention, an intravascular catheter, balloon, stent, stent graft or other intravascular device is coated on one aspect with a composition which promotes fibrosis (and/or restenosis), as well as being coated with a composition or compound which prevents thrombosis on another aspect of the device. Representative examples of agents that inhibit thrombosis include heparin, aspirin, dipyridamole, as well as analogues and derivatives thereof.

Within various embodiments of the invention, an intravascular catheter, balloon, stent or other intravascular device is coated with a composition or compound, which delays the onset of fibrosis. Representative examples of such agents include heparin, PLGA/MePEG, PLA, surfactants, and polyethylene glycol. Within further embodiments the intravascular catheter, balloon, stent or other intravascular device is activated prior to use (e.g., the agent is first activated from a previously inactive agent to an active agent, or, the device is activated from a previously inactive device to one that induces or accelerates an in vivo fibrotic reaction). Such activation may be accomplished either before insertion, during insertion, or, subsequent to insertion.

Specific Stent Embodiments

In one aspect, the intravascular device is an endoluminal stent. A fibrosis-inducing agent or a composition comprising a fibrosis-inducing agent may be incorporated into or onto (e.g., coated) an intravascular stent in a variety of ways.

In certain embodiments, a fibrosing agent or a composition comprising a fibrosing agent may be directly affixed to the device (e.g., by either spraying or dipping the stent in a solution that contains the desired therapeutic agent; by either spraying the stent with a polymer/drug to create a film or coating on all, or parts, of the stent surface; spraying the stent with a polymerized version of the drug to create a film or coating on all, or parts, of the stent surface; by dipping the device into a carrier (polymeric or non-polymeric)/drug solution to coat all, or parts of the stent surface; by dipping the device into a solution of polymerized or polymerizable drug to coat all, or parts, of the stent surface; or by other covalent or noncovalent (e.g., mechanically attached via knotting or the use of an adhesive or thermal treatment, electrostatic, ionic, hydrogen bonded or hydrophobic interactions) attachment of the therapeutic agent to the stent surface).

In some embodiments, the desired fibrosis-inducing therapeutic agent or composition is incorporated into a hydrogel coating, prepared using methods described herein.

The invention also provides a device, comprising an intraluminal stent and a composition that fully or partially covers the stent, wherein the composition releases an agent, wherein the agent induces fibrosis. In addition, the invention provides a device, comprising an intraluminal stent and a covering that fully or partially covers the stent, wherein all or a portion of the outer surface of the covered stent is coated with an agent or a composition comprising an agent, wherein the agent induces fibrosis.

In other embodiments, the desired fibrosis-inducing therapeutic agent or composition containing the fibrosis-inducing agent is directly affixed to the adluminal (outer) stent surface a (e.g., by either spraying the stent with a polymer/drug to create a film on all, or parts, of the adluminal stent surface; spraying the adluminal stent surface with a polymerized version of the drug to create a film on all, or parts, of the outer stent surface; by dipping the stent into a polymer/drug solution to coat all, or parts of the adluminal stent surface; by dipping the device into a solution of polymerized drug to coat all, or parts, of the adluminal stent surface; or by other covalent or non-covalent attachment of the therapeutic agent to the adluminal stent surface) and also directly affixing (in the manners just described) to the luminal (inner) stent surface a therapeutic agent or composition that inhibits restenosis (such as paclitaxel, vincristine, sirolimus, everolimus, biolimus, mycophenolic acid, ABT-578, cervistatin, simvastatin, methylprednisolone, dexamethasone, actinomycin-D, angiopeptin, L-arginine, estradiol, 17-β-estradiol, tranilast, methotrexate, batimistat, halofuginone, BCP-671, QP-2, lantrunculin D, cytochalasin A, nitric oxide and analogues and derivatives thereof), and/or thrombosis (such as heparin, aspirin, or dipyridamole).

In further embodiments, it may be desirable to induce a blood vessel wall reaction or adhesion at each end of an intravascular stent, but not in the central portion, thus excluding the vulnerable plaque from the circulation. This may be accomplished by coating the ends of the stent with an adhesive/fibrosis inducing agent, and the leaving the center portion of the stent bare (which will induce a lesser degree of restenosis/fibrosis).

The stent may comprise a “thread” composed of, or coated with, the therapeutic agent that is woven into the structure of the stent {e.g., a polymeric strand composed of materials that induce fibrosis (e.g., silk, wool, collagen, EVA, PLA, DACRON (E.I. du Pont de Nemours and Company, Wilmington, Del.), ePTFE, polyurethanes, polymerized drug compositions) or polymers which release a fibrosis-inducing agent from the thread.

All or portions of the stent may be covered with a sleeve or cover (i.e., a continuous covering that isolates the plaque from the circulation (see, e.g., U.S. Pat. Nos. 5,603,722; 5,674,242; 6,019,789; 6,168,619; 6,248,129; and 6,530,950, assigned to Quanam Medical Corporation (Mountain View, Calif.); U.S. Pat. No. 6,290,722) or a mesh (i.e., a discontinuous covering such that portions of the plaque are not isolated and arterial side branches are not obstructed) which is composed of a fibrosis-inducing agent (e.g., polymers such as silk, collagen, EVA, PLA, DACRON, ePTFE, polyurethanes, or polymerized compositions of fibrosis-inducing agents), contains or is coated with the desired fibrosis-inducing therapeutic agent or composition;

All or parts of the stent itself may be constructed with the desired agent or composition. In some embodiments, the stent is constructed from polymers such as silk, collagen, EVA, PLA, DACRON, ePTFE, polyurethanes, or polymerized compositions of fibrosis-inducing agents or otherwise impregnated with the desired agent or composition. In other embodiments, all or parts of the stent may be composed from metals or metal alloys that induce fibrosis (e.g., copper). Alternatively, or in addition, the stent may be made from a degradable or non-degradable polymer that releases one or more fibrosis-inducing agents.

The construction of the stent may include, in addition to a fibrosing agent, physical structures such as ridges or indentation (made, e.g., by scoring), which can produce irritation and ultimately fibrosis in the vicinity of the implanted device.

In one aspect, the stent is a specialized multi-drug releasing stent systems (described, e.g., in U.S. Pat. No. 6,562,065, U.S. patent application Nos. 2003/0199970 and 2003/0167085, and WO 03/015664 and WO 02/32347) that is capable of preferentially delivering fibrosis-inducing agents to arterial plaque (i.e., the adluminal surface of the stent) while preventing restenotic tissue from growing on the luminal surface of the stent by releasing anti-restenotic drugs (e.g., paclitaxel, vincristine, sirolimus, everolimus, biolimus, mycophenolic acid, ABT-578, cervistatin, simvastatin, methylprednisolone, dexamethasone, actinomycin-D, angiopeptin, L-arginine, estradiol, 17-β-estradiol, tranilast, methotrexate, batimistat, halofuginone, BCP-671, QP-2, lantrunculin D, cytochalasin A, nitric oxide and analogues and derivatives thereof) and/or thrombosis (such as heparin, aspirin, dipyridamole) on the inner surface.

Specific Stent Graft Embodiments

The present invention further provides for a device that comprises a stent graft, and a fibrosing agent or a composition comprising a fibrosing agent, wherein the fibrosing agent induces a fibrotic response between the device and a patient in which the device is implanted. The stent graft may, in certain aspects, be coated with, or otherwise adapted to release an agent which induces fibrosis or adhesion to the surrounding tissue. A fibrosis-inducing agent or a composition comprising a fibrosis-inducing agent may be incorporated into or onto a stent graft in a variety of ways.

Stent grafts may be adapted to have incorporated into their structure a fibrosis-inducing agent, adapted to have a surface coating of a fibrosis-inducing agent and/or adapted to release a fibrosis-inducing agent by directly affixing to the implant or device a desired fibrosis-inducing agent or composition containing the fibrosis-inducing agent (e.g., by either spraying the medical implant with a drug and/or carrier (polymeric or non-polymeric)-drug composition to create a film or coating on all, or parts of the internal or external surface of the device; by dipping the implant or device into a drug and/or carrier (polymeric or non-polymeric)-drug solution to coat all or parts of the device or implant; or by other covalent or non-covalent (e.g., mechanically attached via knotting or the use of an adhesive or thermal treatment, electrostatic, ionic, hydrogen bonded or hydrophobic interactions) attachment of the therapeutic agent to the device or implant surface.

In some embodiments, the desired fibrosis-inducing therapeutic agent or composition is incorporated into a hydrogel coating, prepared using methods described herein.

All or parts of the stent graft itself may be constructed with the desired agent or composition. In some embodiments, the stent graft is constructed from polymers such as silk, wool, collagen, EVA, PLA, DACRON, ePTFE, polyurethanes, or polymerized compositions of fibrosis-inducing agents or otherwise impregnated with the desired agent or composition. In other embodiments, the stent graft may comprise a metal or metal alloy that induces fibrosis (e.g., copper). Alternatively, or in addition, the stent graft may include portion that is made from a degradable or non-degradable polymer that releases one or more fibrosis-inducing agents.

The construction of the stent may include, in addition to a fibrosing agent, physical structures such as ridges or indentation (made, e.g., by scoring), which can produce irritation and ultimately fibrosis in the vicinity of the implanted device.

In yet another embodiment, the stent graft comprises a “thread” composed of, or coated with, the fibrosis-inducing agent that is interwoven into the medical implant or device (e.g., a polymeric strand composed of materials that induce fibrosis (e.g., silk, wool, collagen, EVA, PLA, polyurethanes, polymerized drug compositions) or polymers which comprise and/or release a fibrosis-inducing agent from the thread). In one aspect, the thread is biodegradable and comprises a material such as, e.g., a polyester, polyanhydride, poly(anhydride ester), poly(ester-amide), poly(ester-urea), polyorthoester, polyphosphoester, polyphosphazine, polycyanoacrylate, collagen, chitosan, hyaluronic acid, chromic cat gut, alginate, starch, cellulose, or cellulose ester. In another aspect, the thread is non-biodegradable and comprises such as a polyester, polyurethane, silicone, polyethylene, polypropylene, polystyrene, polyacrylate, or polymethacrylate. In one aspect, the non-biodegradable thread is or comprises silk (e.g., a silk suture material). In another aspect, the non-biodegradable thread is, or comprises, wool fibers. In other aspect, the thread is coated with a polymer or with a pharmaceutical agent that induces a fibrotic response in the patient.

The invention also provides a stent graft device, comprising an intraluminal stent and a composition that fully or partially covers the stent, wherein the composition releases an agent, wherein the agent induces fibrosis. In addition, the invention provides a device, comprising an intraluminal stent and a covering that fully or partially covers the stent, wherein all or a portion of the outer surface of the covered stent is coated with an agent or a composition comprising an agent, wherein the agent induces fibrosis.

In one embodiment, all or portions of the device are covered with a sleeve, cover or mesh containing a fibrosis-inducing agent (i.e., a covering comprised of a fibrosis-inducing agent—polymers such as silk, wool, collagen, EVA, PLA, polyurethanes or polymerized compositions containing fibrosis-inducing agents) to encourage scarring and anchoring into the surrounding tissue.

In one aspect, the stent graft is covered (all or in part) with a silk mesh or lattice. In another aspect, the stent graft is covered (all or in part) with a wool mesh or lattice. For example, a silk or wool mesh or lattice can be coated onto all or a portion of the surface of the device to encourage scarring and anchoring into the surrounding tissue.

In another aspect, a stent graft can be combined with a starch (e.g., corn starch or maize starch) such that the device produces a fibrotic response to improve adhesion of the device to the tissue and/or to enhance occlusion of an aneurysm. In one embodiment, starch or a starch-containing composition may be coated onto the device by applying starch powder directly to the device surface. Alternatively, the starch can be applied to the device using a solvent process or an extrusion process. The entire device or only a portion of the device may be coated with the starch. For example, starch can be made into a solution (e.g., by placing a 5% aqueous solution in an autoclave for 45 min.) that can be coated onto the outer surface of the device. The solvent then is removed to leave the starch coated on the device. In another approach, the starch can be incorporated into a secondary carrier (e.g., a degradable or non-degradable polymer, wax, lipid, oil, and the like), which may, optionally, be cross-linked. The secondary carrier (e.g., polymer) can be coated onto the device. For example, the starch may be incorporated into or onto a non-degradable polymer (e.g., silk or DACRON) or biodegradable polymer (e.g., PLGA) which is then coated onto the device. As the polymer degrades, the starch is released to the surrounding tissue where it may cause the desired biological response. Alternatively, or in addition, the starch may be incorporated into the materials used to make the graft and/or stent portion of the device.

Within one embodiment of the invention, stent, stent graft, catheter, balloon, aneurysm coil, or embolic agent is adapted to comprise or release an arterial vessel wall irritant. Representative examples of such irritants include talcum powder, metallic beryllium, copper, silk, quartz dust, crystalline silicates and silica. Other agents which may be released by the intravascular catheter, balloon, stent, stent graft, aneurysm coil, embolic agent or other intravascular device include components of extracellular matrix, vitronectin, fibronectin, chondroitin sulphate, laminin, hyaluronic acid, elastin, fibrin, fibrinogen, bitronectin, proteins found in basement membrane, fibrosin, collagen, polylysine, cyclosporine A, poly(vinyl chloride), poly(ethylene-co-vinylacetate), polyurethane, silk, DACRON, and inflammatory cytokines such as TGFβ, PDGF, VEGF (including VEGF-2, VEGF-3, VEGF-A, VEGF-B and VEGFC), aFGF, bFGF, TNFα, NGF, GM-CSF, IGF-a, IL-1, IL-8, IL-6, growth hormone, EDGF (epidermal growth factor), and CTGF (connective tissue growth factor), and analogues and derivatives thereof and adhesives, such as cyanoacrylate or a crosslinked poly(ethylene glycol)—methylated collagen composition. Additional agents suitable for incorporation into and/or release by the intravascular catheter, balloon, stent, stent graft, aneurysm coil, embolic agent or other device include naturally occurring or synthetic peptides containing the RGD (arginine-glycine-aspartic acid) residue sequence, and factors produced by immune cells such as interleukin-2 (IL:-2), interleukin-4 (IL-4), interleukin-1 (IL-1), interleukin-8 (IL-8), interleukin-6 (IL-6), granulocyte-monocyte colony-stimulating-factor (GM-CSM), monocyte chemotactic protein, bleomycin, histamine and cell adhesion molecules including integrins, and bone morphogenic molecules including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15 and BMP-16. Of these, BMP-2, BMP-3, BMP-4, BMP-5, BMP-6 and BMP-7 are of particular utility.

Within various embodiments, a stent graft is coated on one aspect with a composition which promotes fibrosis and/or thrombosis, as well as being coated with another therapeutic composition or compound on another aspect of the device.

Within various embodiments of the invention, a stent graft is coated on one aspect with a composition which promotes fibrosis (and/or restenosis), as well as being coated with a composition or compound which acts to stimulate cellular proliferation to enhance scarring between the device and the surrounding tissue. For example, in one embodiment, a stent graft is coated on one aspect with a composition which promotes fibrosis (and/or restenosis) such as silk, as well as being coated with a composition or compound which acts to stimulate cellular proliferation, such as cyclosporine A. Other examples of agents that stimulate cellular proliferation include, without limitation, dexamethasone, isotretinoin, 17-β-estradiol, diethylstibesterol, and all-trans retinoic acid (ATRA) and analogues and derivatives thereof. In yet another embodiment, threads that are made from silk, or comprise silk can be affixed to the external surface of the stent graft (e.g., to the graft portion). The device comprising the silk threads may be coated on another aspect with a composition or compound which acts to stimulate cellular proliferation, such as cyclosporine A. In another embodiment, threads that are made from wool, or comprise wool can be affixed to the external surface of the stent graft (e.g., to the graft portion). The device comprising the wool threads may be coated on another aspect with a composition or compound which acts to stimulate cellular proliferation, such as cyclosporine A.

Within various embodiments of the invention, a stent graft coated on one aspect with a composition which promotes fibrosis (and/or restenosis), as well as being coated with a composition or compound which prevents restenosis on another aspect of the device. Representative examples of agents that inhibit restenosis (subsequent narrowing of the vascular lumen following initial treatment to open up the obstructed artery by balloon angioplasty, stenting, surgery, cutting balloon, and other plaque ablation therapies) include paclitaxel, sirolimus, everolimus, vincristine, biolimus, mycophenolic acid, ABT-578, cervistatin, simvastatin, methylprednisolone, dexamethasone, actinomycin-D, angiopeptin, L-arginine, estradiol, 17-β-estradiol, tranilast, methotrexate, batimistat, halofuginone, BCP-671, QP-2, lantrunculin D, cytochalasin A, nitric oxide and analogues and derivatives thereof.

In one embodiment, the external surface of a stent graft may be coated with a fibrosing and/or thrombotic agent or composition to promote scarring and/or thrombus formation in the aneurysm sac and the perigraft space, and the internal (luminal) surface of the stent and/or graft portion may be coated with a composition that comprises an agent that inhibits scarring to prevent intimal growth and luminal narrowing (e.g., an anti-microtubule agent such as, e.g., paclitaxel, sirolimus, everolimus, as well as analogues and derivatives thereof).

Within various embodiments of the invention, a stent graft is coated on one aspect with a composition which promotes fibrosis (and/or restenosis), as well as being coated with a composition or compound which prevents thrombosis on another aspect of the device. Representative examples of agents that inhibit thrombosis include heparin, aspirin, dipyridamole, as well as analogues and derivatives thereof. For example, a fibrosing and/or thrombotic agent may be coated on the adluminal surface of the stent graft, and an anti-thrombotic agent (e.g., heparin) may be coated on a luminal surface of the device.

Within various embodiments of the invention, a stent graft is coated with a composition or compound, which delays the onset of fibrosis, such as include heparin, PLGA/MePEG, PLA, surfactants, and polyethylene glycol.

The present invention also provides the following itemized embodiments.

1. A method of inducing fibrosis in a patient, comprising delivering locally to a tissue proximate to a blood vessel lumen in a patient in need thereof, wherein the blood vessel has a luminal surface, a fibrosing agent or a composition comprising a fibrosing agent, wherein the agent induces fibrosis.

2. The method of item 1 wherein the tissue is diseased tissue.

3. The method of item 1 wherein the tissue is a blood vessel wall in the vicinity of a diseased tissue.

4. The method of item 1 wherein the fibrosing agent or the composition comprising the fibrosing agent is delivered to a luminal surface of the blood vessel.

5. The method of item 1 wherein the fibrosing agent or a composition comprising the fibrosing agent is delivered into the tissue.

6. The method of item 1 wherein the blood vessel is an artery.

7. The method of item 1 wherein the blood vessel is an aorta.

8. The method of item 1 wherein the tissue is arterial plaque.

9. The method of item 1 wherein the tissue is unstable arterial plaque.

10. The method of item 1, further comprising deploying an intravascular device within the blood vessel, wherein the device comprises the fibrosing agent or the composition comprising the fibrosing agent, wherein the device is configured to locally deliver the fibrosing agent or composition comprising the fibrosing agent to a tissue in the vicinity of the device once it is deployed, where the fibrosing agent induces fibrosis.

11. The method of item 10 wherein the intravascular device is adapted to release the fibrosing agent after deployment of the device.

12. The method of item 10 wherein the device is a stent.

13. The method of item 10 wherein the device is a self-expandable stent.

14. The method of item 10 wherein the device is a balloon-expandable stent.

15. The method of item 10 wherein the device is a stent, wherein the stent further comprises a covering that fully or partially covers the stent.

16. The method of item 10 wherein the device is a stent, wherein the stent further comprises a covering that fully or partially covers the stent, wherein the covering is in the form of a tube, sleeve, or spiral.

17. The method of item 10 wherein the device is a stent, wherein the stent further comprises a covering that fully or partially covers the stent, wherein the covering is in the form of a mesh or film.

18. The method of item 10 wherein the device is a stent, wherein the stent further comprises a covering that fully or partially covers the stent, wherein the covering is in the form of a mesh or film, wherein the film is a solid film.

19. The method of item 10 wherein the device is a stent, wherein the stent further comprises a covering that fully or partially covers the stent, wherein the covering is in the form of a mesh or film, wherein the film is a porous film.

20. The method of item 10 wherein the device is a balloon over stent device.

21. The method of item 10 wherein the device is a stent, wherein the stent is adapted to release the agent at only the distal ends of the stent.

22. The method of item 10 wherein the device is a stent, wherein the stent is adapted to release the agent along the entire body of the stent.

23. The method of item 10 wherein the device is a stent graft, wherein the stent graft comprises a stent portion and a graft portion.

24. The method of item 10 wherein the device is a stent graft, wherein the stent graft a bifurcated stent graft.

25. The method of item 10 wherein the device is a stent graft, wherein the stent graft comprises a stent portion and a graft portion, wherein the graft portion comprises a polymer.

26. The method of item 10 wherein the device is a stent graft, wherein the stent graft comprises a stent portion and a graft portion, wherein the graft portion comprises a polymer, wherein the polymer comprises a polyester, a polyurethane, poly(tetrafluoroethylene), or polypropylene.

27. The method of item 10 wherein the device is a stent graft, wherein the stent graft comprises a stent portion and a graft portion, wherein the stent graft comprises an external stent.

28. The method of item 10 wherein the device is a stent graft, wherein the stent graft comprises a stent portion and a graft portion, wherein the stent graft is adapted to release the agent along all or a portion of the stent portion of the stent graft.

29. The method of item 10 wherein the device is a stent graft, wherein the stent graft comprises a stent portion and a graft portion, wherein the stent graft is adapted to release the agent along all or a portion of the graft portion of the stent graft.

30. The method of item 10 wherein the device is an intravascular catheter.

31. The method of item 10 wherein the device is an intravascular catheter, wherein the intravascular catheter is selected from the group consisting of balloon catheters, dilitation catheters, infusion catheters, infusion sleeve catheters, needle injection catheters, pressure driven catheters, phonophoresis catheters, and iontophoresis catheters.

32. The method of item 10 wherein the device is a balloon.

33. The method of item 10 wherein the device is a balloon, wherein the balloon is a porous balloon, a channel balloon, a microinjector balloon, a double balloon, a perfusion balloon, or a spiral balloon.

34. The method of item 10 wherein the device is a coronary drug infusion guidewire.

35. The method of item 10 wherein the device is a vascular graft or shunt

36. The method of item 10 wherein the device is an anastomotic connector device.

37. The method of item 10 wherein the device further comprises a coating, wherein the coating comprises the fibrosing agent.

38. The method of item 10 wherein the device further comprises a coating, wherein the coating is disposed on a surface of the device, wherein the coating comprises the fibrosing agent.

39. The method of item 10 wherein the device further comprises a coating, wherein the coating directly contacts the device, wherein the coating comprises the fibrosing agent.

40. The method of item 10 wherein the device further comprises a coating, wherein the coating indirectly contacts the device, wherein the coating comprises the fibrosing agent.

41. The method of item 10 wherein the device further comprises a coating, wherein the coating partially covers the device, wherein the coating comprises the fibrosing agent.

42. The method of item 10 wherein the device further comprises a coating, wherein the coating completely covers the device, wherein the coating comprises the fibrosing agent.

43. The method of item 10 wherein the device further comprises a coating, wherein the coating is a uniform coating, wherein the coating comprises the fibrosing agent.

44. The method of item 10 wherein the device further comprises a coating, wherein the coating is a non-uniform coating, wherein the coating comprises the fibrosing agent.

45. The method of item 10 wherein the device further comprises a coating, wherein the coating is a discontinuous coating, wherein the coating comprises the fibrosing agent.

46. The method of item 10 wherein the device further comprises a coating, wherein the coating is a patterned coating, wherein the coating comprises the fibrosing agent.

47. The method of item 10 wherein the device further comprises a coating, wherein the coating has a thickness of 100 mm or less, wherein the coating comprises the fibrosing agent.

48. The method of item 10 wherein the device further comprises a coating, wherein the coating has a thickness of 10 mm or less, wherein the coating comprises the fibrosing agent.

49. The method of item 10 wherein the device further comprises a coating, wherein the coating adheres to the surface of the device upon deployment of the device, wherein the coating comprises the fibrosing agent.

50. The method of item 10 wherein the device further comprises a coating, wherein the coating is stable at room temperature for a period of at least 1 year, wherein the coating comprises the fibrosing agent.

51. The method of item 10 wherein the device further comprises a coating, wherein the fibrosing agent is present in the coating in an amount ranging between about 0.0001% to about 1% by weight.

52. The method of item 10 wherein the device further comprises a coating, wherein the fibrosing agent is present in the coating in an amount ranging between about 1% to about 10% by weight.

53. The method of item 10 wherein the device further comprises a coating, wherein the fibrosing agent is present in the coating in an amount ranging between about 10% to about 25% by weight.

54. The method of item 10 wherein the device further comprises a coating, wherein the fibrosing agent is present in the coating in an amount ranging between about 25% to about 70% by weight.

55. The method of item 10 wherein the device further comprises a coating, wherein the coating further comprises a polymer.

56. The method of item 10 wherein the device further comprises a first coating having a first composition and the second coating having a second composition.

57. The method of item 10 wherein the device further comprises a first coating having a first composition and the second coating having a second composition, wherein the first composition and the second composition are different.

58. The method of item 10 wherein the device comprises about 0.01 mg to about 10 mg of the fibrosing agent.

59. The method of item 10 wherein the device comprises about 10 mg to about 10 mg of the fibrosing agent.

60. The method of item 10 wherein the device comprises about 10 mg to about 250 mg of the fibrosing agent.

61. The method of item 10 wherein the device comprises about 250 mg to about 1000 mg of the fibrosing agent.

62. The method of item 10 wherein the device comprises about 1000 mg to about 2500 mg of the fibrosing agent.

63. The method of item 10 wherein a surface of the device comprises less than 0.01 mg of the fibrosing agent per mm² of device surface to which the fibrosing agent is applied.

64. The method of item 10 wherein a surface of the device comprises about 0.01 mg to about 1 mg of the fibrosing agent per mm² of device surface to which the fibrosing agent is applied.

65. The method of item 10 wherein a surface of the device comprises about 1 mg to about 10 mg of the fibrosing agent per mm² of device surface to which the fibrosing agent is applied.

66. The method of item 10 wherein a surface of the device comprises about 10 mg to about 250 mg of the fibrosing agent per mm² of device surface to which the fibrosing agent is applied.

67. The method of item 10 wherein a surface of the device comprises about 250 mg to about 1000 mg of the fibrosing agent of fibrosing agent per mm² of device surface to which the fibrosing agent is applied.

68. The method of item 10 wherein a surface of the device comprises about 1000 mg to about 2500 mg of the fibrosing agent per mm² of device surface to which the fibrosing agent is applied.

69. The method of item 1 wherein the composition is in the form of a paste, gel, or liquid.

70. The method of item 1 wherein the fibrosing agent is in the form of tufts.

71. The method of item 1 composition is in the form of microspheres, nanospheres, or micelles.

72. The method of item 1 wherein the composition is in the form of an aqueous solution.

73. The method of item 1 wherein the composition is in the form of an aqueous solution, wherein the aqueous solution is a phosphate buffered saline solution.

74. The method of item 1 wherein the composition comprises a biocompatible solvent.

75. The method of item 1 wherein the composition comprises a biocompatible solvent, wherein the solvent is selected from the group consisting of N-methyl-2-pyrrolidone,-2-pyrrolidone, acetone, methyl acetate, ethyl acetate, methyl ethyl ketone, dimethylformamide, dimethyl sulfoxide, tetrahydrofuran, caprolactam, decylmethylsulfoxide, oleic acid, and 1-dodecylazacycloheptan-2-one, and poly(ethylene) glycol, and mixtures thereof.

76. The method of item 1 wherein the composition comprises a polymer.

77. The method of item 1 wherein the composition comprises a polymer, wherein the polymer provides sustained release for the fibrosing agent.

78. The method of item 1 wherein the composition comprises a polymer, wherein the polymer comprises a copolymer.

79. The method of item 1 wherein the composition comprises a polymer, wherein the polymer comprises a block copolymer.

80. The method of item 1 wherein the composition comprises a polymer, wherein the polymer comprises a random copolymer.

81. The method of item 1 wherein the composition comprises a 20 polymer, wherein the polymer comprises a biodegradable polymer.

82. The method of item 1 wherein the composition comprises a polymer, wherein the polymer comprises a non-biodegradable polymer.

83. The method of item 1 wherein the composition comprises a polymer, wherein the polymer comprises a hydrophilic polymer.

84. The method of item 1 wherein the composition comprises a polymer, wherein the polymer comprises a hydrophobic polymer.

85. The method of item 1 wherein the composition comprises a polymer, wherein the polymer comprises a polymer having hydrophilic domains.

86. The method of item 1 wherein the composition comprises a polymer, wherein the polymer comprises a polymer having hydrophobic domains.

87. The method of item 1 wherein the composition comprises a polymer, wherein the polymer comprises a non-conductive polymer.

88. The method of item 1 wherein the composition comprises a polymer, wherein the polymer comprises an elastomer.

89. The method of item 1 wherein the composition comprises a polymer, wherein the polymer comprises a poly(ethylene glycol)polymer.

90. The method of item 1 wherein the composition comprises a polymer, wherein the polymer comprises an amorphous polymer.

91. The method of item 1 wherein the composition comprises a polymer, wherein the polymer is a crosslinked polymer.

92. The method of item 1 wherein the composition comprises a polymer, wherein the polymer comprises a silicone polymer.

93. The method of item 1 wherein the composition comprises a polymer, wherein the polymer comprises a hydrocarbon polymer.

94. The method of item 1 wherein the composition comprises a polymer, wherein the polymer comprises a styrene-based polymer.

95. The method of item 1 wherein the composition comprises a polymer, wherein the polymer comprises a butadiene polymer.

96. The method of item 1 wherein the composition comprises a polymer, wherein the polymer is or comprises an isobutylene polymer.

97. The method of item 1 wherein the composition comprises a polymer, wherein the polymer is or comprises a member selected from the group consisting of polyurethanes, poly(ethylene-co-vinyl acetate), and acrylic polymers.

98. The method of item 1 wherein the composition comprises a polymer, wherein the polymer is poly(butyl methacrylate), poly(isobutylene), or poly(styrene).

99. The method of item 1 wherein the composition comprises a polymer, wherein the polymer is or comprises collagen.

100. The method of item 1 wherein the composition comprises a polymer, wherein the polymer is or comprises hyaluronic acid.

101. The method of item 1 wherein the composition comprises a polymer, wherein the polymer is or comprises a polyester.

102. The method of item 1 wherein the composition comprises a polymer, wherein the polymer comprises a polyester, wherein the polyester comprises residues from one or more monomers selected from lactide, lactic acid, glycolide, glycolic acid, p-caprolactone, trimethylene carbonate, 1,4-dioxane-2-one, and 1,5-dioxepan-2one.

103. The method of item 1 wherein the composition comprises a polymer, wherein the polymer is or comprises a polyanhydride.

104. The method of item 1 wherein the composition comprises a polymer, wherein the polymer is or comprises poly(alkylene oxide).

105. The method of item 1 wherein the composition comprises a polymer, wherein the polymer is or comprises a polyalkylene oxide block copolymer.

106. The method of item 1 wherein the composition comprises a polymer, wherein the polymer comprises a poly(alkylene oxide)-poly(ester) block copolymer.

107. The method of item 1 wherein the composition comprises a poly(alkylene oxide)-poly(ester) block copolymer having an X—Y, X—Y—X or Y—X—Y structure, wherein X is a poly(alkylene oxide) or a C₁-C₆ monoalkyl ether thereof and Y is a degradable poly(ester).

108. The method of item 1 wherein the composition comprises a material prepared from a 4-armed thiol PEG, a 4-armed NHS PEG, and methylated collagen.

109. The method of item 1 wherein the composition comprises a hydrogel.

110. The method of item 1 wherein the composition comprises a a macromer.

111. The method of item 1 wherein the fibrosing agent promotes regeneration.

112. The method of item 1 wherein the fibrosing agent promotes angiogenesis.

113. The method of item 1 wherein the fibrosing agent promotes fibroblast migration.

114. The method of item 1 wherein the fibrosing agent promotes fibroblast proliferation.

115. The method of item 1 wherein the fibrosing agent promotes deposition of extracellular matrix (ECM).

116. The method of item 1 wherein the fibrosing agent promotes tissue remodeling.

117. The method of item 1 wherein the fibrosing agent promotes adhesion between the device and a host into which the device is implanted.

118. The method of item 1 wherein the fibrosing agent is or comprises an arterial vessel wall irritant.

119. The method of item 1 wherein the fibrosing agent is or comprises an arterial vessel wall irritant selected from the group consisting of talcum powder, metallic beryllium and oxides thereof, copper, silica, crystalline silicates, talc, quartz dust, and ethanol.

120. The method of item 1 wherein the fibrosing agent is or comprises silk.

121. The method of item 1 wherein the fibrosing agent is or comprises silkworm silk.

122. The method of item 1 wherein the fibrosing agent is or comprises spider silk.

123. The method of item 1 wherein the fibrosing agent is or comprises recombinant silk.

124. The method of item 1 wherein the fibrosing agent is or comprises raw silk.

125. The method of item 1 wherein the fibrosing agent is or comprises hydrolyzed silk.

126. The method of item 1 wherein the fibrosing agent is or comprises acid-treated silk.

127. The method of item 1 wherein the fibrosing agent is or comprises acylated silk.

128. The method of item 1 wherein the fibrosing agent is or comprises mineral particles.

129. The method of item I wherein the fibrosing agent is or comprises chitosan.

130. The method of item 1 wherein the fibrosing agent is or comprises polylysine.

131. The method of item 1 wherein the agent is or comprises a component of extracellular matrix.

132. The method of item 1 wherein the agent is or comprises a component of extracellular matrix, wherein the component is selected from collagen, fibrin, and fibrinogen.

133. The method of item 1 wherein the fibrosing agent is or comprises fibronectin.

134. The method of item 1 wherein the fibrosing agent is or comprises bleomycin or an analogue or derivative thereof.

135. The method of item 1 wherein the fibrosing agent is or comprises CTGF.

136. The method of item 1 wherein the agent is or comprises a peptide containing an RGD sequence.

137. The method of item 1 wherein the agent is or comprises poly(ethylene-co-vinylacetate).

138. The method of item 1 wherein the agent is or comprises an adhesive.

139. The method of item 1 wherein the adhesive is or comprises a cyanoacrylate.

140. The method of item 1 wherein the agent is or comprises a crosslinked poly(ethylene glycol)-methylated collagen.

141. The method of item 1 wherein the agent is or comprises an inflammatory cytokine.

142. The method of item 1 wherein the agent is or comprises a growth factor.

143. The method of item 1 wherein the agent is or comprises a member selected from the group consisting of TGFβ, PDGF, VEGF, bFGF, TNFα, NGF, GM-CSF, IGF-a, IL-1, IL-8, IL-6, and growth hormone.

144. The method of item 1 wherein the fibrosing agent is in the form of a thread, or is in contact with a thread.

145. The method of item 1 wherein the fibrosing agent is in the form of a particulate.

146. The method of item 1, further comprising delivering to the patient an inflammatory cytokine.

147. The method of item 1, further comprising delivering to the patient an agent that stimulates cell proliferation.

148. The method of item 1, further comprising delivering to the patient an agent that stimulates cell proliferation, wherein the proliferative agent is selected from the group consisting of dexamethasone, isotretinoin, 17-β-estradiol, estradiol, diethylstibesterol, all-trans retinoic acid (ATRA), and analogues and derivatives thereof.

149. The method of item 1, further comprising delivering to the patient an agent that stimulates cell proliferation, wherein the proliferative agent is cyclosporine A.

150. The method of item 1, further comprising an agent that inhibits infection.

151. The method of item 1, further comprising delivering to the patient an agent that inhibits infection, wherein the agent is an anthracycline.

152. The method of item 1, further comprising delivering to the patient an agent that inhibits infection, wherein the agent is doxorubicin.

153. The method of item 1, further comprising delivering to the patient an agent that inhibits infection, wherein the agent is mitoxantrone.

154. The method of item 1, further comprising delivering to the patient an agent that inhibits infection, wherein the agent is a fluoropyrimidine.

155. The method of item 1, further comprising delivering to the patient an agent that inhibits infection, wherein the agent is 5-fluorouracil (5-FU).

156. The method of item 1, further comprising delivering to the patient an agent that inhibits infection, wherein the agent is a folic acid antagonist.

157. The method of item 1, further comprising delivering to the patient an agent that inhibits infection, wherein the agent is methotrexate.

158. The method of item 1, further comprising delivering to the patient an agent that inhibits infection, wherein the agent is a podophyllotoxin.

159. The method of item 1, further comprising delivering to the patient an agent that inhibits infection, wherein the agent is etoposide.

160. The method of item 1, further comprising delivering to the patient an agent that inhibits infection, wherein the agent is a camptothecin.

161. The method of item 1, further comprising delivering to the patient an agent that inhibits infection, wherein the agent is a hydroxyurea.

162. The method of item 1, further comprising delivering to the patient an agent that inhibits infection, wherein the agent is a platinum complex.

163. The method of item 1, further comprising delivering to the patient an agent that inhibits infection, wherein the agent is cisplatin.

164. The method of item 1, further comprising delivering to the patient a therapeutic agent selected from the group consisting of anti-inflammatory agents, MMP inhibitors, cytokine inhibitors, IMPDH inhibitors, and immunosuppressive agents.

165. The method of item 1, further comprising delivering to the patient an anti-inflammatory agent selected from the group consisting of dexamethasone, cortisone, fludrocortisone, prednisone, prednisolone, 6-methylprednisolone, triamcinolone, and betamethasone. methylprednisolone, 166. The method of item 1, further comprising delivering to the patient an anti-inflammatory agent, wherein the anti-inflammatory agent is a TIMP. methylprednisolone, 167. The method of item 1, further comprising delivering to the patient an anti-inflammatory agent, wherein the anti-inflammatory agent is batimistat, marimistat, doxycycline, tetracycline, minocycline, Ro-1130830, CGS 27023A, or BMS 275291.

168. The method of item 1, further comprising delivering to the patient a cytokine inhibitor selected from the group consisting of chlorpromazine, sirolimus, and 1α-hydroxy vitamin D₃.

169. The method of item 1, further comprising delivering to the patient an IMPDH inhibitor selected from the group consisting of mycophenolic acid, ribaviran, aminothiadiazole, thiophenfurin, tiazofurin, and viramidine.

170. The method of item 1, further comprising a wherein the immunosuppressive agent selected from the group consisting of sirolimus, everolimus, and ABT-578.

171. The method of item 1, further comprising delivering to the patient a compound that inhibits restenosis.

172. The method of item 1, further comprising delivering to the patient a compound that inhibits restenosis, wherein the compound is paclitaxel or an analogue or derivative thereof.

173. The method of item 1, further comprising delivering to the patient a compound that inhibits restenosis, wherein the compound is mycophenolic acid or an analogue or derivative thereof.

174. The method of item 1, further comprising delivering to the patient a compound that inhibits restenosis, wherein the compound is selected from the group consisting of vincristine, biolimus, ABT-578, cervistatin, sirolimus, everolimus, simvastatin, methylprednisolone, actinomycin-D, angiopeptin, L-arginine, tranilast, methotrexate, batimistat, halofuginone, BCP-671, QP-2, lantrunculin D, cytochalasin A, nitric oxide, and analogues and derivatives thereof.

175. The method of item 1, further comprising delivering to the patient a compound that inhibits thrombosis.

176. The method of item 1, further comprising delivering to the patient a compound that inhibits thrombosis.

177. The method of item 1, further comprising delivering to the patient a compound that inhibits thrombosis, wherein the anti-thrombotic agent is selected from the group consisting of heparin, heparin complexes, and analogues and derivatives thereof.

178. The method of item 1, further comprising delivering to the patient a compound that inhibits thrombosis, wherein the anti-thrombotic agent is aspirin or dipyridamole.

179. The method of item 1 wherein the composition further comprises a visualization agent.

180. The method of item 1 wherein the composition further comprises a visualization agent, wherein the visualization agent is a radiopaque material, wherein the radiopaque material comprises a metal, a halogenated compound, or a barium containing compound.

181. The method of item 1 wherein the composition further comprises a visualization agent, wherein the visualization agent is a radiopaque material, wherein the radiopaque material comprises barium, tantalum, or technetium.

182. The method of item 1 wherein the composition further comprises a visualization agent, wherein the visualization agent is a MRI responsive material.

183. The method of item 1 wherein the composition further comprises a visualization agent, wherein the visualization agent comprises a gadolinium chelate.

184. The method of item 1 wherein the composition further comprises a visualization agent, wherein the visualization agent comprises iron, magnesium, manganese, copper, or chromium.

185. The method of item 1 wherein the composition further comprises a visualization agent, wherein the visualization agent comprises an iron oxide compound.

186. The method of item 1 wherein the composition further comprises a visualization agent, wherein the visualization agent comprises a dye, pigment, or colorant.

187. The method of item 1 wherein the composition further comprises an echogenic material.

188. The method of item 1 wherein the fibrosing agent is delivered in effective concentrations from the device over a period ranging from the time of deployment of the device to about 1 year.

189. The method of item 1 wherein the fibrosing agent is delivered in effective concentrations from the device over a period ranging from about 1 month to 6 months.

190. The method of item 1 wherein the fibrosing agent is delivered in effective concentrations from the device over a period ranging from about 1-90 days.

191. The method of item 1 wherein the fibrosing agent is delivered in effective concentrations from the device at a constant rate.

192. The method of item 1 wherein the fibrosing agent is delivered in effective concentrations from the device at an increasing rate.

193. The method of item 1 wherein the fibrosing agent is delivered in effective concentrations from the device at a decreasing rate.

194. The method of item 1 wherein the fibrosing agent is delivered in effective concentrations from the composition comprising the fibrosing agent by diffusion over a period ranging from the time of deployment of the device to about 90 days.

195. The method of item 1 wherein the fibrosing agent is delivered in effective concentrations from the composition comprising the fibrosing agent by erosion of the composition over a period ranging from the time of deployment of the device to about 90 days.

196. A method of inducing fibrosis, comprising:
implanting into a lumen of a blood vessel in a patient in need thereof a device, wherein the device comprises an intravascular device and a fibrosing agent or a composition comprising a fibrosing agent, wherein the device is configured to locally deliver the fibrosing agent or the composition comprising the fibrosing agent to a tissue in the vicinity of the implanted device, wherein the fibrosing agent induces a fibrotic response between the device and the patient in which the device is implanted.

197. The method of item 196 wherein the device is adapted to release the fibrosing agent or composition comprising the fibrosing agent after implantation of the device.

198. The method of item 196 wherein the fibrosing agent or composition comprising the fibrosing agent promotes adhesion between the device and the blood vessel into which the device is implanted.

199. The method of item 196 wherein the intravascular device is an intraluminal stent.

200. The method of item 196 wherein the intravascular device is a self-expandable stent.

201. The method of item 196 wherein the intravascular device is a balloon-expandable stent.

202. The method of item 196 wherein the intravascular device is an intraluminal stent, wherein the stent further comprises a covering that fully or partially covers the stent.

203. The method of item 196 wherein the intravascular device is an intraluminal stent, wherein the stent further comprises a covering that fully or partially covers the stent, wherein the covering is in the form of a tube, sleeve, or spiral.

204. The method of item 196 wherein the intravascular device is an intraluminal stent, wherein the stent further comprises a covering that fully or partially covers the stent, wherein the covering is in the form of a mesh or film.

205. The method of item 196 wherein the intravascular device is an intraluminal stent, wherein the stent further comprises a covering that fully or partially covers the stent, wherein the covering is in the form of a mesh or film, wherein the film is a solid film.

206. The method of item 196 wherein the intravascular device is an intraluminal stent, wherein the stent further comprises a covering that fully or partially covers the stent, wherein the covering is in the form of a mesh or film, wherein the film is a porous film.

207. The method of item 196 wherein the intravascular device is is a balloon over stent device.

208. The method of item 196 wherein the intravascular device is an intraluminal stent, wherein the stent is adapted to release the agent at only the distal ends of the stent.

209. The method of item 196 wherein the intravascular device is an intraluminal stent, wherein the stent is adapted to release the agent along the entire body of the stent.

210. The method of item 196 wherein the intravascular device is a stent graft, wherein the stent graft comprises a stent portion and a graft portion.

211. The method of item 196 wherein the intravascular device is a stent graft, wherein the stent graft comprises a stent portion and a graft portion, wherein the stent graft is a bifurcated stent graft.

212. The method of item 196 wherein the intravascular device is a stent graft, wherein the stent graft comprises a stent portion and a graft portion, wherein the graft portion comprises a polymer.

213. The method of item 196 wherein the intravascular device is a stent graft, wherein the stent graft comprises a stent portion and a graft portion, wherein the graft portion comprises a polymer, wherein the polymer comprises a polyester, a polyurethane, poly(tetrafluoroethylene), or polypropylene.

214. The method of item 196 wherein the intravascular device is a stent graft, wherein the stent graft comprises a stent portion and a graft portion, wherein the stent graft comprises an external stent.

215. The method of item 196 wherein the intravascular device is a stent graft, wherein the stent graft comprises a stent portion and a graft portion, wherein the stent graft is adapted to release the agent along all or a portion of the stent portion of the stent graft.

216. The method of item 196 wherein the intravascular device is a stent graft, wherein the stent graft comprises a stent portion and a graft portion, wherein the stent graft is adapted to release the agent along all or a portion of the graft portion of the stent graft.

217. The method of item 196 wherein the intravascular device is a vascular graft or shunt.

218. The method of item 196 wherein the intravascular device is an anastomotic connector device.

219. The method of item 196 wherein the device further comprises a coating, wherein the coating comprises the fibrosing agent.

220. The method of item 196 wherein the device further comprises a coating, wherein the coating is disposed on a surface of the device, wherein the coating comprises the fibrosing agent.

221. The method of item 196 wherein the device further comprises a coating, wherein the coating directly contacts the device, wherein the coating comprises the fibrosing agent.

222. The method of item 196 wherein the device further comprises a coating, wherein the coating indirectly contacts the device, wherein the coating comprises the fibrosing agent.

223. The method of item 196 wherein the device further comprises a coating, wherein the coating partially covers the device, wherein the coating comprises the fibrosing agent.

224. The method of item 196 wherein the device further comprises a coating, wherein the coating completely covers the device, wherein the coating comprises the fibrosing agent.

225. The method of item 196 wherein the device further comprises a coating, wherein the coating is a uniform coating, wherein the coating comprises the fibrosing agent.

226. The method of item 196 wherein the device further comprises a coating, wherein the coating is a non-uniform coating, wherein the coating comprises the fibrosing agent.

227. The method of item 196 wherein the device further comprises a coating, wherein the coating is a discontinuous coating, wherein the coating comprises the fibrosing agent.

228. The method of item 196 wherein the device further comprises a coating, wherein the coating is a patterned coating, wherein the coating comprises the fibrosing agent.

229. The method of item 196 wherein the device further comprises a coating, wherein the coating has a thickness of 100 mm or less, wherein the coating comprises the fibrosing agent.

230. The method of item 196 wherein the device further comprises a coating, wherein the coating has a thickness of 10 mm or less, wherein the coating comprises the fibrosing agent.

231. The method of item 196 wherein the device further comprises a coating, wherein the coating adheres to the surface of the device upon deployment of the device, wherein the coating comprises the fibrosing agent.

232. The method of item 196 wherein the device further comprises a coating, wherein the coating is stable at room temperature for a period of at least 1 year, wherein the coating comprises the fibrosing agent.

233. The method of item 196 wherein the device further comprises a coating, wherein the fibrosing agent is present in the coating in an amount ranging between about 0.0001% to about 1% by weight.

234. The method of item 196 wherein the device further comprises a coating, wherein the fibrosing agent is present in the coating in an amount ranging between about 1% to about 10% by weight.

235. The method of item 196 wherein the device further comprises a coating, wherein the fibrosing agent is present in the coating in an amount ranging between about 10% to about 25% by weight.

236. The method of item 196 wherein the device further comprises a coating, wherein the fibrosing agent is present in the coating in an amount ranging between about 25% to about 70% by weight.

237. The method of item 196 wherein the device further comprises a coating, wherein the coating further comprises a polymer.

238. The method of item 196 wherein the device further comprises a first coating having a first composition and the second coating having a second composition.

239. The method of item 196 wherein the device further comprises a first coating having a first composition and the second coating having a second composition, wherein the first composition and the second composition are different.

240. The method of item 196 wherein the device comprises about 0.01 mg to about 10 mg of the fibrosing agent.

241. The method of item 196 wherein the device comprises about 10 mg to about 10 mg of the fibrosing agent.

242. The method of item 196 wherein the device comprises about 10 mg to about 250 mg of the fibrosing agent.

243. The method of item 196 wherein the device comprises about 250 mg to about 1000 mg of the fibrosing agent.

244. The method of item 196 wherein the device comprises about 1000 mg to about 2500 mg of the fibrosing agent.

245. The method of item 196 wherein a surface of the device comprises less than 0.01 mg of the fibrosing agent per mm² of device surface to which the fibrosing agent is applied.

246. The method of item 196 wherein a surface of the device comprises about 0.01 mg to about 1 mg of the fibrosing agent per mm² of device surface to which the fibrosing agent is applied.

247. The method of item 196 wherein a surface of the device comprises about 1 mg to about 10 mg of the fibrosing agent per mm² of device surface to which the fibrosing agent is applied.

248. The method of item 196 wherein a surface of the device comprises about 10 mg to about 250 mg of the fibrosing agent per mm² of device surface to which the fibrosing agent is applied.

249. The method of item 196 wherein a surface of the device comprises about 250 mg to about 1000 mg of the fibrosing agent of fibrosing agent per mm² of device surface to which the fibrosing agent is applied.

250. The method of item 196 wherein a surface of the device comprises about 1000 mg to about 2500 mg of the fibrosing agent per mm² of device surface to which the fibrosing agent is applied.

251. The method of item 196 wherein the composition is in the form of a paste, gel, or liquid.

252. The method of item 196 wherein the fibrosing agent is in the form of tufts.

253. The method of item 196 composition is in the form of microspheres, nanospheres, or micelles.

254. The method of item 196 wherein the composition comprises a polymer.

255. The method of item 196 wherein the composition comprises a polymer, wherein the polymer provides sustained release for the fibrosing agent.

256. The method of item 196 wherein the composition comprises a polymer, wherein the polymer comprises a copolymer.

257. The method of item 196 wherein the composition comprises a polymer, wherein the polymer comprises a block copolymer.

258. The method of item 196 wherein the composition comprises a polymer, wherein the polymer comprises a random copolymer.

259. The method of item 196 wherein the composition comprises a polymer, wherein the polymer comprises a biodegradable polymer.

260. The method of item 196 wherein the composition comprises a polymer, wherein the polymer comprises a non-biodegradable polymer.

261. The method of item 196 wherein the composition comprises a polymer, wherein the polymer comprises a hydrophilic polymer.

262. The method of item 196 wherein the composition comprises a polymer, wherein the polymer comprises a hydrophobic polymer.

263. The method of item 196 wherein the composition comprises a polymer, wherein the polymer comprises a polymer having hydrophilic domains.

264. The method of item 196 wherein the composition comprises a polymer, wherein the polymer comprises a polymer having hydrophobic domains.

265. The method of item 196 wherein the composition comprises a polymer, wherein the polymer comprises a non-conductive polymer.

266. The method of item 196 wherein the composition comprises a polymer, wherein the polymer comprises an elastomer.

267. The method of item 196 wherein the composition comprises a polymer, wherein the polymer comprises a poly(ethylene glycol)polymer.

268. The method of item 196 wherein the composition comprises a polymer, wherein the polymer comprises an amorphous polymer.

269. The method of item 196 wherein the composition comprises a polymer, wherein the polymer is a crosslinked polymer.

270. The method of item 196 wherein the composition comprises a polymer, wherein the polymer comprises a silicone polymer.

271. The method of item 196 wherein the composition comprises a polymer, wherein the polymer comprises a hydrocarbon polymer.

272. The method of item 196 wherein the composition comprises a polymer, wherein the polymer comprises a styrene-based polymer.

273. The method of item 196 wherein the composition comprises a polymer, wherein the polymer comprises a butadiene polymer.

274. The method of item 196 wherein the composition comprises a polymer, wherein the polymer is or comprises an isobutylene polymer.

275. The method of item 196 wherein the composition comprises a polymer, wherein the polymer is or comprises a member selected from the group consisting of polyurethanes, poly(ethylene -co-vinyl acetate), and acrylic polymers.

276. The method of item 196 wherein the composition comprises a polymer, wherein the polymer is poly(butyl methacrylate), poly(isobutylene), or poly(styrene).

277. The method of item 196 wherein the composition comprises a polymer, wherein the polymer is or comprises collagen.

278. The method of item 196 wherein the composition comprises a polymer, wherein the polymer is or comprises hyaluronic acid.

279. The method of item 196 wherein the composition comprises a polymer, wherein the polymer is or comprises a polyester.

280. The method of item 196 wherein the composition comprises a polymer, wherein the polymer comprises a polyester, wherein the polyester comprises residues from one or more monomers selected from lactide, lactic acid, glycolide, glycolic acid, k-caprolactone, trimethylene carbonate, 1,4-dioxane-2-one, and 1,5-dioxepan-2one.

281. The method of item 196 wherein the composition comprises a polymer, wherein the polymer is or comprises a polyanhydride.

282. The method of item 196 wherein the composition comprises a polymer, wherein the polymer is or comprises poly(alkylene oxide).

283. The method of item 196 wherein the composition comprises a polymer, wherein the polymer is or comprises a polyalkylene oxide block copolymer.

284. The method of item 196 wherein the composition comprises a polymer, wherein the polymer comprises a poly(alkylene oxide)-poly(ester) block copolymer.

285. The method of item 196 wherein the composition comprises a poly(alkylene oxide)-poly(ester) block copolymer having an X—Y, X—Y—X or Y—X—Y structure, wherein X is a poly(alkylene oxide) or a C₁-C₆ monoalkyl ether thereof and Y is a degradable poly(ester).

286. The method of item 196 wherein the composition comprises a material prepared from a 4-armed thiol PEG, a 4-armed NHS PEG, and methylated collagen.

287. The method of item 196 wherein the composition comprises a hydrogel.

288. The-method of item 196 wherein the composition comprises a a macromer.

289. The method of item 196 wherein the fibrosing agent promotes regeneration.

290. The method of item 196 wherein the fibrosing agent promotes angiogenesis.

291. The method of item 196 wherein the fibrosing agent promotes fibroblast migration.

292. The method of item 196 wherein the fibrosing agent promotes fibroblast proliferation.

293. The method of item 196 wherein the fibrosing agent promotes deposition of extracellular matrix (ECM).

294. The method of item 196 wherein the fibrosing agent promotes tissue remodeling.

295. The method of item 196 wherein the fibrosing agent promotes adhesion between the device and a host into which the device is implanted.

296. The method of item 196 wherein the fibrosing agent is an arterial vessel wall irritant.

297. The method of item 196 wherein the fibrosing agent is an arterial vessel wall irritant selected from the group consisting of talcum powder, metallic beryllium and oxides thereof, copper, silica, crystalline silicates, talc, quartz dust, and ethanol.

298. The method of item 196 wherein the fibrosing agent is or comprises silk.

299. The method of item 196 wherein the fibrosing agent is or comprises silkworm silk.

300. The method of item 196 wherein the fibrosing agent is or comprises spider silk.

301. The method of item 196 wherein the fibrosing agent is or comprises recombinant silk.

302. The method of item 196 wherein the fibrosing agent is or comprises raw silk.

303. The method of item 196 wherein the fibrosing agent is or comprises hydrolyzed silk.

304. The method of item 196 wherein the fibrosing agent is or comprises acid-treated silk.

305. The method of item 196 wherein the fibrosing agent is or comprises acylated silk.

306. The method of item 196 wherein the fibrosing agent is or comprises mineral particles.

307. The method of item 196 wherein the fibrosing agent is or comprises chitosan.

308. The method of item 196 wherein the fibrosing agent is or comprises polylysine.

309. The method of item 196 wherein the agent is a component of extracellular matrix.

310. The method of item 196 wherein the component is selected from collagen, fibrin, and fibrinogen.

311. The method of item 196 wherein the fibrosing agent is or comprises fibronectin.

312. The method of item 196 wherein the fibrosing agent is or comprises bleomycin or an analogue or derivative thereof.

313. The method of item 196 wherein the fibrosing agent is or comprises CTGF.

314. The method of item 196 wherein the agent is or comprises a peptide containing an RGD sequence.

315. The method of item 196 wherein the agent is or comprises poly(ethylene-co-vinylacetate).

316. The method of item 196 wherein the agent is or comprises an adhesive.

317. The method of item 196 wherein the adhesive is or comprises a cyanoacrylate.

318. The method of item 196 wherein the agent is or comprises a crosslinked poly(ethylene glycol)—methylated collagen.

319. The method of item 196 wherein the agent is or comprises an inflammatory cytokine.

320. The method of item 196 wherein the agent is or comprises a growth factor.

321. The method of item 196 wherein the agent is or comprises a member selected from the group consisting of TGFβ, PDGF, VEGF, bFGF, TNFα, NGF, GM-CSF, IGF-a, IL-1, IL-8, IL-6, and growth hormone.

322. The method of item 196 wherein the fibrosing agent is in the form of a thread, or is in contact with a thread.

323. The method of item 196 wherein the fibrosing agent is in the form of a particulate.

324. The method of item 196, further comprising delivering to the patient an inflammatory cytokine.

325. The method of item 196, further comprising delivering to the patient an agent that stimulates cell proliferation.

326. The method of item 196, further comprising delivering to the patient an agent that stimulates cell proliferation, wherein the proliferative agent is selected from the group consisting of dexamethasone, isotretinoin, 17-β-estradiol, estradiol, diethylstibesterol, all-trans retinoic acid (ATRA), and analogues and derivatives thereof.

327. The method of item 196, further comprising delivering to the patient an agent that stimulates cell proliferation, wherein the proliferative agent is cyclosporine A.

328. The method of item 196, further comprising an agent that inhibits infection.

329. The method of item 196, further comprising delivering to the patient an agent that inhibits infection, wherein the agent is an anthracycline.

330. The method of item 196, further comprising delivering to the patient an agent that inhibits infection, wherein the agent is doxorubicin.

331. The method of item 196, further comprising delivering to the patient an agent that inhibits infection, wherein the agent is mitoxantrone.

332. The method of item 196, further comprising delivering to the patient an agent that inhibits infection, wherein the agent is a fluoropyrimidine.

333. The method of item 196, further comprising delivering to the patient an agent that inhibits infection, wherein the agent is 5-fluorouracil (5-FU).

334. The method of item 196, further comprising delivering to the patient an agent that inhibits infection, wherein the agent is a folic acid antagonist.

335. The method of item 196, further comprising delivering to the patient an agent that inhibits infection, wherein the agent is methotrexate.

336. The method of item 196, further comprising delivering to the patient an agent that inhibits infection, wherein the agent is a podophyllotoxin.

337. The method of item 196, further comprising delivering to the patient an agent that inhibits infection, wherein the agent is etoposide.

338. The method of item 196, further comprising delivering to the patient an agent that inhibits infection, wherein the agent is a camptothecin.

339. The method of item 196, further comprising delivering to the patient an agent that inhibits infection, wherein the agent is a hydroxyurea.

340. The method of item 196, further comprising delivering to the patient an agent that inhibits infection, wherein the agent is a platinum complex.

341. The method of item 196, further comprising delivering to the patient an agent that inhibits infection, wherein the agent is cisplatin.

342. The method of item 196, further comprising delivering to the patient a therapeutic agent selected from the group consisting of anti-inflammatory agents, MMP inhibitors, cytokine inhibitors, IMPDH inhibitors, and immunosuppressive agents.

343. The method of item 196, further comprising delivering to the patient an anti-inflammatory agent selected from the group consisting of dexamethasone, cortisone, fludrocortisone, prednisone, prednisolone, 6α-methylprednisolone, triamcinolone, and betamethasone.

344. The method of item 196, further comprising delivering to the patient an anti-inflammatory agent, wherein the anti-inflammatory agent is a TIMP.

345. The method of item 196, further comprising delivering to the patient an anti-inflammatory agent, wherein the anti-inflammatory agent is batimistat, marimistat, doxycycline, tetracycline, minocycline, Ro-1130830, CGS 27023A, or BMS 275291.

346. The method of item 196, further comprising delivering to the patient a cytokine inhibitor selected from the group consisting of chlorpromazine, sirolimus, and 1α-hydroxy vitamin D₃.

347. The method of item 196, further comprising delivering to the patient an IMPDH inhibitor selected from the group consisting of mycophenolic acid, ribaviran, aminothiadiazole, thiophenfurin, tiazofurin, and viramidine.

348. The method of item 196, further comprising a wherein the immunosuppressive agent selected from the group consisting of sirolimus, everolimus, and ABT-578.

349. The method of item 196 wherein the device comprises a tubular structure having a lumen through which blood flows, wherein the device comprises a luminal surface and a non-luminal surface.

350. The method of item 196 further comprising delivering to the patient a compound that inhibits restenosis.

351. The method of item 196 further comprising delivering to the patient a compound that inhibits restenosis, wherein the compound is paclitaxel or an analogue or derivative thereof.

352. The method of item 196 further comprising delivering to the patient a compound that inhibits restenosis, wherein the compound is mycophenolic acid or an analogue or derivative thereof.

353. The method of item 196 further comprising delivering to the patient a compound that inhibits restenosis, wherein the compound is selected from the group consisting of vincristine, biolimus, ABT-578, cervistatin, sirolimus, everolimus, simvastatin, methylprednisolone, actinomycin-D, angiopeptin, L-arginine, tranilast, methotrexate, batimistat, halofuginone, BCP-671, QP-2, lantrunculin D, cytochalasin A, nitric oxide, and analogues and derivatives thereof.

354. The method of item 196 further comprising a compound that inhibits thrombosis.

355. The method of item 196 further comprising a compound that inhibits thrombosis, wherein the anti-thrombotic agent is selected from the group consisting of heparin, heparin complexes, and analogues and derivatives thereof.

356. The method of item 196 further comprising a compound that inhibits thrombosis, wherein the anti-thrombotic agent is aspirin or dipyridamole.

357. The method of item 196 wherein the composition further comprises a visualization agent.

358. The method of item 196 wherein the composition further comprises a visualization agent, wherein the visualization agent is a radiopaque material, wherein the radiopaque material comprises a metal, a halogenated compound, or a barium containing compound.

359. The method of item 196 wherein the composition further comprises a visualization agent, wherein the visualization agent is a radiopaque material, wherein the radiopaque material comprises barium, tantalum, or technetium.

360. The method of item 196 wherein the composition further comprises a visualization agent, wherein the visualization agent is a MRI responsive material.

361. The method of item 196 wherein the composition further comprises a visualization agent, wherein the visualization agent comprises a gadolinium chelate.

362. The method of item 196 wherein the composition further comprises a visualization agent, wherein the visualization agent comprises iron, magnesium, manganese, copper, or chromium.

363. The method of item 196 wherein the composition further comprises a visualization agent, wherein the visualization agent comprises an iron oxide compound.

364. The method of item 196 wherein the composition further comprises a visualization agent, wherein the visualization agent comprises a dye, pigment, or colorant.

365. The method of item 196 wherein the composition further comprises an echogenic material.

366. The method of item 196 wherein the composition further comprises an echogenic material, wherein the echogenic material is in the form of a coating.

367. The method of item 196 wherein the device is adapted to release the compound after deployment of the device.

368. The method of item 196 wherein the fibrosing agent is released in effective concentrations from the device over a period ranging from the time of deployment of the device to about 1 year.

369. The method of item 196 wherein the fibrosing agent is released from the device in effective concentrations from the device over a period ranging from about 1 month to 6 months.

370. The method of item 196 wherein the fibrosing agent is released from the device in effective concentrations from the device over a period ranging from about 1-90 days.

371. The method of item 196 wherein the fibrosing agent is released from the device in effective concentrations from the device at a constant rate.

372. The method of item 196 wherein the fibrosing agent is released from the device in effective concentrations from the device at an increasing rate.

373. The method of item 196 wherein the fibrosing agent is released from the device in effective concentrations from the device at a decreasing rate.

374. The method of item 196 wherein the fibrosing agent is released from the device in effective concentrations from the composition comprising the fibrosing agent by diffusion over a period ranging from the time of deployment of the device to about 90 days.

375. The method of item 196 wherein the fibrosing agent is released from the device in effective concentrations from the composition comprising the fibrosing agent by erosion of the composition over a period ranging from the time of deployment of the device to about 90 days.

376. A device, comprising an intravascular device and a fibrosing agent or a composition comprising a fibrosing agent, wherein the fibrosing agent induces fibrosis, wherein the device is configured to locally deliver the fibrosing agent or composition comprising the fibrosing agent to a tissue in the vicinity of the device once it is deployed, and wherein the device has an external surface and an internal surface.

377. The device of item 376 wherein the tissue is a blood vessel wall.

378. The device of item 376 wherein the blood vessel is an artery.

379. The device of item 376 wherein the blood vessel is an aorta.

380. The device of item 376 wherein the tissue is a diseased tissue.

381. The device of item 376 wherein the tissue is arterial plaque.

382. The device of item 376 wherein the tissue is unstable arterial plaque.

383. The device of item 376 wherein the tissue is an aneurysm.

384. The device of item 376 wherein the device is adapted to release the fibrosing agent or composition comprising the fibrosing agent upon deployment of the device.

385. The device of item 376 wherein the device is configured to deliver the fibrosing agent or the composition comprising the fibrosing agent onto a surface of the tissue.

386. The device of item 376 wherein the device is configured to deliver the fibrosing agent or the composition comprising the fibrosing agent into the tissue.

387. The device of item 376 wherein the intravascular device is a catheter.

388. The device of item 376 wherein the intravascular device is a balloon.

389. The device of item 376 wherein the intravascular device is a stent.

390. The device of item 376 wherein the intravascular device is a stent graft.

391. The device of item 376 wherein the fibrosing agent or the composition comprising the fibrosing agent is in the form of a coating, wherein the coating covers all or part of the external surface of the intravascular device.

392. The device of item 376 wherein the fibrosing agent promotes regeneration.

393. The device of item 376 wherein the fibrosing agent promotes angiogenesis.

394. The device of item 376 wherein the fibrosing agent promotes fibroblast migration.

395. The device of item 376 wherein the fibrosing agent promotes fibroblast proliferation.

396. The device of item 376 wherein the fibrosing agent promotes deposition of extracellular matrix (ECM).

397. The device of item 376 wherein the fibrosing agent promotes tissue remodeling.

398. The device of item 376 wherein the fibrosing agent promotes adhesion between the device and a host into which the device is implanted.

399. The device of item 376 wherein the fibrosing agent is an arterial vessel wall irritant.

400. The device of item 376 wherein the fibrosing agent is an arterial vessel wall irritant selected from the group consisting of talcum powder, metallic beryllium and oxides thereof, copper, silica, crystalline silicates, talc, quartz dust, and ethanol.

401. The device of item 376 wherein the fibrosing agent is or comprises silk.

402. The device of item 376 wherein the fibrosing agent is or comprises silkworm silk.

403. The device of item 376 wherein the fibrosing agent is or comprises spider silk.

404. The device of item 376 wherein the fibrosing agent is or comprises recombinant silk.

405. The device of item 376 wherein the fibrosing agent is or comprises raw silk.

406. The device of item 376 wherein the fibrosing agent is or comprises hydrolyzed silk.

407. The device of item 376 wherein the fibrosing agent is or comprises acid-treated silk.

408. The device of item 376 wherein the fibrosing agent is or comprises acylated silk.

409. The device of item 376 wherein the fibrosing agent is or comprises mineral particles.

410. The device of item 376 wherein the fibrosing agent is or comprises chitosan.

411. The device of item 376 wherein the fibrosing agent is or comprises polylysine.

412. The device of item 376 wherein the agent is or comprises a component of extracellular matrix.

413. The device of item 376 wherein the agent is or comprises a component of extracellular matrix, wherein the component is selected from collagen, fibrin, and fibrinogen.

414. The device of item 376 wherein the fibrosing agent is or comprises fibronectin.

415. The device of item 376 wherein the fibrosing agent is or comprises bleomycin or an analogue or derivative thereof.

416. The device of item 376 wherein the fibrosing agent is or comprises CTGF.

417. The device of item 376 wherein the agent is or comprises a peptide containing an RGD sequence.

418. The device of item 376 wherein the agent is or comprises poly(ethylene-co-vinylacetate).

419. The device of item 376 wherein the agent is or comprises an adhesive.

420. The device of item 376 wherein the adhesive is or comprises a cyanoacrylate.

421. The device of item 376 wherein the agent is or comprises a crosslinked poly(ethylene glycol)—methylated collagen.

422. The device of item 376 wherein the agent is or comprises an inflammatory cytokine.

423. The device of item 376 wherein the agent is or comprises a growth factor.

424. The device of item 376 wherein the agent is or comprises a member selected from the group consisting of TGFβ, PDGF, VEGF, bFGF, TNFα, NGF, GM-CSF, IGF-a, IL-1, IL-8, IL-6, and growth hormone.

425. The device of item 376 wherein the fibrosing agent is in the form of a thread, or is in contact with a thread.

426. The device of item 376 wherein the fibrosing agent is in the form of a particulate.

427. The device of item 376, further comprising a second pharmaceutically active agent.

428. The device of item 376, further comprising an inflammatory cytokine.

429. The device of item 376, further comprising an agent that stimulates cell proliferation.

430. The device of item 376, further comprising an agent that stimulates cell proliferation, wherein the proliferative agent is selected from the group consisting of dexamethasone, isotretinoin, 17-β-estradiol, estradiol, diethylstibesterol, all-trans retinoic acid (ATRA), and analogues and derivatives thereof.

431. The device of item 376, further comprising an agent that stimulates cell proliferation, wherein the proliferative agent is cyclosporine A.

432. The device of item 376, further comprising an agent that inhibits infection.

433. The device of item 376, further comprising an agent that inhibits infection, wherein the agent is an anthracycline.

434. The device of item 376, further comprising an agent that inhibits infection, wherein the agent is doxorubicin.

435. The device of item 376, further comprising an agent that inhibits infection, wherein the agent is mitoxantrone.

436. The device of item 376, further comprising an agent that inhibits infection, wherein the agent is a fluoropyrimidine.

437. The device of item 376, further comprising an agent that inhibits infection, wherein the agent is 5-fluorouracil (5-FU).

438. The device of item 376, further comprising an agent that inhibits infection, wherein the agent is a folic acid antagonist.

439. The device of item 376, further comprising an agent that inhibits infection, wherein the agent is methotrexate.

440. The device of item 376, further comprising an agent that inhibits infection, wherein the agent is a podophyllotoxin.

441. The device of item 376, further comprising an agent that inhibits infection, wherein the agent is etoposide.

442. The device of item 376, further comprising an agent that inhibits infection, wherein the agent is a camptothecin.

443. The device of item 376, further comprising an agent that inhibits infection, wherein the, agent is a hydroxyurea.

444. The device of item 376, further comprising an agent that inhibits infection, wherein the agent is a platinum complex.

445. The device of item 376, further comprising an agent that inhibits infection, wherein the agent is cisplatin.

446. The device of item 376, further comprising an anti-inflammatory agent.

447. The device of item 376, further comprising an anti-inflammatory agent selected from the group consisting of dexamethasone, cortisone, fludrocortisone, prednisone, prednisolone, 6α-methylprednisolone, triamcinolone, and betamethasone.

448. The device of item 376, further comprising an anti-inflammatory agent, wherein the anti-inflammatory agent is a TIMP.

449. The device of item 376, further comprising an anti-inflammatory agent, wherein the anti-inflammatory agent is batimistat, marimistat, doxycycline, tetracycline, minocycline, Ro-1130830, CGS 27023A, or BMS 275291.

450. The device of item 376, further comprising a therapeutic agent selected from the group consisting of MMP inhibitors, cytokine inhibitors, IMPDH inhibitors, and immunosuppressive agents.

451. The device of item 376, further comprising a cytokine inhibitor selected from the group consisting of chlorpromazine, sirolimus, and 1α-hydroxy vitamin D₃.

452. The device of item 376, further comprising an IMPDH inhibitor selected from the group consisting of mycophenolic acid, ribaviran, aminothiadiazole, thiophenfurin, tiazofurin, and viramidine.

453. The device of item 376, further comprising a wherein the immunosuppressive agent selected from the group consisting of sirolimus, everolimus, and ABT-578.

454. The device of item 376, further comprising a compound that inhibits restenosis.

455. The device of item 376, further comprising a compound that inhibits restenosis, wherein the compound is disposed on the internal surface of the device.

456. The device of item 376, further comprising a compound that inhibits restenosis, wherein the compound is paclitaxel or an analogue or derivative thereof.

457. The device of item 376, further comprising a compound that inhibits restenosis, wherein the compound is mycophenolic acid or an analogue or derivative thereof.

458. The device of item 376, further comprising a compound that inhibits restenosis, wherein the compound is selected from the group consisting of vincristine, biolimus, ABT-578, cervistatin, sirolimus, everolimus, simvastatin, methylprednisolone, actinomycin-D, angiopeptin, L-arginine, tranilast, methotrexate, batimistat, halofuginone, BCP-671, QP-2, lantrunculin D, cytochalasin A, nitric oxide, and analogues and derivatives thereof.

459. The device of item 376, further comprising a compound that inhibits thrombosis.

460. The device of item 376, further comprising a compound that inhibits thrombosis, wherein the compound is disposed on the internal surface of the device.

461. The device of item 376, further comprising a compound that inhibits thrombosis, wherein the anti-thrombotic agent is selected from the group consisting of heparin, heparin complexes, and analogues and derivatives thereof.

462. The device of item 376, further comprising a compound that inhibits thrombosis, wherein the anti-thrombotic agent is aspirin or dipyridamole.

463. The device of item 376 wherein the composition is in the form of a gel or paste.

464. The device of item 376 wherein the fibrosing agent is in the form of tufts.

465. The device of item 376, further comprising a coating, wherein the coating comprises the fibrosing agent.

466. The device of item 376, further comprising a coating, wherein the coating is disposed on a surface of the device, wherein the coating comprises the fibrosing agent.

467. The device of item 376, further comprising a coating, wherein the coating directly contacts the device, wherein the coating comprises the fibrosing agent.

468. The device of item 376, further comprising a coating, wherein the coating indirectly contacts the device, wherein the coating comprises the fibrosing agent.

469. The device of item 376, further comprising a coating, wherein the coating partially covers the device, wherein the coating comprises the fibrosing agent.

470. The device of item 376, further comprising a coating, wherein the coating completely covers the device, wherein the coating comprises the fibrosing agent.

471. The device of item 376, further comprising a coating, wherein the coating is a uniform coating, wherein the coating comprises the fibrosing agent.

472. The device of item 376, further comprising a coating, wherein the coating is a non-uniform coating, wherein the coating comprises the fibrosing agent.

473. The device of item 376, further comprising a coating, wherein the coating is a discontinuous coating, wherein the coating comprises the fibrosing agent.

474. The device of item 376, further comprising a coating, wherein the coating is a patterned coating, wherein the coating comprises the fibrosing agent.

475. The device of item 376, further comprising a coating, wherein the coating has a thickness of 100 μm or less, wherein the coating comprises the fibrosing agent.

476. The device of item 376, further comprising a coating, wherein the coating has a thickness of 10 μm or less, wherein the coating comprises the fibrosing agent.

477. The device of item 376, further comprising a coating, wherein the coating adheres to the surface of the device upon deployment of the device, wherein the coating comprises the fibrosing agent.

478. The device of item 376, further comprising a coating, wherein the coating is stable at room temperature for a period of at least 1 year, wherein the coating comprises the fibrosing agent.

479. The device of item 376, further comprising a coating, wherein the fibrosing agent is present in the coating in an amount ranging between about 0.0001% to about 1% by weight.

480. The device of item 376, further comprising a coating, wherein the fibrosing agent is present in the coating in an amount ranging between about 1% to about 10% by weight.

481. The device of item 376, further comprising a coating, wherein the fibrosing agent is present in the coating in an amount ranging between about 10% to about 25% by weight.

482. The device of item 376, further comprising a coating, wherein the fibrosing agent is present in the coating in an amount ranging between about 25% to about 70% by weight.

483. The device of item 376, further comprising a coating, wherein the coating further comprises a polymer.

484. The device of item 376, further comprising a first coating having a first composition and the second coating having a second composition.

485. The device of item 376, further comprising a first coating having a first composition and the second coating having a second composition, wherein the first composition and the second composition are different.

486. The device of item 376, further comprising a polymer.

487. The device of item 376, further comprising a polymeric carrier.

488. The device of item 376 wherein the polymeric carrier provides sustained release for the fibrosing agent.

489. The device of item 376, further comprising a polymeric carrier, wherein the polymeric carrier comprises a copolymer.

490. The device of item 376, further comprising a polymeric carrier, wherein the polymeric carrier comprises a block copolymer.

491. The device of item 376, further comprising a polymeric carrier, wherein the polymeric carrier comprises a random copolymer.

492. The device of item 376, further comprising a polymeric carrier, wherein the polymeric carrier comprises a biodegradable polymer.

493. The device of item 376, further comprising a polymeric carrier, wherein the polymeric carrier comprises a non-biodegradable polymer.

494. The device of item 376, further comprising a polymeric carrier, wherein the polymeric carrier comprises a hydrophilic polymer.

495. The device of item 376, further comprising a polymeric carrier, wherein the polymeric carrier comprises a hydrophobic polymer.

496. The device of item 376, further comprising a polymeric carrier, wherein the polymeric carrier comprises a polymer having hydrophilic domains.

497. The device of item 376, further comprising a polymeric carrier, wherein the polymeric carrier comprises a polymer having hydrophobic domains.

498. The device of item 376, further comprising a polymeric carrier, wherein the polymeric carrier comprises a non-conductive polymer.

499. The device of item 376, further comprising a polymeric carrier, wherein the polymeric carrier comprises an elastomer.

500. The device of item 376, further comprising a polymeric carrier, wherein the polymeric carrier comprises a hydrogel.

501. The device of item 376, further comprising a polymeric carrier, wherein the polymeric carrier comprises a silicone polymer.

502. The device of item 376, further comprising a polymeric carrier, wherein the polymeric carrier comprises a hydrocarbon polymer.

503. The device of item 376, further comprising a polymeric carrier, wherein the polymeric carrier comprises a styrene-derived polymer.

504. The device of item 376, further comprising a polymeric carrier, wherein the polymeric carrier comprises a butadiene polymer.

505. The device of item 376, further comprising a polymeric carrier, wherein the polymeric carrier comprises a macromer.

506. The device of item 376, further comprising a polymeric carrier, wherein the polymeric carrier comprises a poly(ethylene glycol)polymer.

507. The device of item 376, further comprising a polymeric carrier, wherein the polymeric carrier comprises an amorphous polymer.

508. The device of item 376, further comprising a lubricious coating.

509. The device of item 376 wherein the intravascular device comprises a pore or hole, wherein the fibrosing agent is located within the pore or hole of the device.

510. The device of item 376 wherein the intravascular device comprises a channel, lumen, or divet, wherein the fibrosing agent is located within the channel, lumen, or divet of the device.

511. The device of item 376, further comprising a visualization agent.

512. The device of item 376, further comprising a visualization agent, wherein the visualization agent is a radiopaque material, wherein the radiopaque material comprises a metal, a halogenated compound, or a barium containing compound.

513. The device of item 376, further comprising a visualization agent, wherein the visualization agent is a radiopaque material, wherein the radiopaque material comprises barium, tantalum, or technetium.

514. The device of item 376, further comprising a visualization agent, wherein the visualization agent is a MRI responsive material.

515. The device of item 376, further comprising a visualization agent, wherein the visualization agent comprises a gadolinium chelate.

516. The device of item 376, further comprising a visualization agent, wherein the visualization agent comprises iron, magnesium, manganese, copper, or chromium.

517. The device of item 376, further comprising a visualization agent, wherein the visualization agent comprises an iron oxide compound.

518. The device of item 376, further comprising a visualization agent, wherein the visualization agent comprises a dye, pigment, or colorant.

519. The device of item 376, further comprising an echogenic material.

520. The device of item 376, further comprising an echogenic material, wherein the echogenic material is in the form of a coating.

521. The device of item 376 wherein the device is sterile.

522. The device of item 376 wherein the fibrosing agent is released from the device in effective concentrations from the device over a period ranging from the time of deployment of the device to about 1 year.

523. The device of item 376 wherein the fibrosing agent is released from the device in effective concentrations from the device over a period ranging from about 1 month to 6 months.

524. The device of item 376 wherein the fibrosing agent is released from the device in effective concentrations from the device over a period ranging from about 1-90 days.

525. The device of item 376 wherein the fibrosing agent is released from the device in effective concentrations from the device at a constant rate.

526. The device of item 376 wherein the fibrosing agent is released from the device in effective concentrations from the device at an increasing rate.

527. The device of item 376 wherein the fibrosing agent is released from the device in effective concentrations from the device at a decreasing rate.

528. The device of item 376 wherein the fibrosing agent is released from the device in effective concentrations from the composition comprising the fibrosing agent by diffusion over a period ranging from the time of deployment of the device to about 90 days.

529. The device of item 376 wherein the fibrosing agent is released from the device in effective concentrations from the composition comprising the fibrosing agent by erosion of the composition over a period ranging from the time of deployment of the device to about 90 days.

530. The device of item 376 wherein the device comprises about 0.01 μg to about 10 μg of the fibrosing agent.

531. The device of item 376 wherein the device comprises about 10 μg to about 10 mg of the fibrosing agent.

532. The device of item 376 wherein the device comprises about 10 mg to about 250 mg of the fibrosing agent.

533. The device of item 376 wherein the device comprises about 250 mg to about 1000 mg of the fibrosing agent.

534. The device of item 376 wherein the device comprises about 1000 mg to about 2500 mg of the fibrosing agent.

535. The device of item 376 wherein a surface of the device comprises less than 0.01 μg of the fibrosing agent per mm² of device surface to which the fibrosing agent is applied.

536. The device of item 376 wherein a surface of the device comprises about 0.01 μg to about 1 μg of the fibrosing agent per mm² of device surface to which the fibrosing agent is applied.

537. The device of item 376 wherein a surface of the device comprises about 1 μg to about 10 μg of the fibrosing agent per mm² of device surface to which the fibrosing agent is applied.

538. The device of item 376 wherein a surface of the device comprises about 10 μg to about 250 μg of the fibrosing agent per mm² of device surface to which the fibrosing agent is applied.

539. The device of item 376 wherein a surface of the device comprises about 250 μg to about 1000 μg of the fibrosing agent of fibrosing agent per mm² of device surface to which the fibrosing agent is applied.

540. The device of item 376 wherein a surface of the device comprises about 1000 μg to about 2500 μg of the fibrosing agent per mm² of device surface to which the fibrosing agent is applied.

541. A device, comprising an intravascular catheter and a fibrosing agent or a composition comprising a fibrosing agent, wherein the catheter is configured to locally deliver a fibrosing agent or a composition comprising a fibrosing agent, wherein the agent induces fibrosis, to a tissue in the vicinity of the device once it is deployed.

542. The device of item 541 wherein the device is configured to deliver the fibrosing agent or composition comprising the fibrosing agent onto a surface of the tissue.

543. The device of item 541 wherein the device is configured to deliver the fibrosing agent or composition comprising the fibrosing agent into the tissue.

544. The method of item 541 wherein the tissue is a blood vessel wall.

545. The method of item 541 wherein the blood vessel is an artery.

546. The method of item 541 wherein the tissue is arterial plaque.

547. The method of item 541 wherein the tissue is unstable arterial plaque.

548. The device of item 541 wherein the fibrosing agent promotes regeneration.

549. The device of item 541 wherein the fibrosing agent promotes angiogenesis.

550. The device of item 541 wherein the fibrosing agent promotes fibroblast migration.

551. The device of item 541 wherein the fibrosing agent promotes fibroblast proliferation.

552. The device of item 541 wherein the fibrosing agent promotes deposition of extracellular matrix (ECM).

553. The device of item 541 wherein the fibrosing agent promotes tissue remodeling.

554. The device of item 541 wherein the fibrosing agent promotes adhesion between the device and a host into which the device is implanted.

555. The device of item 541 wherein the fibrosing agent is an arterial vessel wall irritant.

556. The device of item 541 wherein the fibrosing agent is an arterial vessel wall irritant selected from the group consisting of talcum powder, metallic beryllium and oxides thereof, copper, silica, crystalline silicates, talc, quartz dust, and ethanol.

557. The device of item 541 wherein the fibrosing agent is or comprises silk.

558. The device of item 541 wherein the fibrosing agent is or comprises silkworm silk.

559. The device of item 541 wherein the fibrosing agent is or comprises spider silk.

560. The device of item 541 wherein the fibrosing agent is or comprises recombinant silk.

561. The device of item 541 wherein the fibrosing agent is or comprises raw silk.

562. The device of item 541 wherein the fibrosing agent is or comprises hydrolyzed silk.

563. The device of item 541 wherein the fibrosing agent is or comprises acid-treated silk.

564. The device of item 541 wherein the fibrosing agent is or comprises acylated silk.

565. The device of item 541 wherein the fibrosing agent is or comprises mineral particles.

566. The device of item 541 wherein the fibrosing agent is or comprises chitosan.

567. The device of item 541 wherein the fibrosing agent is or comprises polylysine.

568. The device of item 541 wherein the agent is a component of extracellular matrix.

569. The device of item 541 wherein the component is selected from collagen, fibrin, and fibrinogen.

570. The device of item 541 wherein the fibrosing agent is or comprises fibronectin.

571. The device of item 541 wherein the fibrosing agent is or comprises bleomycin or an analogue or derivative thereof.

572. The device of item 541 wherein the fibrosing agent is or comprises CTGF.

573. The device of item 541 wherein the agent is or comprises a peptide containing an RGD sequence.

574. The device of item 541 wherein the agent is or comprises poly(ethylene-co-vinylacetate).

575. The device of item 541 wherein the agent is or comprises an adhesive.

576. The device of item 541 wherein the adhesive is or comprises a cyanoacrylate.

577. The device of item 541 wherein the agent is or comprises a crosslinked poly(ethylene glycol)—methylated collagen.

578. The device of item 541 wherein the agent is or comprises an inflammatory cytokine.

579. The device of item 541 wherein the agent is or comprises a growth factor.

580. The device of item 541 wherein the agent is or comprises a member selected from the group- consisting of TGFβ, PDGF, VEGF, bFGF, TNFα, NGF, GM-CSF, IGF-a, IL-1, IL-8, IL-6, and growth hormone.

581. The device of item 541 wherein the fibrosing agent is in the form of a thread, or is in contact with a thread.

582. The device of item 541 wherein the fibrosing agent is in the form of a particulate.

583. The device of item 541, further comprising an inflammatory cytokine.

584. The device of item 541, further comprising an agent that stimulates cell proliferation.

585. The device of item 541, further comprising an agent that stimulates cell proliferation, wherein the proliferative agent is selected from the group consisting of dexamethasone, isotretinoin, 17-β-estradiol, estradiol, diethylstibesterol, all-trans retinoic acid (ATRA), and analogues and derivatives thereof.

586. The device of item 541, further comprising an agent that stimulates cell proliferation, wherein the proliferative agent is cyclosporine A.

587. The device of item 541, further comprising an agent that inhibits infection.

588. The device of item 541, further comprising an agent that inhibits infection, wherein the agent is an anthracycline.

589. The device of item 541, further comprising an agent that inhibits infection, wherein the agent is doxorubicin.

590. The device of item 541, further comprising an agent that inhibits infection, wherein the agent is mitoxantrone.

591. The device of item 541, further comprising an agent that inhibits infection, wherein the agent is a fluoropyrimidine.

592. The device of item 541, further comprising an agent that inhibits infection, wherein the agent is 5-fluorouracil (5-FU).

593. The device of item 541, further comprising an agent that inhibits infection, wherein the agent is a folic acid antagonist.

594. The device of item 541, further comprising an agent that inhibits infection, wherein the agent is methotrexate.

595. The device of item 541, further comprising an agent that inhibits infection, wherein the agent is a podophyllotoxin.

596. The device of item 541, further comprising an agent that inhibits infection, wherein the agent is etoposide.

597. The device of item 541, further comprising an agent that inhibits infection, wherein the agent is a camptothecin.

598. The device of item 541, further comprising an agent that inhibits infection, wherein the agent is a hydroxyurea.

599. The device of item 541, further comprising an agent that inhibits infection, wherein the agent is a platinum complex.

600. The device of item 541, further comprising an agent that inhibits infection, wherein the agent is cisplatin.

601. The device of item 541, further comprising a therapeutic agent selected from the group consisting of anti-inflammatory agents, MMP inhibitors, cytokine inhibitors, IMPDH inhibitors, and immunosuppressive agents.

602. The device of item 541, further comprising an anti-inflammatory agent selected from the group consisting of dexamethasone, cortisone, fludrocortisone, prednisone, prednisolone, 6α-methylprednisolone, triamcinolone, and betamethasone.

603. The device of item 541, further comprising an anti-inflammatory agent, wherein the anti-inflammatory agent is a TIMP.

604. The device of item 541, further comprising an anti-inflammatory agent, wherein the anti-inflammatory agent is batimistat, marimistat, doxycycline, tetracycline, minocycline, Ro-1130830, CGS 27023A, or BMS 275291.

605. The device of item 541, further comprising a cytokine inhibitor selected from the group consisting of chlorpromazine, sirolimus, and 1α-hydroxy vitamin D₃.

606. The device of item 541, further comprising an IMPDH inhibitor selected from the group consisting of mycophenolic acid, ribaviran, aminothiadiazole, thiophenfurin, tiazofurin, and viramidine.

607. The device of item 541, further comprising a wherein the immunosuppressive agent selected from the group consisting of sirolimus, everolimus, and ABT-578.

608. The device of item 541, further comprising a compound that inhibits restenosis.

609. The device of item 541, further comprising a compound that inhibits restenosis, wherein the compound is paclitaxel or an analogue or derivative thereof.

610. The device of item 541, further comprising a compound that inhibits restenosis, wherein the compound is mycophenolic acid or an analogue or derivative thereof.

611. The device of item 541, further comprising a compound that inhibits restenosis, wherein the compound is selected from the group consisting of vincristine, biolimus, ABT-578, cervistatin, sirolimus, everolimus, simvastatin, methylprednisolone, actinomycin-D, angiopeptin, L-arginine, tranilast, methotrexate, batimistat, halofuginone, BCP-671, QP-2, lantrunculin D, cytochalasin A, nitric oxide, and analogues and derivatives thereof.

612. The device of item 541, further comprising a compound that inhibits thrombosis.

613. The device of item 541, further comprising a compound that inhibits thrombosis, wherein the anti-thrombotic agent is selected from the group consisting of heparin, heparin complexes, and analogues and derivatives thereof.

614. The device of item 541, further comprising a compound that inhibits thrombosis, wherein the anti-thrombotic agent is aspirin or dipyridamole.

615. The device of item 541 wherein the composition is in the form of a gel or paste.

616. The device of item 541 wherein the fibrosing agent is in the form of tufts.

617. The device of item 541, further comprising a coating, wherein the coating comprises the fibrosing agent.

618. The device of item 541, further comprising a coating, wherein the coating is disposed on a surface of the device, wherein the coating comprises the fibrosing agent.

619. The device of item 541, further comprising a coating, wherein the coating directly contacts the device, wherein the coating comprises the fibrosing agent.

620. The device of item 541, further comprising a coating, wherein the coating indirectly contacts the device, wherein the coating comprises the fibrosing agent.

621. The device of item 541, further comprising a coating, wherein the-coating partially covers the device, wherein the coating comprises the fibrosing agent.

622. The device of item 541, further comprising a coating, wherein the coating completely covers the device, wherein the coating comprises the fibrosing agent.

623. The device of item 541, further comprising a coating, wherein the coating is a uniform coating, wherein the coating comprises the fibrosing agent.

624. The device of item 541, further comprising a coating, wherein the coating is a non-uniform coating, wherein the coating comprises the fibrosing agent.

625. The device of item 541, further comprising a coating, wherein the coating is a discontinuous coating, wherein the coating comprises the fibrosing agent.

626. The device of item 541, further comprising a coating, wherein the coating is a patterned coating, wherein the coating comprises the fibrosing agent.

627. The device of item 541, further comprising a coating, wherein the coating has a thickness of 100 μm or less, wherein the coating comprises the fibrosing agent.

628. The device of item 541, further comprising a coating, wherein the coating has a thickness of 10 μm or less, wherein the coating comprises the fibrosing agent.

629. The device of item 541, further comprising a coating, wherein the coating adheres to the surface of the device upon deployment of the device, wherein the coating comprises the fibrosing agent.

630. The device of item 541, further comprising a coating, wherein the coating is stable at room temperature for a period of at least 1 year, wherein the coating comprises the fibrosing agent.

631. The device of item 541, further comprising a coating, wherein the fibrosing agent is present in the coating in an amount ranging between about 0.0001% to about 1% by weight.

632. The device of item 541, further comprising a coating, wherein the fibrosing agent is present in the coating in an amount ranging between about 1% to about 10% by weight.

633. The device of item 541, further comprising a coating, wherein the fibrosing agent is present in the coating in an amount ranging between about 10% to about 25% by weight.

634. The device of item 541, further comprising a coating, wherein the fibrosing agent is present in the coating in an amount ranging between about 25% to about 70% by weight.

635. The device of item 541, further comprising a coating, wherein the coating further comprises a polymer.

636. The device of item 541, further comprising a first coating having a first composition and the second coating having a second composition.

637. The device of item 541, further comprising a first coating having a first composition and the second coating having a second composition, wherein the first composition and the second composition are different.

638. The device of item 541, further comprising a polymer.

639. The device of item 541, further comprising a polymeric carrier.

640. The device of item 541 wherein the polymeric carrier provides sustained release for the fibrosing agent.

641. The device of item 541, further comprising a polymeric carrier, wherein the polymeric carrier comprises a copolymer.

642. The device of item 541, further comprising a polymeric carrier, wherein the polymeric carrier comprises a block copolymer.

643. The device of item 541, further comprising a polymeric carrier, wherein the polymeric carrier comprises a random copolymer.

644. The device of item 541, further comprising a polymeric carrier, wherein the polymeric carrier comprises a biodegradable polymer.

645. The device of item 541, further comprising a polymeric carrier, wherein the polymeric carrier comprises a non-biodegradable polymer.

646. The device of item 541, further comprising a polymeric carrier, wherein the polymeric carrier comprises a hydrophilic polymer.

647. The device of item 541, further comprising a polymeric carrier, wherein the polymeric carrier comprises a hydrophobic polymer.

648. The device of item 541, further comprising a polymeric carrier, wherein the polymeric carrier comprises a polymer having hydrophilic domains.

649. The device of item 541, further comprising a polymeric carrier, wherein the polymeric carrier comprises a polymer having hydrophobic domains.

650. The device of item 541, further comprising a polymeric carrier, wherein the polymeric carrier comprises a non-conductive polymer.

651. The device of item 541, further comprising a polymeric carrier, wherein the polymeric carrier comprises an elastomer.

652. The device of item 541, further comprising a polymeric carrier, wherein the polymeric carrier comprises a hydrogel.

653. The device of item 541, further comprising a polymeric carrier, wherein the polymeric carrier comprises a silicone polymer.

654. The device of item 541, further comprising a polymeric carrier, wherein the polymeric carrier comprises a hydrocarbon polymer.

655. The device of item 541, further comprising a polymeric carrier, wherein the polymeric carrier comprises a styrene-derived polymer.

656. The device of item 541, further comprising a polymeric carrier, wherein the polymeric carrier comprises a butadiene polymer.

657. The device of item 541, further comprising a polymeric carrier, wherein the polymeric carrier comprises a macromer.

658. The device of item 541, further comprising a polymeric carrier, wherein the polymeric carrier comprises a poly(ethylene glycol)polymer.

659. The device of item 541, further comprising a polymeric carrier, wherein the polymeric carrier comprises an amorphous polymer.

660. The device of item 541, further comprising a lubricious coating.

661. The device of item 541 wherein the device comprises a pore or hole, wherein the fibrosing agent is located within the pore or hole of the device.

662. The device of item 541 wherein the device comprises a channel, lumen, or divet, wherein the fibrosing agent is located within the channel, lumen, or divet of the device.

663. The device of item 541, further comprising an agent that inhibits infection.

664. The device of item 541, further comprising an agent that inhibits infection, wherein the agent is an anthracycline.

665. The device of item 541, further comprising an agent that inhibits infection, wherein the agent is doxorubicin.

666. The device of item 541, further comprising an agent that inhibits infection, wherein the agent is mitoxantrone.

667. The device of item 541, further comprising an agent that inhibits infection, wherein the agent is a fluoropyrimidine.

668. The device of item 541, further comprising an agent that inhibits infection, wherein the agent is 5-fluorouracil (5-FU).

669. The device of item 541, further comprising an agent that inhibits infection, wherein the agent is a folic acid antagonist.

670. The device of item 541, further comprising an agent that inhibits infection, wherein the agent is methotrexate.

671. The device of item 541, further comprising an agent that inhibits infection, wherein the agent is a podophylotoxin.

672. The device of item 541, further comprising an agent that inhibits infection, wherein the agent is etoposide.

673. The device of item 541, further comprising an agent that inhibits infection, wherein the agent is a camptothecin.

674. The device of item 541, further comprising an agent that inhibits infection, wherein the agent is a hydroxyurea.

675. The device of item 541, further comprising a visualization agent.

676. The device of item 541, further comprising a visualization agent, wherein the visualization agent is a radiopaque material, wherein the radiopaque material comprises a metal, a halogenated compound, or a barium containing compound.

677. The device of item 541, further comprising a visualization agent, wherein the visualization agent is a radiopaque material, wherein the radiopaque material comprises barium, tantalum, or technetium.

678. The device of item 541, further comprising a visualization agent, wherein the visualization agent is a MRI responsive material.

679. The device of item 541, further comprising a visualization agent, wherein the visualization agent comprises a gadolinium chelate.

680. The device of item 541, further comprising a visualization agent, wherein the visualization agent comprises iron, magnesium, manganese, copper, or chromium.

681. The device of item 541, further comprising a visualization agent, wherein the visualization agent comprises an iron oxide compound.

682. The device of item 541, further comprising a visualization agent, wherein the visualization agent comprises a dye, pigment, or colorant.

683. The device of item 541, further comprising an echogenic material.

684. The device of item 541, further comprising an echogenic material, wherein the echogenic material is in the form of a coating.

685. The device of item 541 wherein the device is sterile.

686. The device of item 541 wherein the device is adapted to release the fibrosing agent or composition comprising the fibrosing agent upon deployment of the device.

687. The device of item 541 wherein the fibrosing agent is released from the device in effective concentrations from the device over a period ranging from the time of deployment of the device to about 1 year.

688. The device of item 541 wherein the fibrosing agent is released from the device in effective concentrations from the device over a period ranging from about 1 month to 6 months.

689. The device of item 541 wherein the fibrosing agent is released from the device in effective concentrations from the device over a period ranging from about 1-90 days.

690. The device of item 541 wherein the fibrosing agent is released from the device in effective concentrations from the device at a constant rate.

691. The device of item 541 wherein the fibrosing agent is released from the device in effective concentrations from the device at an increasing rate.

692. The device of item 541 wherein the fibrosing agent is released from the device in effective concentrations from the device at a decreasing rate.

693. The device of item 541 wherein the fibrosing agent is released from the device in effective concentrations from the composition comprising the fibrosing agent by diffusion over a period ranging from the time of deployment of the device to about 90 days.

694. The device of item 541 wherein the fibrosing agent is released from the device in effective concentrations from the composition comprising the fibrosing agent by erosion of the composition over a period ranging from the time of deployment of the device to about 90 days.

695. The device of item 541 wherein the device comprises about 0.01 μg to about 10 μg of the fibrosing agent.

696. The device of item 541 wherein the device comprises about 10 μg to about 10 mg of the fibrosing agent.

697. The device of item 541 wherein the device comprises about 10 mg to about 250 mg of the fibrosing agent.

698. The device of item 541 wherein the device comprises about 250 mg to about 1000 mg of the fibrosing agent.

699. The device of item 541 wherein the device comprises about 1000 mg to about 2500 mg of the fibrosing agent.

700. The device of item 541 wherein a surface of the device comprises less than 0.01 μg of the fibrosing agent per mm² of device surface to which the fibrosing agent is applied.

701. The device of item 541 wherein a surface of the device comprises about 0.01 μg to about 1 μg of the fibrosing agent per mm² of device surface to which the fibrosing agent is applied.

702. The device of item 541 wherein a surface of the device comprises about 1 μg to about 10 μg of the fibrosing agent per mm² of device surface to which the fibrosing agent is applied.

703. The device of item 541 wherein a surface of the device comprises about 10 μg to about 250 μg of the fibrosing agent per mm² of device surface to which the fibrosing agent is applied.

704. The device of item 541 wherein a surface of the device comprises about 250 μg to about 1000 μg of the fibrosing agent of fibrosing agent per mm² of device surface to which the fibrosing agent is applied.

705. The device of item 541 wherein a surface of the device comprises about 1000 μg to about 2500 μg of the fibrosing agent per mm² of device surface to which the fibrosing agent is applied.

706. A device, comprising an intravascular balloon and a fibrosing agent or a composition comprising a fibrosing agent, wherein the device is configured to locally deliver a fibrosing agent or a composition comprising a fibrosing agent, wherein the agent induces fibrosis, in the vicinity of the device once it is deployed.

707. The device of item 706 wherein the device is configured to deliver the fibrosing agent or composition comprising the fibrosing agent onto a surface of the tissue.

708. The device of item 706 wherein the device is configured to deliver the fibrosing agent or composition comprising the fibrosing agent into the tissue.

709. The method of item 706 wherein the tissue is a blood vessel wall.

710. The method of item 706 wherein the blood vessel is an artery.

711. The method of item 706 wherein the tissue is arterial plaque.

712. The method of item 706 wherein the tissue is unstable arterial plaque.

713. The device of item 706 wherein the fibrosing agent promotes regeneration.

714. The device of item 706 wherein the fibrosing agent promotes angiogenesis.

715. The device of item 706 wherein the fibrosing agent promotes fibroblast migration.

716. The device of item 706 wherein the fibrosing agent promotes fibroblast proliferation.

717. The device of item 706 wherein the fibrosing agent promotes deposition of extracellular matrix (ECM).

718. The device of item 706 wherein the fibrosing agent promotes tissue remodeling.

719. The device of item 706 wherein the fibrosing agent promotes adhesion between the device and a host into which the device is implanted.

720. The device of item 706 wherein the fibrosing agent is an arterial vessel wall irritant.

721. The device of item 706 wherein the fibrosing agent is an arterial vessel wall irritant selected from the group consisting of talcum powder, metallic beryllium and oxides thereof, copper, silica, crystalline silicates, talc, quartz dust, and ethanol.

722. The device of item 706 wherein the fibrosing agent is or comprises silk.

723. The device of item 706 wherein the fibrosing agent is or comprises silkworm silk.

724. The device of item 706 wherein the fibrosing agent is or comprises spider silk.

725. The device of item 706 wherein the fibrosing agent is or comprises recombinant silk.

726. The device of item 706 wherein the fibrosing agent is or comprises raw silk.

727. The device of item 706 wherein the fibrosing agent is or comprises hydrolyzed silk.

728. The device of item 706 wherein the fibrosing agent is or comprises acid-treated silk.

729. The device of item 706 wherein the fibrosing agent is or comprises acylated silk.

730. The device of item 706 wherein the fibrosing agent is or comprises mineral particles.

731. The device of item 706 wherein the fibrosing agent is or comprises chitosan.

732. The device of item 706 wherein the fibrosing agent is or comprises polylysine.

733. The device of item 706 wherein the agent is a component of extracellular matrix.

734. The device of item 706 wherein the component is selected from collagen, fibrin, and fibrinogen.

735. The device of item 706 wherein the fibrosing agent is or comprises fibronectin.

736. The device of item 706 wherein the fibrosing agent is or comprises bleomycin or an analogue or derivative thereof.

737. The device of item 706 wherein the fibrosing agent is or comprises CTGF.

738. The device of item 706 wherein the agent is or comprises a peptide containing an RGD sequence.

739. The device of item 706 wherein the agent is or comprises poly(ethylene-co-vinylacetate).

740. The device of item 706 wherein the agent is or comprises an adhesive.

741. The device of item 706 wherein the adhesive is or comprises a cyanoacrylate.

742. The device of item 706 wherein the agent is or comprises a crosslinked poly(ethylene glycol)—methylated collagen.

743. The device of item 706 wherein the agent is or comprises an inflammatory cytokine.

744. The device of item 706 wherein the agent is or comprises a growth factor.

745. The device of item 706 wherein the agent is or comprises a member selected from the group consisting of TGFβ, PDGF, VEGF, bFGF, TNFα, NGF, GM-CSF, IGF-a, IL-1, IL-8, IL-6, and growth hormone.

746. The device of item 706 wherein the fibrosing agent is in the form of a thread, or is in contact with a thread.

747. The device of item 706 wherein the fibrosing agent is in the form of a particulate.

748. The device of item 706, further comprising an inflammatory cytokine.

749. The device of item 706, further comprising an agent that stimulates cell proliferation.

750. The device of item 706, further comprising an agent that stimulates cell proliferation, wherein the proliferative agent is selected from the group consisting of dexamethasone, isotretinoin, 17-β-estradiol, estradiol, diethylstibesterol, all-trans retinoic acid (ATRA), and analogues and derivatives thereof.

751. The device of item 706, further comprising an agent that stimulates cell proliferation, wherein the proliferative agent is cyclosporine A.

752. The device of item 706, further comprising an agent that inhibits infection.

753. The device of item 706, further comprising an agent that inhibits infection, wherein the agent is an anthracycline.

754. The device of item 706, further comprising an agent that inhibits infection, wherein the agent is doxorubicin.

755. The device of item 706, further comprising an agent that inhibits infection, wherein the agent is mitoxantrone.

756. The device of item 706, further comprising an agent that inhibits infection, wherein the agent is a fluoropyrimidine.

757. The device of item 706, further comprising an agent that inhibits infection, wherein the agent is 5-fluorouracil (5-FU).

758. The device of item 706, further comprising an agent that inhibits infection, wherein the agent is a folic acid antagonist.

759. The device of item 706, further comprising an agent that inhibits infection, wherein the agent is methotrexate.

760. The device of item 706, further comprising an agent that inhibits infection, wherein the agent is a podophyllotoxin.

761. The device of item 706, further comprising an agent that inhibits infection, wherein the agent is etoposide.

762. The device of item 706, further comprising an agent that inhibits infection, wherein the agent is a camptothecin.

763. The device of item 706, further comprising an agent that inhibits infection, wherein the agent is a hydroxyurea.

764. The device of item 706, further comprising an agent that inhibits infection, wherein the agent is a platinum complex.

765. The device of item 706, further comprising an agent that inhibits infection, wherein the agent is cisplatin.

766. The device of item 706, further comprising a therapeutic agent selected from the group consisting of anti-inflammatory agents, MMP inhibitors, cytokine inhibitors, IMPDH inhibitors, and immunosuppressive agents.

767. The device of item 706, further comprising an anti-inflammatory agent selected from the group consisting of dexamethasone, cortisone, fludrocortisone, prednisone, prednisolone, 6α-methylprednisolone, triamcinolone, and betamethasone.

768. The device of item 706, further comprising an anti-inflammatory agent, wherein the anti-inflammatory agent is a TIMP.

769. The device of item 706, further comprising an anti-inflammatory agent, wherein the anti-inflammatory agent is batimistat, marimistat, doxycycline, tetracycline, minocycline, Ro-1130830, CGS 27023A, or BMS 275291.

770. The device of item 706, further comprising a cytokine inhibitor selected from the group consisting of chlorpromazine, sirolimus, and 1α-hydroxy vitamin D₃.

771. The device of item 706, further comprising an IMPDH inhibitor selected from the group consisting of mycophenolic acid, ribaviran, aminothiadiazole, thiophenfurin, tiazofurin, and viramidine.

772. The device of item 706, further comprising a wherein the immunosuppressive agent selected from the group consisting of sirolimus, everolimus, and ABT-578.

773. The device of item 706, further comprising a compound that inhibits restenosis.

774. The device of item 706, further comprising a compound that inhibits restenosis, wherein the compound is paclitaxel or an analogue or derivative thereof.

775. The device of item 706, further comprising a compound that inhibits restenosis, wherein the compound is mycophenolic acid or an analogue or derivative thereof.

776. The device of item 706, further comprising a compound that inhibits restenosis, wherein the compound is selected from the group consisting of vincristine, biolimus, ABT-578, cervistatin, sirolimus, everolimus, simvastatin, methylprednisolone, actinomycin-D, angiopeptin, L-arginine, tranilast, methotrexate, batimistat, halofuginone, BCP-671, QP-2, lantrunculin D, cytochalasin A, nitric oxide, and analogues and derivatives thereof.

777. The device of item 706, further comprising a compound that inhibits thrombosis.

778. The device of item 706, further comprising a compound that inhibits thrombosis, wherein the anti-thrombotic agent is selected from the group consisting of heparin, heparin complexes, and analogues and derivatives thereof.

779. The device of item 706, further comprising a compound that inhibits thrombosis, wherein the anti-thrombotic agent is aspirin or dipyridamole.

780. The device of item 706 wherein the composition is in the form of a gel or paste.

781. The device of item 706 wherein the fibrosing agent is in the form of tufts.

782. The device of item 706, further comprising a coating, wherein the coating comprises the fibrosing agent.

783. The device of item 706, further comprising a coating, wherein the coating is disposed on a surface of the device, wherein the coating comprises the fibrosing agent.

784. The device of item 706, further comprising a coating, wherein the coating directly contacts the device, wherein the coating comprises the fibrosing agent.

785. The device of item 706, further comprising a coating, wherein the coating indirectly contacts the device, wherein the coating comprises the fibrosing agent.

786. The device of item 706, further comprising a coating, wherein the coating partially covers the device, wherein the coating comprises the fibrosing agent.

787. The device of item 706, further comprising a coating, wherein the coating completely covers the device, wherein the coating comprises the fibrosing agent.

788. The device of item 706, further comprising a coating, wherein the coating is a uniform coating, wherein the coating comprises the fibrosing agent.

789. The device of item 706, further comprising a coating, wherein the coating is a non-uniform coating, wherein the coating comprises the fibrosing agent.

790. The device of item 706, further comprising a coating, wherein the coating is a discontinuous coating, wherein the coating comprises the fibrosing agent.

791. The device of item 706, further comprising a coating, wherein the coating is a patterned coating, wherein the coating comprises the fibrosing agent.

792. The device of item 706, further comprising a coating, wherein the coating has a thickness of 100 μm or less, wherein the coating comprises the fibrosing agent.

793. The device of item 706, further comprising a coating, wherein the coating has a thickness of 10 μm or less, wherein the coating comprises the fibrosing agent.

794. The device of item 706, further comprising a coating, wherein the coating adheres to the surface of the device upon deployment of the device, wherein the coating comprises the fibrosing agent.

795. The device of item 706, further comprising a coating, wherein the coating is stable at room temperature for a period of at least 1 year, wherein the coating comprises the fibrosing agent.

796. The device of item 706, further comprising a coating, wherein the fibrosing agent is present in the coating in an amount ranging between about 0.0001% to about 1% by weight.

797. The device of item 706, further comprising a coating, wherein the fibrosing agent is present in the coating in an amount ranging between about 1% to about 10% by weight.

798. The device of item 706, further comprising a coating, wherein the fibrosing agent is present in the coating in an amount ranging between about 10% to about 25% by weight.

799. The device of item 706, further comprising a coating, wherein the fibrosing agent is present in the coating in an amount ranging between about 25% to about 70% by weight.

800. The device of item 706, further comprising a coating, wherein the coating further comprises a polymer.

801. The device of item 706, further comprising a first coating having a first composition and the second coating having a second composition.

802. The device of item 706, further comprising a first coating having a first composition and the second coating having a second composition, wherein the first composition and the second composition are different.

803. The device of item 706, further comprising a polymer.

804. The device of item 706, further comprising a polymeric carrier.

805. The device of item 706 wherein the polymeric carrier provides sustained release for the fibrosing agent.

806. The device of item 706, further comprising a polymeric carrier, wherein the polymeric carrier comprises a copolymer.

807. The device of item 706, further comprising a polymeric carrier, wherein the polymeric carrier comprises a block copolymer.

808. The device of item 706, further comprising a polymeric carrier, wherein the polymeric carrier comprises a random copolymer.

809. The device of item 706, further comprising a polymeric carrier, wherein the polymeric carrier comprises a biodegradable polymer.

810. The device of item 706, further comprising a polymeric carrier, wherein the polymeric carrier comprises a non-biodegradable polymer.

811. The device of item 706, further comprising a polymeric carrier, wherein the polymeric carrier comprises a hydrophilic polymer.

812. The device of item 706, further comprising a polymeric carrier, wherein the polymeric carrier comprises a hydrophobic polymer.

813. The device of item 706, further comprising a polymeric carrier, wherein the polymeric carrier comprises a polymer having hydrophilic domains.

814. The device of item 706, further comprising a polymeric carrier, wherein the polymeric carrier comprises a polymer having hydrophobic domains.

815. The device of item 706, further comprising a polymeric carrier, wherein the polymeric carrier comprises a non-conductive polymer.

816. The device of item 706, further comprising a polymeric carrier, wherein the polymeric carrier comprises an elastomer.

817. The device of item 706, further comprising a polymeric carrier, wherein the polymeric carrier comprises a hydrogel.

818. The device of item 706, further comprising a polymeric carrier, wherein the polymeric carrier comprises a silicone polymer.

819. The device of item 706, further comprising a polymeric carrier, wherein the polymeric carrier comprises a hydrocarbon polymer.

820. The device of item 706, further comprising a polymeric carrier, wherein the polymeric carrier comprises a styrene-derived polymer.

821. The device of item 706, further comprising a polymeric carrier, wherein the polymeric carrier comprises a butadiene polymer.

822. The device of item 706, further comprising a polymeric carrier, wherein the polymeric carrier comprises a macromer.

823. The device of item 706, further comprising a polymeric carrier, wherein the polymeric carrier comprises a poly(ethylene glycol)polymer.

824. The device of item 706, further comprising a polymeric carrier, wherein the polymeric carrier comprises an amorphous polymer.

825. The device of item 706, further comprising a lubricious coating.

826. The device of item 706 wherein the device comprises a pore or hole, wherein the fibrosing agent is located within the pore or hole of the device.

827. The device of item 706 wherein the device comprises a channel, lumen, or divet, wherein the fibrosing agent is located within the channel, lumen, or divet of the device.

828. The device of item 706, further comprising an agent that inhibits infection.

829. The device of item 706, further comprising an agent that inhibits infection, wherein the agent is an anthracycline.

830. The device of item 706, further comprising an agent that inhibits infection, wherein the agent is doxorubicin.

831. The device of item 706, further comprising an agent that inhibits infection, wherein the agent is mitoxantrone.

832. The device of item 706, further comprising an agent that inhibits infection, wherein the agent is a fluoropyrimidine.

833. The device of item 706, further comprising an agent that inhibits infection, wherein the agent is 5-fluorouracil (5-FU).

834. The device of item 706, further comprising an agent that inhibits infection, wherein the agent is a folic acid antagonist.

835. The device of item 706, further comprising an agent that inhibits infection, wherein the agent is methotrexate.

836. The device of item 706, further comprising an agent that inhibits infection, wherein the agent is a podophylotoxin.

837. The device of item 706, further comprising an agent that inhibits infection, wherein the agent is etoposide.

838. The device of item 706, further comprising an agent that inhibits infection, wherein the agent is a camptothecin.

839. The device of item 706, further comprising an agent that inhibits infection, wherein the agent is a hydroxyurea.

840. The device of item 706, further comprising a visualization agent.

841. The device of item 706, further comprising a visualization agent, wherein the visualization agent is a radiopaque material, wherein the radiopaque material comprises a metal, a halogenated compound, or a barium containing compound.

842. The device of item 706, further comprising a visualization agent, wherein the visualization agent is a radiopaque material, wherein the radiopaque material comprises barium, tantalum, or technetium.

843. The device of item 706, further comprising a visualization agent, wherein the visualization agent is a MRI responsive material.

844. The device of item 706, further comprising a visualization agent, wherein the visualization agent comprises a gadolinium chelate.

845. The device of item 706, further comprising a visualization agent, wherein the visualization agent comprises iron, magnesium, manganese, copper, or chromium.

846. The device of item 706, further comprising a visualization agent, wherein the visualization agent comprises an iron oxide compound.

847. The device of item 706, further comprising a visualization agent, wherein the visualization agent comprises a dye, pigment, or colorant.

848. The device of item 706, further comprising an echogenic material.

849. The device of item 706, further comprising an echogenic material, wherein the echogenic material is in the form of a coating.

850. The device of item 706 wherein the device is sterile.

851. The device of item 706 wherein the device is adapted to release the fibrosing agent or composition comprising the fibrosing agent upon deployment of the device.

852. The device of item 706 wherein the fibrosing agent is released from the device in effective concentrations from the device over a period ranging from the time of deployment of the device to about 1 year.

853. The device of item 706 wherein the fibrosing agent is released from the device in effective concentrations from the device over a period ranging from about 1 month to 6 months.

854. The device of item 706 wherein the fibrosing agent is released from the device in effective concentrations from the device over a period ranging from about 1-90 days.

855. The device of item 706 wherein the fibrosing agent is released from the device in effective concentrations from the device at a constant rate.

856. The device of item 706 wherein the fibrosing agent is released from the device in effective concentrations from the device at an increasing rate.

857. The device of item 706 wherein the fibrosing agent is released from the device in effective concentrations from the device at a decreasing rate.

858. The device of item 706 wherein the fibrosing agent is released from the device in effective concentrations from the composition comprising the fibrosing agent by diffusion over a period ranging from the time of deployment of the device to about 90 days.

859. The device of item 706 wherein the fibrosing agent is released from the device in effective concentrations from the composition comprising the fibrosing agent by erosion of the composition over a period ranging from the time of deployment of the device to about 90 days.

860. The device of item 706 wherein the device comprises about 0.01 μg to about 10 μg of the fibrosing agent.

861. The device of item 706 wherein the device comprises about 10 μg to about 10 mg of the fibrosing agent.

862. The device of item 706 wherein the device comprises about 10 mg to about 250 mg of the fibrosing agent.

863. The device of item 706 wherein the device comprises about 250 mg to about 1000 mg of the fibrosing agent.

864. The device of item 706 wherein the device comprises about 1000 mg to about 2500 mg of the fibrosing agent.

865. The device of item 706 wherein a surface of the device comprises less than 0.01 μg of the fibrosing agent per mm² of device surface to which the fibrosing agent is applied.

866. The device of item 706 wherein a surface of the device comprises about 0.01 μg to about 1 μg of the fibrosing agent per mm² of device surface to which the fibrosing agent is applied.

867. The device of item 706 wherein a surface of the device comprises about 1 μg to about 10 μg of the fibrosing agent per mm² of device surface to which the fibrosing agent is applied.

868. The device of item 706 wherein a surface of the device comprises about 10 μg to about 250 μg of the fibrosing agent per mm² of device surface to which the fibrosing agent is applied.

869. The device of item 706 wherein a surface of the device comprises about 250 μg to about 1000 μg of the fibrosing agent of fibrosing agent per mm² of device surface to which the fibrosing agent is applied.

870. The device of item 706 wherein a surface of the device comprises about 1000 μg to about 2500 μg of the fibrosing agent per mm² of device surface to which the fibrosing agent is applied.

871. A method for treating vulnerable plaque, comprising contacting i) vulnerable plaque in a patient, or tissue adjacent to vulnerable plaque in a patient, with ii) an agent or a composition comprising an agent, where the agent induces fibrosis.

872. The method of item 871 wherein the fibrosing agent promotes regeneration.

873. The method of item 871 wherein the fibrosing agent promotes angiogenesis.

874. The method of item 871 wherein the fibrosing agent promotes fibroblast migration.

875. The method of item 871 wherein the fibrosing agent promotes fibroblast proliferation.

876. The method of item 871 wherein the fibrosing agent promotes deposition of extracellular matrix (ECM).

877. The method of item 871 wherein the fibrosing agent promotes tissue remodeling.

878. The method of item 871 wherein the fibrosing agent is an arterial vessel wall irritant.

879. The method of item 871 wherein the fibrosing agent is or comprises silk.

880. The method of item 871 wherein the fibrosing agent is or comprises mineral particles.

881. The method of item 871 wherein the fibrosing agent is or comprises chitosan.

882. The method of item 871 wherein the fibrosing agent is or comprises polylysine.

883. The method of item 871 wherein the fibrosing agent is or comprises fibronectin.

884. The method of item 871 wherein the fibrosing agent is or comprises bleomycin.

885. The method of item 871 wherein the fibrosing agent is or comprises CTGF.

886. The method of item 871 wherein the fibrosing agent is in the form of a thread, or is in contact with a thread.

887. The method of item 871 wherein the fibrosing agent is in the form of a particulate.

888. The method of item 871 wherein the composition further comprises an inflammatory cytokine.

889. The method of item 871 wherein the composition further comprises an agent that stimulates cell proliferation.

890. The method of item 871 wherein the composition is in the form of a gel or paste.

891. The method of item 871 wherein the fibrosing agent is in the form of tufts.

892. The method of item 871, wherein the agent is associated with an intravascular implant prior to contacting i).

893. The method of item 871, wherein the agent is associated with an intravascular implant prior to contacting i), and the fibrosing agent promotes adhesion between the implant and the patient.

894. The method of item 871, wherein the agent is associated with an intravascular implant prior to contacting i), and wherein the implant delivers the fibrosing agent locally to tissue proximate to the implant.

895. The method of item 871, wherein the agent is associated with an intravascular implant prior to contacting i), and wherein the implant and fibrosing agent are combined so as to provide a coating on the implant.

896. The method of item 871, wherein the agent is associated with an intravascular implant, to form a-device, prior to contacting i), and wherein the implant and fibrosing agent are combined so as to provide a coating on the implant, and the coating directly contacts the device.

897. The method of item 871, wherein the agent is associated with an intravascular implant, to form a device, prior to contacting i), and wherein the implant and fibrosing agent are combined so as to provide a coating on the implant, and the coating indirectly contacts the device.

898. The method of item 871, wherein the agent is associated with an intravascular implant, to form a device, prior to contacting i), and wherein the implant and fibrosing agent are combined so as to provide a coating on the implant, and the coating partially covers the device.

899. The method of item 871, wherein the agent is associated with an intravascular implant, to form a device, prior to contacting i), and wherein the implant and fibrosing agent are combined so as to provide a coating on the implant, and the coating completely covers the device.

900. The method of item 871, wherein the agent is associated with an intravascular implant, to form a device, prior to contacting i), and wherein the implant and fibrosing agent are combined so as to provide a coating on the implant, where the coating is a uniform coating.

901. The method of item 871, wherein the agent is associated with an intravascular implant, to form a device, prior to contacting i), and wherein the implant and fibrosing agent are combined so as to provide a coating on the implant, where the coating is a non-uniform coating.

902. The method of item 871, wherein the agent is associated with an intravascular implant, to form a device, prior to contacting i), and wherein the implant and fibrosing agent are combined so as to provide a coating on the implant, where the coating is a discontinuous coating.

903. The method of item 871, wherein the agent is associated with an intravascular implant, to form a device, prior to contacting i), and wherein the implant and fibrosing agent are combined so as to provide a coating on the implant, where the coating is a patterned coating.

904. The method of item 871, wherein the agent is associated with an intravascular implant, to form a device, prior to contacting i), and wherein the implant and fibrosing agent are combined so as to provide a coating on the implant, where the coating has a thickness of 100 μm or less.

905. The method of item 871, wherein the agent is associated with an intravascular implant, to form a device, prior to contacting i), and wherein the implant and fibrosing agent are combined so as to provide a coating on the implant, where the coating has a thickness of 10 μm or less.

906. The method of item 871, wherein the agent is associated with an intravascular implant, to form a device, prior to contacting i), and wherein the implant and fibrosing agent are combined so as to provide a coating on the implant, and the coating is stable at room temperature for a period of at least 1 year.

907. The method of item 871, wherein the agent is associated with an intravascular implant, to form a device, prior to contacting i), and wherein the implant and fibrosing agent are combined so as to provide a coating on the implant, and the fibrosing agent is present in the coating in an amount ranging between about 0.0001% to about 1% by weight.

908. The method of item 871, wherein the agent is associated with an intravascular implant, to form a device, prior to contacting i), and wherein the implant and fibrosing agent are combined so as to provide a coating on the implant, and the fibrosing agent is present in the coating in an amount ranging between about 1% to about 10% by weight.

909. The method of item 871, wherein the agent is associated with an intravascular implant, to form a device, prior to contacting i), and wherein the implant and fibrosing agent are combined so as to provide a coating on the implant, and the fibrosing agent is present in the coating in an amount ranging between about 10% to about 25% by weight.

910. The method of item 871, wherein the agent is associated with an intravascular implant, to form a device, prior to contacting i), and wherein the implant and fibrosing agent are combined so as to provide a coating on the implant, and the fibrosing agent is present in the coating in an amount ranging between about 25% to about 70% by weight.

911. The method of item 871, wherein the agent is associated with an intravascular implant, to form a device, prior to contacting i), and wherein the implant and fibrosing agent are combined so as to provide a coating on the implant, wherein the device comprises a first coating having a first composition and a second coating having a second composition.

912. The method of item 871, wherein the agent is associated with an intravascular implant, to form a device, prior to contacting i), and wherein the implant and fibrosing agent are combined so as to provide a coating on the implant, wherein the device comprises a first coating having a first composition and a second coating having a second composition, and where the first composition and the second composition are different.

913. The method of item 871, wherein the agent is associated with an intravascular implant, to form a device, prior to contacting i), and wherein the implant and fibrosing agent are combined so as to provide a coating on the implant, and the coating further comprises a polymer.

914. The method of item 871, wherein the agent is associated with an intravascular implant, to form a device, prior to contacting i), and wherein the implant and fibrosing agent are combined so as to provide a coating on the implant, and the coating further comprises a polymer, and the polymer is a copolymer.

915. The method of item 871, wherein the agent is associated with an intravascular implant, to form a device, prior to contacting i), and wherein the implant and fibrosing agent are combined so as to provide a coating on the implant, and the coating further comprises a block copolymer.

916. The method of item 871, wherein the agent is associated with an intravascular implant, to form a device, prior to contacting i), and wherein the implant and fibrosing agent are combined so as to provide a coating on the implant, and the coating further comprises a random copolymer.

917. The method of item 871, wherein the agent is associated with an intravascular implant, to form a device, prior to contacting i), and wherein the implant and fibrosing agent are combined so as to provide a coating on the implant, and the coating further comprises a biodegradable polymer.

918. The method of item 871, wherein the agent is associated with an intravascular implant, to form a device, prior to contacting i), and wherein the implant and fibrosing agent are combined so as to provide a coating on the implant, and the coating further comprises a non-biodegradable polymer.

919. The method of item 871, wherein the agent is associated with an intravascular implant, to form a device, prior to contacting i), and wherein the implant and fibrosing agent are combined so as to provide a coating on the implant, and the coating further comprises a hydrophilic polymer.

920. The method of item 871, wherein the agent is associated with an intravascular implant, to form a device, prior to contacting i), and wherein the implant and fibrosing agent are combined so as to provide a coating on the implant, and the coating further comprises a hydrophobic polymer.

921. The method of item 871, wherein the agent is associated with an intravascular implant, to form a device, prior to contacting i), and wherein the implant and fibrosing agent are combined so as to provide a coating on the implant, and the coating further comprises a polymer having hydrophilic domains.

922. The method of item 871, wherein the agent is associated with an intravascular implant, to form a device, prior to contacting i), and wherein the implant and fibrosing agent are combined so as to provide a coating on the implant, and the coating further comprises a polymer having hydrophobic domains.

923. The method of item 871, wherein the agent is associated with an intravascular implant, to form a device, prior to contacting i), and wherein the implant and fibrosing agent are combined so as to provide a coating on the implant, and the coating further comprises a non-conductive polymer.

924. The method of item 871, wherein the agent is associated with an intravascular implant, to form a device, prior to contacting i), and wherein the implant and fibrosing agent are combined so as to provide a coating on the implant, and the coating further comprises an elastomer.

925. The method of item 871, wherein the agent is associated with an intravascular implant, to form a device, prior to contacting i), and wherein the implant and fibrosing agent are combined so as to provide a coating on the implant, and the coating further comprises a hydrogel.

926. The method of item 871, wherein the agent is associated with an intravascular implant, to form a device, prior to contacting i), and wherein the implant and fibrosing agent are combined so as to provide a coating on the implant, and the coating further comprises a silicone polymer.

927. The method of item 871, wherein the agent is associated with an intravascular implant, to form a device, prior to contacting i), and wherein the implant and fibrosing agent are combined so as to provide a coating on the implant, and the coating further comprises a hydrocarbon polymer.

928. The method of item 871, wherein the agent is associated with an intravascular implant, to form a device, prior to contacting i), and wherein the implant and fibrosing agent are combined so as to provide a coating on the implant, and the coating further comprises a styrene-derived polymer.

929. The method of item 871, wherein the agent is associated with an intravascular implant, to form a device, prior to contacting i), and wherein the implant and fibrosing agent are combined so as to provide a coating on the implant, and the coating further comprises a butadiene-derived polymer.

930. The method of item 871, wherein the agent is associated with an intravascular implant, to form a device, prior to contacting i), and wherein the implant and fibrosing agent are combined so as to provide a coating on the implant, and the coating further comprises a macromer.

931. The method of item 871, wherein the agent is associated with an intravascular implant, to form a device, prior to contacting i), and wherein the implant and fibrosing agent are combined so as to provide a coating on the implant, and the coating further comprises a poly(ethylene glycol)polymer.

932. The method of item 871, wherein the agent is associated with an intravascular implant, to form a device, prior to contacting i), and wherein the implant and fibrosing agent are combined so as to provide a coating on the implant, and the coating further comprises an amorphous polymer.

933. The method of item 871, wherein the agent is associated with an intravascular implant, to form a device, prior to contacting i), and wherein the implant and fibrosing agent are combined so as to provide a coating on the implant, and the coating is a lubricious coating.

934. The method of item 871, wherein the agent is associated with an intravascular implant, to form a device, prior to contacting i), and wherein the implant and fibrosing agent are combined so as to provide a coating on the implant, and the coating is located within pores or holes of the implant.

935. The method of item 871, wherein the agent is associated with an intravascular implant, to form a device, prior to contacting i), and wherein the implant and fibrosing agent are combined so as to provide a coating on the implant, and the coating is located solely within pores or holes of the implant.

936. The method of item 871, wherein the agent is associated with an intravascular implant, to form a device, prior to contacting i), and wherein the implant and fibrosing agent are combined so as to provide a coating on the implant, and the coating is located within a channel, lumen, or divet of the implant.

937. The method of item 871, wherein the agent is associated with an intravascular implant, to form a device, prior to contacting i), and wherein the implant is combined with a second pharmaceutically active agent.

938. The method of item 871, wherein the agent is associated with an intravascular implant, to form a device, prior to contacting i), and wherein the implant is further combined with an anti-inflammatory agent.

939. The method of item 871, wherein the agent is associated with an intravascular implant, to form a device, prior to contacting i), and wherein the implant is further combined with an agent that inhibits infection.

940. The method of item 871, wherein the agent is associated with an intravascular implant, to form a device, prior to contacting i), and wherein the implant is further combined with an anthracycline.

941. The method of item 871, wherein the agent is associated with an intravascular implant, to form a device, prior to contacting i), and wherein the implant is further combined with doxorubicin.

942. The method of item 871, wherein the agent is associated with an intravascular implant, to form a device, prior to contacting i), and wherein the implant is further combined with mitoxantrone.

943. The method of item 871, wherein the agent is associated with an intravascular implant, to form a device, prior to contacting i), and wherein the implant is further combined with a fluoropyrimidine.

944. The method of item 871, wherein the agent is associated with an intravascular implant, to form a device, prior to contacting i), and wherein the implant is further combined with 5-fluorouracil (5-FU).

945. The method of item 871, wherein the agent is associated with an intravascular implant, to form a device, prior to contacting i), and wherein the implant is further combined with a folic acid antagonist.

946. The method of item 871, wherein the agent is associated with an intravascular implant, to form a device, prior to contacting i), and wherein the implant is further combined with methotrexate.

947. The method of item 871, wherein the agent is associated with an intravascular implant, to form a device, prior to contacting i), and wherein the implant is further combined with a podophylotoxin.

948. The method of item 871, wherein the agent is associated with an intravascular implant, to form a device, prior to contacting i), and wherein the implant is further combined with etoposide.

949. The method of item 871, wherein the agent is associated with an intravascular implant, to form a device, prior to contacting i), and wherein the implant is further combined with a camptothecin.

950. The method of item 871, wherein the agent is associated with an intravascular implant, to form a device, prior to contacting i), and wherein the implant is further combined with a hydroxyurea.

951. The method of item 871, wherein the agent is associated with an intravascular implant, to form a device, prior to contacting i), and wherein the implant is further combined with a platinum complex.

952. The method of item 871, wherein the agent is associated with an intravascular implant, to form a device, prior to contacting i), and wherein the implant is further combined with cisplatin.

953. The method of item 871, wherein the agent is associated with an intravascular implant, to form a device, prior to contacting i); and wherein the implant is further combined with an anti-thrombotic agent.

954. The method of item 871,;wherein the agent is associated with an intravascular implant, to form a device, prior to contacting i), and wherein the device further comprises a visualization agent.

955. The method of item 871, wherein the agent is associated with an intravascular implant, to form a device, prior to contacting i), and wherein the device further comprises a visualization agent, wherein the visualization agent is a radiopaque material, wherein the radiopaque material comprises a metal, a halogenated compound, or a barium containing compound.

956. The method of item 871, wherein the agent is associated with an intravascular implant, to form a device, prior to contacting i), and wherein the device further comprises a visualization agent, wherein the visualization agent is a radiopaque material, wherein the radiopaque material comprises barium, tantalum, or technetium.

957. The method of item 871, wherein the agent is associated with an intravascular implant, to form a device, prior to contacting i), and wherein the device further comprises a visualization agent, wherein the visualization agent is a MRI responsive material.

958. The method of item 871, wherein the agent is associated with an intravascular implant, to form a device, prior to contacting i), and wherein the device further comprises a visualization agent, wherein the visualization agent comprises a gadolinium chelate.

959. The method of item 871, wherein the agent is associated with an intravascular implant, to form a device, prior to contacting i), and wherein the device further comprises a visualization agent, wherein the visualization agent comprises iron, magnesium, manganese, copper, or chromium.

960. The method of item 871, wherein the agent is associated with an intravascular implant, to form a device, prior to contacting i), and wherein the device further comprises a visualization agent, wherein the visualization agent comprises an iron oxide compound.

961. The method of item 871, wherein the agent is associated with an intravascular implant, to form a device, prior to contacting i), and wherein the device further comprises a visualization agent, wherein the visualization agent is or comprises a dye, pigment, or colorant.

962. The method of item 871, wherein the agent is associated with an intravascular implant, to form a device, prior to contacting i), and wherein the device further comprises an echogenic material.

963. The method of item 871, wherein the agent is associated with an intravascular implant, to form a device, prior to contacting i), and wherein the device further comprises an echogenic material, and the echogenic material is in the form of a coating.

964. The method of item 871, wherein the agent is associated with an intravascular implant, to form a device, prior to contacting i), and wherein the device is sterilized.

965. The method of item 871, wherein the agent is associated with an intravascular implant, to form a device, prior to contacting i), and wherein the fibrosing agent is associated with the implant to provide for release of the fibrosing agent into tissue in the vicinity of the device after deployment of the device in a patient.

966. The method of item 871, wherein the agent is associated with an intravascular implant, to form a device, prior to contacting i), and wherein the fibrosing agent is associated with the implant to provide for release of the fibrosing agent into tissue in the vicinity of the device after deployment of the device in a patient, wherein the fibrosing agent is released in effective concentrations from the device over a period ranging from the time of deployment of the device to about at least 1 year.

967. The method of item 871, wherein the agent is associated with an intravascular implant, to form a device, prior to contacting i), and wherein the fibrosing agent is associated with the implant to provide for release of the fibrosing agent into tissue in the vicinity of the device after deployment of the device in a patient, wherein the fibrosing agent is released in effective concentrations from the device over a period ranging from the time of deployment of the device to at least about 6 months.

968. The method of item 871, wherein the agent is associated with an intravascular implant, to form a device, prior to contacting i), and wherein the fibrosing agent is associated with the implant to provide for release of the fibrosing agent into tissue in the vicinity of the device after deployment of the device in a patient, wherein the fibrosing agent is released in effective concentrations from the device over a period ranging from the time of deployment of the device to at least about 90 days.

969. The method of item 871, wherein the agent is associated with an intravascular implant, to form a device, prior to contacting i), and wherein the fibrosing agent is associated with the implant to provide for release of the fibrosing agent into tissue in the vicinity of the device after deployment of the device in a patient, wherein the fibrosing agent is released in effective concentrations from the device at a constant rate.

970. The method of item 871, wherein the agent is associated with an intravascular implant, to form a device, prior to contacting i), and wherein the fibrosing agent is associated with the implant to provide for release of the fibrosing agent into tissue in the vicinity of the device after deployment of the device in a patient, wherein the fibrosing agent is released in effective concentrations from the device at an increasing rate.

971. The method of item 871, wherein the agent is associated with an intravascular implant, to form a device, prior to contacting i), and wherein the fibrosing agent is associated with the implant to provide for release of the fibrosing agent into tissue in the vicinity of the device after deployment of the device in a patient, wherein the fibrosing agent is released in effective concentrations from the device at a decreasing rate.

972. The method of item 871, wherein the agent is associated with an intravascular implant, to form a device, prior to contacting i), and wherein the fibrosing agent is associated with the implant to provide for release of the fibrosing agent into tissue in the vicinity of the device after deployment of the device in a patient, wherein the fibrosing agent is released in effective concentrations from the composition by diffusion over a period ranging from the time of deployment of the device to at least about 90 days from deployment.

973. The method of item 871, wherein the agent is associated with an intravascular implant, to form a device, prior to contacting i), and wherein the fibrosing agent is associated with the implant to provide for release of the fibrosing agent into tissue in the vicinity of the device after deployment of the device in a patient, wherein the fibrosing agent is released in effective concentrations from the composition by erosion of the composition over a period ranging from the time of deployment of the device to at least about 90 days from deployment.

974. The method of item 871, wherein the agent is associated with an intravascular implant, to form a device, prior to contacting i), and wherein the device comprises about 0.01 μg to about 10 μg of the fibrosing agent.

975. The method of item 871, wherein the agent is associated with an intravascular implant, to form a device, prior to contacting i), and wherein the device comprises about 10 μg to about 10 mg of the fibrosing agent.

976. The method of item 871, wherein the agent is associated with an intravascular implant, to form a device, prior to contacting i), and wherein the device comprises about 10 mg to about 250 mg of the fibrosing agent.

977. The method of item 871, wherein the agent is associated with an intravascular implant, to form a device, prior to contacting i), and wherein the device comprises about 250 mg to about 1000 mg of the fibrosing agent.

978. The method of item 871, wherein the agent is associated with an intravascular implant, to form a device, prior to contacting i), and wherein the device comprises about 1000 mg to about 2500 mg of the fibrosing agent.

979. The method of item 871, wherein the agent is associated with an intravascular implant, to form a device, prior to contacting i), and wherein a surface of the device comprises less than 0.01 μg of the fibrosing agent per mm² of device surface occupied by fibrosing agent.

980. The method of item 871, wherein the agent is associated with an intravascular implant, to form a device, prior to contacting i), and wherein a surface of the device comprises about 0.01 μg to about 1 μg of the fibrosing agent per mm² of device surface occupied by fibrosing agent.

981. The method of item 871, wherein the agent is associated with an intravascular implant, to form a device, prior to contacting i), and wherein a surface of the device comprises about 1 μg to about 10 μg of the fibrosing agent per mm² of device surface occupied by fibrosing agent.

982. The method of item 871, wherein the agent is associated with an intravascular implant, to form a device, prior to contacting i), and wherein a surface of the device comprises about 10 μg to about 250 μg of the fibrosing agent per mm² of device surface occupied by fibrosing agent.

983. The method of item 871, wherein the agent is associated with an intravascular implant, to form a device, prior to contacting i), and wherein a surface of the device comprises about 250 μg to about 1000 μg of the fibrosing agent of fibrosing agent per mm² of device surface occupied by fibrosing agent.

984. The method of item 871, wherein the agent is associated with an intravascular implant, to form a device, prior to contacting i), and wherein a surface of the device comprises about 1000 μg to about 2500 μg of the fibrosing agent per mm² of device surface occupied by fibrosing agent.

985. The method of item 871, wherein the agent is associated with an intravascular implant, to form a device, prior to contacting i), and wherein the intravascular implant is a catheter.

986. The method of item 871, wherein the agent is associated with an intravascular implant, to form a device, prior to contacting i), and wherein the intravascular implant is a balloon.

987. The method of item 871, wherein the agent is associated with an intravascular implant, to form a device, prior to-contacting i), and wherein the intravascular implant is a stent.

988. The method of item 871, wherein the agent is associated with an intravascular implant, to form a device, prior to contacting i), and wherein the intravascular implant is a stent graft.

989. The method of item 871, wherein the agent is associated with an intravascular implant, to form a device, prior to contacting i), and wherein the intravascular implant is a tubular structure that comprises a lumen through which blood may flow, wherein the tubular structure comprises a luminal surface and a non-luminal surface.

990. The method of item 871, wherein the agent is associated with an intravascular implant, to form a device, prior to contacting i), and wherein the intravascular implant is a tubular structure that comprises a lumen through which blood may flow, wherein the tubular structure comprises a luminal surface and a non-luminal surface, and wherein the agent or the composition comprising the agent is coated onto the non-luminal surface of the implant.

991. The method of item 871, wherein the agent is associated with an intravascular implant, to form a device, prior to contacting i), and wherein the intravascular implant is a tubular structure that comprises a lumen through which blood may flow, wherein the tubular structure comprises a luminal surface and a non-luminal surface, and wherein the agent or the composition comprising the agent is directly affixed to the non-luminal surface of the implant.

992. The method of item 871, wherein the agent is associated with an intravascular implant, to form a device, prior to contacting i), and wherein the intravascular implant is a tubular structure that comprises a lumen through which blood may flow, wherein the tubular structure comprises a luminal surface and a non-luminal surface, and wherein all or a portion of the non-luminal surface of the structure is covered with the fibrosing agent or the composition comprising the fibrosing agent.

993. The method of item 871, wherein the agent is associated with an intravascular implant, to form a device, prior to contacting i), and wherein the intravascular implant is a tubular structure that comprises a lumen through which blood may flow, wherein the tubular structure comprises a luminal surface and a non-luminal surface, and wherein all or a portion of the non-luminal surface of the intraluminal device is coated with a proliferative agent.

994. The method of item 871, wherein the agent is associated with an intravascular implant, to form a device, prior to contacting i), and wherein the intravascular implant is a tubular structure that comprises a lumen through which blood may flow, wherein the tubular structure comprises a luminal surface and a non-luminal surface, and wherein all or a portion of the luminal surface of the structure is coated with an agent that inhibits restenosis.

995. The method of item 871, wherein the agent is associated with an intravascular implant, to form a device, prior to contacting i), and wherein the intravascular implant is a tubular structure that comprises a lumen through which blood may flow, wherein the tubular structure comprises a luminal surface and a non-luminal surface, where the method comprises attaching a thread to a non-luminal surface of the structure, wherein the thread is, or comprises, the fibrosing agent or the composition comprising the fibrosing agent.

996. A method of inducing fibrosis to contain vulnerable plaque i, comprising covering the outer surface of the plaque in a patient in need thereof with an agent or a composition comprising an agent, wherein the agent induces fibrosis.

997. The method of item 996 wherein the fibrosing agent promotes regeneration.

998. The method of item 996 wherein the fibrosing agent promotes angiogenesis.

999. The method of item 996 wherein the fibrosing agent promotes fibroblast migration.

1000. The method of item 996 wherein the fibrosing agent promotes fibroblast proliferation.

1001. The method of item 996 wherein the fibrosing agent promotes deposition of extracellular matrix (ECM).

1002. The method of item 996 wherein the fibrosing agent promotes tissue remodeling.

1003. The method of item 996 wherein the fibrosing agent is an arterial vessel wall irritant.

1004. The method of item 996 wherein the fibrosing agent is or comprises silk.

1005. The method of item 996 wherein the fibrosing agent is or comprises mineral particles.

1006. The method of item 996 wherein the fibrosing agent is or comprises chitosan.

1007. The method of item 996 wherein the fibrosing agent is or comprises polylysine.

1008. The method of item 996 wherein the fibrosing agent is or comprises fibronectin.

1009. The method of item 996 wherein the fibrosing agent is or comprises bleomycin.

1010. The method of item 996 wherein the fibrosing agent is or comprises CTGF.

1011. The method of item 996 wherein the fibrosing agent is in the form of a thread, or is in contact with a thread.

1012. The method of item 996 wherein the fibrosing agent is in the form of a particulate.

1013. The method of item 996 wherein the composition further comprises an inflammatory cytokine.

1014. The method of item 996 wherein the composition further comprises an agent that stimulates cell proliferation.

1015. The method of item 996 wherein the composition is in the form of a gel or paste.

1016. The method of item 996 wherein the fibrosing agent is in the form of tufts.

1017. The method of item 996, wherein the agent is associated with an intravascular implant prior to contacting i).

1018. The method of item 996, wherein the agent is associated with an intravascular implant prior to contacting i), and the fibrosing agent promotes adhesion between the implant and the patient.

1019. The method of item 996, wherein the agent is associated with an intravascular implant prior to contacting i), and wherein the implant delivers the fibrosing agent locally to tissue proximate to the implant.

1020. The method of item 996, wherein the agent is associated with an intravascular implant prior to contacting i), and wherein the implant and fibrosing agent are combined so as to provide a coating on the implant.

1021. The method of item 996, wherein the agent is associated with an intravascular implant, to form a device, prior to contacting i), and wherein the implant and fibrosing agent are combined so as to provide a coating on the implant, and the coating directly contacts the device.

1022. The method of item 996, wherein the agent is associated with an intravascular implant, to form a device, prior to contacting i), and wherein the implant and fibrosing agent are combined so as to provide a coating on the implant, and the coating indirectly contacts the device.

1023. The method of item 996, wherein the agent is associated with an intravascular implant, to form a device, prior to contacting i), and wherein the implant and fibrosing agent are combined so as to provide a coating on the implant, and the coating partially covers the device.

1024. The method of item 996, wherein the agent is associated with an intravascular implant, to form a device, prior to contacting i), and wherein the implant and fibrosing agent are combined so as to provide a coating on the implant, and the coating completely covers the device.

1025. The method of item 996, wherein the agent is associated with an intravascular implant, to form a device, prior to contacting i), and wherein the implant and fibrosing agent are combined so as to provide a coating on the implant, where the coating is a uniform coating.

1026. The method of item 996, wherein the agent is associated with an intravascular implant, to form a device, prior to contacting i), and wherein the implant and fibrosing agent are combined so as to provide a coating on the implant, where the coating is a non-uniform coating.

1027. The method of item 996, wherein the agent is associated with an intravascular implant, to form a device, prior to contacting i), and wherein the implant and fibrosing agent are combined so as to provide a coating on the implant, where the coating is a discontinuous coating.

1028. The method of item 996, wherein the agent is associated with an intravascular implant, to form a device, prior to contacting i), and wherein the implant and fibrosing agent are combined so as to provide a coating on the implant, where the coating is a patterned coating.

1029. The method of item 996, wherein the agent is associated with an intravascular implant, to form a device, prior to contacting i), and wherein the implant and fibrosing agent are combined so as to provide a coating on the implant, where the coating has a thickness of 100 μm or less.

1030. The method of item 996, wherein the agent is associated with an intravascular implant, to form a device, prior to contacting i), and wherein the implant and fibrosing agent are combined so as to provide a coating on the implant, where the coating has a thickness of 10 μm or less.

1031. The method of item 996, wherein the agent is associated with an intravascular implant, to form a device, prior to contacting i), and wherein the implant and fibrosing agent are combined so as to provide a coating on the implant, and the coating is stable at room temperature for a period of at least 1 year.

1032. The method of item 996, wherein the agent is associated with an intravascular implant, to form a device, prior to contacting i), and wherein the implant and fibrosing agent are combined so as to provide a coating on the implant, and the fibrosing agent is present in the coating in an amount ranging between about 0.0001% to about 1% by weight.

1033. The method of item 996, wherein the agent is associated with an intravascular implant, to form a device, prior to contacting i), and wherein the implant and fibrosing agent are combined so as to provide a coating on the implant, and the fibrosing agent is present in the coating in an amount ranging between about 1% to about 10% by weight.

1034. The method of item 996, wherein the agent is associated with an intravascular implant, to form a device, prior to contacting i), and wherein the implant and fibrosing agent are combined so as to provide a coating on the implant, and the fibrosing agent is present in the coating in an amount ranging between about 10% to about 25% by weight.

1035. The method of item 996, wherein the agent is associated with an intravascular implant, to form a device, prior to contacting i), and wherein the implant and fibrosing agent are combined so as to provide a coating on the implant, and the fibrosing agent is present in the coating in an amount ranging between about 25% to about 70% by weight.

1036. The method of item 996, wherein the agent is associated with an intravascular implant, to form a device, prior to contacting i), and wherein the implant and fibrosing agent are combined so as to provide a coating on the implant, wherein the device comprises a first coating having a first composition and a second coating having a second composition.

1037. The method of item 996, wherein the agent is associated with an intravascular implant, to form a device, prior to contacting i), and wherein the implant and fibrosing agent are combined so as to provide a coating on the implant, wherein the device comprises a first coating having a first composition and a second coating having a second composition, and where the first composition and the second composition are different.

1038. The method of item 996, wherein the agent is associated with an intravascular implant, to form a device, prior to contacting i), and wherein the implant and fibrosing agent are combined so as to provide a coating on the implant, and the coating further comprises a polymer.

1039. The method of item 996, wherein the agent is associated with an intravascular implant, to form a device, prior to contacting i), and wherein the implant and fibrosing agent are combined so as to provide a coating on the implant, and the coating further comprises a polymer, and the polymer is a copolymer.

1040. The method of item 996, wherein the agent is associated with an intravascular implant, to form a device, prior to contacting i), and wherein the implant and fibrosing agent are combined so as to provide a coating on the implant, and the coating further comprises a block copolymer.

1041. The method of item 996, wherein the agent is associated with an intravascular implant, to form a device, prior to contacting i), and wherein the implant and fibrosing agent are combined so as to provide a coating on the implant, and the coating further comprises a random copolymer.

1042. The method of item 996, wherein the agent is associated with an intravascular implant, to form a device, prior to contacting i), and wherein the implant and fibrosing agent are combined so as to provide a coating on the implant, and the coating further comprises a biodegradable polymer.

1043. The method of item 996, wherein the agent is associated with an intravascular implant, to form a device, prior to contacting i), and wherein the implant and fibrosing agent are combined so as to provide a coating on the implant, and the coating further comprises a non-biodegradable polymer.

1044. The method of item 996, wherein the agent is associated with an intravascular implant, to form a device, prior to contacting i), and wherein the implant and fibrosing agent are combined so as to provide a coating on the implant, and the coating further comprises a hydrophilic polymer.

1045. The method of item 996, wherein the agent is associated with an intravascular implant, to form a device, prior to contacting i), and wherein the implant and fibrosing agent are combined so as to provide a coating on the implant, and the coating further comprises a hydrophobic polymer.

1046. The method of item 996, wherein the agent is associated with an intravascular implant, to form a device, prior to contacting i), and wherein the implant and fibrosing agent are combined so as to provide a coating on the implant, and the coating further comprises a polymer having hydrophilic domains.

1047. The method of item 996, wherein the agent is associated with an intravascular implant, to form a device, prior to contacting i), and wherein the implant and fibrosing agent are combined so as to provide a coating on the implant, and the coating further comprises a polymer having hydrophobic domains.

1048. The method of item 996, wherein the agent is associated with an intravascular implant, to form a device, prior to contacting i), and wherein the implant and fibrosing agent are combined so as to provide a coating on the implant, and the coating further comprises a non-conductive polymer.

1049. The method of item 996, wherein the agent is associated with an intravascular implant, to form a device, prior to contacting i), and wherein the implant and fibrosing agent are combined so as to provide a coating on the implant, and the coating further comprises an elastomer.

1050. The method of item 996, wherein the agent is associated with an intravascular implant, to form a device, prior to contacting i), and wherein the implant and fibrosing agent are combined so as to provide a coating on the implant, and the coating further comprises a hydrogel.

1051. The method of item 996, wherein the agent is associated with an intravascular implant, to form a device, prior to contacting i), and wherein the implant and fibrosing agent are combined so as to provide a coating on the implant, and the coating further comprises a silicone polymer.

1052. The method of item 996, wherein the agent is associated with an intravascular implant, to form a device, prior to contacting i), and wherein the implant and fibrosing agent are combined so as to provide a coating on the implant, and the coating further comprises a hydrocarbon polymer.

1053. The method of item 996, wherein the agent is associated with an intravascular implant, to form a device, prior to contacting i), and wherein the implant and fibrosing agent are combined so as to provide a coating on the implant, and the coating further comprises a styrene-derived polymer.

1054. The method of item 996, wherein the agent is associated with an intravascular implant, to form a device, prior to contacting i), and wherein the implant and fibrosing agent are combined so as to provide a coating on the implant, and the coating further comprises a butadiene-derived polymer.

1055. The method of item 996, wherein the agent is associated with an intravascular implant, to form a device, prior to contacting i), and wherein the implant and fibrosing agent are combined so as to provide a coating on the implant, and the coating further comprises a macromer.

1056. The method of item 996, wherein the agent is associated with an intravascular implant, to form a device, prior to contacting i), and wherein the implant and fibrosing agent are combined so as to provide a coating on the implant, and the coating further comprises a poly(ethylene glycol)polymer.

1057. The method of item 996, wherein the agent is associated with an intravascular implant, to form a device, prior to contacting i), and wherein the implant and fibrosing agent are combined so as to provide a coating on the implant, and the coating further comprises an amorphous polymer.

1058. The method of item 996, wherein the agent is associated with an intravascular implant, to form a device, prior to contacting i), and wherein the implant and fibrosing agent are combined so as to provide a coating on the implant, and the coating is a lubricious coating.

1059. The method of item 996, wherein the agent is associated with an intravascular implant, to form a device, prior to contacting i), and wherein the implant and fibrosing agent are combined so as to provide a coating on the implant, and the coating is located within pores or holes of the implant.

1060. The method of item 996, wherein the agent is associated with an intravascular implant, to form a device, prior to contacting i), and wherein the implant and fibrosing agent are combined so as to provide a coating on the implant, and the coating is located solely within pores or holes of the implant.

1061. The method of item 996, wherein the agent is associated with an intravascular implant, to form a device, prior to contacting i), and wherein the implant and fibrosing agent are combined so as to provide a coating on the implant, and the coating is located within a channel, lumen, or divet of the implant.

1062. The method of item 996, wherein the agent is associated with, an intravascular implant, to form a device, prior to contacting i), and wherein the implant is combined with a second pharmaceutically active agent.

1063. The method of item 996, wherein the agent is associated with an intravascular implant, to form a device, prior to contacting i), and wherein the implant is further combined with an anti-inflammatory agent.

1064. The method of item 996, wherein the agent is associated with an intravascular implant, to form a device, prior to contacting i), and wherein the implant is further combined with an agent that inhibits infection.

1065. The method of item 996, wherein the agent is associated with an intravascular implant, to form a device, prior to contacting i), and wherein the implant is further combined with an anthracycline.

1066. The method of item 996, wherein the agent is associated with an intravascular implant, to form a device, prior to contacting i), and wherein the implant is further combined with doxorubicin.

1067. The method of item 996, wherein the agent is associated with an intravascular implant, to form a device, prior to contacting i), and wherein the implant is further combined with mitoxantrone.

1068. The method of item 996, wherein the agent is associated with an intravascular implant, to form a device, prior to contacting i), and wherein the implant is further combined with a fluoropyrimidine.

1069. The method of item 996, wherein the agent is associated with an intravascular implant, to form a device, prior to contacting i), and wherein the implant is further combined with 5-fluorouracil (5-FU).

1070. The method of item 996, wherein the agent is associated with an intravascular implant, to form a device, prior to contacting i), and wherein the implant is further combined with a folic acid antagonist.

1071. The method of item 996, wherein the agent is associated with an intravascular implant, to form a device, prior to contacting i), and wherein the implant is further combined with methotrexate.

1072. The method of item 996, wherein the agent is associated with an intravascular implant, to form a device, prior to contacting i), and wherein the implant is further combined with a podophylotoxin.

1073. The method of item 996, wherein the agent is associated with an intravascular implant, to form a device, prior to contacting i), and wherein the implant is further combined with etoposide.

1074. The method of item 996, wherein the agent is associated with an intravascular implant, to form a device, prior to contacting i), and wherein the implant is further combined with a camptothecin.

1075. The method of item 996, wherein the agent is associated with an intravascular implant, to form a device, prior to contacting i), and wherein the implant is further combined with a hydroxyurea.

1076. The method of item 996, wherein the agent is associated with an intravascular implant, to form a device, prior to contacting i), and wherein the implant is further combined with a platinum complex.

1077. The method of item 996, wherein the agent is associated with an intravascular implant, to form a device, prior to contacting i), and wherein the implant is further combined with cisplatin.

1078. The method of item 996, wherein the agent is associated with an intravascular implant, to form a device, prior to contacting i), and wherein the implant is further combined with an anti-thrombotic agent.

1079. The method of item 996, wherein the agent is associated with an intravascular implant, to form a device, prior to contacting i), and wherein the device further comprises a visualization agent.

1080. The method of item 996, wherein the agent is associated with an intravascular implant, to form a device, prior to contacting i), and wherein the device further comprises a visualization agent, wherein the visualization agent is a radiopaque material, wherein the radiopaque material comprises a metal, a halogenated compound, or a barium containing compound.

1081. The method of item 996, wherein the agent is associated with an intravascular implant, to form a device, prior to contacting i), and wherein the device further comprises a visualization agent, wherein the visualization agent is a radiopaque material, wherein the radiopaque material comprises barium, tantalum, or technetium.

1082. The method of item 996, wherein the agent is associated with an intravascular implant, to form a device, prior to contacting i), and wherein the device further comprises a visualization agent, wherein the visualization agent is a MRI responsive material.

1083. The method of item 996, wherein the agent is associated with an intravascular implant, to form a device, prior to contacting i), and wherein the device further comprises a visualization agent, wherein the visualization agent comprises a gadolinium chelate.

1084. The method of item 996, wherein the agent is associated with an intravascular implant, to form a device, prior to contacting i), and wherein the device further comprises a visualization agent, wherein the visualization agent comprises iron, magnesium, manganese, copper, or chromium.

1085. The method of item 996, wherein the agent is associated with an intravascular implant, to form a device, prior to contacting i), and wherein the device further comprises a visualization agent, wherein the visualization agent comprises an iron oxide compound.

1086. The method of item 996, wherein the agent is associated with an intravascular implant, to form a device, prior to contacting i), and wherein the device further comprises a visualization agent, wherein the visualization agent is or comprises a dye, pigment, or colorant.

1087. The method of item 996, wherein the agent is associated with an intravascular implant, to form a device, prior to contacting i), and wherein the device further comprises an echogenic material.

1088. The method of item 996, wherein the agent is associated with an intravascular implant, to form a device, prior to contacting i), and wherein the device further comprises an echogenic material, and the echogenic material is in the form of a coating.

1089. The method of item 996, wherein the agent is associated with an intravascular implant, to form a device, prior to contacting i), and wherein the device is sterilized.

1090. The method of item 996, wherein the agent is associated with an intravascular implant, to form a device, prior to contacting i), and wherein the fibrosing agent is associated with the implant to provide for release of the fibrosing agent into tissue in the vicinity of the device after deployment of the device in a patient.

1091. The method of item 996, wherein the agent is associated with an intravascular implant, to form a device, prior to contacting i), and wherein the fibrosing agent is associated with the implant to provide for release of the fibrosing agent into tissue in the vicinity of the device after deployment of the device in a patient, wherein the fibrosing agent is released in effective concentrations from the device over a period ranging from the time of deployment of the device to about at least 1 year.

1092. The method of item 996, wherein the agent is associated with an intravascular implant, to form a device, prior to contacting i), and wherein the fibrosing agent is associated with the implant to provide for release of the fibrosing agent into tissue in the vicinity of the device after deployment of the device in a patient, wherein the fibrosing agent is released in effective concentrations from the device over a period ranging from the time of deployment of the device to at least about 6 months.

1093. The method of item 996, wherein the agent is associated with an intravascular implant, to form a device, prior to contacting i), and wherein the fibrosing agent is associated with the implant to provide for release of the fibrosing agent into tissue in the vicinity of the device after deployment of the device in a patient, wherein the fibrosing agent is released in effective concentrations from the device over a period ranging from the time of deployment of the device to at least about 90 days.

1094. The method of item 996, wherein the agent is associated with an intravascular implant, to form a device, prior to contacting i), and wherein the fibrosing agent is associated with the implant to provide for release of the fibrosing agent into tissue in the vicinity of the device after deployment of the device in a patient, wherein the fibrosing agent is released in effective concentrations from the device at a constant rate.

1095. The method of item 996, wherein the agent is associated with an intravascular implant, to form a device, prior to contacting i), and wherein the fibrosing agent is associated with the implant to provide for release of the fibrosing agent into tissue in the vicinity of the device after deployment of the device in a patient, wherein the fibrosing agent is released in effective concentrations from the device at an increasing rate.

1096. The method of item 996, wherein the agent is associated with an intravascular implant, to form a device, prior to contacting i), and wherein the fibrosing agent is associated with the implant to provide for release of the fibrosing agent into tissue in the vicinity of the device after deployment of the device in a patient, wherein the fibrosing agent is released in effective concentrations from the device at a decreasing rate.

1097. The method of item 996, wherein the agent is associated with an intravascular implant, to form a device, prior to contacting i), and wherein the fibrosing agent is associated with the implant to provide for release of the fibrosing agent into tissue in the vicinity of the device after deployment of the device in a patient, wherein the fibrosing agent is released in effective concentrations from the composition by diffusion over a period ranging from the time of deployment of the device to at least about 90 days from deployment.

1098. The method of item 996, wherein the agent is associated with an intravascular implant, to form a device, prior to contacting i), and wherein the fibrosing agent is associated with the implant to provide for release of the fibrosing agent into tissue in the vicinity of the device after deployment of the device in a patient, wherein the fibrosing agent is released in effective concentrations from the composition by erosion of the composition over a period ranging from the time of deployment of the device to at least about 90 days from deployment.

1099. The method of item 996, wherein the agent is associated with an intravascular implant, to form a device, prior to contacting i), and wherein the device comprises about 0.01 μg to about 10 μg of the fibrosing agent.

1100. The method of item 996, wherein the agent is associated with an intravascular implant, to form a device, prior to contacting i), and wherein the device comprises about 10 μg to about 10 mg of the fibrosing agent.

1101. The method of item 996, wherein the agent is associated with an intravascular implant, to form a device, prior to contacting i), and wherein the device comprises about 10 mg to about 250 mg of the fibrosing agent.

1102. The method of item 996, wherein the agent is associated with an intravascular implant, to form a device, prior to contacting i), and wherein the device comprises about 250 mg to about 1000 mg of the fibrosing agent.

1103. The method of item 996, wherein the agent is associated with an intravascular implant, to form a device, prior to contacting i), and wherein the device comprises about 1000 mg to about 2500 mg of the fibrosing agent.

1104. The method of item 996, wherein the agent is associated with an intravascular implant, to form a device, prior to contacting i), and wherein a surface of the device comprises less than 0.01 μg of the fibrosing agent per mm² of device surface occupied by fibrosing agent.

1105. The method of item 996, wherein the agent is associated with an intravascular implant, to form a device, prior to contacting i), and wherein a surface of the device comprises about 0.01 μg to about 1 μg of the fibrosing agent per mm² of device surface occupied by fibrosing agent.

1106. The method of item 996, wherein the agent is associated with an intravascular implant, to form a device, prior to contacting i), and wherein a surface of the device comprises about 1 μg to about 10 μg of the fibrosing agent per mm² of device surface occupied by fibrosing agent.

1107. The method of item 996, wherein the agent is associated with an intravascular implant, to form a device, prior to contacting i), and wherein a surface of the device comprises about 10 μg to about 250 μg of the fibrosing agent per mm² of device surface occupied by fibrosing agent.

1108. The method of item 996, wherein the agent is associated with an intravascular implant, to form a device, prior to contacting i), and wherein a surface of the device comprises about 250 μg to about 1000 μg of the fibrosing agent of fibrosing agent per mm² of device surface occupied by fibrosing agent.

1109. The method of item 996, wherein the agent is associated with an intravascular implant, to form a device, prior to contacting i), and wherein a surface of the device comprises about 1000 μg to about 2500 μg of the fibrosing agent per mm² of device surface occupied by fibrosing agent.

1110. The method of item 996, wherein the agent is associated with an intravascular implant, to form a device, prior to contacting i), and wherein the intravascular implant is a catheter.

1111. The method of item 996, wherein the agent is associated with an intravascular implant, to form a device, prior to contacting i), and wherein the intravascular implant is a balloon.

1112. The method of item 996, wherein the agent is associated with an intravascular implant, to form a device, prior to contacting i), and wherein the intravascular implant is a stent.

1113. The method of item 996, wherein the agent is associated with an intravascular implant, to form a device, prior to contacting i), and wherein the intravascular implant is a stent graft.

1114. The method of item 996, wherein the agent is associated with an intravascular implant, to form a device, prior to contacting i), and wherein the intravascular implant is a tubular structure that comprises a lumen through which blood may flow, wherein the tubular structure comprises a luminal surface and a non-luminal surface.

1115. The method of item 996, wherein the agent is associated with an intravascular implant, to form a device, prior to contacting i), and wherein the intravascular implant is a tubular structure that comprises a lumen through which blood may flow, wherein the tubular structure comprises a luminal surface and a non-luminal surface, and wherein the agent or the composition comprising the agent is coated onto the non-luminal surface of the implant.

1116. The method of item 996, wherein the agent is associated with an intravascular implant, to form a device, prior to contacting i), and wherein the intravascular implant is a tubular structure that comprises a lumen through which blood may flow, wherein the tubular structure comprises a luminal surface and a non-luminal surface, and wherein the agent or the composition comprising the agent is directly affixed to the non-luminal surface of the implant.

1117. The method of item 996, wherein the agent is associated with an intravascular implant, to form a device, prior to contacting i), and wherein the intravascular implant is a tubular structure that comprises a lumen through which blood may flow, wherein the tubular structure comprises a luminal surface and a non-luminal surface, and wherein all or a portion of the non-luminal surface of the structure is covered with the fibrosing agent or the composition comprising the fibrosing agent.

1118. The method of item 996, wherein the agent is associated with an intravascular implant, to form a device, prior to contacting i), and wherein the intravascular implant is a tubular structure that comprises a lumen through which blood may flow, wherein the tubular structure comprises a luminal surface and a non-luminal surface, and wherein all or a portion of the non-luminal surface of the intraluminal device is coated with a proliferative agent.

1119. The method of item 996, wherein the agent is associated with an intravascular implant, to form a device, prior to contacting i), and wherein the intravascular implant is a tubular structure that comprises a lumen through which blood may flow, wherein the tubular structure comprises a luminal surface and a non-luminal surface, and wherein all or a portion of the luminal surface of the structure is coated with an agent that inhibits restenosis.

1120. The method of item 996, wherein the agent is associated with an intravascular implant, to form a device, prior to contacting i), and wherein the intravascular implant is a tubular structure that comprises a lumen through which blood may flow, wherein the tubular structure comprises a luminal surface and a non-luminal surface, where the method comprises attaching a thread to a non-luminal surface of the structure, wherein the thread is, or comprises, the fibrosing agent or the composition comprising the fibrosing agent.

1121. A method for treating a patient having an aneurysm, comprising delivering to a patient in need thereof a stent graft, wherein the stent graft comprises i) a stent graft and ii) a fibrosing agent or a composition comprising a fibrosing agent, wherein the agent induces fibrosis.

1122. The method of item 1121 wherein the aneurysm is an aortic aneurysm.

1123. The method of item 1121 wherein the aneurysm is an abdominal, thoracic, or iliac aortic aneurysm.

1124. The method of item 1121 wherein the fibrosing agent promotes regeneration.

1125. The method of item 1121 wherein the fibrosing agent promotes angiogenesis.

1126. The method-of item 1121 wherein the fibrosing agent promotes fibroblast migration.

1127. The method of item 1121 wherein the fibrosing agent promotes fibroblast proliferation.

1128. The method of item 1121 wherein the fibrosing agent promotes deposition of extracellular matrix (ECM).

1129. The method of item 1121 wherein the fibrosing agent promotes tissue remodeling.

1130. The method of item 1121 wherein the fibrosing agent is an arterial vessel wall irritant.

1131. The method of item 1121 wherein the fibrosing agent is or comprises silk.

1132. The method of item 1121 wherein the fibrosing agent is or comprises mineral particles.

1133. The method of item 1121 wherein the fibrosing agent is or comprises chitosan.

1134. The method of item 1121 wherein the fibrosing agent is or comprises polylysine.

1135. The method of item 1121 wherein the fibrosing agent is or comprises fibronectin.

1136. The method of item 1121 wherein the fibrosing agent is or comprises bleomycin.

1137. The method of item 1121 wherein the fibrosing agent is or comprises CTGF.

1138. The method of item 1121 wherein the fibrosing agent is in the form of a thread, or is in contact with a thread.

1139. The method of item 1121 wherein the fibrosing agent is in the form of a particulate.

1140. The method of item 1121 wherein the composition further comprises an inflammatory cytokine.

1141. The method of item 1121 wherein the composition further comprises an agent that stimulates cell proliferation.

1142. The method of item 1121 wherein the composition is in the form of a gel or paste.

1143. The method of item 1121 wherein the fibrosing agent is in the form of tufts.

1144. The method of item 1121, wherein the fibrosing agent promotes adhesion between the stent graft and the patient.

1145. The method of item 1121, wherein the stent graft delivers the fibrosing agent locally to tissue proximate to the stent graft.

1146. The method of item 1121, wherein the stent graft and fibrosing agent are combined so as to provide a coating on the stent graft.

1147. The method of item 1121, wherein the stent graft and fibrosing agent are combined so as to provide a coating on the stent graft, and the coating directly contacts the stent graft.

1148. The method of item 1121, wherein the stent graft and fibrosing agent are combined so as to provide a coating on the stent graft, and the coating indirectly contacts the stent graft.

1149. The method of item 1121, wherein the stent graft and fibrosing agent are combined so as to provide a coating on the stent graft, and the coating partially covers the stent graft.

1150. The method of item 1121, wherein the stent graft and fibrosing agent are combined so as to provide a coating on the stent graft, and the coating completely covers the stent graft.

1151. The method of item 1121, wherein the stent graft and fibrosing agent are combined so as to provide a coating on the stent graft, where the coating is a uniform coating.

1152. The method of item 1121, wherein the stent graft and fibrosing agent are combined so as to provide a coating on the stent graft, where the coating is a non-uniform coating.

1153. The method of item 1121, wherein the stent graft and fibrosing agent are combined so as to provide a coating on the stent graft, where the coating is a discontinuous coating.

1154. The method of item 1121, wherein the stent graft and fibrosing agent are combined so as to provide a coating on the stent graft, where the coating is a patterned coating.

1155. The method of item 1121, wherein the stent graft and fibrosing agent are combined so as to provide a coating on the stent graft, where the coating has a thickness of 100 μm or less.

1156. The method of item 1121, wherein the stent graft and fibrosing agent are combined so as to provide a coating on the stent graft, where the coating has a thickness of 10 μm or less.

1157. The method of item 1121, wherein the stent graft and fibrosing agent are combined so as to provide a coating on the stent graft, and the coating is stable at room temperature for a period of at least 1 year.

1158. The method of item 1121, wherein the stent graft and fibrosing agent are combined so as to provide a coating on the stent graft, and the fibrosing agent is present in the coating in an amount ranging between about 0.0001% to about 1% by weight.

1159. The method of item 1121, wherein the stent graft and fibrosing agent are combined so as to provide a coating on the stent graft, and the fibrosing agent is present in the coating in an amount ranging between about 1% to about 10% by weight.

1160. The method of item 1121, wherein the stent graft and fibrosing agent are combined so as to provide a coating on the stent graft, and the fibrosing agent is present in the coating in an amount ranging between about 10% to about 25% by weight.

1161. The method of item 1121, wherein the stent graft and fibrosing agent are combined so as to provide a coating on the stent graft, and the fibrosing agent is present in the coating in an amount ranging between about 25% to about 70% by weight.

1162. The method of item 1121, wherein the stent graft and fibrosing agent are combined so as to provide a coating on the stent graft, wherein the stent graft comprises a first coating having a first composition and a second coating having a second composition.

1163. The method of item 1121, wherein the stent graft and fibrosing agent are combined so as to provide a coating on the stent graft, wherein the coated stent graft comprises a first coating having a first composition and a second coating having a second composition, and where the first composition and the second composition are different.

1164. The method of item 1121, wherein the stent graft and fibrosing agent are combined so as to provide a coating on the stent graft, and the coating further comprises a polymer.

1165. The method of item 1121, wherein the stent graft and fibrosing agent are combined so as to provide a coating on the stent graft, and the coating further comprises a copolymer.

1166. The method of item 1121, wherein the stent graft and fibrosing agent are combined so as to provide a coating on the stent graft, and the coating further comprises a block copolymer.

1167. The method of item 1121, wherein the stent graft and fibrosing agent are combined so as to provide a coating on the stent graft, and the coating further comprises a random copolymer.

1168. The method of item 1121, wherein the stent graft and fibrosing agent are combined so as to provide a coating on the stent graft, and the coating further comprises a biodegradable polymer.

1169. The method of item 1121, wherein the stent graft and fibrosing agent are combined so as to provide a coating on the stent graft, and the coating further comprises a non-biodegradable polymer.

1170. The method of item 1121, wherein the stent graft and fibrosing agent are combined so as to provide a coating on the stent graft, and the coating further comprises a hydrophilic polymer.

1171. The method of item 1121, wherein the stent graft and fibrosing agent are combined so as to provide a coating on the stent graft, and the coating further comprises a hydrophobic polymer.

1172. The method of item 1121, wherein the stent graft and fibrosing agent are combined so as to provide a coating on the stent graft, and the coating further comprises a polymer having hydrophilic domains.

1173. The method of item 1121, wherein the stent graft and fibrosing agent are combined so as to provide a coating on the stent graft, and the coating further comprises a polymer having hydrophobic domains.

1174. The method of item 1121, wherein the stent graft and fibrosing agent are combined so as to provide a coating on the stent graft, and the coating further comprises a non-conductive polymer.

1175. The method of item 1121, wherein the stent graft and fibrosing agent are combined so as to provide a coating on the stent graft, and the coating further comprises an elastomer.

1176. The method of item 1121, wherein the stent graft and fibrosing agent are combined so as to provide a coating on the stent graft, and the coating further comprises a hydrogel.

1177. The method of item 1121, wherein the stent graft and fibrosing agent are combined so as to provide a coating on the stent graft, and the coating further comprises a silicone polymer.

1178. The method of item 1121, wherein the stent graft and fibrosing agent are combined so as to provide a coating on the stent graft, and the coating further comprises a hydrocarbon polymer.

1179. The method of item 1121, wherein the stent graft and fibrosing agent are combined so as to provide a coating on the stent graft, and the coating further comprises a styrene-derived polymer.

1180. The method of item 1121, wherein the stent graft and fibrosing agent are combined so as to provide a coating on the stent graft, and the coating further comprises a butadiene-derived polymer.

1181. The method of item 1121, wherein the stent graft and fibrosing agent are combined so as to provide a coating on the stent graft, and the coating further comprises a macromer.

1182. The method of item 1121, wherein the stent graft and fibrosing agent are combined so as to provide a coating on the stent graft, and the coating further comprises a poly(ethylene glycol)polymer.

1183. The method of item 1121, wherein the stent graft and fibrosing agent are combined so as to provide a coating on the stent graft, and the coating further comprises an amorphous polymer.

1184. The method of item 1121, wherein the stent graft and fibrosing agent are combined so as to provide a coating on the stent graft, and the coating is a lubricious coating.

1185. The method of item 1121, wherein the stent graft and fibrosing agent are combined so as to provide a coating on the stent graft, and the coating is located within pores or holes of the stent graft.

1186. The method of item 1121, wherein the stent graft and fibrosing agent are combined so as to provide a coating on the stent graft, and the coating is located solely within pores or holes of the stent graft.

1187. The method of item 1121, wherein the stent graft and fibrosing agent are combined so as to provide a coating on the stent graft, and the coating is located within a channel, lumen, or divet of the stent graft.

1188. The method of item 1121, wherein the stent graft is combined with a second pharmaceutically active agent.

1189. The method of item 1121, wherein the device comprises further an anti-inflammatory agent.

1190. The method of item 1121, wherein the device further comprises an agent that inhibits infection.

1191. The method of item 1121, wherein the device further comprises an anthracycline.

1192. The method of item 1121, wherein the device further comprises doxorubicin.

1193. The method of item 1121, wherein the device further comprises mitoxantrone.

1194. The method of item 1121, wherein the device further comprises a fluoropyrimidine.

1195. The method of item 1121, wherein the device further comprises 5-fluorouracil (5-FU).

1196. The method of item 1121, wherein the device further comprises a folic acid antagonist.

1197. The method of item 1121, wherein the device further comprises methotrexate.

1198. The method of item 1121, wherein the device further comprises a podophylotoxin.

1199. The method of item 1121, wherein the device further comprises etoposide.

1200. The method of item 1121 wherein the device further comprises a camptothecin.

1201. The method of item 1121, wherein the device further comprises a hydroxyurea.

1202. The method of item 1121, wherein the device further comprises a platinum complex.

1203. The method of item 1121, wherein the device further comprises cisplatin.

1204. The method of item 1121 wherein the device further comprises an anti-thrombotic agent.

1205. The method of item 1121 wherein the device further comprises a visualization agent.

1206. The method of item 1121, wherein the device further comprises a visualization agent, wherein the visualization agent is a radiopaque material, wherein the radiopaque material comprises a metal, a halogenated compound, or a barium containing compound.

1207. The method of item 1121, wherein the device further comprises a visualization agent, wherein the visualization agent is a radiopaque material, wherein the radiopaque material comprises barium, tantalum, or technetium.

1208. The method of item 1121, wherein the device further comprises a visualization agent, wherein the visualization agent is a MRI responsive material.

1209. The method of item 1121, wherein the device further comprises a visualization agent, wherein the visualization agent comprises a gadolinium chelate.

1210. The method of item 1121, wherein the device further comprises a visualization agent, wherein the visualization agent comprises iron, magnesium, manganese, copper, or chromium.

1211. The method of item 1121, wherein the device further comprises a visualization agent, wherein the visualization agent comprises an iron oxide compound.

1212. The method of item 1121, wherein the device further comprises a visualization agent, wherein the visualization agent is or comprises a dye, pigment, or colorant.

1213. The method of item 1121, wherein the device further comprises an echogenic material.

1214. The method of item 1121, wherein the device further comprises comprises an echogenic material, and the echogenic material is in the form of a coating.

1215. The method of item 1121, wherein device is sterilized.

1216. The method of item 1121, wherein the device releases the fibrosing agent into tissue in the vicinity of the device after deployment of the device in a patient.

1217. The method of item 1121, wherein the device releases fibrosing agent into tissue in the vicinity of the device after deployment of the device in a patient, wherein the fibrosing agent is released in effective concentrations from the device over a period ranging from the time of deployment of the device to about at least 1 year.

1218. The method of item 1121, wherein the device releases fibrosing agent into tissue in the vicinity of the device after deployment of the device in a patient, wherein the fibrosing agent is released in effective concentrations from the device over a period ranging from the time of deployment of the device to at least about 6 months from deployment.

1219. The method of item 1121, wherein the device releases fibrosing agent into tissue in the vicinity of the device after deployment of the device in a patient, wherein the fibrosing agent is released in effective concentrations from the device over a period ranging from the time of deployment of the device to at least about 90 days from deployment.

1220. The method of item 1121, wherein the device releases fibrosing agent into tissue in the vicinity of the device after deployment of the device in a patient, wherein the fibrosing agent is released in effective concentrations from the device at a constant rate.

1221. The method of item 1121, wherein the device releases fibrosing agent into tissue in the vicinity of the device after deployment of the device in a patient, wherein the fibrosing agent is released in effective concentrations from the device at an increasing rate.

1222. The method of item 1121, wherein the device releases fibrosing agent into tissue in the vicinity of the device after deployment of the device in a patient, wherein the fibrosing agent is released in effective concentrations from the device at a decreasing rate.

1223. The method of item 1121, wherein the device releases fibrosing agent into tissue in the vicinity of the device after deployment of the device in a patient, wherein the fibrosing agent is released in effective concentrations from the device by diffusion over a period ranging from the time of deployment of the stent graft to at least about 90 days from deployment.

1224. The method of item 1121, wherein the device releases fibrosing agent into tissue in the vicinity of the device after deployment of the device in a patient, wherein the fibrosing agent is released in effective concentrations from the device by erosion of the composition over a period ranging from the time of deployment of the stent graft to at least about 90 days from deployment.

1225. The method of item 1121, wherein the device comprises about 0.01 μg to about 10 μg of the fibrosing agent.

1226. The method of item 1121, wherein the device comprises about 10 μg to about 10 mg of the fibrosing agent.

1227. The method of item 1121, wherein the device comprises about 10 mg to about 250 mg of the fibrosing agent.

1228. The method of item 1121, wherein the device comprises about 250 mg to about 1000 mg of the fibrosing agent.

1229. The method of item 1121, wherein the device comprises about 1000 mg to about 2500 mg of the fibrosing agent.

1230. The method of item 1121, wherein a surface of the device comprises less than 0.01 μg of the fibrosing agent per mm² of device surface occupied by fibrosing agent.

1231. The method of item 1121, wherein a surface of the device comprises about 0.01 μg to about 1 μg of the fibrosing agent per mm² of device surface occupied by fibrosing agent.

1232. The method of item 1121, wherein a surface of the device comprises about 1 μg to about 10 μg of the fibrosing agent per mm² of device surface occupied by fibrosing agent.

1233. The method of item 1121, wherein a surface of the device comprises about 10μg to about 250 μg of the fibrosing agent per mm² of device surface occupied by fibrosing agent.

1234. The method of item 1121, wherein a surface of the device comprises about 250 μg to about 1000 μg of fibrosing agent per mm² of device surface occupied by fibrosing agent.

1235. The method of item 1121, wherein a surface of the device comprises about 1000 μg to about 2500 μg of the fibrosing agent per mm² of device surface occupied by fibrosing agent.

1236. The method of item 1121, wherein the stent graft is a tubular structure that comprises a lumen through which blood may flow, wherein the tubular structure comprises a luminal surface and a non-luminal surface, and wherein the agent or the composition comprising the agent is coated onto the non-luminal surface of the stent graft.

1237. The method of item 1121, wherein the stent graft is a tubular structure that comprises a lumen through which blood may flow, wherein the tubular structure comprises a luminal surface and a non-luminal surface, and wherein the agent or the composition comprising the agent is directly affixed to the non-luminal surface of the stent graft.

1238. The method of item 1121, wherein the stent graft is a tubular structure that comprises a lumen through which blood may flow, wherein the tubular structure comprises a luminal surface and a non-luminal surface, and wherein all or a portion of the non-luminal surface of the structure is covered with the fibrosing agent or the composition comprising the fibrosing agent.

1239. The method of item 1121, wherein the stent graft is a tubular structure that comprises a lumen through which blood may flow, wherein the tubular structure comprises a luminal surface and a non-luminal surface, and wherein all or a portion of the non-luminal surface of the intraluminal stent graft is coated with a proliferative agent.

1240. The method of item 1121, wherein the stent graft is a tubular structure that comprises a lumen through which blood may flow, wherein the tubular structure comprises a luminal surface and a non-luminal surface, and wherein all or a portion of the luminal surface of the structure is coated with an agent that inhibits restenosis.

1241. The method of item 1121, wherein the stent graft is a tubular structure that comprises a lumen through which blood may flow, wherein the tubular structure comprises a luminal surface and a non-luminal surface, where the method comprises attaching a thread to a non-luminal surface of the structure, wherein the thread is, or comprises, the fibrosing agent or the composition comprising the fibrosing agent.

1242. A method of adhering a stent graft to a patient, comprising inserting into a patient in need thereof a device, wherein the device comprises i) a stent graft and ii) a fibrosing agent or a composition comprising a fibrosing agent, wherein the agent induces fibrosis.

1243. The method of item 1242 wherein the fibrosing agent promotes regeneration.

1244. The method of item 1242 wherein the fibrosing agent promotes angiogenesis.

1245. The method of item 1242 wherein the fibrosing agent promotes fibroblast migration.

1246. The method of item 1242 wherein the fibrosing agent promotes fibroblast proliferation.

1247. The method of item 1242 wherein the fibrosing agent promotes deposition of extracellular matrix (ECM).

1248. The method of item 1242 wherein the fibrosing agent promotes tissue remodeling.

1249. The method of item 1242 wherein the fibrosing agent is an arterial vessel wall irritant.

1250. The method of item 1242 wherein the fibrosing agent is or comprises silk.

1251. The method of item 1242 wherein the fibrosing agent is or comprises mineral particles.

1252. The method of item 1242 wherein the fibrosing agent is or comprises chitosan.

1253. The method of item 1242 wherein the fibrosing agent is or comprises polylysine.

1254. The method of item 1242 wherein the fibrosing agent is or comprises fibronectin.

1255. The method of item 1242 wherein the fibrosing agent is or comprises bleomycin.

1256. The method of item 1242 wherein the fibrosing agent is or comprises CTGF.

1257. The method of item 1242 wherein the fibrosing agent is in the form of a thread, or is in contact with a thread.

1258. The method of item 1242 wherein the fibrosing agent is in the form of a particulate.

1259. The method of item 1242 wherein the composition further comprises an inflammatory cytokine.

1260. The method of item 1242 wherein the composition further comprises an agent that stimulates cell proliferation.

1261. The method of item 1242 wherein the composition is in the form of a gel or paste.

1262. The method of item 1242 wherein the fibrosing agent is in the form of tufts.

1263. The method of item 1242, wherein the fibrosing agent promotes adhesion between the stent graft and the patient.

1264. The method of item 1242, wherein the stent graft delivers the fibrosing agent locally to tissue proximate to the stent graft.

1265. The method of item 1242, wherein the stent graft and fibrosing agent are combined so as to provide a coating on the stent graft.

1266. The method of item 1242, wherein the stent graft and fibrosing agent are combined so as to provide a coating on the stent graft, and the coating directly contacts the stent graft.

1267. The method of item 1242, wherein the stent graft and fibrosing agent are combined so as to provide a coating on the stent graft, and the coating indirectly contacts the stent graft.

1268. The method of item 1242, wherein the stent graft and fibrosing agent are combined so as to provide a coating on the stent graft, and the coating partially covers the stent graft.

1269. The method of item 1242, wherein the stent graft and fibrosing agent are combined so as to provide a coating on the stent graft, and the coating completely covers the stent graft.

1270. The method of item 1242, wherein the stent graft and, fibrosing agent are combined so as to provide a coating on the stent graft, where the coating is a uniform coating.

1271. The method of item 1242, wherein the stent graft and fibrosing agent are combined so as to provide a coating on the stent graft, where the coating is a non-uniform coating.

1272. The method of item 1242, wherein the stent graft and fibrosing agent are combined so as to provide a coating on the stent graft, where the coating is a discontinuous coating.

1273. The method of item 1242, wherein the stent graft and fibrosing agent are combined so as to provide a coating on the stent graft, where the coating is a patterned coating.

1274. The method of item 1242, wherein the stent graft and fibrosing agent are combined so as to provide a coating on the stent graft, where the coating has a thickness of 100 μm or less.

1275. The method of item 1242, wherein the stent graft and fibrosing agent are combined so as to provide a coating on the stent graft, where the coating has a thickness of 10 μm or less.

1276. The method of item 1242, wherein the stent graft and fibrosing agent are combined so as to provide a coating on the stent graft, and the coating is stable at room temperature for a period of at least 1 year.

1277. The method of item 1242, wherein the stent graft and fibrosing agent are combined so as to provide a coating on the stent graft, and the fibrosing agent is present in the coating in an amount ranging between about 0.0001% to about 1% by weight.

1278. The method of item 1242, wherein the stent graft and fibrosing agent are combined so as to provide a coating on the stent graft, and the fibrosing agent is present in the coating in an amount ranging between about 1% to about 10% by weight.

1279. The method of item 1242, wherein the stent graft and fibrosing agent are combined so as to provide a coating on the stent graft, and the fibrosing agent is present in the coating in an amount ranging between about 10% to about 25% by weight.

1280. The method of item 1242, wherein the stent graft and fibrosing agent are combined so as to provide a coating on the stent graft, and the fibrosing agent is present in the coating in an amount ranging between about 25% to about 70% by weight.

1281. The method of item 1242, wherein the stent graft and fibrosing agent are combined so as to provide a coating on the stent graft, wherein the stent graft comprises a first coating having a first composition and a second coating having a second composition.

1282. The method of item 1242, wherein the stent graft and fibrosing agent are combined so as to provide a coating on the stent graft, wherein the coated stent graft comprises a first coating having a first composition and a second coating having a second composition, and where the first composition and the second composition are different.

1283. The method of item 1242, wherein the stent graft and fibrosing agent are combined so as to provide a coating on the stent graft, and the coating further comprises a polymer.

1284. The method of item 1242, wherein the stent graft and fibrosing agent are combined so as to provide a coating on the stent graft, and the coating further comprises a copolymer.

1285. The method of item 1242, wherein the stent graft and fibrosing agent are combined so as to provide a coating on the stent graft, and the coating further comprises a block copolymer.

1286. The method of item 1242, wherein the stent graft and fibrosing agent are combined so as to provide a coating on the stent graft, and the coating further comprises a random copolymer.

1287. The method of item 1242, wherein the stent graft and fibrosing agent are combined so as to provide a coating on the stent graft, and the coating further comprises a biodegradable polymer.

1288. The method of item 1242, wherein the stent graft and fibrosing agent are combined so as to provide a coating on the stent graft, and the coating further comprises a non-biodegradable polymer.

1289. The method of item 1242, wherein the stent graft and fibrosing agent are combined so as to provide a coating on the stent graft, and the coating further comprises a hydrophilic polymer.

1290. The method of item 1242, wherein the stent graft and fibrosing agent are combined so as to provide a coating on the stent graft, and the coating further comprises a hydrophobic polymer.

1291. The method of item 1242, wherein the stent graft and fibrosing agent are combined so as to provide a coating on the stent graft, and the coating further comprises a polymer having hydrophilic domains.

1292. The method of item 1242, wherein the stent graft and fibrosing agent are combined so as to provide a coating on the stent graft, and the coating further comprises a polymer having hydrophobic domains.

1293. The method of item 1242, wherein the stent graft and fibrosing agent are combined so as to provide a coating on the stent graft, and the coating further comprises a non-conductive polymer.

1294. The method of item 1242, wherein the stent graft and fibrosing agent are combined so as to provide a coating on the stent graft, and the coating further comprises an elastomer.

1295. The method of item 1242, wherein the stent graft and fibrosing agent are combined so as to provide a coating on the stent graft, and the coating further comprises a hydrogel.

1296. The method of item 1242, wherein the stent graft and fibrosing agent are combined so as to provide a coating on the stent graft, and the coating further comprises a silicone polymer.

1297. The method of item 1242, wherein the stent graft and fibrosing agent are combined so as to provide a coating on the stent graft, and the coating further comprises a hydrocarbon polymer.

1298. The method of item 1242, wherein the stent graft and fibrosing agent are combined so as to provide a coating on the stent graft, and the coating further comprises a styrene-derived polymer.

1299. The method of item 1242, wherein the stent graft and fibrosing agent are combined so as to provide a coating on the stent graft, and the coating further comprises a butadiene-derived polymer.

1300. The method of item 1242, wherein the stent graft and fibrosing agent are combined so as to provide a coating on the stent graft, and the coating further comprises a macromer.

1301. The method of item 1242, wherein the stent graft and fibrosing agent are combined so as to provide a coating on the stent graft, and the coating further comprises a poly(ethylene glycol)polymer.

1302. The method of item 1242, wherein the stent graft and fibrosing agent are combined so as to provide a coating on the stent graft, and the coating further comprises an amorphous polymer.

1303. The method of item 1242, wherein the stent graft and fibrosing agent are combined so as to provide a coating on the stent graft, and the coating is a lubricious coating.

1304. The method of item 1242, wherein the stent graft and fibrosing agent are combined so as to provide a coating on the stent graft, and the coating is located within pores or holes of the stent graft.

1305. The method of item 1242, wherein the stent graft and fibrosing agent are combined so as to provide a coating on the stent graft, and the coating is located solely within pores or holes of the stent graft.

1306. The method of item 1242, wherein the stent graft and fibrosing agent are combined so as to provide a coating on the stent graft, and the coating is located within a channel, lumen, or divet of the stent graft.

1307. The method of item 1242, wherein the stent graft is combined with a second pharmaceutically active agent.

1308. The method of item 1242, wherein the device comprises further an anti-inflammatory agent.

1309. The method of item 1242, wherein the device further comprises an agent that inhibits infection.

1310. The method of item 1242, wherein the device further comprises an anthracycline.

1311. The method of item 1242, wherein the device further comprises doxorubicin.

1312. The method of item 1242, wherein the device further comprises mitoxantrone.

1313. The method of item 1242, wherein the device further comprises a fluoropyrimidine.

1314. The method of item 1242, wherein the device further comprises 5-fluorouracil (5-FU).

1315. The method of item 1242, wherein the device further comprises a folic acid antagonist.

1316. The method of item 1242, wherein the device further comprises methotrexate.

1317. The method of item 1242, wherein the device further comprises a podophylotoxin.

1318. The method of item 1242, wherein the device further comprises etoposide.

1319. The method of item 1242 wherein the device further comprises a camptothecin.

1320. The method of item 1242, wherein the device further comprises a hydroxyurea.

1321. The method of item 1242, wherein the device further comprises a platinum complex.

1322. The method of item 1242, wherein the device further comprises cisplatin.

1323. The method of item 1242 wherein the device further comprises an anti-thrombotic agent.

1324. The method of item 1242 wherein the device further comprises a visualization agent.

1325. The method of item 1242, wherein the device further comprises a visualization agent, wherein the visualization agent is a radiopaque material, wherein the radiopaque material comprises a metal, a halogenated compound, or a barium containing compound.

1326. The method of item 1242, wherein the device further comprises a visualization agent, wherein the visualization agent is a radiopaque material, wherein the radiopaque material comprises barium, tantalum, or technetium.

1327. The method of item 1242, wherein the device further comprises a visualization agent, wherein the visualization agent is a MRI responsive material.

1328. The method of item 1242, wherein the device further comprises a visualization agent, wherein the visualization agent comprises a gadolinium chelate.

1329. The method of item 1242, wherein the device further comprises a visualization agent, wherein the visualization agent comprises iron, magnesium, manganese, copper, or chromium.

1330. The method of item 1242, wherein the device further comprises a visualization agent, wherein the visualization agent comprises an iron oxide compound.

1331. The method of item 1242, wherein the device further comprises a visualization agent, wherein the visualization agent is or comprises a dye, pigment, or colorant.

1332. The method of item 1242, wherein the device further comprises an echogenic material.

1333. The method of item 1242, wherein the device further comprises comprises an echogenic material, and the echogenic material is in the form of a coating.

1334. The method of item 1242, wherein device is sterilized.

1335. The method of item 1242, wherein the device releases the fibrosing agent into tissue in the vicinity of the device after deployment of the device in a patient.

1336. The method of item 1242, wherein the device releases fibrosing agent into tissue in the vicinity of the device after deployment of the device in a patient, wherein the fibrosing agent is released in effective concentrations from the device over a period ranging from the time of deployment of the device to about at least 1 year.

1337. The method of item 1242, wherein the device releases fibrosing agent into tissue in the vicinity of the device after deployment of the device in a patient, wherein the fibrosing agent is released in effective concentrations from the device over a period ranging from the time of deployment of the device to at least about 6 months from deployment.

1338. The method of item 1242, wherein the device releases fibrosing agent into tissue in the vicinity of the device after deployment of the device in a patient, wherein the fibrosing agent is released in effective concentrations from the device over a period ranging from the time of deployment of the device to at least about 90 days from deployment.

1339. The method of item 1242, wherein the device releases fibrosing agent into tissue in the vicinity of the device after deployment of the device in a patient, wherein the fibrosing agent is released in effective concentrations from the device at a constant rate.

1340. The method of item 1242, wherein the device releases fibrosing agent into tissue in the vicinity of the device after deployment of the device in a patient, wherein the fibrosing agent is released in effective concentrations from the device at an increasing rate.

1341. The method of item 1242, wherein the device releases fibrosing agent into tissue in the vicinity of the device after deployment of the device in a patient, wherein the fibrosing agent is released in effective concentrations from the device at a decreasing rate.

1342. The method of item 1242, wherein the device releases fibrosing agent into tissue in the vicinity of the device after deployment of the device in a patient, wherein the fibrosing agent is released in effective concentrations from the device by diffusion over a period ranging from the time of deployment of the stent graft to at least about 90 days from deployment.

1343. The method of item 1242, wherein the device releases fibrosing agent into tissue in the vicinity of the device after deployment of the device in a patient, wherein the fibrosing agent is released in effective concentrations from the device by erosion of the composition over a period ranging from the time of deployment of the stent graft to at least about 90 days from deployment.

1344. The method of item 1242, wherein the device comprises about 0.01 μg to about 10 μg of the fibrosing agent.

1345. The method of item 1242, wherein the device comprises about 10 μg to about 10 mg of the fibrosing agent.

1346. The method of item 1242, wherein the device comprises about 10 mg to about 250 mg of the fibrosing agent.

1347. The method of item 1242, wherein the device comprises about 250 mg to about 1000 mg of the fibrosing agent.

1348. The method of item 1242, wherein the device comprises about 1000 mg to about 2500 mg of the fibrosing agent.

1349. The method of item 1242, wherein a surface of the device comprises less than 0.01 μg of the fibrosing agent per mm² of device surface occupied by fibrosing agent.

1350. The method of item 1242, wherein a surface of the device comprises about 0.01 μg to about 1 μg of the fibrosing agent per mm² of device surface occupied by fibrosing agent.

1351. The method of item 1242, wherein a surface of the device comprises about 1 μg to about 10 μg of the fibrosing agent per mm² of device surface occupied by fibrosing agent.

1352. The method of item 1242, wherein a surface of the device comprises about 10 μg to about 250 μg of the fibrosing agent per mm² of device surface occupied by fibrosing agent.

1353. The method of item 1242, wherein a surface of the device comprises about 250 μg to about 1000 μg of fibrosing agent per mm² of device surface occupied by fibrosing agent.

1354. The method of item 1242, wherein a surface of the device comprises about 1000 μg to about 2500 μg of the fibrosing agent per mm² of device surface occupied by fibrosing agent.

1355. The method of item 1242, wherein the stent graft is a tubular structure that comprises a lumen through which blood may flow, wherein the tubular structure comprises a luminal surface and a non-luminal surface, and wherein the agent or the composition comprising the agent is coated onto the non-luminal surface of the stent graft.

1356. The method of item 1242, wherein the stent graft is a tubular structure that comprises a lumen through which blood may flow, wherein the tubular structure comprises a luminal surface and a non-luminal surface, and wherein the agent or the composition comprising the agent is directly affixed to the non-luminal surface of the stent graft.

1357. The method of item 1242, wherein the stent graft is a tubular structure that comprises a lumen through which blood may flow, wherein the tubular structure comprises a luminal surface and a non-luminal surface, and wherein all or a portion of the non-luminal surface of the structure is covered with the fibrosing agent or the composition comprising the fibrosing agent.

1358. The method of item 1242, wherein the stent graft is a tubular structure that comprises a lumen through which blood may flow, wherein the tubular structure comprises a luminal surface and a non-luminal surface, and wherein all or a portion of the non-luminal surface of the intraluminal stent graft is coated with a proliferative agent.

1359. The method of item 1242, wherein the stent graft is a tubular structure that comprises a lumen through which blood may flow, wherein the tubular structure comprises a luminal surface and a non-luminal surface, and wherein all or a portion of the luminal surface of the structure is coated with an agent that inhibits restenosis.

1360. The method of item 1242, wherein the stent graft is a tubular structure that comprises a lumen through which blood may flow, wherein the tubular structure comprises a luminal surface and a non-luminal surface, where the method comprises attaching a thread to a non-luminal surface of the structure, wherein the thread is, or comprises, the fibrosing agent or the composition comprising the fibrosing agent.

1361. A method for reducing perigraft leakage associated with stent graft delivery in a patient, comprising delivering a device to a patient in need thereof, wherein the device comprises i) a stent graft and ii) a fibrosing agent or a composition comprising a fibrosing agent, wherein the agent induces fibrosis.

1362. The method of item 1361 wherein the fibrosing agent promotes regeneration.

1363. The method of item 1361 wherein the fibrosing agent promotes angiogenesis.

1364. The method of item 1361 wherein the fibrosing agent promotes fibroblast migration.

1365. The method of item 1361 wherein the fibrosing agent promotes fibroblast proliferation.

1366. The method of item 1361 wherein the fibrosing agent promotes deposition of extracellular matrix (ECM).

1367. The method of item 1361 wherein the fibrosing agent promotes tissue remodeling.

1368. The method of item 1361 wherein the fibrosing agent is an arterial vessel wall irritant.

1369. The method of item 1361 wherein the fibrosing agent is or comprises silk.

1370. The method of item 1361 wherein the fibrosing agent is or comprises mineral particles.

1371. The method of item 1361 wherein the fibrosing agent is or comprises chitosan.

1372. The method of item 1361 wherein the fibrosing agent is or comprises polylysine.

1373. The method of item 1361. wherein the fibrosing agent is or comprises fibronectin.

1374. The method of item 1361 wherein the fibrosing agent is or comprises bleomycin.

1375. The method of item 1361 wherein the fibrosing agent is or comprises CTGF.

1376. The method of item 1361 wherein the fibrosing agent is in the form of a thread, or is in contact with a thread.

1377. The method of item 1361 wherein the fibrosing agent is in the form of a particulate.

1378. The method of item 1361 wherein the composition further comprises an inflammatory cytokine.

1379. The method of item 1361 wherein the composition further comprises an agent that stimulates cell proliferation.

1380. The method of item 1361 wherein the composition is in the form of a gel or paste.

1381. The method of item 1361 wherein the fibrosing agent is in the form of tufts.

1382. The method of item 1361, wherein the fibrosing agent promotes adhesion between the stent graft and the patient.

1383. The method of item 1361, wherein the stent graft delivers the fibrosing agent locally to tissue proximate to the stent graft.

1384. The method of item 1361, wherein the stent graft and fibrosing agent are combined so as to provide a coating on the stent graft.

1385. The method of item 1361, wherein the stent graft and fibrosing agent are combined so as to provide a coating on the stent graft, and the coating directly contacts the stent graft.

1386. The method of item 1361, wherein the stent graft and fibrosing agent are combined so as to provide a coating on the stent graft, and the coating indirectly contacts the stent graft.

1387. The method of item 1361, wherein the stent graft and fibrosing agent are combined so as to provide a coating on the stent graft, and the coating partially covers the stent graft.

1388. The method of item 1361, wherein the stent graft and fibrosing agent are combined so as to provide a coating on the stent graft, and the coating completely covers the stent graft.

1389. The method of item 1361, wherein the stent graft and fibrosing agent are combined so as to provide a coating on the stent graft, where the coating is a uniform coating.

1390. The method of item 1361, wherein the stent graft and fibrosing agent are combined so as to provide a coating on the stent graft, where the coating is a non-uniform coating.

1391. The method of item 1361, wherein the stent graft and fibrosing agent are combined so as to provide a coating on the stent graft, where the coating is a discontinuous coating.

1392. The method of item 1361, wherein the stent graft and fibrosing agent are combined so as to provide a coating on the stent graft, where the coating is a patterned coating.

1393. The method of item 1361, wherein the stent graft and fibrosing agent are combined so as to provide a coating on the stent graft, where the coating has a thickness of 100 μm or less.

1394. The method of item 1361, wherein the stent graft and fibrosing agent are combined so as to provide a coating on the stent graft, where the coating has a thickness of 10 μm or less.

1395. The method of item 1361, wherein the stent graft and fibrosing agent are combined so as to provide a coating on the stent graft, and the coating is stable at room temperature for a period of at least 1 year.

1396. The method of item. 1361, wherein the stent graft and fibrosing agent are combined so as to provide a coating on the stent graft, and the fibrosing agent is present in the coating in an amount ranging between about 0.0001% to about 1% by weight.

1397. The method of item 1361, wherein the stent graft and fibrosing agent are combined so as to provide a coating on the stent graft, and the fibrosing agent is present in the coating in an amount ranging between about 1% to about 10% by weight.

1398. The method of item 1361, wherein the stent graft and fibrosing agent are combined so as to provide a coating on the stent graft, and the fibrosing agent is present in the coating in an amount ranging between about 10% to about 25% by weight.

1399. The method of item 1361, wherein the stent graft and fibrosing agent are combined so as to provide a coating on the stent graft, and the fibrosing agent is present in the coating in an amount ranging between about 25% to about 70% by weight.

1400. The method of item 1361, wherein the stent graft and fibrosing agent are combined so as to provide a coating on the stent graft, wherein the stent graft comprises a first coating having a first composition and a second coating having a second composition.

1401. The method of item 1361, wherein the stent graft and fibrosing agent are combined so as to provide a coating on the stent graft, wherein the coated stent graft comprises a first coating having a first composition and a second coating having a second composition, and where the first composition and the second composition are different.

1402. The method of item 1361, wherein the stent graft and fibrosing agent are combined so as to provide a coating on the stent graft, and the coating further comprises a polymer.

1403. The method of item 1361, wherein the stent graft and fibrosing agent are combined so as to provide a coating on the stent graft, and the coating further comprises a copolymer.

1404. The method of item 1361, wherein the stent graft and fibrosing agent are combined so as to provide a coating on the stent graft, and the coating further comprises a block copolymer.

1405. The method of item 1361, wherein the stent graft and fibrosing agent are combined so as to provide a coating on the stent graft, and the coating further comprises a random copolymer.

1406. The method of item 1361, wherein the stent graft and fibrosing agent are combined so as to provide a coating on the stent graft, and the coating further comprises a biodegradable polymer.

1407. The method of item 1361, wherein the stent graft and fibrosing agent are combined so as to provide a coating on the stent graft, and the coating further comprises a non-biodegradable polymer.

1408. The method of item 1361, wherein the stent graft and fibrosing agent are combined so as to provide a coating on the stent graft, and the coating further comprises a hydrophilic polymer.

1409. The method of item 1361, wherein the stent graft and fibrosing agent are combined so as to provide a coating on the stent graft, and the coating further comprises a hydrophobic polymer.

1410. The method of item 1361, wherein the stent graft and fibrosing agent are combined so as to provide a coating on the stent graft, and the coating further comprises a polymer having hydrophilic domains.

1411. The method of item 1361, wherein the stent graft and fibrosing agent are combined so as to provide a coating on the stent graft, and the coating further comprises a polymer having hydrophobic domains.

1412. The method of item 1361, wherein the stent graft and fibrosing agent are combined so as to provide a coating on the stent graft, and the coating further comprises a non-conductive polymer.

1413. The method of item 1361, wherein the stent graft and fibrosing agent are combined so as to provide a coating on the stent graft, and the coating further comprises an elastomer.

1414. The method of item 1361, wherein the stent graft and fibrosing agent are combined so as to provide a coating on the stent graft, and the coating further comprises a hydrogel.

1415. The method of item 1361, wherein the stent graft and fibrosing agent are combined so as to provide a coating on the stent graft, and the coating further comprises a silicone polymer.

1416. The method of item 1361, wherein the stent graft and fibrosing agent are combined so as to provide a coating on the stent graft, and the coating further comprises a hydrocarbon polymer.

1417. The method of item 1361, wherein the stent graft and fibrosing agent are combined so as to provide a coating on the stent graft, and the coating further comprises a styrene-derived polymer.

1418. The method of item 1361, wherein the stent graft and fibrosing agent are combined so as to provide a coating on the stent graft, and the coating further comprises a butadiene-derived polymer.

1419. The method of item 1361, wherein the stent graft and fibrosing agent are combined so as to provide a coating on the stent graft, and the coating further comprises a macromer.

1420. The method of item 1361, wherein the stent graft and fibrosing agent are combined so as to provide a coating on the stent graft, and the coating further comprises a poly(ethylene glycol)polymer.

1421. The method of item 1361, wherein the stent graft and fibrosing agent are combined so as to provide a coating on the stent graft, and the coating further comprises an amorphous polymer.

1422. The method of item 1361, wherein the stent graft and fibrosing agent are combined so as to provide a coating on the stent graft, and the coating is a lubricious coating.

1423. The method of item 1361, wherein the stent graft and fibrosing agent are combined so as to provide a coating on the stent graft, and the coating is located within pores or holes of the stent graft.

1424. The method of item 1361, wherein the stent graft and fibrosing agent are combined so as to provide a coating on the stent graft, and the coating is located solely within pores or holes of the stent graft.

1425. The method of item 1361, wherein the stent graft and fibrosing agent are combined so as to provide a coating on the stent graft, and the coating is located within a channel, lumen, or divet of the stent graft.

1426. The method of item 1361, wherein the stent graft is combined with a second pharmaceutically active agent.

1427. The method of item 1361, wherein the device comprises further an anti-inflammatory agent.

1428. The method of item 1361, wherein the device further comprises an agent that inhibits infection.

1429. The method of item 1361, wherein the device further comprises an anthracycline.

1430. The method of item 1361, wherein the device further comprises doxorubicin.

1431. The method of item 1361, wherein the device further comprises mitoxantrone.

1432. The method of item 1361, wherein the device further comprises a fluoropyrimidine.

1433. The method of item 1361, wherein the device further comprises 5-fluorouracil (5-FU).

1434. The method of item 1361, wherein the device further comprises a folic acid antagonist.

1435. The method of item 1361, wherein the device further comprises methotrexate.

1436. The method of item 1361, wherein the device further comprises a podophylotoxin.

1437. The method of item 1361, wherein the device further comprises etoposide.

1438. The method of item 1361 wherein the device further comprises a camptothecin.

1439. The method of item 1361, wherein the device further comprises a hydroxyurea.

1440. The method of item 1361, wherein the device further comprises a platinum complex.

1441. The method of item 1361, wherein the device further comprises cisplatin.

1442. The method of item 1361 wherein the device further comprises an anti-thrombotic agent.

1443. The method of item 1361 wherein the device further comprises a visualization agent.

1444. The method of item 1361, wherein the device further comprises a visualization agent, wherein the visualization agent is a radiopaque material, wherein the radiopaque material comprises a metal, a halogenated compound, or a barium containing compound.

1445. The method of item 1361, wherein the device further comprises a visualization agent, wherein the visualization agent is a radiopaque material, wherein the radiopaque material comprises barium, tantalum, or technetium.

1446. The method of item 1361, wherein the device further comprises a visualization agent, wherein the visualization agent is a MRI responsive material.

1447. The method of item 1361, wherein the device further comprises a visualization agent, wherein the visualization agent comprises a gadolinium chelate.

1448. The method of item 1361, wherein the device further comprises a visualization agent, wherein the visualization agent comprises iron, magnesium, manganese, copper, or chromium.

1449. The method of item 1361, wherein the device further comprises a visualization agent, wherein the visualization agent comprises an iron oxide compound.

1450. The method of item 1361, wherein the device further comprises a visualization agent, wherein the visualization agent is or comprises a dye, pigment, or colorant.

1451. The method of item 1361, wherein the device further comprises an echogenic material.

1452. The method of item 1361, wherein the device further comprises comprises an echogenic material, and the echogenic material is in the form of a coating.

1453. The method of item 1361, wherein device is sterilized. 1454. The method of item 1361, wherein the device releases the fibrosing agent into tissue in the vicinity of the device after deployment of the device in a patient.

1455. The method of item 1361, wherein the device releases fibrosing agent into tissue in the vicinity of the device after deployment of the device in a patient, wherein the fibrosing agent is released in effective concentrations from the device over a period ranging from the time of deployment of the device to about at least 1 year.

1456. The method of item 1361, wherein the device releases fibrosing agent into tissue in the vicinity of the device after deployment of the device in a patient, wherein the fibrosing agent is released in effective concentrations from the device over a period ranging from the time of deployment of the device to at least about 6 months from deployment.

1457. The method of item 1361, wherein the device releases fibrosing agent into tissue in the vicinity of the device after deployment of the device in a patient, wherein the fibrosing agent is released in effective concentrations from the device over a period ranging from the time of deployment of the device to at least about 90 days from deployment.

1458. The method of item 1361, wherein the device releases fibrosing agent into tissue in the vicinity of the device after deployment of the device in a patient, wherein the fibrosing agent is released in effective concentrations from the device at a constant rate.

1459. The method of item 1361, wherein the device releases fibrosing agent into tissue in the vicinity of the device after deployment of the device in a patient, wherein the fibrosing agent is released in effective concentrations from the device at an increasing rate.

1460. The method of item 1361, wherein the device releases fibrosing agent into tissue in the vicinity of the device after deployment of the device in a patient, wherein the fibrosing agent is released in effective concentrations from the device at a decreasing rate.

1461. The method of item 1361, wherein the device releases fibrosing agent into tissue in the vicinity of the device after deployment of the device in a patient, wherein the fibrosing agent is released in effective concentrations from the device by diffusion over a period ranging from the time of deployment of the stent graft to at least about 90 days from deployment.

1462. The method of item 1361, wherein the device releases fibrosing agent into tissue in the vicinity of the device after deployment of the device in a patient, wherein the fibrosing agent is released in effective concentrations from the device by erosion of the composition over a period ranging from the time of deployment of the stent graft to at least about 90 days from deployment.

1463. The method of item 1361, wherein the device comprises about 0.01 μg to about 10 μg of the fibrosing agent.

1464. The method of item 1361, wherein the device comprises about 10 μg to about 10 mg of the fibrosing agent.

1465. The method of item 1361, wherein the device comprises about 10 mg to about 250 mg of the fibrosing agent.

1466. The method of item 1361, wherein the device comprises about 250 mg to about 1000 mg of the fibrosing agent.

1467. The method of item 1361, wherein the device comprises about 1000 mg to about 2500 mg of the fibrosing agent.

1468. The method of item 1361, wherein a surface of the device comprises less than 0.01 μg of the fibrosing agent per mm² of device surface occupied by fibrosing agent.

1469. The method of item 1361, wherein a surface of the device comprises about 0.01 μg to about 1 μg of the fibrosing agent per mm² of device surface occupied by fibrosing agent.

1470. The method of item 1361, wherein a surface of the device comprises about 1 μg to about 10 μg of the fibrosing agent per mm² of device surface occupied by fibrosing agent.

1471. The method of item 1361, wherein a surface of the device comprises about 10 μg to about 250 μg of the fibrosing agent per mm² of device surface occupied by fibrosing agent.

1472. The method of item 1361, wherein a surface of the device comprises about 250 μg to about 1000 μg of fibrosing agent per mm² of device surface occupied by fibrosing agent.

1473. The method of item 1361, wherein a surface of the device comprises about 1000 μg to about 2500 μg of the fibrosing agent per mm² of device surface occupied by fibrosing agent.

1474. The method of item 1361, wherein the stent graft is a tubular structure that comprises a lumen through which blood may flow, wherein the tubular structure comprises a luminal surface and a non-luminal surface, and wherein the agent or the composition comprising the agent is coated onto the non-luminal surface of the stent graft.

1475. The method of item 1361, wherein the stent graft is a tubular structure that comprises a lumen through which blood may flow, wherein the tubular structure comprises a luminal surface and a non-luminal surface, and wherein the agent or the composition comprising the agent is directly affixed to the non-luminal surface of the stent graft.

1476. The method of item 1361, wherein the stent graft is a tubular structure that comprises a lumen through which blood may flow, wherein the tubular structure comprises a luminal surface and a non-luminal surface, and wherein all or a portion of the non-luminal surface of the structure is covered with the fibrosing agent or the composition comprising the fibrosing agent.

1477. The method of item 1361, wherein the stent graft is a tubular structure that comprises a lumen through which blood may flow, wherein the tubular structure comprises a luminal surface and a non-luminal surface, and wherein all or a portion of the non-luminal surface of the intraluminal stent graft is coated with a proliferative agent.

1478. The method of item 1361, wherein the stent graft is a tubular structure that comprises a lumen through which blood may flow, wherein the tubular structure comprises a luminal surface and a non-luminal surface, and wherein all or a portion of the luminal surface of the structure is coated with an agent that inhibits restenosis.

1479. The method of item 1361, wherein the stent graft is a tubular structure that comprises a lumen through which blood may flow, wherein the tubular structure comprises a luminal surface and a non-luminal surface, where the method comprises attaching a thread to a non-luminal surface of the structure, wherein the thread is, or comprises, the fibrosing agent or the composition comprising the fibrosing agent.

1480. A method for treating a patient having an aneurysm, comprising:
delivering into the aneurysm a fibrosing agent or a composition comprising a fibrosing agent; and
delivering into the patient a stent graft.

1481. The method of item 1480 wherein the fibrosing agent promotes regeneration.

1482. The method of item 1480 wherein the fibrosing agent promotes angiogenesis.

1483. The method of item 1480 wherein the fibrosing agent promotes fibroblast migration.

1484. The method of item 1480 wherein the fibrosing agent promotes fibroblast proliferation.

1485. The method of item 1480 wherein the fibrosing agent promotes deposition of extracellular matrix (ECM).

1486. The method of item 1480 wherein the fibrosing agent promotes tissue remodeling.

1487. The method of item 1480 wherein the fibrosing agent is an arterial vessel wall irritant.

1488. The method of item 1480 wherein the fibrosing agent is or comprises silk.

1489. The method of item 1480 wherein the fibrosing agent is or comprises mineral particles.

1490. The method of item 1480 wherein the fibrosing agent is or comprises chitosan.

1491. The method of item 1480 wherein the fibrosing agent is or comprises polylysine.

1492. The method of item 1480 wherein the fibrosing agent is or comprises fibronectin.

1493. The method of item 1480 wherein the fibrosing agent is or comprises bleomycin.

1494. The method of item 1480 wherein the fibrosing agent is or comprises CTGF.

1495. The method of item 1480 wherein the fibrosing agent is in the form of a thread, or is in contact with a thread.

1496. The method of item 1480 wherein the fibrosing agent is in the form of a particulate.

1497. The method of item 1480 wherein the composition further comprises an inflammatory cytokine.

1498. The method of item 1480 wherein the composition further comprises an agent that stimulates cell proliferation.

1499. The method of item 1480 wherein the composition is in the form of a gel or paste.

1500. The method of item 1480 wherein the fibrosing agent is in the form of tufts.

1501. The method of item 1480, wherein the stent graft comprises a fibrosing agent, and the fibrosing agent promotes adhesion between the stent graft and the patient.

1502. The method of item 1480, wherein the stent graft comprises a fibrosing agent, and the stent graft delivers the fibrosing agent locally to tissue proximate to the stent graft.

1503. The method of item 1480, wherein the stent graft is in contact with a coating that comprises a fibrosing agent.

1504. The method of item 1480, wherein the stent graft is in contact with a coating that comprises a fibrosing agent, where the coating directly contacts the stent graft.

1505. The method of item 1480, wherein the stent graft is in contact with a coating that comprises a fibrosing agent, where the coating indirectly contacts the stent graft.

1506. The method of item 1480, wherein the stent graft is in contact with a coating that comprises a fibrosing agent, where the coating partially covers the stent graft.

1507. The method of item 1480, wherein the stent graft is in contact with a coating that comprises a fibrosing agent, where the coating completely covers the stent graft.

1508. The method of item 1480, wherein the stent graft is in contact with a coating that comprises a fibrosing agent, where the coating is a uniform coating.

1509. The method of item 1480, wherein the stent graft is in contact with a coating that comprises a fibrosing agent, where the coating is a non-uniform coating.

1510. The method of item 1480, wherein the stent graft is in contact with a coating that comprises a fibrosing agent, where the coating is a discontinuous coating.

1511. The method of item 1480, wherein the stent graft is in contact with a coating that comprises a fibrosing agent, where the coating is a patterned coating.

1512. The method of item 1480, wherein the stent-graft is in contact with a coating that comprises a fibrosing agent, where the coating has a thickness of 100 μm or less.

1513. The method of item 1480, wherein the stent graft is in contact with a coating that comprises a fibrosing agent, where the coating has a thickness of 10 μm or less.

1514. The method of item 1480, wherein the stent graft is in contact with a coating that comprises a fibrosing agent, where the coating is stable at room temperature for a period of at least 1 year.

1515. The method of item 1480, wherein the stent graft is in contact with a coating that comprises a fibrosing agent, and the fibrosing agent is present in the coating in an amount ranging between about 0.0001% to about 1% by weight.

1516. The method of item 1480, wherein the stent graft is in contact with a coating that comprises a fibrosing agent, and the fibrosing agent is present in the coating in an amount ranging between about 1% to about 10% by weight.

1517. The method of item 1480, wherein the stent graft is in contact with a coating that comprises a fibrosing agent, and the fibrosing agent is present in the coating in an amount ranging between about 10% to about 25% by weight.

1518. The method of item 1480, wherein the stent graft is in contact with a coating that comprises a fibrosing agent, and the fibrosing agent is present in the coating in an amount ranging between about 25% to about 70% by weight.

1519. The method of item 1480, wherein the stent graft is in contact with a coating that comprises a fibrosing agent, wherein the stent graft comprises a first coating having a first composition and a second coating having a second composition.

1520. The method of item 1480, wherein the stent graft is in contact with a coating that comprises a fibrosing agent, wherein the stent graft comprises a first coating having a first composition and a second coating having a second composition, and where the first composition and the second composition are different.

1521. The method of item 1480, wherein the stent graft is in contact with a coating that comprises a fibrosing agent, and the coating further comprises a polymer.

1522. The method of item 1480, wherein the stent graft is in contact with a coating that comprises a fibrosing agent, and the coating further comprises a copolymer.

1523. The method of item 1480, wherein the stent graft is in contact with a coating that comprises a fibrosing agent, and the coating further comprises a block copolymer.

1524. The method of item 1480, wherein the stent graft is in contact with a coating that comprises a fibrosing agent, and the coating further comprises a random copolymer.

1525. The method of item 1480, wherein the stent graft is in contact with a coating that comprises a fibrosing agent, and the coating further comprises a biodegradable polymer.

1526. The method of item 1480, wherein the stent graft is in contact with a coating that comprises a fibrosing agent, and the coating further comprises a non-biodegradable polymer.

1527. The method of item 1480, wherein the stent graft is in contact with a coating that comprises a fibrosing agent, and the coating further comprises a hydrophilic polymer.

1528. The method of item 1480, wherein the stent graft is in contact with a coating that comprises a fibrosing agent, and the coating further comprises a hydrophobic polymer.

1529. The method of item 1480, wherein the stent graft is in contact with a coating that comprises a fibrosing agent, and the coating further comprises a polymer having hydrophilic domains.

1530. The method of item 1480, wherein the stent graft is in contact with a coating that comprises a fibrosing agent, and the coating further comprises a polymer having hydrophobic domains.

1531. The method of item 1480, wherein the stent graft is in contact with a coating that comprises a fibrosing agent, and the coating further comprises a non-conductive polymer.

1532. The method of item 1480, wherein the stent graft is in contact with a coating that comprises a fibrosing agent, and the coating further comprises an elastomer.

1533. The method of item 1480, wherein the stent graft is in contact with a coating that comprises a fibrosing agent, and the coating further comprises a hydrogel.

1534. The method of item 1480, wherein the stent graft is in contact with a coating that comprises a fibrosing agent, and the coating further comprises a silicone polymer.

1535. The method of item 1480, wherein the stent graft is in contact with a coating that comprises a fibrosing agent, and the coating further comprises a hydrocarbon polymer.

1536. The method of item 1480, wherein the stent graft is in contact with a coating that comprises a fibrosing agent, and the coating further comprises a styrene-derived polymer.

1537. The method of item 1480, wherein the stent graft is in contact with a coating that comprises a fibrosing agent, and the coating further comprises a butadiene-derived polymer.

1538. The method of item 1480, wherein the stent graft is in contact with a coating that comprises a fibrosing agent, and the coating further comprises a macromer.

1539. The method of item 1480, wherein the stent graft is in contact with a coating that comprises a fibrosing agent, and the coating further comprises a poly(ethylene glycol)polymer.

1540. The method of item 1480, wherein the stent graft is in contact with a coating that comprises a fibrosing agent, and the coating further comprises an amorphous polymer.

1541. The method of item 1480, wherein the stent graft is in contact with a coating that comprises a fibrosing agent, and the coating is a lubricious coating.

1542. The method of item 1480, wherein the stent graft is in contact with a coating that comprises a fibrosing agent, and the coating is located within pores or holes of the stent graft.

1543. The method of item 1480, wherein the stent graft is in contact with a coating that comprises a fibrosing agent, and the coating is located solely within pores or holes of the stent graft.

1544. The method of item 1480, wherein the stent graft is in contact with a coating that comprises a fibrosing agent, and the coating is located within a channel, lumen, or divet of the stent graft.

1545. The method of item 1480, wherein the stent graft is in contact with a second pharmaceutically active agent.

1546. The method of item 1480, wherein the stent graft is in contact with an anti-inflammatory agent.

1547. The method of item 1480, wherein the stent graft is in contact with an agent that inhibits infection.

1548. The method of item 1480, wherein the stent graft is in contact with an anthracycline.

1549. The method of item 1480, wherein the stent graft is in contact with doxorubicin.

1550. The method of item 1480, wherein the stent graft is in contact with mitoxantrone.

1551. The method of item 1480, wherein the stent graft is in contact with a fluoropyrimidine.

1552. The method of item 1480, wherein the stent graft is in contact with 5-fluorouracil (5-FU).

1553. The method of item 1480, wherein the stent graft is in contact with a folic acid antagonist.

1554. The method of item 1480, wherein the stent graft is in contact with methotrexate.

1555. The method of item 1480, wherein the stent graft is in contact with a podophylotoxin.

1556. The method of item 1480, wherein the stent graft is in contact with etoposide.

1557. The method of item 1480 wherein the stent graft is in contact with camptothecin.

1558. The method of item 1480, wherein the stent graft is in contact with a hydroxyurea.

1559. The method of item 1480, wherein the stent graft is in contact with a platinum complex.

1560. The method of item 1480, wherein the stent graft is in contact with cisplatin.

1561. The method of item 1480, wherein the stent graft is in contact with an anti-thrombotic agent.

1562. The method of item 1480, wherein the stent graft is in contact with a visualization agent.

1563. The method of item 1480, wherein the stent graft is in contact with a fibrosing agent and a second pharmaceutically active agent.

1564. The method of item 1480, wherein the stent graft is in contact with a fibrosing agent and an anti-inflammatory agent.

1565. The method of item 1480, wherein the stent graft is in contact with a fibrosing agent and an agent that inhibits infection.

1566. The method of item 1480, wherein the stent graft is in contact with a fibrosing agent and an anthracycline.

1567. The method of item 1480, wherein the stent graft is in contact with a fibrosing agent and doxorubicin.

1568. The method of item 1480, wherein the stent graft is in contact with a fibrosing agent and mitoxantrone.

1569. The method of item 1480, wherein the stent graft is in contact with a fibrosing agent and a fluoropyrimidine.

1570. The method of item 1480, wherein the stent graft is in contact with a fibrosing agent and 5-fluorouracil (5-FU).

1571. The method of item 1480, wherein the stent graft is in contact with a fibrosing agent and a folic acid antagonist.

1572. The method of item 1480, wherein the stent graft is in contact with a fibrosing agent and methotrexate.

1573. The method of item 1480, wherein the stent graft is in contact with a fibrosing agent and a podophylotoxin.

1574. The method of item 1480, wherein the stent graft is in contact with a fibrosing agent and etoposide.

1575. The method of item 1480 wherein the stent graft is in contact with a fibrosing agent and camptothecin.

1576. The method of item 1480, wherein the stent graft is in contact with a fibrosing agent and a hydroxyurea.

1577. The method of item 1480, wherein the stent graft is in contact with a fibrosing agent and a platinum complex.

1578. The method of item 1480, wherein the stent graft is in contact with a fibrosing agent and cisplatin.

1579. The method of item 1480, wherein the stent graft is in contact with a fibrosing agent and an anti-thrombotic agent.

1580. The method of item 1480, wherein the stent graft is in contact with a fibrosing agent and a visualization agent.

1581. The method of item 1480, wherein the stent graft is in contact with a visualization agent, wherein the visualization agent is a radiopaque material, wherein the radiopaque material comprises a metal, a halogenated compound, or a barium containing compound.

1582. The method of item 1480, wherein the stent graft is in contact with a visualization agent, wherein the visualization agent is a radiopaque material, wherein the radiopaque material comprises barium, tantalum, or technetium.

1583. The method of item 1480, wherein the stent graft is in contact with a visualization agent, wherein the visualization agent is a MRI responsive material.

1584. The method of item 1480, wherein the stent graft is in contact with a visualization agent, wherein the visualization agent comprises a gadolinium chelate.

1585. The method of item 1480, wherein the stent graft is in contact with a visualization agent, wherein the visualization agent comprises iron, magnesium, manganese, copper, or chromium.

1586. The method of item 1480, wherein the stent graft is in contact with a visualization agent, wherein the visualization agent comprises an iron oxide compound.

1587. The method of item 1480, wherein the stent graft is in contact with a visualization agent, wherein the visualization agent is or comprises a dye, pigment, or colorant.

1588. The method of item 1480, wherein the stent graft is in contact with an echogenic material.

1589. The method of item 1480, wherein the stent graft is in contact with an echogenic material, and the echogenic material is in the form of a coating.

1590. The method of item 1480, wherein stent graft is sterile.

1591. The method of item 1480, wherein the stent graft is in contact with a fibrosing agent, and the fibrosing agent is released into tissue in the vicinity of the stent graft after deployment of the stent graft in a patient.

1592. The method of item 1480, wherein the stent graft is in contact with a fibrosing agent, and the fibrosing agent is released in effective concentrations from the stent graft over a period ranging from the time of deployment of the stent graft to about at least 1 year.

1593. The method of item 1480, wherein the stent graft is in contact with a fibrosing agent, and the fibrosing agent is released in effective concentrations from the device over a period ranging from the time of deployment of the device to at least about 6 months from deployment.

1594. The method of item 1480, wherein the stent graft is in contact with a fibrosing agent, and the fibrosing agent is released in effective concentrations from the stent graft over a period ranging from the time of deployment of the stent graft to at least about 90 days from deployment.

1595. The method of item 1480, wherein the stent graft is in contact with a fibrosing agent, and the fibrosing agent is released in effective concentrations from the stent graft at a constant rate.

1596. The method of item 1480, wherein the stent graft is in contact with a fibrosing agent, and the fibrosing agent is released in effective concentrations from the stent graft at an increasing rate.

1597. The method of item 1480, wherein the stent graft is in contact with a fibrosing agent, and the fibrosing agent is released in effective concentrations from the stent graft at a decreasing rate.

1598. The method of item 1480, wherein the stent graft is in contact with a fibrosing agent, and the fibrosing agent is released in effective concentrations from the stent graft by diffusion from a polymer over a period ranging from the time of deployment of the stent graft to at least about 90 days from deployment.

1599. The method of item 1480, wherein the stent graft is in contact with a fibrosing agent, and the fibrosing agent is released in effective concentrations from the stent graft by erosion of a polymer-agent composition over a period ranging from the time of deployment of the stent graft to at least about 90 days from deployment.

1600. The method of item 1480, wherein the stent graft is in contact with about 0.01 μg to about 10 μg of a fibrosing agent.

1601. The method of item 1480, wherein the stent graft is in contact with about 10 μg to about 10 mg of a fibrosing agent.

1602. The method of item 1480, wherein the stent graft is in contact with about 10 mg to about 250 mg of a fibrosing agent.

1603. The method of item 1480, wherein the stent graft is in contact with about 250 mg to about 1000 mg of a fibrosing agent.

1604. The method of item 1480, wherein the stent graft is in contact with about 1000 mg to about 2500 mg of a fibrosing agent.

1605. The method of item 1480, wherein the stent graft is a tubular structure that comprises a lumen through which blood may flow, wherein the tubular structure comprises a luminal surface and a non-luminal surface, and wherein a fibrosing agent or a composition comprising a fibrosing agent is coated onto the non-luminal surface of the stent graft.

1606. The method of item 1480, wherein the stent graft is a tubular structure that comprises a lumen through which blood may flow, wherein the tubular structure comprises a luminal surface and a non-luminal surface, and wherein a fibrosing agent or a composition comprising a fibrosing agent is directly affixed to the non-luminal surface of the stent graft.

1607. The method of item 1480, wherein the stent graft is a tubular structure that comprises a lumen through which blood may flow, wherein the tubular structure comprises a luminal surface and a non-luminal surface, and wherein all or a portion of the non-luminal surface of the structure is covered with a fibrosing agent or a composition comprising a fibrosing agent.

1608. The method of item 1480, wherein the stent graft is a tubular structure that comprises a lumen through which blood may flow, wherein the tubular structure comprises a luminal surface and a non-luminal surface, and wherein all or a portion of the non-luminal surface of the stent graft is coated with a proliferative agent.

1609. The method of item 1480, wherein the stent graft is a tubular structure that comprises a lumen through which blood may flow, wherein the tubular structure comprises a luminal surface and a non-luminal surface, and wherein all or a portion of the luminal surface of the structure is coated with an agent that inhibits restenosis.

1610. The method of item 1480, wherein the stent graft-is a tubular structure that comprises a lumen through which blood may flow, wherein the tubular structure comprises a luminal surface and a non-luminal surface, where the method comprises attaching a thread to a non-luminal surface of the structure, wherein the thread is, or comprises, a fibrosing agent or a composition comprising the fibrosing agent.

1611. The method of item 1480, wherein the fibrosing agent or composition comprising a fibrosing agent is injected into the aneurysm.

1612. The method of item 1480 wherein the stent graft is delivered into a patient in a constrained form, and self-expands into place after release of a constraining device.

1613. The method of item 1480 wherein the stent graft is delivered to the patient by balloon catheter.

1614. A method of making a medical device comprising combining i) an intravascular implant and ii) a fibrosing agent or a composition comprising a fibrosing agent, where the fibrosing agent induces a fibrotic response between the device and a patient in which the device is implanted.

1615. The method of item 1614 wherein the fibrosing agent promotes regeneration.

1616. The method of item 1614 wherein the fibrosing agent promotes angiogenesis.

1617. The method of item 1614 wherein the fibrosing agent promotes fibroblast migration.

1618. The method of item 1614 wherein the fibrosing agent promotes fibroblast proliferation.

1619. The method of item 1614 wherein the fibrosing agent promotes deposition of extracellular matrix (ECM).

1620. The method of item 1614 wherein the fibrosing agent promotes tissue remodeling.

1621. The method of item 1614 wherein the fibrosing agent is an arterial vessel wall irritant.

1622. The method of item 1614 wherein the fibrosing agent is or comprises silk.

1623. The method of item 1614 wherein the fibrosing agent is or comprises mineral particles.

1624. The method of item 1614 wherein the fibrosing agent is or comprises chitosan.

1625. The method of item 1614 wherein the fibrosing agent is or comprises polylysine.

1626. The method of item 1614 wherein the fibrosing agent is or comprises fibronectin.

1627. The method of item 1614 wherein the fibrosing agent is or comprises bleomycin.

1628. The method of item 1614 wherein the fibrosing agent is or comprises CTGF.

1629. The method of item 1614 wherein the fibrosing agent is in the form of a thread, or is in contact with a thread.

1630. The method of item 1614 wherein the fibrosing agent is in the form of a particulate.

1631. The method of item 1614 wherein the composition further comprises an inflammatory cytokine.

1632. The method of item 1614 wherein the composition further comprises an agent that stimulates cell proliferation.

1633. The method of item 1614 wherein the composition is in the form of a gel or paste.

1634. The method of item 1614 wherein the fibrosing agent is in the form of tufts.

1635. The method of item 1614 wherein the fibrosing agent promotes adhesion between the device and a host into which the device is implanted.

1636. The method of item 1614 wherein the device delivers the fibrosing agent locally to tissue proximate to the device.

1637. The method of item 1614, wherein the implant and fibrosing agent are combined so as to provide a coating on the implant.

1638. The method of item 1614, wherein the implant and fibrosing agent are combined so as to provide a coating on the implant, and the coating directly contacts the device.

1639. The method of item 1614, wherein the implant and fibrosing agent are combined so as to provide a coating on the implant, and the coating indirectly contacts the device.

1640. The method of item 1614, wherein the implant and fibrosing agent are combined so as to provide a coating on the implant, and the coating partially covers the device.

1641. The method of item 1614, wherein the implant and fibrosing agent are combined so as to provide a coating on the implant, and the coating completely covers the device.

1642. The method of item 1614, wherein the implant and fibrosing agent are combined so as to provide a coating on the implant, and the coating is a uniform coating.

1643. The method of item 1614, wherein the implant and fibrosing agent are combined so as to provide a coating on the implant, and the coating is a non-uniform coating.

1644. The method of item 1614, wherein the implant and fibrosing agent are combined so as to provide a coating on the implant, and the coating is a discontinuous coating.

1645. The method of item 1614, wherein the implant and fibrosing agent are combined so as to provide a coating on the implant, and the coating is a patterned coating.

1646. The method of item 1614, wherein the implant and fibrosing agent are combined so as to provide a coating on the implant, and the coating has a thickness of 100 μm or less.

1647. The method of item 1614, wherein the implant and fibrosing agent are combined so as to provide a coating on the implant, and the coating has a thickness of 10 μm or less.

1648. The method of item 1614, wherein the implant and fibrosing agent are combined so as to provide a coating on the implant, and the coating adheres to the surface of the device upon deployment of the device.

1649. The method of item 1614, wherein the implant and fibrosing agent are combined so as to provide a coating on the implant, and the coating is stable at room temperature for a period of at least 1 year.

1650. The method of item 1614, wherein the implant and fibrosing agent are combined so as to provide a coating on the implant, and the fibrosing agent is present in the coating in an amount ranging between about 0.0001% to about 1% by weight.

1651. The method of item 1614, wherein the implant and fibrosing agent are combined so as to provide a coating on the implant, and the fibrosing agent is present in the coating in an amount ranging between about 1% to about 10% by weight.

1652. The method of item 1614, wherein the implant and fibrosing agent are combined so as to provide a coating on the implant, and the fibrosing agent is present in the coating in an amount ranging between about 10% to about 25% by weight.

1653. The method of item 1614, wherein the implant and fibrosing agent are combined so as to provide a coating on the implant, and the fibrosing agent is present in the coating in an amount ranging between about 25% to about 70% by weight.

1654. The method of item 1614, wherein the implant and fibrosing agent are combined so as to provide a coating on the implant, wherein the device comprises a first coating having a first composition and a second coating having a second composition.

1655. The method of item 1614, wherein the implant and fibrosing agent are combined so as to provide a coating on the implant, wherein the device comprises a first coating having a first composition and a second coating having a second composition, and where the first composition and the second composition are different.

1656. The method of item 1614, wherein the implant and fibrosing agent are combined so as to provide a coating on the implant, and the coating further comprises a polymer.

1657. The method of item 1614, wherein the implant and fibrosing agent are combined so as to provide a coating on the implant, and the coating further comprises a polymer, and the polymer is a copolymer.

1658. The method of item 1614, wherein the implant and fibrosing agent are combined so as to provide a coating on the implant, and the coating further comprises a block copolymer.

1659. The method of item 1614, wherein the implant and fibrosing agent are combined so as to provide a coating on the implant, and the coating further comprises a random copolymer.

1660. The method of item 1614, wherein the implant and fibrosing agent are combined so as to provide a coating on the implant, and the coating further comprises a biodegradable polymer.

1661. The method of item 1614, wherein the implant and fibrosing agent are combined so as to provide a coating on the implant, and the coating further comprises a non-biodegradable polymer.

1662. The method of item 1614, wherein the implant and fibrosing agent are combined so as to provide a coating on the implant, and the coating further comprises a hydrophilic polymer.

1663. The method of item 1614, wherein the implant and fibrosing agent are combined so as to provide a coating on the implant, and the coating further comprises a hydrophobic polymer.

1664. The method of item 1614, wherein the implant and fibrosing agent are combined so as to provide a coating on the implant, and the coating further comprises a polymer having hydrophilic domains.

1665. The method of item 1614, wherein the implant and fibrosing agent are combined so as to provide a coating on the implant, and the coating further comprises a polymer having hydrophobic domains.

1666. The method of item 1614, wherein the implant and fibrosing agent are combined so as to provide a coating on the implant, and the coating further comprises a non-conductive polymer.

1667. The method of item 1614, wherein the implant and fibrosing agent are combined so as to provide a coating on the implant, and the coating further comprises an elastomer.

1668. The method of item 1614, wherein the implant and fibrosing agent are combined so as to provide a coating on the implant, and the coating further comprises a hydrogel.

1669. The method of item 1614, wherein the implant and fibrosing agent are combined so as to provide a coating on the implant, and the coating further comprises a silicone polymer.

1670. The method of item 1614, wherein the implant and fibrosing agent are combined so as to provide a coating on the implant, and the coating further comprises a hydrocarbon polymer.

1671. The method of item 1614, wherein the implant and fibrosing agent are combined so as to provide a coating on the implant, and the coating further comprises a styrene-derived polymer.

1672. The method of item 1614, wherein the implant and fibrosing agent are combined so as to provide a coating on the implant, and the coating further comprises a butadiene-derived polymer.

1673. The method of item 1614, wherein the implant and fibrosing agent are combined so as to provide a coating on the implant, and the coating further comprises a macromer.

1674. The method of item 1614, wherein the implant and fibrosing agent are combined so as to provide a coating on the implant, and the coating further comprises a poly(ethylene glycol)polymer.

1675. The method of item 1614, wherein the implant and fibrosing agent are combined so as to provide a coating on the implant, and the coating further comprises an amorphous polymer.

1676. The method of item 1614, wherein the implant and fibrosing agent are combined so as to provide a coating on the implant, and the coating is a lubricious coating.

1677. The method of item 1614, wherein the implant and fibrosing agent are combined so as to provide a coating on the implant, and the coating is located within pores or holes of the implant.

1678. The method of item 1614, wherein the implant and fibrosing agent are combined so as to provide a coating on the implant, and the coating is located solely within pores or holes of the implant.

1679. The method of item 1614, wherein the implant and fibrosing agent are combined so as to provide a coating on the implant, and the coating is located within a channel, lumen, or divet of the implant.

1680. The method of item 1614, wherein the implant is combined with a second pharmaceutically active agent.

1681. The method of item 1614, wherein the implant is further combined with an anti-inflammatory agent.

1682. The method of item 1614 wherein- the implant is further combined with an agent that inhibits infection.

1683. The method of item 1614, wherein the implant is further combined with an anthracycline.

1684. The method of item 1614, wherein the implant is further combined with doxorubicin.

1685. The method of item 1614, wherein the implant is further combined with mitoxantrone.

1686. The method of item 1614, wherein the implant is further combined with a fluoropyrimidine.

1687. The method of item 1614, wherein the implant is further combined with 5-fluorouracil (5-FU).

1688. The method of item 1614, wherein the implant is further combined with a folic acid antagonist.

1689. The method of item 1614, wherein the implant is further combined with methotrexate.

1690. The method of item 1614, wherein the implant is further combined with a podophylotoxin.

1691. The method of item 1614, wherein the implant is further combined with etoposide.

1692. The method of item 1614 wherein the implant is further combined with a camptothecin.

1693. The method of item 1614, wherein the implant is further combined with a hydroxyurea.

1694. The method of item 1614, wherein the implant is further combined with a platinum complex.

1695. The method of item 1614, wherein the implant is further combined with cisplatin.

1696. The method of item 1614, wherein the implant is further combined with an anti-thrombotic agent.

1697. The method of item 1614, wherein the implant is further combined with a visualization agent.

1698. The method of item 1614, wherein the implant is further combined with a visualization agent, wherein the visualization agent is a radiopaque material, wherein the radiopaque material comprises a metal, a halogenated compound, or a barium containing compound.

1699. The method of item 1614, wherein the implant is further combined with a visualization agent, wherein the visualization agent is a radiopaque material, wherein the radiopaque material comprises barium, tantalum, or technetium.

1700. The method of item 1614, wherein the implant is further combined with a visualization agent, wherein the visualization agent is a MRI responsive material.

1701. The method of item 1614, wherein the implant is further combined with a visualization agent, wherein the visualization agent comprises a gadolinium chelate.

1702. The method of item 1614, wherein the implant is further combined with a visualization agent, wherein the visualization agent comprises iron, magnesium, manganese, copper, or chromium.

1703. The method of item 1614, wherein the implant is further combined with a visualization agent, wherein the visualization agent comprises an iron oxide compound.

1704. The method of item 1614, wherein the implant is further combined with a visualization agent, wherein the visualization agent comprises a dye, pigment, or colorant.

1705. The method of item 1614, wherein the implant is further combined with an echogenic material.

1706. The method of item 1614, wherein the implant is further combined with an echogenic material, and the echogenic material is in the form of a coating.

1707. The method of item 1614, wherein the device is sterilized.

1708. The method of item 1614, wherein the fibrosing agent is combined with the implant in a manner that provides for release of the fibrosing agent into tissue in the vicinity of the device after deployment of the device in a patient.

1709. The method of item 1614, wherein the fibrosing agent is combined with the implant in a manner that provides for release of the fibrosing agent into tissue in the vicinity of the device after deployment of the device in a patient, wherein the fibrosing agent is released in effective concentrations from the device over a period ranging from the time of deployment of the device to about at least 1 year.

1710. The method of item 1614, wherein the fibrosing agent is combined with the implant in a manner that provides for release of the fibrosing agent into tissue in the vicinity of the device after deployment of the device in a patient, wherein the fibrosing agent is released in effective concentrations from the device over a period ranging from the time of deployment of the device to at least about 6 months.

1711. The method of item 1614, wherein the fibrosing agent is combined with the implant in a manner that provides for release of the fibrosing agent into tissue in the vicinity of the device after deployment of the device in a patient, wherein the fibrosing agent is released in effective concentrations from the device over a period ranging from the time of deployment of the device to at least about 90 days.

1712. The method of item 1614, wherein the fibrosing agent is combined with the implant in a manner that provides for release of the fibrosing agent into tissue in the vicinity of the device after deployment of the device in a patient, wherein the fibrosing agent is released in effective concentrations from the device at a constant rate.

1713. The method of item 1614, wherein the fibrosing agent is combined with the implant in a manner that provides for release of the fibrosing agent into tissue in the vicinity of the device after deployment of the device in a patient, wherein the fibrosing agent is released in effective concentrations from the device at an increasing rate.

1714. The method of item 1614, wherein the fibrosing agent is combined with the implant in a manner that provides for release of the fibrosing agent into tissue in the vicinity of the device after deployment of the device in a patient, wherein the fibrosing agent is released in effective concentrations from the device at a decreasing rate.

1715. The method of item 1614, wherein the fibrosing agent is combined with the implant in a manner that provides for release of the fibrosing agent into tissue in the vicinity of the device after deployment of the device in a patient, wherein the fibrosing agent is released in effective concentrations from the composition by diffusion over a period ranging from the time of deployment of the device to at least about 90 days.

1716. The method of item 1614, wherein the fibrosing agent is combined with the implant in a manner that provides for release of the fibrosing agent into tissue in the vicinity of the device after deployment of the device in a patient, wherein the fibrosing agent is released in effective concentrations from the composition by erosion of the composition over a period ranging from the time of deployment of the device to at least about 90 days.

1717. The method of item 1614 wherein the device comprises about 0.01 μg to about 10 μg of the fibrosing agent.

1718. The method of item 1614 wherein the device comprises about 10 μg to about 10 mg of the fibrosing agent.

1719. The method of item 1614 wherein the device comprises about 10 mg to about 250 mg of the fibrosing agent.

1720. The method of item 1614 wherein the device comprises about 250 mg to about 1000 mg of the fibrosing agent.

1721. The method of item 1614 wherein the device comprises about 1000 mg to about 2500 mg of the fibrosing agent.

1722. The method of item 1614 wherein a surface of the device comprises less than 0.01 μg of the fibrosing agent per mm² of device surface occupied by fibrosing agent.

1723. The method of item 1614 wherein a surface of the device comprises about 0.01 μg to about 1 μg of the fibrosing agent per mm² of device surface occupied by fibrosing agent.

1724. The method of item 1614 wherein a surface of the device comprises about 1 μg to about 10 μg of the fibrosing agent per mm² of device surface occupied by fibrosing agent.

1725. The method of item 1614 wherein a surface of the device comprises about 10 μg to about 250 μg of the fibrosing agent per mm² of device surface occupied by fibrosing agent.

1726. The method of item 1614 wherein a surface of the device comprises about 250 μg to about 1000 μg of the fibrosing agent of fibrosing agent per mm² of device surface occupied by fibrosing agent.

1727. The method of item 1614 wherein a surface of the device comprises about 1000 μg to about 2500 μg of the fibrosing agent per mm² of device surface occupied by fibrosing agent.

1728. The method of item 1614, wherein the intravascular implant is a catheter.

1729. The method of item 1614, wherein the intravascular implant is a balloon.

1730. The method of item 1614, wherein the intravascular implant is a stent.

1731. The method of item 1614, wherein the intravascular implant is a stent graft.

1732. The method of item 1614, wherein the intravascular implant is a tubular structure that comprises a lumen through which blood may flow, wherein the tubular structure comprises a luminal surface and a non-luminal surface.

1733. The method of item 1614, wherein the intravascular implant is a tubular structure that comprises a lumen through which blood may flow, wherein the tubular structure comprises a luminal surface and a non-luminal surface, and wherein the agent or the composition comprising the agent is coated onto the non-luminal surface of the implant.

1734. The method of item 1614, wherein the intravascular implant is a tubular structure that comprises a lumen through which blood may flow, wherein the tubular structure comprises a luminal surface and a non-luminal surface, and wherein the agent or the composition comprising the agent is directly affixed to the non-luminal surface of the implant.

1735. The method of item 1614, wherein the intravascular implant is a tubular structure that comprises a lumen through which blood may flow, wherein the tubular structure comprises a luminal surface and a non-luminal surface, and wherein all or a portion of the non-luminal surface of the structure is covered with the fibrosing agent or the composition comprising the fibrosing agent.

1736. The method of item 1614, wherein the intravascular implant is a tubular structure that comprises a lumen through which blood may flow, wherein the tubular structure comprises a luminal surface and a non-luminal surface, and wherein all or a portion of the non-luminal surface of the intraluminal device is coated with a proliferative agent.

1737. The method of item 1614, wherein the intravascular implant is a tubular structure that comprises a lumen through which blood may flow, wherein the tubular structure comprises a luminal surface and a non-luminal surface, and wherein all or a portion of the luminal surface of the structure is coated with an agent that inhibits restenosis.

1738. The method of item 1614, wherein the intravascular implant is a tubular structure that comprises a lumen through which blood may flow, wherein the tubular structure comprises a luminal surface and a non-luminal surface, where the method comprises attaching a thread to a non-luminal surface of the structure, wherein the thread is, or comprises, the fibrosing agent or the composition comprising the fibrosing agent.

All of the U.S. patents, U.S. patent application publications, U.S. patent applications, foreign patents, foreign patent applications and non-patent publications referred to in this specification are incorporated herein by reference, in their entirety. The invention having been described, the following examples are intended to illustrate, and not limit, the invention.

### EXAMPLES

### Example 1

### Coating of Stents with Fibronectin

The coating apparatus consisted of an overhead stirrer (Fisher Scientific) orientated horizontally. A conical stainless steel head was attached to the revolving chuck of the stirrer. One end of the covered stent was pulled up onto the conical head until held firmly. The other end was attached to a clip-swivel device that held the covered stent in a horizontal position, but allowed the covered stent to rotate along its axis. The stirrer was then set to rotate at 30 rpm so that the whole covered stent rotated along the horizontal axis at this speed. A 1% (w/w) fibronectin (Calbiochem Corporation, San Diego, Calif.) solution in sterile water was prepared. Two hundred microlitres of this solution was slowly pipetted as a 3 mm wide ring located 5 mm from the end of the covered stent fixed in the conical steel head over a period of 2 minutes as the covered stent rotated. The fibronectin was then dried under a stream of nitrogen as the covered stent continued to rotate. When dry, the covered stent was removed, turned around and the other end of the covered stent coated in the same manner. Using this method a flexible ring of fibronectin was deposited on both ends of the covered stent without compromise of the physical characteristics of the covered stent.

### Example 2

### Coating of a Covered Stent with Poly-L-Lysine

The coating apparatus consisted of a Fisher overhead stirrer orientated horizontally. A conical stainless steel head was attached to the revolving chuck of the stirrer. One end of the covered stent was pulled up onto the conical head until held firmly. The other end was attached to a clip-swivel device that held the covered stent in a horizontal position, but allowed the covered stent covered stent to rotate along its axis. The stirrer was set to rotate at 30 rpm so that the whole covered stent rotated along the horizontal axis at this speed. A 1% (w/w) poly-L-Lysine (Sigma, St. Louis, Mo.) solution in sterile water was prepared. Two hundred microliters of this solution was slowly pipetted as a 3 mm wide ring located 5 mm from the end of the covered stent fixed in the conical steel head over a period of 2 minutes as the covered stent rotated. The poly-L-Lysine was then dried under a stream of nitrogen as the covered stent continued to rotate. When dry, the covered stent was removed, turned around and the other end of the covered stent coated in the same manner. Using this method a flexible ring of poly-L-Lysine was deposited on both ends of the graft covered stent without compromise of the physical characteristics of the covered stent.

### Example 3

### Coating of Covered Stents with N-Carboxybutyl Chitosan

The coating apparatus consists of a Fisher overhead stirrer orientated horizontally. A conical stainless steel head is attached to the revolving chuck of the stirrer. One end of the covered stent is pulled up onto the conical head until held firmly. The other end is attached to a clip-swivel device that holds the covered stent in a horizontal position, but allows the covered stent to rotate along its axis. The stirrer is set to rotate at 30 rpm so that the whole covered stent rotates along the horizontal axis at this speed. A 1% (w/w) n-carboxybutyl chitosan (Carbomer, Westborough, Mass.) solution in sterile water is prepared. Two hundred microlitres of this solution is slowly pipetted as a 3 mm wide ring located 5 mm from the end of the covered stent fixed in the conical steel head over a period of 2 minutes as the covered stent rotates. The n-carboxybutyl chitosan is dried under a stream of nitrogen as the covered stent continues to rotate. When dry, the covered stent is removed, turned around and the other end coated in the same manner. Using this method a flexible ring of n-carboxybutyl chitosan is deposited on both ends of the covered stent without compromise of the physical characteristics of the covered stent.

### Example 4

### Coating of Covered Stents with Bromocriptine in Poly(ethylene vinyl acetate)

The coating apparatus consists of a Fisher overhead stirrer orientated horizontally. A conical stainless steel head is attached to the revolving chuck of the stirrer. One end of the covered stent is pulled up onto the conical head until held firmly. The other end is attached to a clip-swivel device that holds the covered stent in a horizontal position, but allows the covered stent to rotate along its axis. The stirrer is set to rotate at 30 rpm so that the whole covered stent rotates along the horizontal axis at this speed. A 4.5% w/w solution of EVA (60/40 ratio ethylene to vinyl acetate) (Polysciences, Inc. Warrington, Pa.) is prepared in dichloromethane. Bromocriptine mesylate (Sigma, St. Louis, Mo.) is dissolved/suspended in this solution at 5 mg/ml. Two hundred microlitres of this solution is slowly pipetted as a 3 mm wide ring located 5 mm from the end of the covered stent fixed in the conical steel head over a period of 2 minutes as the covered stent rotates. The EVA/bromocriptine is dried under a stream of nitrogen as the covered stent continues to rotate. When dry, the covered stent is removed, turned around and the other end of the covered stent coated in the same manner. Using this method a flexible ring of EVA/bromocriptine is deposited on both ends of the covered stent without compromise of the physical characteristics of the covered stent.

### Example 5

### Preparation of Inflammatory Microcrystals (Monosodium Urate Monohydrate and Calcium Pyrophosphate Dihydrate)

Monosodium urate monohydrate (MSUM) microcrystals were grown. A solution of uric acid (certified A.C.S., Fisher Scientific) and sodium hydroxide at 55° C. and pH 8.9 was left to stand overnight at room temperature. The crystals were rinsed several times with cold (4° C.) distilled water and dried at 60° C. for 12 hours in a circulating hot-air oven (Fisher, Isotemp).

Triclinic calcium pyrophosphate dihydrate (CPPD) crystals were prepared as follows. A 250 ml beaker containing 103 ml distilled water was heated in a water bath to 60±2° C., and stirred constantly with a Teflon-coated stir bar. The stirring was slowed and 0.71 ml of concentrated hydrochloric acid and 0.32 ml of glacial acetic acid were added, followed by 0.6 g of calcium acetate (Fisher Certified Reagent). A 150 ml beaker containing 20 ml distilled water was heated to 60° C. in the water bath, and 0.6 g calcium acetate added. The rate of stir was increased in the 250 ml beaker, and 2 g of calcium acid pyrophosphate added rapidly. When the CaH₂P₂O₇ had nearly all dissolved, the rate of stirring was reduced for 5 minutes, then over a period of 15 seconds, the contents of the small beaker were poured into the large beaker with vigorous stirring. In the preparation of subsequent batches, a minute amount of triclinic CPPD crystals was added to the large beaker as seed material. Stirring was discontinued, leaving a white gel. This was allowed to remain undisturbed in the cooling water bath. The pH of the supernatant was always less than 3.0. The gel collapsed as CPPD crystals formed in 24 hours. The crystals were washed in distilled water 3 times, washed in ethanol then acetone, and air dried.

### Example 6

### Coating of Covered Stents with Inflammatory Microcrystals (Monosodium Urate Monohydrate or Calcium Pyrophosphate Dihydrate)

The coating apparatus consists of a Fisher overhead stirrer orientated horizontally. A conical stainless steel head is attached to the revolving chuck of the stirrer. One end of the covered stent is pulled up onto the conical head until it is held firmly. The other end is attached to a clip-swivel device that holds the covered stent in a horizontal position, but allows the covered stent to rotate along its axis. The stirrer is set to rotate at 30 rpm so that the whole covered stent rotates along the horizontal axis at this speed. A 4.5% w/w solution of EVA (60/40 ratio ethylene to vinyl acetate) (Polysciences, Inc., Warrington, Pa.) is prepared in dichloromethane. Inflammatory microcrystals (MSUM or CPPD) are ground in a pestle and mortar to a particle size of 10 to 50 micrometers and suspended in the solution at 5 mg/ml. Two hundred microlitres of this suspension is slowly pipetted as a 3 mm wide ring located 5 mm from the end of the covered stent fixed in the conical steel head over a period of 2 minutes as the covered stent rotates. The EVA/microcrystals is then dried under a stream of nitrogen as the covered stent continues to rotate. When dry, the covered stent is removed, turned around and the other end of the covered stent coated in the same manner. Using this method a flexible ring of EVA/microcrystals is deposited on both ends of the covered stent without compromise of the physical characteristics of the covered stent.

### Example 7

### Coating of Aortic Covered Stents with Inflammatory Microcrystals (Monosodium Urate Monohydrate or Calcium Pyrophosphate Dihydrate)

A 1% w/w solution of Polyurethane (PU) (Medical grade, Thermomedics, Woburn, Mass.) is prepared in dichloromethane. Inflammatory microcrystals are ground in a pestle and mortar to a particle size of 10 to 50 micrometers and suspended in the solution at 2 mg/ml. Immediately prior to surgical insertion each end of the covered stent is inserted into the shaken suspension to a depth of approximately 5 mm for 2 seconds. The covered stent is air-dried (gently rotated by hand for 3 minutes). Using this method a flexible ring of EVA/microcrystals is deposited on both ends of the covered stent without compromise of the physical characteristics of the covered stent.

### Example 8

### Coating of Intra-Anatomic Aortic Covered Stents with Bromocriptine in Polyurethane

A 1% w/w solution of Polyurethane (PU) (Medical grade, Thermomedics, Woburn, Mass.) is prepared in dichloromethane. Bromocriptine mesylate (Sigma, St. Louis, Mo.) at 5% w/w to PU is dissolved/suspended in this solution. The solution is placed in a 5 ml Fisher TLC atomizer (Fisher Scientific). Prior to surgery the covered stent is suspended vertically in a fume hood and 1 ml of the solution sprayed (using nitrogen propellant) onto the bottom 1 cm of the covered stent by revolving the covered stent through 360 degrees. The covered stent is dried for 2 minutes and then the other end of the covered stent is sprayed in a similar manner. The covered stent is then further air dried (gently rotated by hand for 3 minutes). Using this method a flexible ring of bromocriptine/PU is deposited on both ends of the covered stent without compromise of the physical characteristics of the covered stent. It is envisaged that ultimately a bromocriptine/PU solution in DCM would be available to the surgeon in the form of a small aerosol can for the above procedure.

### Example 9

### Coating of covered Stents with Inflammatory Microcrystals (Monosodium Urate Monohydrate or Calcium Pyrophosphate Dihydrate)

The coating apparatus consists of a Fisher overhead stirrer orientated horizontally. A conical stainless steel head is attached to the revolving chuck of the stirrer. One end of the covered stent is pulled up onto the conical head until it is held firmly. The other end is attached to a clip-swivel device that holds the covered stent in a horizontal position, but allows the covered stent to rotate along its axis. The stirrer is set to rotate at 30 rpm so that the whole covered stent rotates along the horizontal axis at this speed. A 4.5% w/w solution of Poly(lactide co-glycolide) (85:15) (IV 0.61) (Birmingham Polymers, Birmingham, Ala.) blended with methoxypolyethylene glycol 350 (MePEG 350) (Union Carbide, Danbury, Conn.) in a ratio of 80:20 w/w (PLGA:MePEG) is prepared in dichloromethane. Inflammatory microcrystals are suspended in the solution at 5 mg/ml. Two hundred microlitres of this suspension is slowly pipetted as a 3 mm wide ring located 5 mm from the end of the covered stent fixed in the conical steel head over a period of 2 minutes as the covered stent rotates. The PLGA/MePEG/inflammatory crystals are then dried under a stream of nitrogen as the covered stent continues to rotate. When dry, the covered stent is removed, turned around and the other end of the covered stent coated in the same manner. Using this method a flexible ring of PLGA/MePEG/microcrystals is deposited on both ends of the covered stent without compromise of the physical characteristics of the covered stent.

### Example 10

### Coating of Covered Stents with Angiotensin 2 Encapsulated in Polyethylene Glycol (PEG)

1.8 grams of polyethylene glycol 1475 (Union Carbide, Danbury, Conn.) is placed in a flat-bottomed 20 ml glass scintillation vial and warmed to 50° C. to melt the PEG in a water bath, 200 mg of glycerol (Fisher Scientific, Pittsburgh, Pa.) is added 2 mg of angiotensin 2 (Sigma, St. Louis, Mo.) is weighed into the vial and blended/dissolved into the melted PEG at 50° C. The vial is angled at 10 degrees in a water bath by use of a clamp. Each end of the covered stent is rotated in the molten formulation, so that a ring of material is deposited on the bottom 5 mm of the exterior surface of the covered stent. The covered stent is then cooled and stored at 4° C. until use. Alternatively, to enable dipping immediately prior to surgery the PEG/angiotensin mixture is stored at 4° C. until use. Immediately prior to surgery, the vial of PEG/angiotensin is warmed to 50° C. for 2 minutes to melt and the covered stent is coated as described above.

### Example 11

### Coating of Covered Stents with Transforming Growth Factor-β (TGF-β) in Crosslinked Hyaluronic Acid

The coating apparatus consists of a Fisher overhead stirrer orientated horizontally. A conical stainless steel head is attached to the revolving chuck of the stirrer. One end of the covered stent is pulled up onto the conical head until held firmly. The other end is attached to a clip-swivel device that holds the covered stent in a horizontal position, but allows the covered stent to rotate along its axis. The stirrer is set to rotate at 30 rpm so that the whole covered stent rotates along the horizontal axis at this speed. A 1% solution of hyaluronic acid (HA) (Sodium salt, Sigma, St. Louis, Mo.) in water, containing 30% glycerol (w/w to HA) (Fisher Scientific, Pittsburgh, Pa.) and 8 mM 1-ethyl-3-(-3 dimethylaminopropyl) carbodiimide (EDAC) (Sigma, St. Louis, Mo.) is prepared by dissolution overnight. TGF-β (Calbiochem, San Diego, Calif.) is dissolved at 0.01 mg/ml in this solution. Two hundred microlitres of this solution is slowly pipetted as a 3 mm wide ring located 5 mm from the end of the covered stent fixed in the conical steel head over a period of 2 minutes as the covered stent rotates. The HA/glycerol/TGF-β solution is dried under a stream of nitrogen as the covered stent continues to rotate. When dry, the covered stent is removed, turned around and the other end coated in the same manner. Using this method a flexible ring of HA/glycerol/TGF-βis deposited on both ends of the covered stent without compromise of the physical characteristics of the covered stent.

### Example 12

### Coating of Covered Stents with Fibroblast Growth Factor (FGF) in Crosslinked Chitosan

The coating apparatus consists of a Fisher overhead stirrer orientated horizontally. A conical stainless steel head is attached to the revolving chuck of the stirrer. One end of the covered stent is pulled up onto the conical head until held firmly. The other end is attached to a clip-swivel device that holds the covered stent in a horizontal position, but allows the covered stent to rotate along its axis. The stirrer is set to rotate at 30 rpm so that the whole covered stent rotates along the horizontal axis at this speed. A 1% solution of chitosan (Medical grade, Carbomer, Westborough, Mass.) in dilute acetic acid (pH 5), containing 30% glycerol (w/w to chitosan) (Fisher Scientific, Pittsburgh, Pa.) and 0.5% glutaraldehyde (Sigma, St. Louis, Mo.) is prepared by dissolution overnight. FGF (Calbiochem, San Diego, Calif.) is dissolved at 0.01 mg/ml in this solution. Two hundred microlitres of this solution is slowly pipetted as a 3 mm wide ring located 5 mm from the end of the covered stent fixed in the conical steel head over a period of 2 minutes as the covered stent rotates. The chitosan/glycerol/FGF solution is dried under a stream of nitrogen as the covered stent continues to rotate. When dry, the covered stent is removed, turned around and the other end coated in the same manner. Using this method a flexible ring of chitosan/glycerol/FGF is deposited on both ends of the covered stent without compromise of the physical characteristics of the covered stent.

### Example 13

### Screening Procedure for Assessment of Perigraft Reaction

A rabbit perivascular model is described for identifying arterial vessel wall irritants. Large domestic rabbits are placed under general anesthetic. Using aseptic precautions, the infrarenal abdominal aorta is exposed and clamped at its superior and inferior aspects. A longitudinal arterial wall arteriotomy is performed and a 2 millimeter diameter, 1 centimeter long segment of PTFE graft is inserted within the aorta and the proximal and distal aspect of the graft is sewn so that the entire aortic blood flow is through the graft which is contained in the abdominal aorta in the manner of open surgical abdominal aortic repair in humans (except that no aneurysm is present in this model). The aortotomy is then surgically closed and the abdominal wound closed and the animal recovered.

The animals are randomized to receive standard PTFE grafts or grafts of which the middle 1 cm is coated alone circumferentially with nothing, or with an agent that induces a vessel wall reaction or adhesion between a stent graft and vessel wall alone or contained in a slow release, polymer such as polycaprolactone or polylactic acid.

The animals are sacrificed between 1 and 6 weeks post surgery, the aorta is removed en bloc and the area in relation to the graft is grossly examined for adhesive reaction. Any difference in morphology or histology of the vessel wall from portions of the artery which contain no graft, portion which contain graft without coating, and portion which contained graft with coating is noted.

### Example 14

### Animal Abdominal Aortic Aneurysm Model

An animal model is described for determining whether a stent graft containing a biologically active or irritative substance stimulates fibrosis. Pigs or sheep are placed under general anesthetic. Using aseptic precautions the abdominal aorta is exposed. The animal is heparinized and the aorta is cross clamped below the renal arteries and above the bifurcation. Collaterals are temporarily controlled with vessel loops or clips that are removed upon completion of the procedure. A longitudinal aortotomy is created in the arterial aspect of the aorta, and an elliptical shaped patch of rectus sheath from the same animal is sutured into the aortotomy to create an aneurysm. The aortic clamps from the lumbar arteries and collaterals are removed and the abdomen closed. After 30 days, the animal is reanesthesized and the abdominal wall again opened. A cutdown is performed on the iliac artery and through this, a stent graft is positioned across the infrarenal abdominal aorta aneurysm extending from normal infrarenal abdominal aorta above to normal infrarenal abdominal aorta below the surgically created aneurysm and the device is released in a conventional way.

Animals are randomized into groups of 5 receiving uncoated stent grafts, stent graft containing slow release polymer alone, and stent graft containing a biologically active or irritative substance as determined by the previously mentioned screening exam. After closure of the arteriotomy and of the abdominal wound, the animal is allowed to recover. At 6 weeks and 3 months post stent graft insertion, the animal is sacrificed and the aorta removed en bloc. The infrarenal abdominal aorta is examined for evidence of histologic reaction and perigraft leaking.

### Example 16

### Screening Assay for Assessing the Effect of Cyclosporine A on Cell Proliferation

An in vitro assay is described for determining whether a substance stimulates cell (fibroblast) proliferation. Smooth muscle cells at 70-90% confluency are trypsinized, replated at 600 cells/well in media in 96-well plates and allowed to attachment overnight. Cyclosporine A is prepared in DMSO at a concentration of 10⁻² M and diluted 10-fold to give a range of stock concentrations (10⁻⁸ M to 10⁻² M). Drug dilutions are diluted 1/1000 in media and added to cells to give a total volume of 200 μL/well. Each drug concentration is tested in triplicate wells. Plates containing smooth muscle cells and cyclosporine A are incubated at 37° C. for 72 hours.

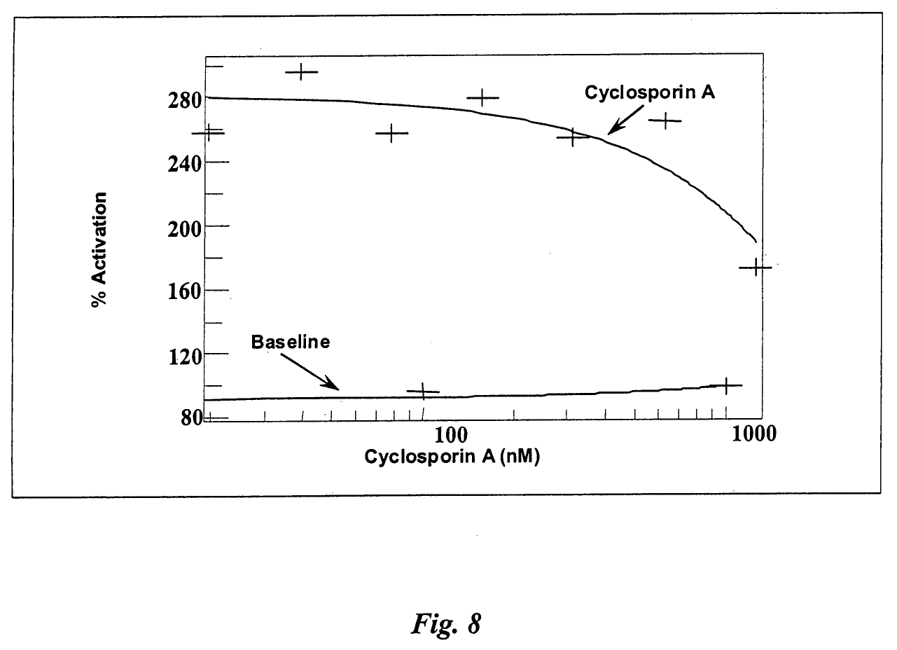

To terminate the assay, the media is removed by gentle aspiration. A 1/400 dilution of CYQUANT 400× GR dye indicator (Molecular Probes; Eugene, Oreg.) is added to 1× Cell Lysis buffer, and 200 μL of the mixture is added to the wells of the plate. Plates are incubated at room temperature, protected from light for 3-5 minutes. Fluorescence is read in a fluorescence microplate reader at ˜480 nm excitation wavelength and ˜520 nm emission maxima. Activation of proliferation is determined by taking the average of triplicate wells and comparing average relative fluorescence units to the DMSO control (see FIG. 8). References: In vitro toxicol. (1990) 3: 219; Biotech. Histochem. (1993) 68: 29; Anal. Biochem. (1993)213:426.

### Example 17

### Screening Assay for Assessing the Effect of Dexamthasone on Cell Proliferation

An in vitro assay is described for determining whether a substance stimulates cell (fibroblast) proliferation. Fibroblasts at 70-90% confluency are trypsinized, replated at 600 cells/well in media in 96-well plates and allowed to attachment overnight. Dexamethasone is prepared in DMSO at a concentration of 10⁻² M and diluted 10-fold to give a range of stock concentrations (10⁻⁸ M to 10⁻² M). Drug dilutions are diluted 1/1000 in media and added to cells to give a total volume of 200 μL/well. Each drug concentration is tested in triplicate wells. Plates containing fibroblasts and dexamethasone are incubated at 37° C. for 72 hours.

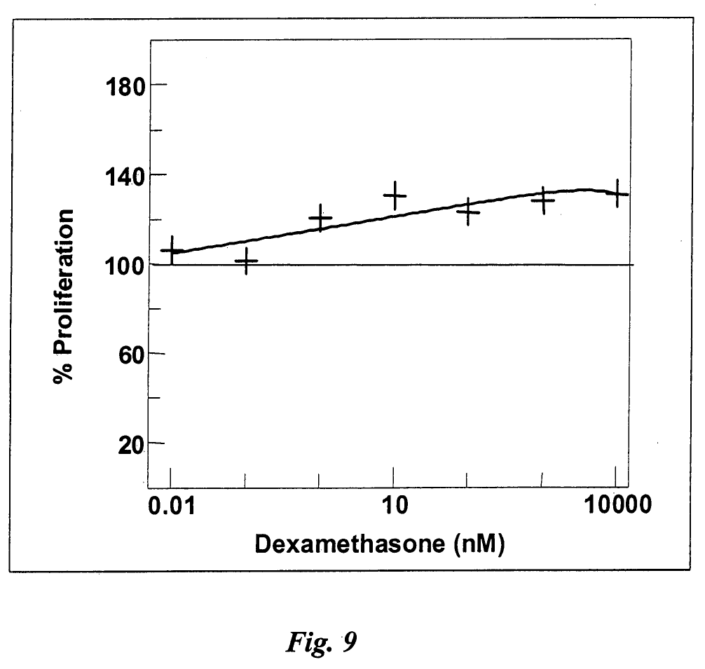

To terminate the assay, the media is removed by gentle aspiration. A 1/400 dilution of CYQUANT 400× GR dye indicator is added to 1× Cell Lysis buffer, and 200 μL of the mixture is added to the wells of the plate. Plates are incubated at room temperature, protected from light for 3-5 minutes. Fluorescence is read in a fluorescence microplate reader at ˜480 nm excitation wavelength and ˜520 nm emission maxima. Activation of proliferation is determined by taking the average of triplicate wells and comparing average relative fluorescence units to the DMSO control (see FIG. 9). References: In vitro toxicol. (1990) 3: 219; Biotech. Histochem. (1993) 68: 29; Anal. Biochem. (1993) 213: 426.

### Example 18

### Screening Assay for Assessing the Effect of All-Trans Retinoic Acid on Cell Proliferation

An in vitro assay is described for determining whether a substance stimulates cell (fibroblast) proliferation. Smooth muscle cells at 70-90% confluency are trypsinized, replated at 600 cells/well in media in 96-well plates and allowed to attachment overnight. All-trans retinoic acid is prepared in DMSO at a concentration of 10⁻² M and diluted 10-fold to give a range of stock concentrations (10⁻⁸ M to 10⁻² M). Drug dilutions are diluted 1/1000 in media and added to cells to give a total volume of 200 μL/well. Each drug concentration is tested in triplicate wells. Plates containing smooth muscle cells and all-trans retinoic acid are incubated at 37° C. for 72 hours.

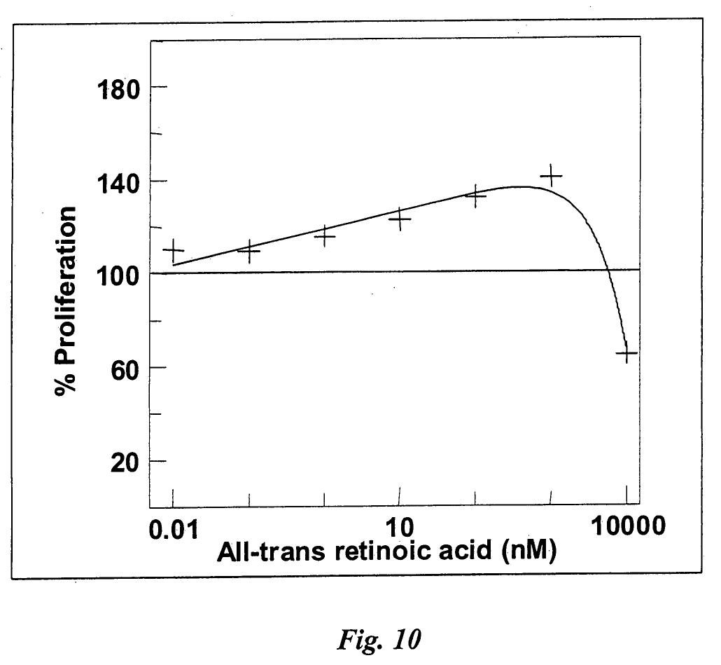

To terminate the assay, the media is removed by gentle aspiration. A 1/400 dilution of CYQUANT 400× GR dye indicator is added to 1× Cell Lysis buffer, and 200 μL of the mixture is added to the wells of the plate. Plates are incubated at room temperature, protected from light for 3-5 minutes. Fluorescence is read in a fluorescence microplate reader at ˜480 nm excitation wavelength and ˜520 nm emission maxima. Activation of proliferation is determined by taking the average of triplicate wells and comparing average relative fluorescence units to the DMSO control (see FIG. 10). References: In vitro toxicol (1990) 3: 219; Biotech. Histochem. (1993) 68: 29; Anal. Biochem. (1993) 213: 426.

### Example 19

### Screening Assay for Assessing the Effect of Isotretinoin on Cell Proliferation

An in vitro assay is described for determining whether a substance stimulates cell (fibroblast) proliferation. Smooth muscle cells at 70-90% confluency are trypsinized, replated at 600 cells/well in media in 96-well plates and allowed to attachment overnight. Isotretinoin is prepared in DMSO at a concentration of 10⁻² M and diluted 10-fold to give a range of stock concentrations (10⁻⁸ M to 10⁻² M). Drug dilutions are diluted 1/1000 in media and added to cells to give a total volume of 200 μL/well. Each drug concentration is tested in triplicate wells. Plates containing smooth muscle cells and isotretinoin are incubated at 37° C. for 72 hours.

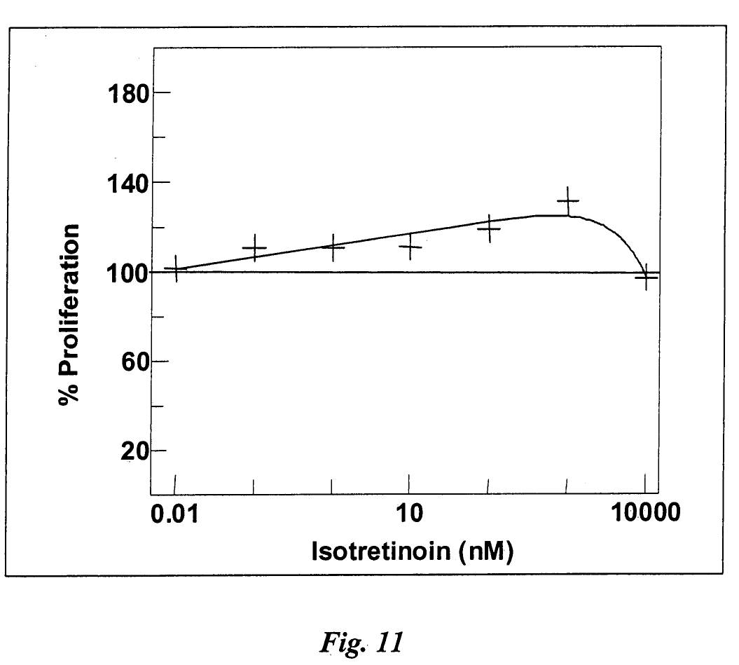

To terminate the assay, the media is removed by gentle aspiration. A 1/400 dilution of CYQUANT 400× GR dye indicator is added to 1× Cell Lysis buffer, and 200 μL of the mixture is added to the wells of the plate. Plates are incubated at room temperature, protected from light for 3-5 minutes. Fluorescence is read in a fluorescence microplate reader at ˜480 nm excitation wavelength and ˜520 nm emission maxima. Activation of proliferation is determined by taking the average of triplicate wells and comparing average relative fluorescence units to the DMSO control (see FIG. 11). References: In vitro toxicol. (1990) 3: 219; Biotech. Histochem. (1993) 68: 29; Anal. Biochem. (1993) 213: 426.

### Example 20

### Screening Assay for Assessing the Effect of 17-?-Estradiol on Cell Proliferation

An in vitro assay is described for determining whether a substance stimulates cell (fibroblast) proliferation. Fibroblasts at 70-90% confluency are trypsinized, replated at 600 cells/well in media in 96-well plates and allowed to attachment overnight. 17-β-estradiol is prepared in DMSO at a concentration of 10⁻² M and diluted 10-fold to give a range of stock concentrations (10⁻⁸ M to 10⁻² M). Drug dilutions are diluted 1/1000 in media and added to cells to give a total volume of 200 μL/well. Each drug concentration is tested in triplicate wells. Plates containing fibroblasts and 17-β-estradiol are incubated at 37° C. for 72 hours.

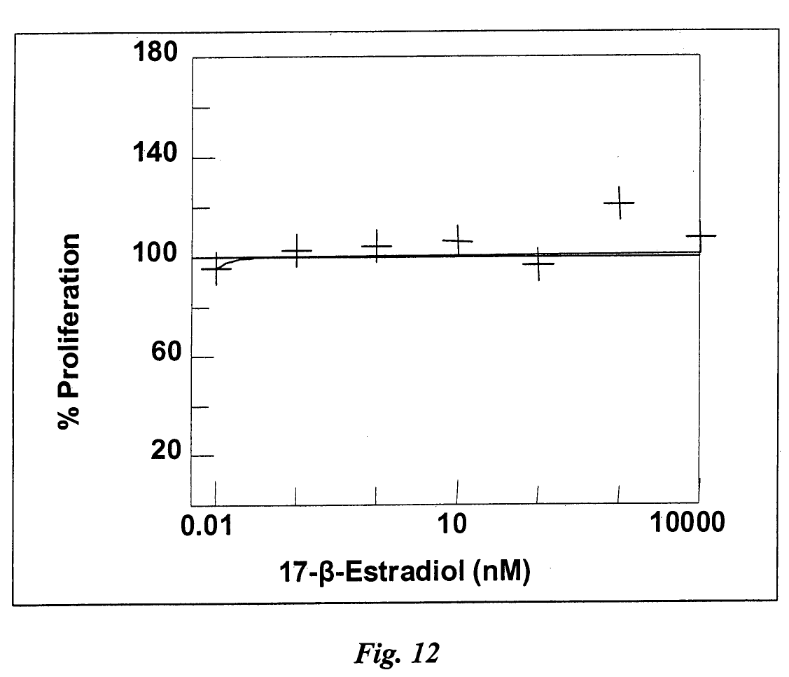

To terminate the assay, the media is removed by gentle aspiration. A 1/400 dilution of CYQUANT 400× GR dye indicator is added to 1× Cell Lysis buffer, and 200 μL of the mixture is added to the wells of the plate. Plates are incubated at room temperature, protected from light for 3-5 minutes. Fluorescence is read in a fluorescence microplate reader at ˜480 nm excitation wavelength and ˜520 nm emission maxima. Activation of proliferation is determined by taking the average of triplicate wells and comparing average relative fluorescence units to the DMSO control (see FIG. 12). References: In vitro toxicol. (1990) 3: 219; Biotech. Histochem. (1993) 68: 29; Anal. Biochem. (1993) 213: 426.

### Example 21

### Screening Assay for Assessing the Effect of 1?,25-Dihydroxy-Vitamin D₃ on Cell Proliferation

An in vitro assay is described for determining whether a substance stimulates cell (fibroblast) proliferation. Smooth muscle cells at 70-90% confluency are trypsinized, replated at 600 cells/well in media in 96-well plates and allowed to attachment overnight. 1a,25-Dihydroxy-vitamin D3 is prepared in DMSO at a concentration of 10⁻² M and diluted 10-fold to give a range of stock concentrations (10⁻⁸ M to 10⁻² M). Drug dilutions are diluted 1/1000 in media and added to cells to give a total volume of 200 μL/well. Each drug concentration is tested in triplicate wells. Plates containing smooth muscle cells and 1a,25-dihydroxy-vitamin D₃ are incubated at 37° C. for 72 hours.

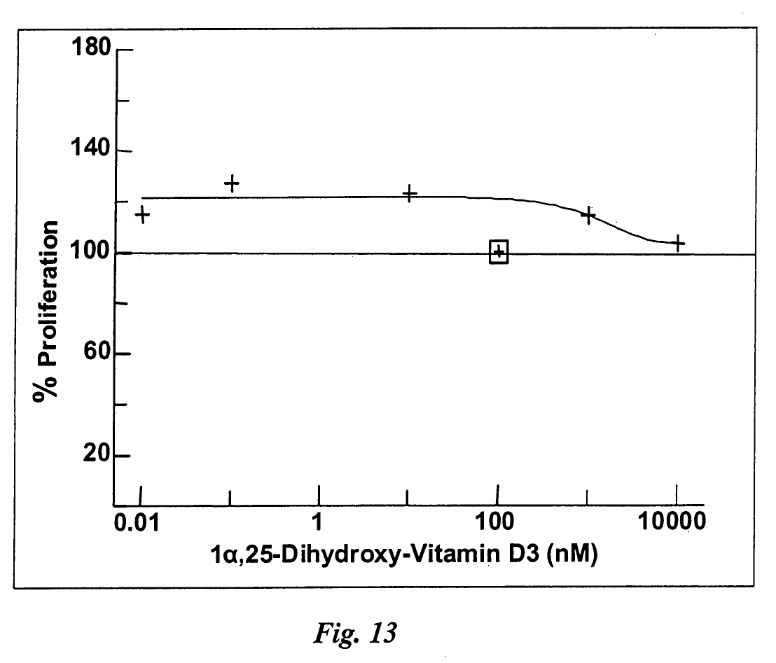

To terminate the assay, the media is removed by gentle aspiration. A 1/400 dilution of CYQUANT 400× GR dye indicator is added to 1× Cell Lysis buffer, and 200 μL of the mixture is added to the wells of the plate. Plates are incubated at room temperature, protected from light for 3-5 minutes. Fluorescence is read in a fluorescence microplate reader at ˜480 nm excitation wavelength and ˜520 nm emission maxima. Activation of proliferation is determined by taking the average of triplicate wells and comparing average relative fluorescence units to the DMSO control (see FIG. 13). References: In vitro toxicol. (1990) 3: 219; Biotech. Histochem. (1993) 68: 29; Anal. Biochem. (1993) 213: 426.

### Example 22

### Screening Assay for Assessing the Effect of PDGF on Smooth Muscle Cell Migration

An in vitro assay is described for determining whether a substance stimulates cell (fibroblast) migration. Primary human smooth muscle cells are starved of serum in smooth muscle cell basal media containing insulin and human basic fibroblast growth factor (bFGF) for 16 hours prior to the assay. For the migration assay, cells are trypsinized to remove cells from flasks, washed with migration media and diluted to a concentration of 2-2.5×10⁵ cells/ml in migration media. Migration media consists of phenol red free Dulbecco's Modified Eagle Medium (DMEM) containing 0.35% human serum albumin. A 100 μL volume of smooth muscle cells (approximately 20,000-25,000 cells) is added to the top of a Boyden chamber assembly (Chemicon QCM Chemotaxis 96-well migration plate). To the bottom wells, the chemotactic agent, recombinant human platelet derived growth factor (rhPDGF-BB) is added at a concentration of 10 ng/ml in a total volume of 150 μL. Paclitaxel is prepared in DMSO at a concentration of 10⁻² M and serially diluted 10-fold to give a range of stock concentrations (10⁻⁸ M to 10⁻² M). Paclitaxel is added to cells by directly adding paclitaxel DMSO stock solutions, prepared earlier, at a 1/1000 dilution, to the cells in the top chamber. Plates are incubated for 4 hours to allow cell migration.

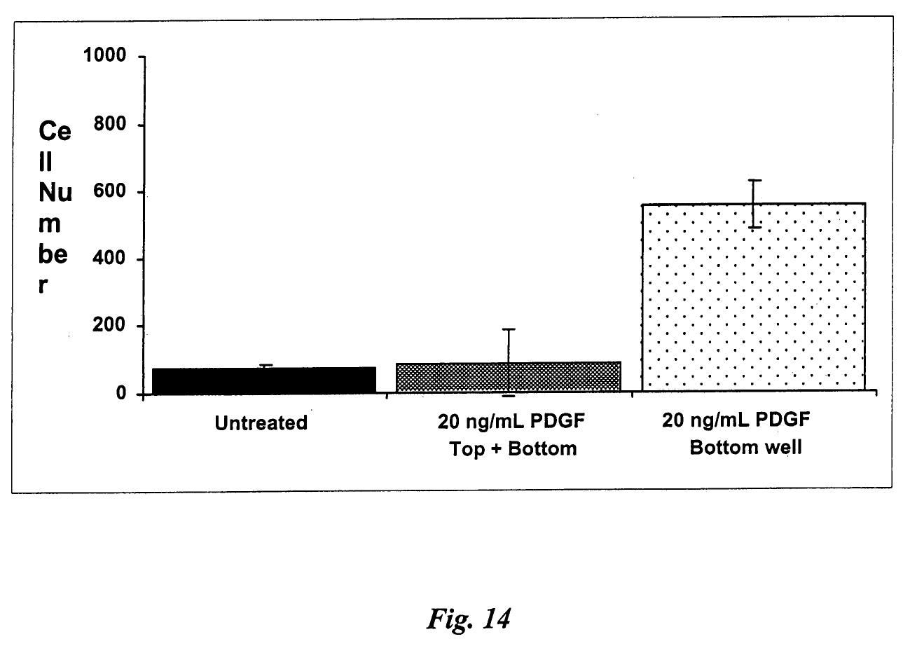

At the end of the 4 hour period, cells in the top chamber are discarded and the smooth muscle cells attached to the underside of the filter are detached for 30 minutes at 37° C. in Cell Detachment Solution (Chemicon). Dislodged cells are lysed in lysis buffer containing the DNA binding CYQUANT GR dye and incubated at room temperature for 15 minutes. Fluorescence is read in a fluorescence microplate reader at ˜480 nm excitation wavelength and ˜520 nm emission maxima. Relative fluorescence units from triplicate wells are averaged after subtracting background fluorescence (control chamber without chemoattractant) and average number of cells migrating is obtained from a standard curve of smooth muscle cells serially diluted from 25,000 cells/well down to 98 cells/well. Inhibitory concentration of 50% (IC₅₀) is determined by comparing the average number of cells migrating in the presence of paclitaxel to the positive control (smooth muscle cell chemotaxis in response to rhPDGF-BB). See FIG. 14. References: Biotechniques (2000) 29: 81; J. Immunol Methods (2001) 254: 85.

### Example 23

### In vivo Evaluation of Silk Coated Perivascular PU Films to Assess Scarring

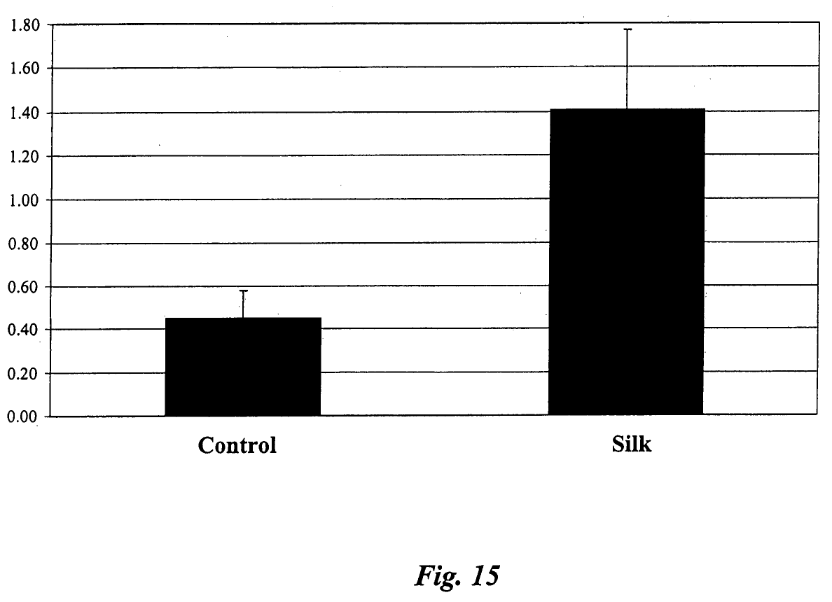

A rat carotid artery model is described for determining whether a substance stimulates fibrosis. Wistar rats weighing 300 g to 400 g are anesthetized with halothane. The skin over the neck region is shaved and the skin is sterilized. A vertical incision is made over the trachea and the left carotid artery is exposed. A polyurethane film covered with silk strands or a control uncoated PU film is wrapped around a distal segment of the common carotid artery. The wound is closed and the animal is recovered. After 28 days, the rats are sacrificed with carbon dioxide and pressure-perfused at 100 mmHg with 10% buffered formaldehyde. Both carotid arteries are harvested and processed for histology. Serial cross-sections will be cut every 2 mm in the treated left carotid artery and at corresponding levels in the untreated right carotid artery. Sections are stained with H&E and Movat's stains to evaluate tissue growth around the carotid artery. Area of perivascular granulation tissue is quantified by computer-assisted morphometric analysis. Area of the granulation tissue is significantly higher in the silk coated group than in the control uncoated group. See FIG. 15.

### Example 24

### In vivo Evaluation of Perivascular PU Films Coated with Different Silk Suture Material to Assess Scarring

A rat carotid artery model is described for determining whether a substance stimulates fibrosis. Wistar rats weighing 300 g to 400 g are anesthetized with halothane. The skin over the neck region is shaved and the skin is sterilized. A vertical incision is made over the trachea and the left carotid artery is exposed. A polyurethane film covered with silk sutures from one of three different manufacturers (3-0 Silk—Black Braided (Davis & Geck), 3-0 SOFSILK (U.S. Surigical/Davis & Geck), and 3-0—Silk-Black Braided (LIGAPAK) (Ethicon, Inc., Sommerville, N.J.)) is wrapped around a distal segment of the common carotid artery. (The polyurethane film can also be coated with other agents which can induce fibrosis.) The wound is closed and the animal is allowed to recover.

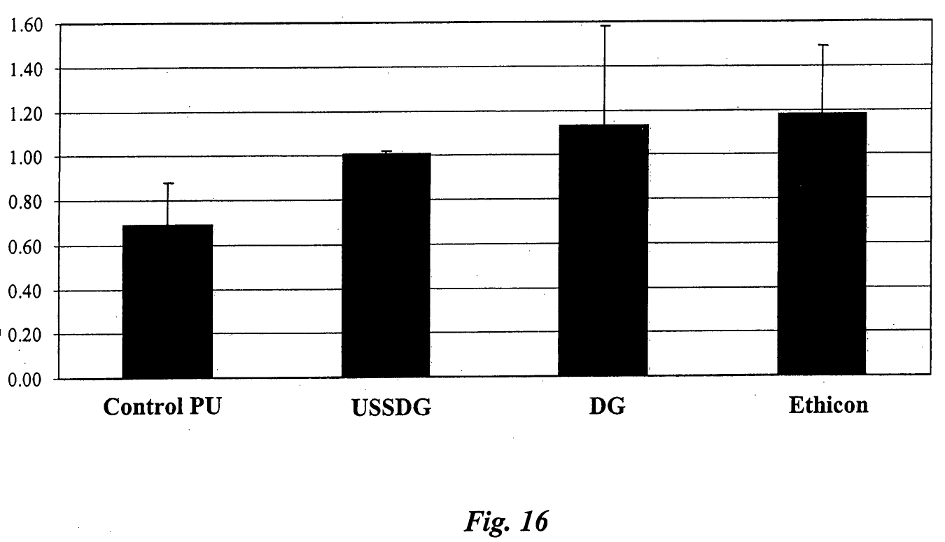

After 28 days, the rats are sacrificed with carbon dioxide and pressure-perfused at 100 mmHg with 10% buffered formaldehyde. Both carotid arteries are harvested and processed for histology. Serial cross-sections will be cut every 2 mm in the treated left carotid artery and at corresponding levels in the untreated right carotid artery. Sections are stained with H&E and Movat's stains to evaluate tissue growth around the carotid artery. Area of perivascular granulation tissue is quantified by computer-assisted morphometric analysis. Thickness of the granulation tissue is the same in the three groups showing that tissue proliferation around silk suture is independent of manufacturing processes. See FIG. 16.

### Example 25

### In vivo Evaluation of Perivascular Silk Powder to Assess Scarring

A rat carotid artery model is described for determining whether a substance stimulates fibrosis. Wistar rats weighing 300 g to 400 g are anesthetized with halothane. The skin over the neck region is shaved and the skin is sterilized. A vertical incision is made over the trachea and the left carotid artery is exposed. Silk powder is sprinkled on the exposed artery that is then wrapped with a PU film. Natural silk powder or purified silk powder (without contaminant proteins) is used in different groups of animals. Carotids wrapped with PU films only are used as a control group. The wound is closed and the animal is recovered. After 28 days, the rats are sacrificed with carbon dioxide and pressure-perfused at 100 mmHg with 10% buffered formaldehyde. Both carotid arteries are harvested and processed for histology. Serial cross-sections will be cut every 2 mm in the treated left carotid artery and at corresponding levels in the untreated right carotid artery. Sections are stained with H&E and Movat's stains to evaluate tissue growth around the carotid artery. Area of tunica intima, tunica media and perivascular granulation tissue is quantified by computer-assisted morphometric analysis.

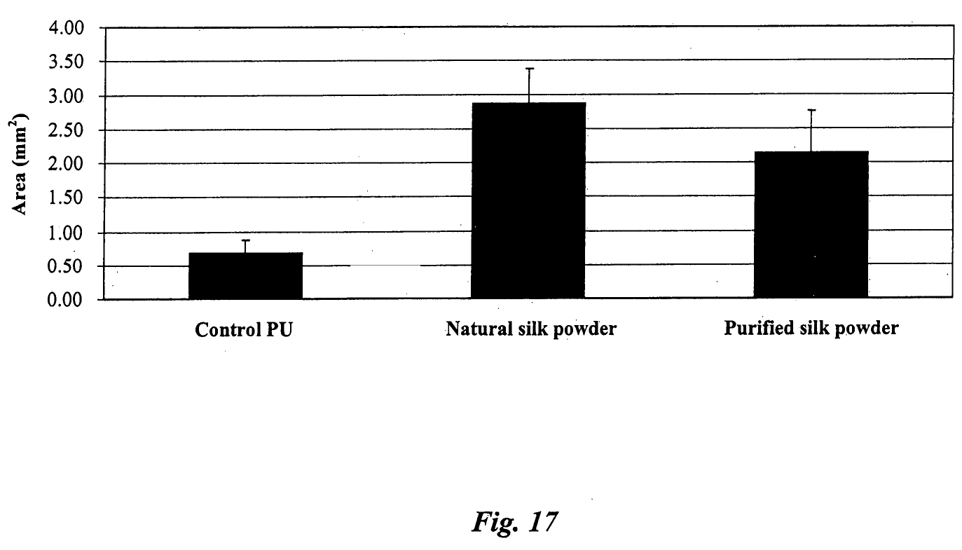

The natural silk caused a severe cellular inflammation consisting mainly of a neutrophil and lymphocyte infiltrate in a fibrin network without any extracellular matrix or blood vessels. In addition, the treated arteries were seriously damaged with hypocellular media, fragmented elastic laminae and thick intimal hyperplasia. Intimal hyperplasia contained many inflammatory cells and was occlusive in 2/6 cases. This severe immune response was likely triggered by antigenic proteins coating the silk protein in this formulation. On the other end, the regenerated silk powder triggered only a mild foreign body response surrounding the treated artery. This tissue response was characterized by inflammatory cells in extracellular matrix, giant cells and blood vessels. The treated artery was intact. These results show that removing the coating proteins from natural silk prevents the immune response and promotes benign tissue growth. Degradation of the regenerated silk powder was underway in some histology sections indicating that the tissue response will likely mature and heal over time. See FIG. 17.

### Example 26

### In vivo Evaluation of Perivascular Talcum Powder to Assess Scarring

A rat carotid artery model is described for determining whether a substance stimulates fibrosis. Wistar rats weighing 300 g to 400 g are anesthetized with halothane. The skin over the neck region is shaved and the skin is sterilized. A vertical incision is made over the trachea and the left carotid artery is exposed. Talcum powder is sprinkled on the exposed artery that is then wrapped with a PU film. Carotids wrapped with PU films only are used as a control group. The wound is closed and the animal is recovered. After 1 or 3 months, the rats are sacrificed with carbon dioxide and pressure-perfused at 100 mmHg with 10% buffered formaldehyde. Both carotid arteries are harvested and processed for histology. Serial cross-sections will be cut every 2 mm in the treated left carotid artery and at corresponding levels in the untreated right carotid artery. Sections are stained with H&E and Movat's stains to evaluate tissue growth around the carotid artery. Thickness of tunica intima, tunica media and perivascular granulation tissue is quantified by computer-assisted morphometric analysis.

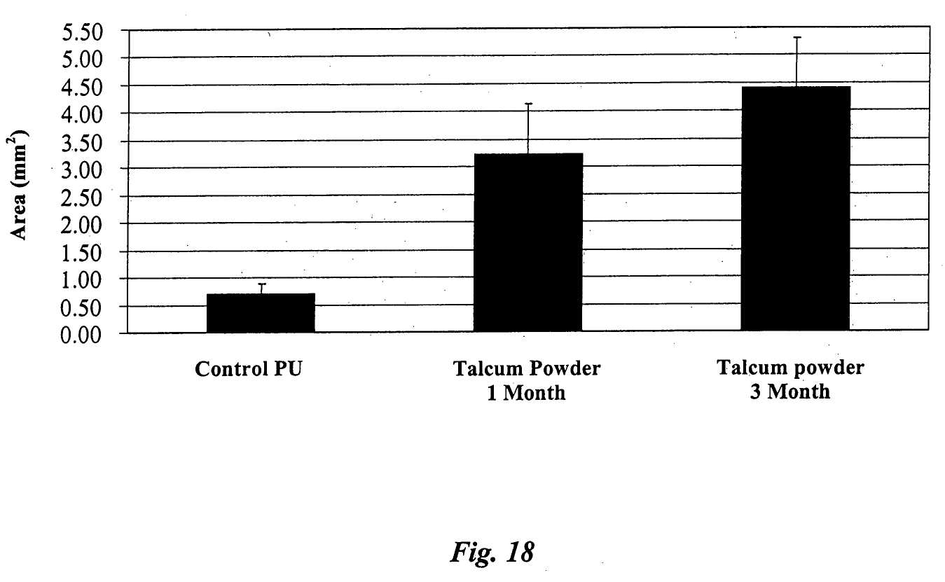

Histopathology results and morphometric analysis showed the same local response to talcum powder at 1 month and 3 months. A large tissue reaction trapped the talcum powder at the site of application around the blood vessel. This tissue was characterized by a large number of macrophages within a dense extracellular matrix with few neutrophiles, lymphocytes and blood vessels. The treated blood vessel appeared intact and unaffected by the treatment. Overall, this result showed that talcum powder induced a mild long-lasting fibrotic reaction that was subclinical in nature and did not harm any adjacent tissue. See FIG. 18.

### Example 27

### Preparation of Silk Powder

Several pieces of silk braid (Ethicon, 4-0, 638) are cut into lengths of approx 0.4 cm. These cut pieces are placed in a 100 ml round bottom flask that contains 50 ml 2M NaOH. The sample is stirred using a magnetic stirrer at room temperature for 24 h. The sample is neutralized using concentrated HCl. The neutralized contents are then dialyzed against deionized water using Spectrum cellulose-based dialysis tubing (WMCO apprpx 3000). The sample is dialyzed for 48 hours with 5 water changes. The dialyzed sample is then poured into a 100 ml round bottom flask. The sample is frozen and freeze-dried to yield a fluffy powdered material.

### Example 28

### Coating of the Stent Graft with a Powdered Silk/PLGA Coating

A stent graft (WALLGRAFT Endoprosthesis, Ref: 50019, Boston Scientific) is pushed onto a 1 ml plastic pipette tip. The open end of the pipette tip is attached to a stainless steel rod that is attached to a Fisher overhead stirrer that is orientated horizontally. The stirrer is set to rotate at 30 rpm. A 2% PLGA (9K, 50:50, Birmingham Polymers) solution (ethyl acetate) that contains the powdered silk is sprayed onto the rotating stent graft using an airbrush spray device. The concentration of the powdered silk in the PLGA solution is altered from 0.1% to 50%. After the spraying process, the stent graft is allowed to air dry for 30 minutes while still rotating. The stent graft is then removed from the pipette tip and is further dried under vacuum for 24 h.

### Example 29

### Coating of Stent-Graft with a Powdered Silk/Polyurethane Coating

A stent-graft is pushed onto a plastic pipette. The open end of the pipette is attached to a stainless steel rod that is attached to a Fisher overhead stirrer that is orientated horizontally. The stirrer is set to rotate at 30 rpm. A 2% CHRONOFLEX AL 85A (CT Biomaterials) solution (THF) that contains the powdered silk is sprayed onto the rotating stent-graft using a TLC spray device. The concentration of the powdered silk in the polyurethane solution is altered from 0.1% to 50%. After the spraying process, the stent-graft is allowed to air dry for 30 minutes while still rotating. The stent-graft is then removed from the pipette tip and is further dried under vacuum for 24 h.

### Example 30

### Top-Coating of a Coated Stent-Graft with a Degradable Coating

The coated stent-graft from Example 29, is reattached to the overhead stirrer and is rotated at 30 rpm. A 10% 20:80 MePEG(750)-PLA block copolymer solution (acetone) is sprayed onto the rotating stent-graft using an TLC spray device. After the spraying process, the stent-graft is allowed to air dry for 30 minutes while still rotating. To obtain a thicker coating, the spray process is repeated. The spray coating process can be repeated until the desired thickness or uniformity of coating is obtained. The stent-graft is then removed from the pipette tip and is further dried under vacuum for 24 h.

### Example 31

### Top-Coating of a Coated Stent-Graft with a Heparin-Containing Degradable Coating

The coated stent-graft from Example 29 is reattached to the overhead stirrer and is rotated at 30 rpm. A 10% 20:80 MePEG(750)-PLA block copolymer solution (acetone)that contains various amounts of a Heparin benzalkonium chloride complex (PolySciences) is sprayed onto the rotating stent-graft using a TLC spray device. After the spraying process, the stent-graft is allowed to air dry for 30 minutes while still rotating. To obtain a thicker coating, the spray process is repeated. The spray coating process can be repeated until the desired thickness or uniformity of coating is obtained. The stent-graft is then removed from the pipette tip and is further dried under vacuum for 24 h.

### Example 32

### Coating of a Coated Stent-Graft with a Heparin Coating

The coated stent-graft from Example 29, is reattached to the overhead stirrer and is rotated at 30 rpm. A solution (IPA) that contains various amounts of a Heparin benzalkonium chloride complex (PolySciences) is sprayed onto the rotating stent-graft using a TLC spray device. After the spraying process, the stent-graft is allowed to air dry for 30 minutes while still rotating. The spray coating process can be repeated until the desired thickness or uniformity of coating is obtained. The stent-graft is then removed from the pipette tip and is further dried under vacuum for 24 h.

### Example 33

### Coating of Stent-Graft with a Powdered Silk/Cyclosporine A/Polyurethane Coating

A stent-graft is pushed onto a plastic pipette. The open end of the pipette is attached to a stainless steel rod that is attached to a Fisher overhead stirrer that is orientated horizontally. The stirrer is set to rotate at 30 rpm. A 2% CHRONOFLEX AL 85A (solution (THF) that contains the powdered silk and Cyclosporine A is sprayed onto the rotating stent-graft using a TLC spray device. The concentration of the powdered silk in the polyurethane solution is altered from 0.1% to 50% (w/w relative to the polymer) and the concentration of the Cyclosporine A is altered from 0.1% to 10% (w/w relative to the polymer). After the spraying process, the stent-graft is allowed to air dry for 30 minutes while still rotating. The stent-graft is then removed from the pipette tip and is further dried under vacuum for 24 h.

### Example 34

### Film Impregnated with Silk Fibers

A 20% CHRONOFLEX AL 85A solution (THF) was cast onto a silicone-coated release liner. The solvent was allowed to dry. Pieces of 3-0 Silk—Black Braided (LIGAPAK) [Ethicon, Inc.] were placed on the surface of the polyurethane film. Drops of THF were then added to the surface of the polyurethane film. Using a glass scintillation vial as a roller, the silk strands were embedded into the surface of the polyurethane film.

### Example 35

### In situ Forming Silk-Containing Gel

Methylated collagen is prepared by the following process: bovine corium collagen is solubilized using pepsin and purified as described in U.S. Pat. No. 4,233,360. This purified, solubilized collagen is precipitated by neutralization into 0.2 M sodium phosphate, pH 7.2. The precipitate is isolated by centrifugation to a final concentration of 70 mg/ml. The material is dried for two days, and then pulverized. Dry methanol containing HCl (to 0.1 N) is added (40 ml) and stirred for four days. Collagen is separated from the acidic methanol, vacuum dried and sterilized by irradiation. The final product is dissolved in water at a pH of 3-4.

For delivery as a gel, 10 mg of the methylated collagen, 100 mg of a tetra-functional sulfhydryl-PEG [pentaerythritol poly(ethylene glycol)ether tetra-sulfhydryl], 10,000 mol. wt., and 100 mg of a tetra-functional succinimidyl PEG [pentaerythritol poly(ethylene glycol)ether tetra-succinimidyl glutarate], 10,000 mol. wt., are dissolved in water at pH 3-4 to a final volume of 1 ml (first component). The second component is 1 ml of Phosphate/Carbonate Buffer (300 mM sodium monobasic phosphate is mixed with 300 mM sodium carbonate. If required, the pH is adjusted with NaOH or HCL to achieve pH 9.6. The final molarity is approximately 117 mm phosphate and 183 mM carbonate). Various amounts (1 mg to 100 mg) of the silk powder are added to the Phosphate/carbonate buffer. Each component is placed in a syringe and mixed and sprayed on the desired test site using a manual dual-syringe delivery system or an air-assisted dual syringe delivery system (FibriJet, Micromedics).

### Example 36

### Coating of the Silk Braid with a Polymer/Biologically Agent—Direct Dipping

Silk braid (Ethicon, 4-0, 638) is cut into approx 10 cm lengths. The silk braid is dipped into a chloroform solution of poly(lactide-co-glycolide) [PLGA] (9K, 50:50, Birmingham Polymers) and cyclosporine A. The concentration of the PLGA is altered from 0.1% to 20% (w/v) and concentration of the cyclosporine A in the solution is altered from 0.1% to a saturated solution. The silk braid is immersed in the PLGA/cyclosporine A solution for 5 minutes. The silk braid is then removed and air-dried. The cyclosporine A loaded silk braid is then further dried under vacuum. The silk braid is then attached to a polyurethane film by placing the coated-braids on the polyurethane film and then pressing the film/braids in a heat press for about 10 seconds such that the coated braid is embedded in the polyurethane film.

### Example 37

### In situ Forming Silk-Containing Gel

For delivery as a gel, 200 mg of a tetra-functional succinimidyl PEG [pentaerythritol poly(ethylene glycol)ether tetra-succinimidyl glutarate], 10,000 mol. wt., is dissolved in water at pH 2.5 (adjusted with HCl) to a final volume of 1 ml (first component). The second component is 1 ml of Phosphate/Carbonate Buffer (300 mM sodium monobasic phosphate is mixed with 300 mM sodium carbonate. If required, the pH is adjusted with NaOH or HCL to achieve pH 9.6. The final molarity is approximately 117 mm phosphate and 183 mM carbonate) that contains 200 mg of a tetra-functional amino-PEG [pentaerythritol poly(ethylene glycol)ether tetra-amino], 10,000 mol. wt. Various amounts (1 mg to 200 mg) of the silk powder are added to the acidic buffer. Each component is placed in a syringe and is sprayed on the desired test site using a manual dual-syringe delivery system or an air-assisted dual syringe delivery system (FibriJet, Micromedics).

### Example 38

### Cyclosporine A—Containing Coating

A 5% CHRONOFLEX AL 85A solution (chloroform) containing from 0.1% to 10% cyclosporine A is prepared. A piece of polyurethane tubing is immersed in and then withdrawn from the coating solution. The coated sample is air-dried in the fume-hood. Samples of different coating thicknesses are prepared by repeating the dip-coating process. The coated sample is then dried under vacuum for 24 hours.

### Example 39

### Collagen Synthesis Assay

An in vitro assay is described for determining whether a substance promotes deposition of extracellular matrix (ECM). Normal human dermal fibroblasts were trypzanized, then re-plated in medium containing ascorbic acid-2-phospate at 150,000 cells per well in a 12-well plate. The cells were cultured at 37° C. and 5% CO₂ for 2-3 weeks with media changes every three days so that they formed a 3-D matrix of cells and collagen. After 14-21 days of culture, the medium was replaced with serum free medium and the cells allowed to rest for 24 hours.

Drug was diluted in DMSO at 10⁻²M, and then diluted 10 fold to give a range of stock concentrations from 10⁻²M to 10⁻⁸M. Drug was then diluted 1000 times in fresh serum free medium and added to the wells in a total volume of 3 ml per well. The plate(s) were then incubated for 72 hrs at 37° C. After 72 hrs the media was removed from the wells and put into microcentrifuge tubes and frozen at −20° C. until assayed.

The amount of collagen synthesized was measured using a Procollagen Type 1 C-Peptide (PIP) EIA kit (Takara), where the amount of collagen produced is stoichiometrically represented by the amount of pro-peptide cleaved from the collagen when it is secreted. Anti-PIP monoclonal antibodies are immobilized on an ELISA plate, the samples added, then a second PIP monoclonal antibody conjugated to horseradish peroxidase is added to the wells and incubated. Following incubation the wells are washed, a substrate solution is added and the absorbance measured in a plate reader at 450 nm and compared to a standard curve of PIP (ng/ml).

### Example 40

### Chick Chorioallantoic Membrane (“CAM”) Assay

This Example describes an in vitro assay for determining whether a substance promotes angiogenesis. Fertilized, domestic chick embryos are incubated for 3 days prior to shell-less culturing. In this procedure, the egg contents are emptied by removing the shell located around the air space. The interior shell membrane is then severed and the opposite end of the shell is perforated to allow the contents of the egg to gently slide out from the blunted end. The egg contents are emptied into round-bottom sterilized glass bowls and covered with petri dish covers. These are then placed into an incubator at 90% relative humidity and 3% CO₂ and incubated for 3 days. (Alternatively, egg contents can remain in the shell with the opening covered with parafilm.)

The agent (Sigma, St. Louis, Mich.) can be mixed at concentrations of 0.25, 0.5, 1, 5, 10, 30 μg per 10 μl aliquot of 0.5% aqueous methylcellulose. Concentrations can be altered depending on the agent. Agents can be mixed with other compatible materials as appropriate depending on the solubility of the agent. Ten microliter aliquots of this solution are dried on parafilm for 1 hour forming disks 2 mm in diameter. The dried disks containing agent are then carefully placed at the growing edge of each CAM at day 6 of incubation. The day of disc placement can be altered depending on the amount of angiogenesis stimulation by the agent beyond control. Controls are obtained by placing agent-free methylcellulose disks on the CAMs over the same time course. After a 2 day exposure (day 8 of incubation) the vasculature is examined with the aid of a stereomicroscope. Liposyn II, a white opaque solution, is injected into the CAM to increase the visibility of the vascular details. The vasculature of unstained, living embryos were imaged using a Zeiss stereomicroscope which is interfaced with a video camera (Dage-MTI Inc., Michigan City, Ind.). These video signals are then displayed at 160× magnification and captured using an image analysis system (Vidas, Kontron; Etching, Germany). Image negatives are then made on a graphics recorder (Model 3000; Matrix Instruments, Orangeburg, N.Y.).

The membranes of the 8 day-old shell-less embryos are flooded with 2% glutaraldehyde in 0.1 M sodium cacodylate buffer; additional fixative is injected under the CAM. After 10 minutes in situ, the CAM is removed and placed into fresh fixative for 2 hours at room temperature. The tissue is then washed overnight in cacodylate buffer containing 6% sucrose. The areas of interest are postfixed in 1% osmium tetroxide for 1.5 hours at 4° C. The tissues are then dehydrated in a graded series of ethanols, solvent exchanged with propylene oxide, and embedded in Spurr resin. Thin sections are cut with a diamond knife, placed on copper grids, stained, and examined in a Joel 1200EX electron microscope. Similarly, 0.5 mm sections are cut and stained with toluene blue for light microscopy.

At day 11 of development, chick embryos are used for the corrosion casting technique. MERCOX resin (Ted Pella, Inc., Redding, Calif.) is injected into the CAM vasculature using a 30-gauge hypodermic needle. The casting material consists of 2.5 grams of MERCOX CL-2B polymer and 0.05 grams of catalyst (55% benzoyl peroxide) having a 5 minute polymerization time. After injection, the plastic is allowed to sit in situ for an hour at room temperature and then overnight in an oven at 65° C. The CAM is then placed in 50% aqueous solution of sodium hydroxide to digest all organic components. The plastic casts are washed extensively in distilled water, air-dried, coated with gold/palladium, and viewed with the Philips 501 B scanning electron microscope.

At day 6 of incubation, the embryo is centrally positioned to a radially expanding network of blood vessels; the CAM develops adjacent to the embryo. These growing vessels lie close to the surface and are readily visible making this system an idealized model for the study of angiogenesis. Living, unstained capillary networks of the CAM can be imaged non-invasively with a stereomicroscope.

Transverse sections through the CAM show an outer ectoderm consisting of a double cell layer, a broader mesodermal layer containing capillaries which lie subjacent to the ectoderm, adventitial cells, and an inner, single endodermal cell layer. At the electron microscopic level, the typical structural details of the CAM capillaries are demonstrated. Typically, these vessels lie in close association with the inner cell layer of ectoderm.

After 48 hours exposure to an agent at concentrations of 0.25, 0.5, 1, 5, 10, or 30 μg, each CAM is examined under living conditions with a stereomicroscope equipped with a video/computer interface to evaluate the effects on angiogenesis. This imaging setup is used at a magnification of 160× which permits the direct visualization of blood cells within the capillaries; thereby blood flow in areas of interest can be easily assessed and recorded. The change in the amount of angiogenesis is defined as an area of the CAM (measuring 2-6 mm in diameter) with increased capillary network and vascular blood flow. Throughout the experiments, zones are assessed on a 4 point gradient (Table 1). This scale represents the degree of increase in angiogenesis with maximal increase represented as a 3 on the vascular gradient scale. Scores of agents are compared with scores of controls.

TABLE 1
VASCULAR GRADIENT

| | |
|---|---|
| 0 | no vascularity |
| 1 | some microvascular movement |
| 2* | richly vascularized zone approximately 2 mm in diameter |
| 3* | richly vascularized zone extending beyond the disk(6 mm |
| | in diameter) |

*indicates a positive angiogenesis response

### Example 41

### Preparation of Injectable Silk Powder

100 mg of the silk powder prepared in Example 27, was weighed into a glass vial. The vial was capped with a septum which was then held in place with a crimp seal. The product was sterilized using e-beam radiation. Prior to use, the silk powder was resuspended using a 50:50 (v/v) solution of sterile saline and a water soluble x-ray contrast agent (OPTIRAY 320).

### Example 42

### Preparation of Injectable Silk Powder

100 mg of the silk powder prepared in Example 27, was weighed into a glass vial. The vial was capped with a septum which was then held in place with a crimp seal. The product was sterilized using e-beam radiation. Prior to use, the silk powder was resuspended using a solution a water soluble x-ray contrast agent (OPTIRAY 320). In a sample prepared in a similar manner, heparin was added to the composition.

### Example 43

### Preparation of 79/21 (by Weight) Block Copolymer of 60/40 Dl-Lactide/Glycolide and Polyethylene Glycol 400 [Polymer A]

A suitable flask-was thoroughly cleaned, flame-dried, and charged dry with polyethylene glycol (MW-400; 5 g, 0.0125 mole), dl-lactide (12 g, 0.083 mole), glycolide (6.4 g, 0.056 mole), stannous octoate catalyst (0.4M in toluene; 34.7 μL, 0.014 mmole), and a magnetic stirrer under nitrogen condition. The reactor was placed in an oil bath and heated to 170° C. under a positive nitrogen pressure for 16 hours. The flask was removed and stored open in a vacuum oven.

### Example 44

### Preparation of 14/86 (by Weight) of Block Copolymer of 60/40 Dl-Lactide/Glycolide and Polyethylene Glycol 400 [Polymer B]

Polyethylene glycol (MW=400; 20 g, 0.05 mole), dl-lactide (2.12 g, 0.015 mole), glycolide (1.14 g, 0.010 mole), and stannous octoate catalyst (0.4M in toluene; 25 μL, 0.05 mmole) were added under dry conditions to a glass rector containing a magnetic stirrer. The reactor was heated to 130° C. to melt the reactants and then increased to 170° C. to start the reaction. After 5 hours, the system was cooled-and stored in a vacuum oven.

### Example 45

### Preparation of Silk-Containing Degradable Formulation

A series of formulations are prepared by mixing various amounts of degradable polymer A (Example 45) and polymer B (Example 46). The ratios of polymer A to polymer B were 10:90, 20:80, 30:70, 40:60, 50:50, 60:40, 70:30, 80:20 and 90:10. Silk powder (Example 1) is then added to each of the compositions. The loading of silk powder in the compositions ranges from a silk:polymer ratio of 0.1% to 50%. The viscosity of the silk-loaded samples is modulated using various amounts of PEG 300. The amounts of PEG 300 added range from 0% to 75% (w/w) of the total composition.

### Example 46

### Preparation of Silk-Containing Non-Degradable Formulation

A 7% solution of poly(ethylene-co-vinyl alcohol) (EVOH) in DMSO was prepared by adding 7 g EVOH to 100 ml DMSO in a 250 ml round bottom flask. The flask was sealed with a septum and was placed under a positive pressure of oxygen free nitrogen using a nitrogen tank that was connected to a needle and a oil bubbled in a T-configuration. The solution was placed in a water bath and was heated to 50° C. The solution was stirred using a stirrer/hotplate. Once the polymer had dissolved, the solution was removed from the water bath and was allowed to cool to room temperature. The solution was aliquoted into 10 ml aliquots and various amounts (1%, 2.5%, 5%, 7.5%, 10%, 20%, 30%, 40%) of silk were added to the solution. The solutions were then stirred until a homogeneous suspension was obtained. In a second set of samples, various amounts (20%, 40%, 60%, 80% tantalum:tantalum+polymer) of tantalum powder (approx. 3 um) is added to the formulation.

### Example 47

### Animal Abdominal Aortic Aneurysm Model

An animal model is described for determining whether a stent graft containing a biologically active or irritative substance stimulates fibrosis. Pigs or sheep are placed under general anesthetic. Using aseptic precautions the abdominal aorta is exposed. The animal is heparinized and the aorta is cross clamped below the renal arteries and above the bifurcation. Collaterals are temporarily controlled with vessel loops or clips that are removed upon completion of the procedure. A longitudinal aortotomy is created in the arterial aspect of the aorta, and an elliptical shaped patch of rectus sheath from the same animal is sutured into the aortotomy to create an aneurysm. The aortic clamps from the lumbar arteries and collaterals are removed and the abdomen closed. After 30 days, the animal is reanesthesized and the abdominal wall again opened. A cutdown is performed on the iliac artery. A guidewire in then introduced into the artery and moved forward until the end resides in the aneurysm. A stent graft is then inserted through the iliac artery and is positioned across the infrarenal abdominal aorta aneurysm extending from normal infrarenal abdominal aorta above to normal infrarenal abdominal aorta below the surgically created aneurysm and the device is released in a conventional way. A catheter is then inserted over the guidewire and advanced along between the vessel and the stent-graft until the catheter tip was positioned in the aneurysm. Once the catheter is in the correct position in the aneurysm, the guidewire is removed and approx. 2-3 ml of the silk powder formulation (Example 43) is injected into the aneurysm. The catheter is removed and the site is closed. After closure of the arteriotomy and of the abdominal wound, the animal is allowed to recover.

Animals are randomized into two groups of 5 with one group receiving uncoated stent grafts, and the second group receiving a stent graft with subsequent silk formulation injection. At 6 weeks and 3 months post stent graft insertion, the animal is sacrificed and the aorta removed en bloc. The infrarenal abdominal aorta is examined for evidence of histologic reaction and perigraft leaking.

### Example 48

### Sheep Aneurysm Model

A sheep was placed under general anesthetic. Using aseptic precautions the left carotid artery is exposed. The animal is heparinized and the artery is cross clamped. A longitudinal arteriotomy is created in the artery, and an elliptical shaped patch of vein (Left external jugular) from the same animal is sutured into the arteriotomy to create an aneurysm. The aortic clamps are removed and the surgical site is closed. After 2 weeks, the animal was reanesthesized and the neck was again opened. A cutdown is performed on the carotid artery about 10 cm distal to the previously created aneurysm. A guidewire in then introduced into the artery and moved forward until the end resides in the surgically created aneurysm. A delivery system containing the stent-graft (WALLGRAFT, 9F, 8/30 mm) was then inserted through carotid artery and is positioned across the surgically created aneurysm. The device was then deployed and the delivery system is removed. A catheter was then insert over the guidewire and advanced along between the vessel and the stent-graft until the catheter tip was positioned in the aneurysm. Once the catheter was in the correct position in the aneurysm, the guidewire is removed and approx. 2-3 ml of the silk powder formulation (Example 43) is injected into the aneurysm. The catheter is removed and the site is closed. After closure of the arteriotomy and of the neck wound, the animal is allowed to recover.

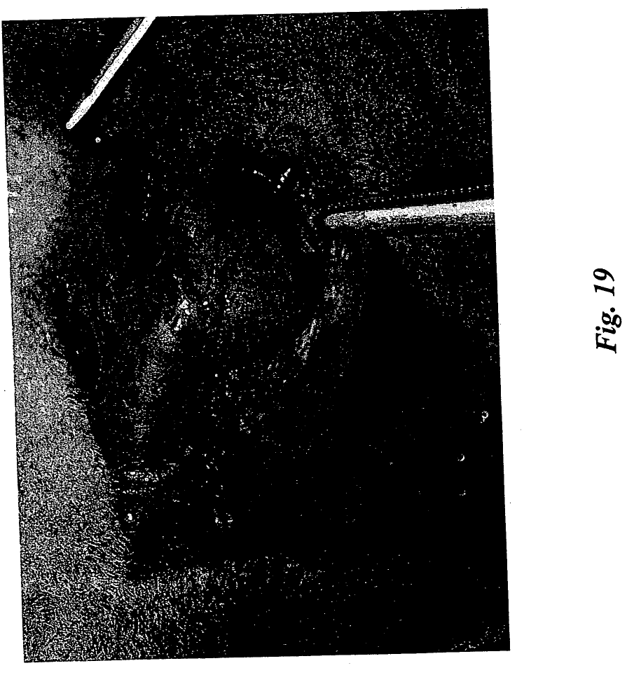

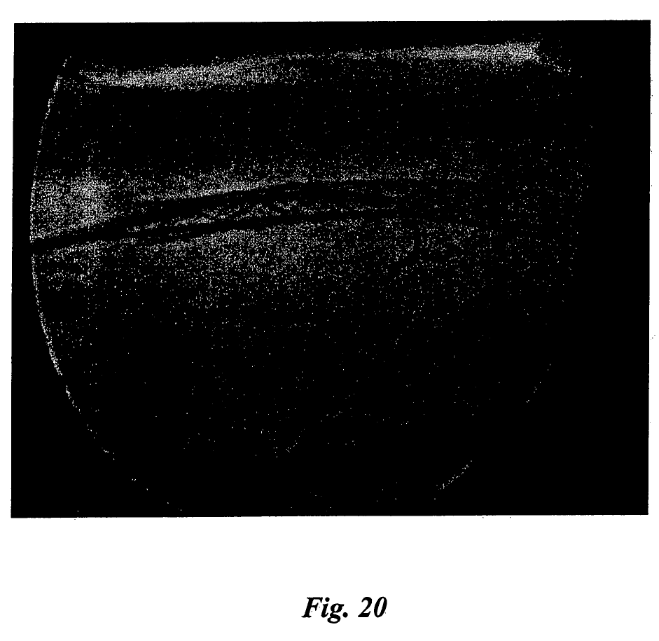

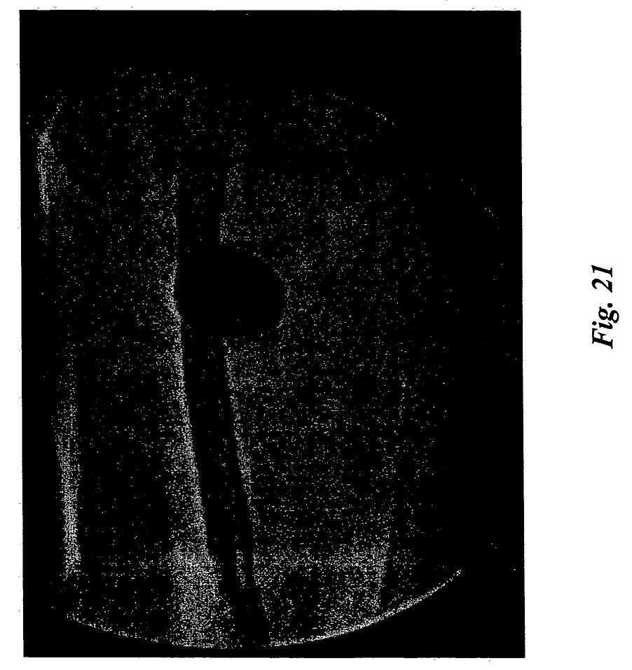

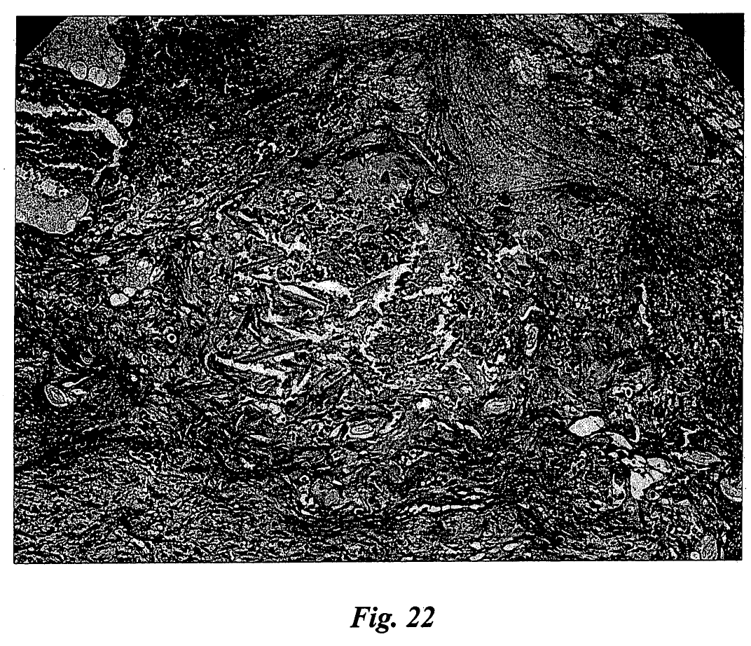

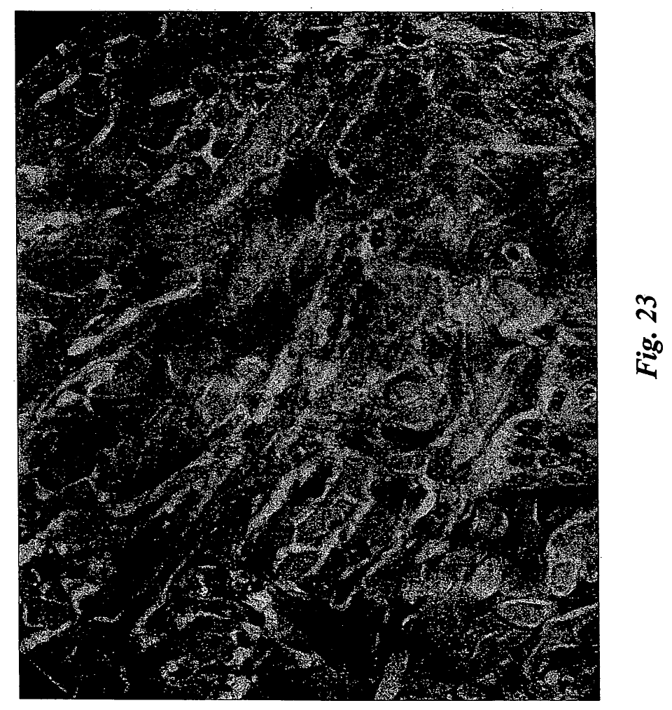

Animals were randomized into two groups of 6 with one group receiving uncoated stent grafts, and the second group receiving a stent graft with subsequent silk formulation injection. At 4 weeks post stent graft insertion, the animal is sacrificed and the aneurysm portion of the carotid artery was removed en bloc. The samples were sent for histological preparation and analysis (see FIGS. 19-23).

### Example 49

### Silk Suture Coated with Magnetically Active Particles

The end of a piece of silk 5-0 suture was immersed in a THF solution of CHRONOFLEX AL 85A polyurethane solution (about 10% w/v). The silk was removed, and the coated end was dipped into a vial containing magnetically active microparticles. The coated silk end was removed, and the particles were further embedded into the polyurethane coating by rolling the end between two fingertips. The solvent was removed by air-drying.

### Example 50

### Silk Suture Coated with Magnetically Active Beads

The end of a piece of silk 5-0 suture was immersed in a THF solution of CHRONOFLEX AL 85A polyurethane solution (about 10% w/v) that contained approximately 5% w/w (beads to polymer) magnetic beads. The silk was removed, and the coated end was dipped into a vial containing magnetically active microparticles. The coated silk end was removed, and the particles were further embedded into the polyurethane coating by rolling the end between two fingertips. The solvent was removed by air-drying.

### Example 51

### In-vivo Evaluation of Perivascular PU Films Coated with Degummed or Virgin Silk Strands

Wistar rats weighing 300 g to 400 g are anesthetized with halothane. The skin over the neck region is shaved and the skin is sterilized. A vertical incision is made over the trachea and the left carotid artery is exposed. A polyurethane film covered with degummed silk strands, virgin silk strands or a control uncoated PU film is wrapped around a distal segment of the common carotid artery. The wound is closed and the animal is recovered.

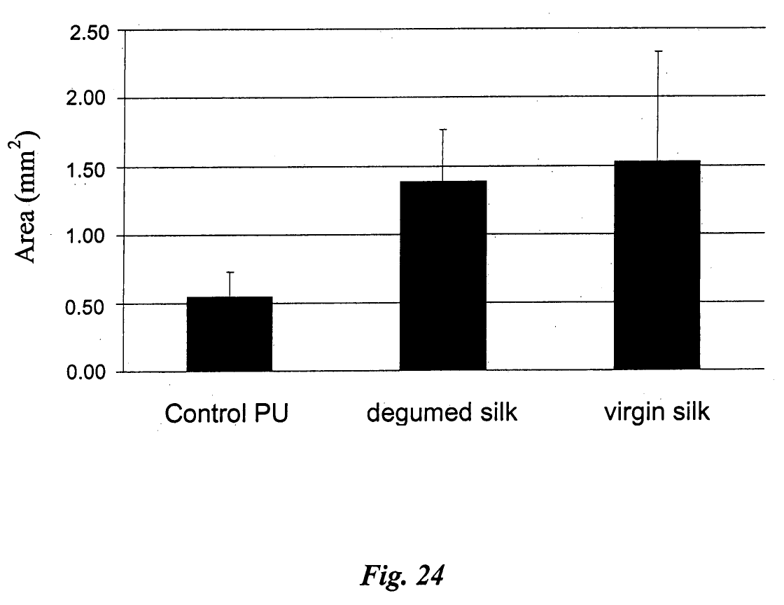

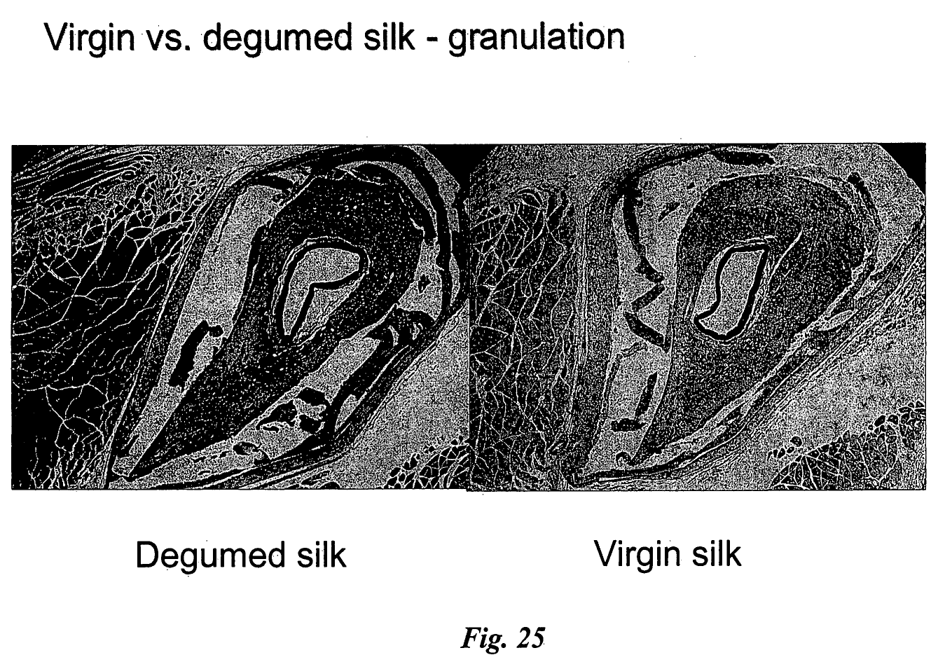

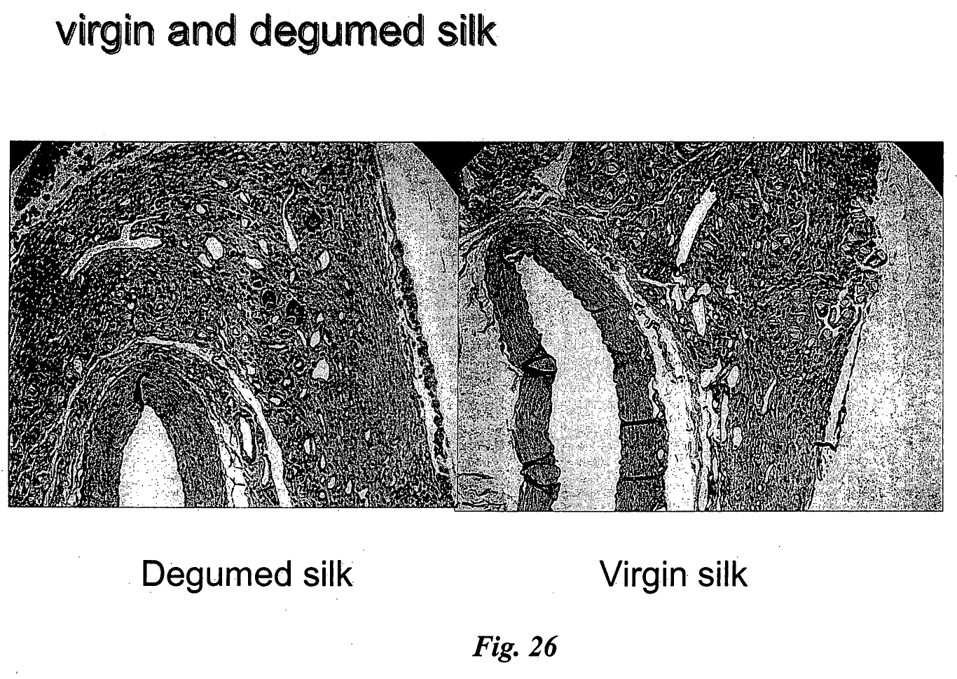

After 28 days, the rats are sacrificed with carbon dioxide and pressure-perfused at 100 mmHg with 10% buffered formaldehyde. Both carotid arteries are harvested and~processed for histology. Serial cross-sections will be cut every 2 mm in the treated left carotid artery and at corresponding levels in the untreated right carotid artery. Sections are stained with H&E and Movat's stains to evaluate tissue growth around the carotid artery. Thickness of perivascular granulation tissue is quantified by computer-assisted morphometric analysis. Both types of silk markedly increased granulation tissue growth around the blood vessel to the same extent. The silk strands in both groups has broken down into small particles (approximately 30 um in diameter) scattered around the blood vessel and surrounded by giant cells, macrophages, proteoglycan matrix and blood vessels. These features are typical of a foreign body response. The area covered by the foreign body response was more variable in the virgin silk group than in the degummed silk group. As shown in FIG. 24 and FIG. 25, both types of silk markedly increased granulation tissue growth around the blood vessel to the same extent, and both types of silk induced a marked tissue reaction around the treated blood vessel. As shown in FIG. 26, the silk strands have broken down into small particles surrounded by giant cells and macrophages. The granulation tissue is highly vascularized and contains numerous-inflammatory cells and fibroblasts. Extracellular matrix deposition is also extensive.

### Example 52

### Preparation of Silk Powder Using a Cryomill

Fibers of degummed silk were cut into pieces approximately 1-2 cm in length. The material was then milled to a powder using a cryomill (Spex Certiprep Freezer/Mill—Model 6850). A portion of the milled powder was then sieved through a series of different sized metal sieves to obtain silk powder of different size ranges.

### Example 53

### Electrospinning of Silk-Loaded Material

20% solutions of PLGA (50:50, Mw~ 54,000) are prepared by dissolving 2 g PLGA into 10 mL DCM. Various amounts of silk powder (25-53 um) are added to each solution such that the silk percentage of the polymers ranges from 2% to 50%. Each solution is then loaded into a 10 ml syringe fitted with an 18 gauge needle. The syringe is then loaded into a syringe pump and 20 kV positive high voltage (by Glassman High Voltage, Inc., High Bridge, N.J.) is applied on the syringe needle. The grounded target drum is a rotating drum that has a diameter of about 12 cm. The syringe pump is set to pump at 25 uL per minute and the drum is rotated at approximately 250 rpm. The distance from the tip of the needle to the outside of the drum surface is about 14 cm. The rotating drum is moved from side to side during the spinning process such that the drum is virtually completely covered in the spun material. After the spinning process is completed, a razor blade is used to make a cut through the entire length of the spun material. The material is removed from the drum and is further dried in a vacuum oven for 24 hours.

### Example 54

### Attachment of Silk-Loaded Material to a Graft Material

The silk-loaded electrospun material (prepared as in Example 55) is cut into strips that are approximately 0.5 cm×2 cm. The strips are then placed on the external surface of the graft portion of a stent-graft. Drops of a cyanoacrylate glue are used to glue the strips onto the graft surface.

### Example 55

### Grafts with Silk Sleeves

A 3 ply yarn of virgin silk fibers is knitted into a sleeve using a circular knitting machine (Lawson-Hemphill Bak Knitter). The diameter of the knitted sleeve is approximately 8 mm. The silk sleeve is cut into lengths that are approximately 75% the length of a stent-graft to which they are to be attached. The silk sleeve is then slid over the outer surface of the stent-graft. The sleeve is then attached to the graft at several different attachment points using several 7-0 prolene sutures. Alternatively, a silk sleeve is attached to the graft at several different attachment points using small drop(s) of a cyanoacrylate glue.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1.-1360. (canceled)

1361. A method for reducing perigraft leakage associated with stent graft delivery in a patient, comprising delivering a device to a patient in need thereof, wherein the device comprises i) a stent graft and ii) a fibrosing agent or a composition comprising a fibrosing agent, wherein the agent induces fibrosis.

1362. The method of claim 1361 wherein the fibrosing agent promotes regeneration.

1363. The method of claim 1361 wherein the fibrosing agent promotes angiogenesis.

1364. The method of claim 1361 wherein the fibrosing agent promotes fibroblast migration.

1365. The method of claim 1361 wherein the fibrosing agent promotes fibroblast proliferation.

1366. The method of claim 1361 wherein the fibrosing agent promotes deposition of extracellular matrix (ECM).

1367. The method of claim 1361 wherein the fibrosing agent promotes tissue remodeling.

1368. The method of claim 1361 wherein the fibrosing agent is an arterial vessel wall irritant.

1369. The method of claim 1361 wherein the fibrosing agent is or comprises silk.

1370. The method of claim 1361 wherein the fibrosing agent is or comprises mineral particles.

1371. The method of claim 1361 wherein the fibrosing agent is or comprises chitosan.

1372. The method of claim 1361 wherein the fibrosing agent is or comprises polylysine.

1373. The method of claim 1361 wherein the fibrosing agent is or comprises fibronectin.

1374. The method of claim 1361 wherein the fibrosing agent is or comprises bleomycin.

1375. The method of claim 1361 wherein the fibrosing agent is or comprises CTGF.

1376. The method of claim 1361 wherein the fibrosing agent is in the form of a thread, or is in contact with a thread.

1377. The method of claim 1361 wherein the fibrosing agent is in the form of a particulate.

1378. The method of claim 1361 wherein the composition further comprises an inflammatory cytokine.

1379. The method of claim 1361 wherein the composition further comprises an agent that stimulates cell proliferation.

1380. The method of claim 1361 wherein the composition is in the form of a gel or paste.

1381. The method of claim 1361 wherein the fibrosing agent is in the form of tufts.

1382. The method of claim 1361, wherein the fibrosing agent promotes adhesion between the stent graft and the patient.

1383. The method of claim 1361, wherein the stent graft delivers the fibrosing agent locally to tissue proximate to the stent graft.

1384. The method of claim 1361, wherein the stent graft and fibrosing agent are combined so as to provide a coating on the stent graft.

1385. (canceled)

1386. (canceled)

1387. The method of claim 1361, wherein the stent graft and fibrosing agent are combined so as to provide a coating on the stent graft, and the coating partially covers the stent graft.

1388. The method of claim 1361, wherein the stent graft and fibrosing agent are combined so as to provide a coating on the stent graft, and the coating completely covers the stent graft.

1389. The method of claim 1361, wherein the stent graft and fibrosing agent are combined so as to provide a coating on the stent graft, where the coating is a uniform coating.

1390. The method of claim 1361, wherein the stent graft and fibrosing agent are combined so as to provide a coating on the stent graft, where the coating is a non-uniform coating.

1391. The method of claim 1361, wherein the stent graft and fibrosing agent are combined so as to provide a coating on the stent graft, where the coating is a discontinuous coating.

1392. (canceled)

1393. The method of claim 1361, wherein the stent graft and fibrosing agent are combined so as to provide a coating on the stent graft, where the coating has a thickness of 100 mm or less.

1394. The method of claim 1361, wherein the stent graft and fibrosing agent are combined so as to provide a coating on the stent graft, where the coating has a thickness of 10 mm or less.

1395. The method of claim 1361, wherein the stent graft and fibrosing agent are combined so as to provide a coating on the stent graft, and the coating is stable at room temperature for a period of at least 1 year.

1396. The method of claim 1361, wherein the stent graft and fibrosing agent are combined so as to provide a coating on the stent graft, and the fibrosing agent is present in the coating in an amount ranging between about 0.0001% to about 1% by weight.

1397. The method of claim 1361, wherein the stent graft and fibrosing agent are combined so as to provide a coating on the stent graft, and the fibrosing agent is present in the coating in an amount ranging between about 1% to about 10% by weight.

1398. The method of claim 1361, wherein the stent graft and fibrosing agent are combined so as to provide a coating on the stent graft, and the fibrosing agent is present in the coating in an amount ranging between about 10% to about 25% by weight.

1399. The method of claim 1361, wherein the stent graft and fibrosing agent are combined so as to provide a coating on the stent graft, and the fibrosing agent is present in the coating in an amount ranging between about 25% to about 70% by weight.

1400. The method of claim 1361, wherein the stent graft and fibrosing agent are combined so as to provide a coating on the stent graft, wherein the stent graft comprises a first coating having a first composition and a second coating having a second composition.

1401. The method of claim 1361, wherein the stent graft and fibrosing agent are combined so as to provide a coating on the stent graft, wherein the coated stent graft -comprises a first coating having a first composition and a second coating having a second composition, and where the first composition and the second composition are different.

1402. The method of claim 1361, wherein the stent graft and fibrosing agent are combined so as to provide a coating on the stent graft, and the coating further comprises a polymer.

1403. The method of claim 1361, wherein the stent graft and fibrosing agent are combined so as to provide a coating on the stent graft, and the coating further comprises a copolymer.

1404. The method of claim 1361, wherein the stent graft and fibrosing agent are combined so as to provide a coating on the stent graft, and the coating further comprises a block copolymer.

1405. The method of claim 1361, wherein the stent graft and fibrosing agent are combined so as to provide a coating on the stent graft, and the coating further comprises a random copolymer.

1406. The method of claim 1361, wherein the stent graft and fibrosing agent are combined so as to provide a coating on the stent graft, and the coating further comprises a biodegradable polymer.

1407. The method of claim 1361, wherein the stent graft and fibrosing agent are combined so as to provide a coating on the stent graft, and the coating further comprises a non-biodegradable polymer.

1408. The method of claim 1361, wherein the stent graft and fibrosing agent are combined so as to provide a coating on the stent graft, and the coating further comprises a hydrophilic polymer.

1409. The method of claim 1361, wherein the stent graft and fibrosing agent are combined so as to provide a coating on the stent graft, and the coating further comprises a hydrophobic polymer.

1410. The method of claim 1361, wherein the stent graft and fibrosing agent are combined so as to provide a coating on the stent graft, and the coating further comprises a polymer having hydrophilic domains.

1411. The method of claim 1361, wherein the stent graft and fibrosing agent are combined so as to provide a coating on the stent graft, and the coating further comprises a polymer having hydrophobic domains.

1412. The method of claim 1361, wherein the stent graft and fibrosing agent are combined so as to provide a coating on the stent graft, and the coating further comprises a non-conductive polymer.

1413. The method of claim 1361, wherein the stent graft and fibrosing agent are combined so as to provide a coating on the stent graft, and the coating further comprises an elastomer.

1414. The method of claim 1361, wherein the stent graft and fibrosing agent are combined so as to provide a coating on the stent graft, and the coating further comprises a hydrogel.

1415. The method of claim 1361, wherein the stent graft and fibrosing agent are combined so as to provide a coating on the stent graft, and the coating further comprises a silicone polymer.

1416. The method of claim 1361, wherein the stent graft and fibrosing agent are combined so as to provide a coating on the stent graft, and the coating further comprises a hydrocarbon polymer.

1417. The method of claim 1361, wherein the stent graft and fibrosing agent are combined so as to provide a coating on the stent graft, and the coating further comprises a styrene-derived polymer.

1418. The method of claim 1361, wherein the stent graft and fibrosing agent are combined so as to provide a coating on the stent graft, and the coating further comprises a butadiene-derived polymer.

1419. (canceled)

1420. The method of claim 1361, wherein the stent graft and fibrosing agent are combined so as to provide a coating on the stent graft, and the coating further comprises a poly(ethylene glycol) polymer.

1421. The method of claim 1361, wherein the stent graft and fibrosing agent are combined so as to provide a coating on the stent graft, and the coating further comprises an amorphous polymer.

1422. The method of claim 1361, wherein the stent graft and fibrosing agent are combined so as to provide a coating on the stent graft, and the coating is a lubricious coating.

1423. The method of claim 1361, wherein the stent graft and fibrosing agent are combined so as to provide a coating on the stent graft, and the coating is located within pores or holes of the stent graft.

1424. (canceled)

1425. The method of claim 1361, wherein the stent graft and fibrosing agent are combined so as to provide a coating on the stent graft, and the coating is located within a channel, lumen, or divet of the stent graft.

1426. The method of claim 1361, wherein the stent graft is combined with a second pharmaceutically active agent.

1427. The method of claim 1361, wherein the device comprises further an anti-inflammatory agent.

1428. The method of claim 1361, wherein the device further comprises an agent that inhibits infection.

1429. The method of claim 1361, wherein the device further comprises an anthracycline.

1430. The method of claim 1361, wherein the device further comprises doxorubicin.

1431. The method of claim 1361, wherein the device further comprises mitoxantrone.

1432. The method of claim 1361, wherein the device further comprises a fluoropyrimidine.

1433. The method of claim 1361, wherein the device further comprises 5-fluorouracil (5-FU).

1434. The method of claim 1361, wherein the device further comprises a folic acid antagonist.

1435. The method of claim 1361, wherein the device further comprises methotrexate.

1436. The method of claim 1361, wherein the device further comprises a podophylotoxin.

1437. The method of claim 1361, wherein the device further comprises etoposide.

1438. The method of claim 1361 wherein the device further comprises a camptothecin.

1439. The method of claim 1361, wherein the device further comprises a hydroxyurea.

1440. The method of claim 1361, wherein the device further comprises a platinum complex.

1441. The method of claim 1361, wherein the device further comprises cisplatin.

1442. The method of claim 1361 wherein the device further comprises an anti-thrombotic agent.

1443. The method of claim 1361 wherein the device further comprises a visualization agent.

1444.-1452. (canceled)

1453. The method of claim 1361, wherein device is sterilized.

1454. The method of claim 1361, wherein the device releases the fibrosing agent into tissue in the vicinity of the device after deployment of the device in a patient.

1455. The method of claim 1361, wherein the device releases fibrosing agent into tissue in the vicinity of the device after deployment of the device in a patient, wherein the fibrosing agent is released in effective concentrations from the device over a period ranging from the time of deployment of the device to about at least 1 year.

1456. The method of claim 1361, wherein the device releases fibrosing agent into tissue in the vicinity of the device after deployment of the device in a patient, wherein the fibrosing agent is released in effective concentrations from the device over a period ranging from the time of deployment of the device to at least about 6 months from deployment.

1457. The method of claim 1361, wherein the device releases fibrosing agent into tissue in the vicinity of the device after deployment of the device in a patient, wherein the fibrosing agent is released in effective concentrations from the device over a period ranging from the time of deployment of the device to at least about 90 days from deployment.

1458. The method of claim 1361, wherein the device releases fibrosing agent into tissue in the vicinity of the device after deployment of the device in a patient, wherein the fibrosing agent is released in effective concentrations from the device at a constant rate.

1459. The method of claim 1361, wherein the device releases fibrosing agent into tissue in the vicinity of the device after deployment of the device in a patient, wherein the fibrosing agent is released in effective concentrations from the device at an increasing rate.

1460. The method of claim 1361, wherein the device releases fibrosing agent into tissue in the vicinity of the device after deployment of the device in a patient, wherein the fibrosing agent is released in effective concentrations from the device at a decreasing rate.

1461. (canceled)

1462. (canceled)

1463. The method of claim 1361, wherein the device comprises about 0.01 mg to about 10 mg of the fibrosing agent.

1464. The method of claim 1361, wherein the device comprises about 10 mg to about 10 mg of the fibrosing agent.

1465. The method of claim 1361, wherein the device comprises about 10 mg to about 250 mg of the fibrosing agent.

1466. The method of claim 1361, wherein the device comprises about 250 mg to about 1000 mg of the fibrosing agent.

1467. The method of claim 1361, wherein the device comprises about 1000 mg to about 2500 mg of the fibrosing agent.

1468. The method of claim 1361, wherein a surface of the device comprises less than 0.01 mg of the fibrosing agent per mm2 of device surface occupied by fibrosing agent.

1469. The method of claim 1361, wherein a surface of the device comprises about 0.01 mg to about 1 mg of the fibrosing agent per mm2 of device surface occupied by fibrosing agent.

1470. The method of claim 1361, wherein a surface of the device comprises about 1 mg to about 10 mg of the fibrosing agent per mm2 of device surface occupied by fibrosing agent.

1471. The method of claim 1361, wherein a surface of the device comprises about 10 mg to about 250 mg of the fibrosing agent per mm2 of device surface occupied by fibrosing agent.

1472. The method of claim 1361, wherein a surface of the device comprises about 250 mg to about 1000 mg of fibrosing agent per mm2 of device surface occupied by fibrosing agent.

1473. The method of claim 1361, wherein a surface of the device comprises about 1000 mg to about 2500 mg of the fibrosing agent per mm2 of device surface occupied by fibrosing agent.

1474. The method of claim 1361, wherein the stent graft is a tubular structure that comprises a lumen through which blood may flow, wherein the tubular structure comprises a luminal surface and a non-luminal surface, and wherein the agent or the composition comprising the agent is coated onto the non-luminal surface of the stent graft.

1475. The method of claim 1361, wherein the stent graft is a tubular structure that comprises a lumen through which blood may flow, wherein the tubular structure comprises a luminal surface and a non-luminal surface, and wherein the agent or the composition comprising the agent is directly affixed to the non-luminal surface of the stent graft.

1476. (canceled)

1477. The method of claim 1361, wherein the stent graft is a tubular structure that comprises a lumen through which blood may flow, wherein the tubular structure comprises a luminal surface and a non-luminal surface, and wherein all or a portion of the non-luminal surface of the intraluminal stent graft is coated with a proliferative agent.

1478. (canceled)

1479. The method of claim 1361, wherein the stent graft is a tubular structure that comprises a lumen through which blood may flow, wherein the tubular structure comprises a luminal- surface and a non-luminal surface, where the method comprises attaching a thread to a non-luminal surface of the structure, wherein the thread is, or comprises, the fibrosing agent or the composition comprising the fibrosing agent.

1480.-1738. (canceled)
